(19) United States
(12) Patent Application Publication
(10) Pub. No.: US 2026/0355142 A1
(43) Pub. Date: Nov. 12, 2026

(54) AMORPHOUS OTENAPROXESUL AND COMPOSITIONS AND USES THEREOF AND DOSAGE REGIMENS FOR OTENAPROXESUL

(71) Applicant: ANTIBE THERAPEUTICS INC., Toronto, Ontario (CA)

(72) Inventors: David J. VAUGHAN, Toronto, Ontario (CA); Raymond E. DAGGER, Mount Airy, MD (US); Daniel M. LEGAULT, Toronto, Ontario (CA); Joseph W. STAUFFER, Sarasota, FL (US); France GUAY, Quebec (CA)

(21) Appl. No.: 19/119,690

(22) PCT Filed: Oct. 11, 2023
(86) PCT No.: PCT/CA2023/051347

§ 371 Date: Apr. 9, 2025

Related U.S. Application Data
(60) Provisional application No. 63/379,118, filed on Oct. 11, 2022.

(51) Int. Cl. A61K 31/216; A61K 9/00; A61K 9/10; A61K 9/20; A61P 29/00

(52) U.S. Cl. CPC A61K 31/216; A61K 9/0053; A61K 9/10; A61K 9/2022; A61P 29/00

(57) ABSTRACT

The present application relates to amorphous otenaproxesul and to amorphous solid dispersions and compositions comprising amorphous solid dispersions of amorphous otenaproxesul or a pharmaceutically acceptable salt thereof and a polymer, as well as uses thereof, for example, for the treatment of acute pain. Methods of treating acute pain using dosing regimens comprising one or more of the compositions comprising the amorphous solid dispersions or comprising crystalline otenaproxesul are also provided. The present application further relates to packages and kits comprising the pharmaceutical compositions. (Otenaproxesul)

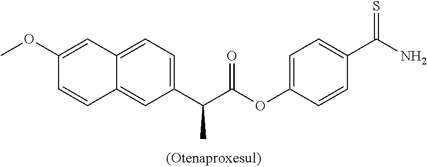 [SMILES: COc1ccc2cc([C@H](C)C(=O)Oc3ccc(C(N)=S)cc3)ccc2c1]

### RELATED APPLICATIONS

The present application claims the benefit of priority of co-pending U.S. provisional patent application No. 63/379,118 filed on Oct. 11, 2022, the contents of which are incorporated herein by reference in their entirety.

### FIELD

The present application relates to amorphous otenaproxesul and compositions comprising amorphous otenaproxesul and a polymer. In particular, the present application relates to amorphous solid dispersions of otenaproxesul and compositions comprising amorphous solid dispersions of otenaproxesul and uses thereof, for example, for the treatment of pain. The present application further relates to packages and kits comprising the pharmaceutical compositions. The present application also relates method of treating acute pain using dosing regimens of otenaproxesul and pharmaceutical compositions thereof.

### BACKGROUND

Otenaproxesul is chemically known as(S)-4-carbamothioylphenyl 2-(6-methoxynaphthalen-2-yl) propanoate or(S)-2-(6-methoxy-naphthalen-2-yl)-propionic acid 4-thiocarbamoyl-phenyl ester and is a hydrogen sulfide-releasing analogue of naproxen with a strikingly different safety and efficacy profile in humans than naproxen itself. U.S. Pat. No. 8,541,398 discloses various non-steroidal anti-inflammatory drug (NSAID) derivatives covalently linked to H₂S-releasing moieties including otenaproxesul, and found that the anti-inflammatory activity of a variety of NSAIDs is significantly enhanced when covalently linked to, or NSAID salts are formed with, an H₂S-releasing moiety.

Hydrogen sulfide (H₂S) has been shown to be produced in the GI tract and to contribute to gastrointestinal mucosal defense and the healing of gastrointestinal ulcers. In the intestine, H₂S modulates epithelial secretion and promotes resolution of colitis. H₂S inhibits leukocyte adherence to the vascular endothelium and appears to play an important role in the regulation of systemic blood pressure. Endogenous H₂S and H₂S donors have also been reported to have a potential role in protecting against viral infections [Li H et al. J. Virol. 2015; 89:5557-5568, Bazhanov N et al. Sci. Rep. 2017; 7:41029, Yang G. Am. J. Physiol. Cell Physiol. 2020; 318: C244-C249, Citi V. et al. Brit. J. Pharmacol. 2020; 177:4931-4941, Kim J Z J Et al. 2020, Journal of Translational Medicine, volume 18, Article number: 257257].

Otenaproxesul exhibits anti-inflammatory, analgesic, antinociceptive, immunomodulatory, and neuroprotective activities and was found to be effective at reducing pain in osteoarthritis patients with an efficacy equal to or better than naproxen or celecoxib in comparable studies. Advantageously, linking an H₂S-releasing moiety to naproxen also greatly reduces the gastrointestinal (GI) damaging effects of this NSAID compared to naproxen as demonstrated in human clinical trials, including trial NCT03978208 listed on clinicaltrials.gov.

Processes for the synthesis of otenaproxesul disclosed in U.S. Pat. No. 8,541,398 provide crystalline otenaproxesul.

### SUMMARY

The present application includes a pharmaceutical composition comprising otenaproxesul or pharmaceutically acceptable salt thereof and a pharmaceutically acceptable excipient, wherein at least about 95 wt % of the otenaproxesul, or the pharmaceutically acceptable salt thereof, is in amorphous form.

The present application also includes an amorphous solid dispersion comprising amorphous otenaproxesul or pharmaceutically acceptable salt thereof and a polymer.

In some embodiments, the amorphous solid dispersion of the application further comprises one or more sustaining agents.

The present application also includes a pharmaceutical composition comprising an amorphous solid dispersion of the application and one or more pharmaceutically acceptable excipients.

In some embodiments, the one or more pharmaceutically acceptable excipients comprise one or more intragranular pharmaceutically acceptable excipients and one or more extragranular pharmaceutically acceptable excipients.

The present application also includes a pharmaceutical package or kit comprising:
one or more pharmaceutical compositions of the application comprising a loading dose of amorphous otenaproxesul or a pharmaceutically acceptable salt thereof, and
instructions for administration of the one or more pharmaceutical compositions comprising the loading dose of the amorphous otenaproxesul or the pharmaceutically acceptable salt thereof, to a subject in need thereof.

The present application also includes a pharmaceutical package or kit comprising:
a pharmaceutical composition of the application comprising a loading dose of amorphous otenaproxesul or a pharmaceutically acceptable salt thereof, and
one or more pharmaceutical compositions of the application comprising a maintenance dose of amorphous otenaproxesul or a pharmaceutically acceptable salt thereof.

The present application also includes a method for treating diseases, disorders or conditions treatable by inhibition of COX-1 and/or COX-2 comprising administering an effective amount of one or more pharmaceutical compositions of the application to a subject in need thereof.

In some embodiments, the disease, disorder or condition treatable by inhibition of COX-1 and/or COX-2 is pain.

In some embodiments, the method comprises administering one or more pharmaceutical compositions of the application comprising a loading dose of amorphous otenaproxesul or a pharmaceutically acceptable salt thereof.

In some embodiments, the method further comprises, following administering the one or more pharmaceutical compositions comprising a loading dose of otenaproxesul or a pharmaceutically acceptable salt thereof, for example amorphous otenaproxesul or a pharmaceutically acceptable salt thereof, administration of one or more pharmaceutical compositions of the application comprising maintenance doses of otenaproxesul or a pharmaceutically acceptable salt thereof, for example, the amorphous otenaproxesul or a pharmaceutically acceptable salt thereof, wherein administration of the compositions comprising otenaproxesul or a pharmaceutically acceptable salt thereof, for example amorphous otenaproxesul or a pharmaceutically acceptable salt thereof, is for a treatment period of 14 days or less.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

The embodiments of the application will now be described in greater detail with reference to the attached drawings in which:

FIG. 1 shows levels in plasma of naproxen (a metabolite of otenaproxesul) after single ascending dose (SAD) otenaproxesul (ATB-346) oral administration in healthy subjects. The single dosage amounts of otenaproxesul were (from the top line to the bottom line) 2000 mg, 1500 mg, 1000 mg, 500 mg, 300 mg, 150 mg, 75 mg and 25 mg.

FIG. 2 shows an XRPD of exemplary amorphous solid dispersion no. 1 (bottom line), exemplary amorphous solid dispersion no. 2 (top line) and exemplary amorphous solid dispersion no. 3 (middle line) after being kept at 40° C. and 75% relative humidity (RH) under closed conditions for one month.

FIG. 3 shows dissolution profiles of various exemplary amorphous solid dispersions on Day 1 (initial) and after 1-month 40° C. at 75% relative humidity under closed conditions. Data were obtained using an in vitro gastric to intestinal transfer test using 0.1 N HCl gastric media with an addition of intestinal media after 30 minutes for a final composition of 0.5% simulated intestinal fluid (SIF) in phosphate buffer saline (PBS) at pH 6.5.

FIG. 4 contains (A) Day 1 pharmacokinetic profiles which show naproxen metabolite (M25) plasma levels in Beagle dogs after a single dose administration of crystalline otenaproxesul (Group 1), milled crystalline otenaproxesul (Group 2), micro-milled crystalline otenaproxesul (Group 3), exemplary amorphous solid dispersion no. 1 (Group 4) and exemplary amorphous solid dispersion no. 4 (Group 5); (B) the Day 1 24-hour plasma naproxen exposure after a single dose administration of crystalline otenaproxesul (Group 1), milled crystalline otenaproxesul (Group 2), micro-milled crystalline otenaproxesul (Group 3), exemplary amorphous solid dispersion no. 1 (Group 4) and exemplary amorphous solid dispersion no. 4 (Group 5); (C) the Day 4 24-hour plasma naproxen (M25) exposure after the fourth single dose administration of crystalline otenaproxesul (Group 1), milled crystalline otenaproxesul (Group 2), micro-milled crystalline otenaproxesul (Group 3), exemplary amorphous solid dispersion no. 1 (Group 4) and exemplary amorphous solid dispersion no. 4 (Group 5); and (D) Day 4 pharmacokinetic profiles showing naproxen metabolite (M25) plasma levels after the fourth single dose administration of crystalline otenaproxesul (Group 1), milled crystalline otenaproxesul (Group 2), micro-milled crystalline otenaproxesul (Group 3), exemplary amorphous solid dispersion no. 1 (Group 4) and exemplary amorphous solid dispersion no. 4 (Group 5).

FIG. 5 contains pharmacokinetic profiles showing naproxen metabolite (M25) plasma levels in Beagle dogs after (A) administration of exemplary amorphous solid dispersion no. 4; (B) exemplary amorphous solid dispersion no. 1; (C) crystalline otenaproxesul (each line represents the results for one individual dog); and (D) a graph showing the accumulation of plasma naproxen at certain time intervals (30 minutes, 60 minutes and 120 minutes) after administration of crystalline otenaproxesul (first bar), milled crystalline otenaproxesul (second bar), micro-milled crystalline otenaproxesul (third bar), exemplary amorphous solid dispersion no. 1 (fourth bar) and exemplary amorphous solid dispersion no. 4 (fifth bar).

FIG. 6 contains graphs showing the pharmacodynamic effect on TXB2 levels in Beagle dogs of crystalline otenaproxesul (Group 1, top graph), exemplary amorphous solid dispersion 1 (Group 4, middle graph) and exemplary amorphous solid dispersion 4 (Group 5, bottom graph).

FIG. 7 are graphs showing the pharmacodynamic effect on PGE₂ levels in Beagle dogs of crystalline otenaproxesul (Group 1, top graph), exemplary solid dispersion no. 1 (Group 4, middle graph) and exemplary solid dispersion no. 4 (Group 5, bottom graph).

FIG. 8 are graphs showing (A) the mean plasma concentrations of naproxen metabolite of otenaproxesul versus time following oral administration on day 1 (left side panel) and on day 4 (right side panel) of crystalline otenaproxesul and exemplary solid dispersion no. 1 in rats-gender combined, and (B) the group mean plasma concentrations of M25 (naproxen metabolite of otenaproxesul) at 60 minutes post administration of exemplary solid dispersion no. 1 (solid bars) or crystalline otenaproxesul (diagonal striped bars).

FIG. 9 are graphs showing (A) exemplary solid dispersion no. 1 treatment dose proportionality on Day 1 and Day 4 (steady state)−(Solid markers=exemplary solid dispersion no. 1; Patterned markers=crystalline otenaproxesul) and (B) exemplary solid dispersion no. 1 treatment dose proportionality of Cmax on Day 1 and Day 4 (steady state)

FIG. 10 are graphs showing (A) male rat (top panel) and female rat (bottom panel) plasma M25 Day 1 and Day 4 AUC of exemplary solid dispersion no. 1 (solid bars) or crystalline otenaproxesul (diagonal striped bars); Day 1 is first bar from left, and Day 4 is second bar from left at each dose, and (B) exemplary solid dispersion no. 1 treatment dose proportionality of AUC on Day 1 and Day 4 (steady state) (solid markers) with reference crystalline otenaproxesul at 260 mg human equivalent dose (box, diagonal stripes).

FIG. 11 are graphs showing the mean plasma concentrations versus time following oral administration on day 1 (left side panel) and on day 4 (right side panel) of crystalline otenaproxesul and amorphous solid dispersion formulation in male beagle dogs.

FIG. 12 are graphs showing (A) the group mean plasma concentrations of M25 at 60 minutes post drug administration of amorphous solid dispersion formulation (solid bars) or crystalline otenaproxesul (diagonal striped bar), and (B) Day 1 and Day 4 group mean plasma concentrations of M25 at 60 minutes post drug administration of otenaproxesul amorphous solid dispersion formulation (solid bars) or crystalline otenaproxesul (diagonal striped bars); Day 1 is first bar from left, and Day 4 is second bar from left at each dose.

FIG. 13 are graphs showing (A) male dog M25 treatment group average of C₆₀ M25 plasma concentration (squares=amorphous; Black circle=Crystalline otenaproxesul), and (B) Amorphous solid dispersion treatment dose proportionality on Day 1 and Day 4 (steady state); Cmax Day 1 bottom line, Cmax Day 4 top line.

FIG. 14 are graphs showing (A) M25 Day 1 and Day 4 AUC of amorphous solid dispersion otenaproxesul (solid bars) or crystalline otenaproxesul (diagonal striped bars) in male Beagle dogs; Day 1 is first bar from left, and Day 4 is second bar from left at each dose, and (B) AUC exposure dose proportionality of M25 on Day 1 and Day 4 of amorphous solid dispersion otenaproxesul treatment in male dogs; AUC Day 1 bottom line, AUC Day 4 top line.

### DETAILED DESCRIPTION

### I. Definitions

Unless otherwise indicated, the definitions and embodiments described in this, and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

In understanding the scope of the present application, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “amorphous solid dispersions of the application” and the like as used herein refers to solid dispersions comprising amorphous, i.e., non-crystalline, otenaproxesul, or pharmaceutically acceptable salt thereof, and a polymer.

The term “composition(s) of the application” and the like as used herein refers to a composition, such a pharmaceutical composition, comprising an amorphous solid dispersion of the application.

The term “methods of the application” as used herein refers to any method for treating acute pain by administration or use of otenaproxesul or a pharmaceutically acceptable salt thereof described herein.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end-result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J.F.W. Ed., Plenum Press, 1973, in Greene, T. W. and Wuts, P.G.M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

As used herein, the term “effective amount” or “therapeutically effective amount” means an amount of otenaproxesul or a pharmaceutically acceptable salt thereof, or compositions comprising otenaproxesul or a pharmaceutically acceptable salt thereof, that is effective, at dosages and for periods of time necessary to achieve a desired result.

The term “administered” as used herein means administration of a therapeutically effective amount of otenaproxesul or a pharmaceutically acceptable salt thereof, or compositions comprising otenaproxesul or a pharmaceutically acceptable salt thereof to a cell either in cell culture or in a subject.

The term “subject” as used herein includes all members of the animal kingdom, including mammals, and suitably refers to humans. In a clinical setting, a subject may also be referred to, interchangeably, as a patient.

The term “pharmaceutical composition” as used herein refers to a composition of matter for pharmaceutical use.

The term “pharmaceutically acceptable” means compatible with the treatment of subjects.

The term “parenteral” as used herein means taken into the body or administered in a manner other than through the gastrointestinal tract.

The terms “to treat”, “treating” and “treatment” as used herein and as is well understood in the art, means an approach for obtaining beneficial or desired results, including clinical results. Examples of beneficial or desired clinical results include, but are not limited to diminishment of extent of disease, stabilized (i.e., not worsening) state of disease, preventing spread of disease, delay or slowing of disease progression, amelioration or palliation of the disease state, and remission (whether partial or total), whether detectable or undetectable. “To treat”, “treating” and “treatment” can also mean prolonging survival as compared to expected survival if not receiving treatment. “To treat”, “treating” and “treatment” as used herein also includes prophylactic treatment.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition

As used herein, the term “effective amount” or “therapeutically effective amount” means an amount of the one or more amorphous solid dispersions of the application or compositions of the application, that is effective, at dosages and for periods of time necessary to achieve a desired result.

The expression “inhibiting COX-1 and/or COX-2” as used herein refers to inhibiting, blocking and/or disrupting COX-1 and/or COX-2 enzymatic activity in a cell. The inhibiting, blocking and/or disrupting causes a therapeutic effect in the cell.

By “inhibiting, blocking and/or disrupting” it is meant any detectable inhibition, block and/or disruption in the presence of a compound or composition compared to otherwise the same conditions, except for in the absence in the compound or composition.

The term “disease, disorder or condition treatable by inhibition of COX-1 and/or COX-2” means that the disease, disorder or condition to be treated is affected by, modulated by and/or has some biological basis, either direct or indirect, that includes COX-1 and/or COX-2 activity, in particular, increased COX-1 and/or COX-2 activity. These diseases respond favourably when COX-1 and/or COX-2 activity associated with the disease, disorder or condition is inhibited by the otenaproxesul, or a pharmaceutically acceptable salt thereof, or the amorphous solid dispersions and compositions of the application comprising otenaproxesul or a pharmaceutically acceptable salt thereof.

The term “diseases, disorders or conditions associated with inflammation” means that the disease, disorder or condition to be treated is affected by, modulated by and/or has some biological basis, either direct or indirect, that includes inflammation.

The term “administered” as used herein means administration of a therapeutically effective amount of one or more amorphous solid dispersions of the application or one or more compositions of the application to a cell either in cell culture or in a subject.

When used in reference to a peak in the DSC thermogram, the term “about” means that the peak may vary by +/−1° C. of the subject value.

As used herein when referring to a diffractogram, spectrum and/or to data presented in a graph, the term “peak” refers to a feature that one skilled in the art would recognize as not attributing to background noise.

The term “enhanced bioavailability” as used herein refers to any detectable increase (e.g., plasma levels) of otenaproxesul or metabolite thereof following administration one or more amorphous solid dispersions of the application or one or more compositions of the application compared to the levels (e.g., plasma levels) of otenaproxesul or metabolite thereof following administration of crystalline otenaproxesul under otherwise identical conditions.

The term “treating acute pain” as used herein means any detectable reduction, for example, in the sensation or duration of acute pain after administration or use of a compound, in this case otenaproxesul, compared to otherwise the same conditions except in the absence of administration or use of the compound, for example, otenaproxesul.

The term “treating chronic pain” as used herein means any detectable reduction, for example, in the sensation or duration of chronic pain after administration or use of a compound, in this case otenaproxesul, compared to otherwise the same conditions except in the absence of administration or use of the compound, for example, otenaproxesul.

When used, for example, with respect to the methods of treatment, uses, compositions, packages and/or kits of the application, a subject, for example a subject “in need thereof” is a subject experiencing or expected to be experiencing pain or any subject that would benefit from administration of otenaproxesul, or a pharmaceutically acceptable salt thereof.

The term “otenaproxesul” as used herein refers to a compound having the chemical name: (S)-4-carbamothioylphenyl 2-(6-methoxynaphthalen-2-yl) propanoate, and having the structural formula:

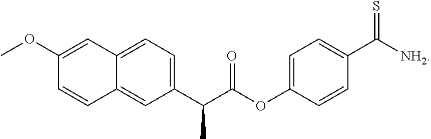 [SMILES: COc1ccc2cc([C@H](C)C(=O)Oc3ccc(C(N)=S)cc3)ccc2c1]

Otenaproxesul is also known as “ATB-346” and under the chemical name(S)-2-(6-methoxy-naphthalen-2-yl)-propionic acid 4-thiocarbamoyl-phenyl ester.

The term “otenaproxesul-amide” as used herein refers to a compound having the chemical name: 4-carbamoylphenyl(S)-2-(6-methoxynaphthalen-2-yl) propanoate and having the structural formula:

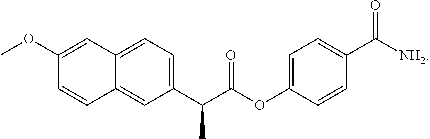 [SMILES: COc1ccc2cc([C@H](C)C(=O)Oc3ccc(C(N)=O)cc3)ccc2c1]

The term “alanine aminotransferase” or “ALT” as used herein refers to an enzyme that catalyzes the transfer of an amino group from L-alanine to α-ketoglutarate to produce pyruvate and L-glutamate.

The term “aspartate aminotransferase” or “AST” as used herein refers to an enzyme that catalyzes the transfer of an amino group from aspartate to α-ketoglutarate to produce oxaloacetate and glutamate.

The term “bilirubin” as used herein refers to a catabolite of heme that is cleared from the body by the liver.

The term “alkaline phosphatase” or “ALP” as used herein refers to an enzyme that hydrolyzes phosphate groups from various molecules and is present in the cells lining the biliary ducts of the liver.

The term “thromboxane B₂” or “TXB₂” as used herein refers to an inactive metabolite of thromboxane A₂.

The term “thromboxane A₂” as used herein refers to a type of thromboxane synthesized in platelets, via cyclo-oxygenase-1, that causes blood clotting and constriction of blood vessels.

The term “AUC” as used herein refers to the area under the curve of a concentration versus time graph (a concentration-time curve) which represents levels of a compound in the blood (e.g., plasma levels) as a function of time following administration and can be determined by measuring plasma levels of the compound or metabolite at various time points following administration.

The term “AUC₀₋ₗₐₛₜ” as used herein describes the area under the concentration-time curve up to the last quantifiable time-point post-administration of a dose. AUC₀₋ₗₐₛₜ” is “AUC₀₋ₜ” when time t is the last quantifiable time-point.

The term “AUC₀₋ₜ” as used herein describes the area under the concentration-time curve from time zero (e.g., dosing) to time “t” post-dosing.

The term “AUC_{0-inf}” or “AUC_{0-∞}” as used herein means an estimate of area under the concentration-time curve from time zero (e.g., dosing) to infinity.

The term “Tmax” as used herein refers to the time which elapses after administration of a compound at which the concentration of the compound or metabolite thereof in the blood (e.g., plasma level) attains the maximum concentrations.

The term “Cmax” refers to the maximal concentration of a compound in the blood (e.g., plasma levels), and can be determined by measuring levels of the compound at various time points following administration.

The term “C₆₀” refers to the concentration of a compound in the blood (e.g., plasma levels) at 60 minutes following administration of the compound.

The term “steady state” as used herein refers to a state in which the amount of the compound reaching the system is approximately the same as the amount of the compound leaving the system.

The term “naproxen” as used herein refers to a compound having the IUPAC name: (2S)-2-(6-methoxynaphthalen-2-yl) propanoic acid, and having the chemical formula:

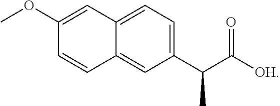 [SMILES: COc1ccc2cc([C@H](C)C(=O)O)ccc2c1]

and which is a metabolite of otenaproxesul.

The term “cumulative drug exposure” or “cumulative naproxen metabolite exposure” as used herein means the total amount of the compound or metabolite thereof (e.g., naproxen) in the blood (e.g., plasma levels) after administration of the compound which may be available for uptake by tissues over a specific time course. Cumulative drug exposure is measured in μg*hr/mL as a function of the concentration of drug times volume of a subject's plasma, over time, and can be estimated using the formula:

(AUC₀₋₂₄×T−1)+AUC_{0-inf}

wherein T is the number of treatment days. For example, the cumulative drug exposure resulting from a 7 day treatment period providing a daily steady state AUC₀₋₂₄ of 250 μg*hr/mL and a AUC_{0-inf} on the last day of treatment of 400 μg*hr/ml is 1900 μg*hr/ml (e.g., (250 μg*hr/mL×6)+400 μg*hr/mL).

The term “loading dose” as used herein refers to an initial dose of a drug which is typically given before, at the time of and/or shortly after the onset of the acute pain.

The term “maintenance dose” as used herein refers to a dose of a drug that follows a loading dose and that is typically lower than the loading dose.

The term “tapering” as used herein with respect to doses means to gradually decrease the dosage amount of a drug over time.

The term “spray-dried dispersion” as used herein means a product of a spray-drying process wherein the product comprises a dispersion of one or more compounds and at least one other component, such as a polymer.

The term “solid dispersion” as used herein refers to a compound molecularly dispersed with a polymer.

The term “molecularly dispersed”, as used herein, refers to the random distribution of a compound with a polymer.

The term “amorphous solid dispersion” as used herein means that the solid dispersion contains the compound in a substantially amorphous solid state form.

As used herein, the term “amorphous” or “amorphous form” “amorphous solid state form” refers to a compound that is in a non-crystalline state. It is noted that the phrase “amorphous solid” is sometimes used in the art to refer to a crystal form with disorganized small crystals (typically resulting from rapid small scale drying). This latter meaning is not the intended usage in this application as such an amorphous solid comprises crystals and not the substantially amorphous form of the invention.

The term “excipient” as used herein means a therapeutically non-active ingredient which facilitates aspects of manufacturing, stability, dissolution of tablets.

The term “HPMCAS” as used herein refers to the polymer hydroxypropylmethylcellulose acetate succinate which is a mixture of acetic acid and monosuccinic acid esters of hydroxypropylmethylcellulose.

The term “PVPVA” or “PVP/VA” or “polyvinylpyrrolidone-vinyl acetate” as used herein refers to the polymer poly(1-vinylpyrrolidone-co-vinyl acetate).

The term “sustaining agent” as used herein means an agent that sustains the dissolution rate of the compound and may include a polymer.

The term “D50” as used herein refers the diameter value for the particles in the material at which about 50% of the particles have a diameter below the diameter value.

The term “D90” as used herein refers the diameter value for the particles in the material at which about 90% of the particles have a diameter below the diameter value.

The acronym “XRPD” means X-ray powder diffraction, an analytical technique which measures the diffraction of X-rays in the presence of a solid component. Materials which are crystalline and have regular repeating arrays of atoms generate a distinctive powder pattern. Materials with similar unit cells will give powder patterns that are similar in position as measured in ° 2θ (theta). The intensity of the reflections varies according to the electron density causing diffraction as well as sample, sample preparation, and instrument parameters. Analysis of XRPD data is based upon the general appearance of the measured powder pattern(s) with respect to the known response of the X-ray diffraction system used to collect the data. For diffraction peaks that may be present in the powder pattern, their positions, shapes, widths and relative intensity distributions can be used to characterize the type of solid state order in the powder sample. The position, shape and intensity of any broad diffuse scatter (halos) on top of the instrumental background can be used to characterize the level and type of solid state disorder. The combined interpretation of the solid state order and disorder present in a powder sample provides a qualitative measure of the macro-structure of the sample.

### II. Amorphous Otenaproxesul, Amorphous Solid Dispersions, Compositions and Kits of the Application

The present application includes amorphous solid dispersions of otenaproxesul and compositions comprising amorphous solid dispersions of otenaproxesul, both of which show significantly enhanced bioavailability of otenaproxesul, when compared with crystalline otenaproxesul, including milled crystalline otenaproxesul. For example, amorphous solid dispersions of otenaproxesul molecularly dispersed within a polymer and compositions comprising an amorphous solid dispersion of otenaproxesul molecularly dispersed within a polymer, and optionally comprising further sustaining agents are disclosed.

Otenaproxesul is known to exist in crystalline form. Initial studies carried out with formulations comprising crystalline otenaproxesul demonstrated limited initial absorption (e.g., within the first 60 minutes post treatment) of otenaproxesul. The amorphous solid dispersions of the present application comprising amorphous otenaproxesul have been shown to have an unexpectedly large increase in solubility in biologically relevant media as well as a faster release profile and enhanced bioavailability (for example as measured using levels of naproxen metabolite) compared to crystalline otenaproxesul, even when the crystalline form was milled or micro-milled. For example, the amorphous solid dispersions of the application were found to provide an otenaproxesul solubility in 0.5 wt % simulated intestinal fluid (SIF) that is about 4.6-fold that of crystalline otenaproxesul. Further, the amorphous solid dispersions and compositions of the application have been shown to provide about a 7-to-9-fold improvement in Day 1 AUC₀₋₂₄ of naproxen metabolite (M25) compared to crystalline otenaproxesul when a single dose administration of 15 mg/kg of crystalline otenaproxesul or an amorphous solid dispersion of the application was administered to Beagle dogs. Similarly, the amorphous solid dispersions and compositions of the application have been shown to provide about a 3- to 4.5-fold improvement compared to crystalline otenaproxesul in Day 4 AUC₀₋₂₄ of naproxen metabolite (M25) after a fourth single dose administration of 15 mg/kg of crystalline otenaproxesul or the amorphous solid dispersions of the application was administered to Beagle dogs. Further still, the amorphous solid dispersions of the application were also shown to provide about an average naproxen metabolite (M25) plasma concentration one hour (1 hour average) after administration that was 22-fold higher than that of crystalline otenaproxesul.

The Applicants have further found that although the amorphous solid dispersions of otenaproxesul demonstrate an enhanced absorption and initial plasma exposures on Day 1 of naproxen compared to crystalline otenaproxesul, the amorphous solid dispersions of otenaproxesul surprisingly demonstrated lower steady state (e.g, Day 4) exposures compared to crystalline otenaproxesul. While not wishing to be bound by theory, these results suggest that administration or use of amorphous solid dispersions of otenaproxesul of the application would advantageously provide enhanced initial naproxen plasma exposures but overall lower naproxen plasma exposures. By extrapolation, H₂S (hydrogen sulfide) cumulative exposure in plasma over time would be expected to be lower compared to the administration or use of crystalline otenaproxesul. With a lower overall cumulative naproxen and H₂S exposure in plasma, concomitant lower liver exposure to exogenous H₂S associated with otenaproxesul and in turn fewer liver-adverse events, would be expected with the use of amorphous solid dispersions of otenaproxesul compared to administration or use of crystalline otenaproxesul.

Since the amorphous solid dispersions and compositions of the application have an increased solubility and provide a faster release profile and enhanced bioavailability compared to crystalline otenaproxesul, then lower amounts of otenaproxesul compared to crystalline otenaproxesul are required to achieve the same beneficial effects. Therefore, the compositions of the application further advantageously are expected to improve patient compliance by providing a reduced pill burden (e.g., lower number of oral compositions of the application required per day).

Otenaproxesul has a great tendency to crystallize. The amorphous solid dispersions of the present application have also been shown to be stable to crystallization of otenaproxesul over time. Crystallization of otenaproxesul in amorphous solid dispersions of otenaproxesul would affect bioavailability due to the advantage of enhanced bioavailability of the amorphous form in the amorphous solid dispersions. Crystallization of otenaproxesul in amorphous solid dispersions of otenaproxesul can also lead to improper dosing due to the difference in bioavailability of otenaproxesul in an amorphous solid dispersion compared to crystalline otenaproxesul. Various polymers for forming amorphous solid dispersion of otenaproxesul were investigated. Exemplary amorphous solid dispersions of the present application are stable to crystallization. In exemplary embodiments, a number of amorphous solid dispersions comprising otenaproxesul and a polymer, such as hydroxypropylmethylcellulose acetate succinate (HPMCAS) or polyvinylpyrrolidone-vinyl acetate (PVPVA), and compositions thereof optionally with further sustaining agents, have been developed. The Applicant has also shown the amorphous solid dispersions of the application are stable to crystallization, for example, for at least one month.

The Applicant has further investigated and developed dosage regimens for treating acute pain. The dosage regimens comprise administrating a loading dose of otenaproxesul that was found to provide levels of naproxen (otenaproxesul metabolite) expected to be concomitant with pain relief within 60 minutes after administration, and further optionally administering one or more maintenance doses after administration of the loading dose in amounts including tapering amounts sufficient to sustain pain relief while advantageously mitigating the liver adverse events observed with sustained higher doses of otenaproxesul.

### Amorphous Otenaproxesul

In some embodiments, the present application includes amorphous otenaproxesul, or pharmaceutically acceptable salt thereof, comprising, consisting essentially of or consisting of at least about 99.5 wt % amorphous otenaproxesul or pharmaceutically acceptable salt thereof. In some embodiments, the amorphous otenaproxesul or pharmaceutically acceptable salt thereof comprises at least about 99.5 wt %, at least about 99.6 wt %, at least about 99.7 wt %, at least about 99.8 wt % or at least about 99.9 wt % amorphous otenaproxesul or pharmaceutically acceptable salt thereof. In some embodiments, the wt % is based on the total weight of otenaproxesul.

In some embodiments, the present application includes otenaproxesul, or a pharmaceutically acceptable salt thereof, wherein at least about 95 wt % of the otenaproxesul, or the pharmaceutically acceptable salt thereof, is in amorphous form. In some embodiments, the otenaproxesul, or a pharmaceutically acceptable salt thereof, is at least about 96 wt %, at least about 97 wt %, at least about 98 wt % or at least about 99 wt % in amorphous form. In some embodiments, at least about 100 wt %, at least about 99.5 wt %, at least about 99.6 wt %, at least about 99.7 wt %, at least about 99.8 wt is in amorphous form. In some embodiments, 0.5 wt % or less, about 0.4 wt % or less, about 0.3 wt % or less, about 0.2 wt % or less or about 0.1 wt % or less of the otenaproxesul is in a crystalline form. In some embodiments, within the limits of detection, using for example, XRPD, the otenaproxesul, or a pharmaceutically acceptable salt thereof, is 100% in the amorphous form, or the is 0% in a crystalline form.

In some embodiments, the amorphous otenaproxesul or pharmaceutically acceptable salt thereof about 0.5 wt % or less, about 0.4 wt % or less, about 0.3 wt % or less, about 0.2 wt % or less or about 0.1 wt % or less crystalline otenaproxesul, wherein the wt % is based on the total weight of otenaproxesul. In some embodiments, the amorphous otenaproxesul or pharmaceutically acceptable salt thereof comprises 0.5 wt %, 0.4 wt %, 0.3 wt %, 0.2 wt % or 0.1 wt % or less crystalline otenaproxesul. In some embodiments, the amorphous otenaproxesul or pharmaceutically acceptable salt thereof comprises 0.1 wt % or less crystalline otenaproxesul. In some embodiments, the amorphous otenaproxesul or pharmaceutically acceptable salt thereof comprises non detectable amounts of crystalline otenaproxesul as measured, for example, by XPRD. In some embodiments, the wt % is based on the total weight of otenaproxesul. In some embodiments, the application includes a pharmaceutical composition comprising otenaproxesul or pharmaceutically acceptable salt thereof and a pharmaceutically acceptable excipient, wherein at least about 95 wt % of the otenaproxesul, or the pharmaceutically acceptable salt thereof, is in amorphous form. In some embodiments, at least about 99.5 wt %, at least about 99.6 wt %, at least about 99.7 wt %, at least about 99.8 wt is in amorphous form.

In some embodiments, the application includes a pharmaceutical composition comprising amorphous otenaproxesul or pharmaceutically acceptable salt thereof and a pharmaceutically acceptable excipient, wherein the amorphous otenaproxesul or pharmaceutically acceptable salt thereof comprises, consists essentially of or consists of at least about 99.5 wt % amorphous otenaproxesul or pharmaceutically acceptable salt thereof. In some embodiments, the amorphous otenaproxesul or pharmaceutically acceptable salt thereof comprises, consists essentially of or consists of at least about 95 wt % amorphous otenaproxesul or pharmaceutically acceptable salt thereof. In some embodiments, the wt % is based on the total weight of otenaproxesul.

### Amorphous Solid Dispersions and Compositions Thereof.

The Applicant has developed amorphous solid dispersions of otenaproxesul in a polymer matrix which have been shown to be stable to crystallization over at least one month. The amorphous solid dispersions have also been shown to provide a significantly enhanced bioavailability of otenaproxesul, when compared with crystalline otenaproxesul.

Therefore, in some embodiments, the application includes an amorphous solid dispersion comprising amorphous otenaproxesul or pharmaceutically acceptable salt thereof and a polymer.

In some embodiments, the amorphous otenaproxesul or pharmaceutically acceptable salt thereof is molecularly dispersed in the polymer. Therefore, in some embodiments, the application includes an amorphous solid dispersion comprising amorphous otenaproxesul or pharmaceutically acceptable salt thereof molecularly dispersed in a polymer.

In some embodiments, amorphous otenaproxesul is in a neutral form (i.e., not a salt). Therefore, in some embodiments, the application includes an amorphous solid dispersion comprising amorphous otenaproxesul and a polymer.

The Applicant has developed amorphous solid dispersions of otenaproxesul in a dispersed in a polymer which have been shown to be stable to crystallization. Therefore, in some embodiments, the application includes a stable amorphous solid dispersion.

By “stable” or “stable to crystallization” as used herein in reference to an amorphous solid dispersion means no significant increase in amount of crystalline otenaproxesul over a given length of time. In some embodiments, there is less than about 10%, less than about 8%, less than about 6%, less than about 5%, less than about 4%, less than about 3%, less than about 2%, or less than about 1% increase in amount of crystalline otenaproxesul over a given length of time. In some embodiments, there is less than about 5%, less than about 4%, less than about 3%, less than about 2%, or less than about 1% increase in amount of crystalline otenaproxesul over a given length of time. In some embodiments, there is no detectable amounts of crystalline otenaproxesul as measured by XRPD over a given length of time.

In some embodiments, the amorphous solid dispersion is stable to crystallization for at least one month, at least two months, at least three months, at least four months, at least five months, at least six months, at least eight months, at least ten months, at least twelve months, at least fourteen months, at least eighteen months, at least twenty months or at least at least twenty-four months.

In some embodiments, the polymer provides stability of the amorphous solid dispersion. Therefore, in some embodiments, the polymer is a stabilizing polymer, and the application includes an amorphous solid dispersion comprising amorphous otenaproxesul or pharmaceutically acceptable salt thereof and a stabilizing polymer.

By “stabilizing” as used herein in reference to a polymer means no significant increase in amount of crystalline otenaproxesul in amorphous solid dispersion comprising the polymer over a given length of time. In some embodiments, there is less than about 10%, less than about 8%, less than about 6%, less than about 5%, less than about 4%, less than about 3%, less than about 2%, or less than about 1% increase in amount of crystalline otenaproxesul over a given length of time. In some embodiments, there is less than about 5%, less than about 4%, less than about 3%, less than about 2%, or less than about 1% increase in amount of crystalline otenaproxesul over a given length of time. In some embodiments, there is no detectable amounts of crystalline otenaproxesul within the limits of detection as measured by XRPD over a given length of time.

In some embodiments, the amorphous otenaproxesul or pharmaceutically acceptable salt thereof is present in the amorphous solid dispersion in a form that is about 90 wt % or more, about 92 wt % or more, about 94 wt % or more, about 95 wt % or more, about 96 wt %, about 97 wt %, about 98 wt %, or about 99 wt % or more amorphous form. In some embodiments, the amorphous otenaproxesul or pharmaceutically acceptable salt thereof is present in the amorphous solid dispersion in a form that is about 99.5 wt % or more, about 99.6 wt % or more, about 99.7 wt % or more, about 99.8 wt % or more, or about 99.9 wt % or more amorphous form. In some embodiments, the amorphous otenaproxesul or pharmaceutically acceptable salt thereof is present in the amorphous solid dispersion in a form that is about 99.7 wt % or more, about 99.8 wt % or more, or about 99.9 wt % or more amorphous form. In some embodiments, the amorphous otenaproxesul or pharmaceutically acceptable salt thereof is present in the amorphous solid dispersion in a form that is at least 99.5 wt %, at least 99.6 wt %, at least 99.7 wt %, at least 99.8 wt % or at least 99.9 wt % amorphous form. In some embodiments, the amorphous otenaproxesul or pharmaceutically acceptable salt thereof is present in the amorphous solid dispersion in a form that is at least 99.7 wt %, at least 99.8 wt % or at least 99.9 wt % amorphous form.

In some embodiments, the amorphous otenaproxesul or pharmaceutically acceptable salt thereof in the amorphous solid dispersion comprises 10 wt % or less, 8 wt % or less, 6 wt % or less, 5 wt % or less, 4 wt % or less, 3 wt % or less, 2 wt % or less, or 1 wt % or less crystalline otenaproxesul, wherein the wt % is based on the total weight of otenaproxesul. In some embodiments, the amorphous otenaproxesul or pharmaceutically acceptable salt thereof in the amorphous solid dispersion comprises 2 wt % or less or 1 wt % or less crystalline otenaproxesul, wherein the wt % is based on the total weight of otenaproxesul.

In some embodiments, the amorphous otenaproxesul or pharmaceutically acceptable salt thereof in the amorphous solid dispersion consists of 0.5 wt %, 0.4 wt %, 0.3 wt %, 0.2 wt % or 0.1 wt % or less crystalline otenaproxesul. In some embodiments, the amorphous otenaproxesul or pharmaceutically acceptable salt thereof in the amorphous solid dispersion consists of 0.1 wt % or less crystalline otenaproxesul. In some embodiments, the amorphous otenaproxesul or pharmaceutically acceptable salt thereof consists of non detectable amounts of crystalline otenaproxesul by XPRD.

In some embodiments, the solid-state form of otenaproxesul or pharmaceutically acceptable salt thereof, such as the otenaproxesul, or pharmaceutically acceptable salt thereof, substance in the amorphous dispersion, is determined by methods known in art, for example, by Polarized Light Microscopy, X-Ray Powder Diffraction (XPRD), Differential Scanning calorimetry (DSC), or other standard techniques known in the art.

In some embodiments, amorphous otenaproxesul or pharmaceutically acceptable salt thereof is present in the amorphous solid dispersion in an amount of about 15% to about 60%, about 15% to about 50%, about 15% to about 50%, about 20% to about 50%, about 30% to about 50% or about 35% to about 50% by weight of the amorphous solid dispersion. In some embodiments, amorphous otenaproxesul or pharmaceutically acceptable salt thereof is present in the amorphous solid dispersion in an amount of about 20% to about 50%, about 30% to about 50%, about 35% to about 50% or about 35% to about 45% by weight of the amorphous solid dispersion. In some embodiments, amorphous otenaproxesul or pharmaceutically acceptable salt thereof is present in the amorphous solid dispersion in an amount of about 15%, about 20%, about 25%, about 30%, about 35%, about 40%, about 45%, about 50% or about 55% by weight of the amorphous solid dispersion. In some embodiments, amorphous otenaproxesul or pharmaceutically acceptable salt thereof is present in the amorphous solid dispersion in an amount of about 20%, about 25%, about 30%, about 35%, about 40%, about 45%, or about 50% by weight of the amorphous solid dispersion. In some embodiments, amorphous otenaproxesul or pharmaceutically acceptable salt thereof is present in the amorphous solid dispersion in an amount of about 35% by weight of the amorphous solid dispersion.

In some embodiments, the polymer is present in the amorphous solid dispersion in an amount of about 40% to about 80%, 40% to about 70%, about 40% to about 65%, about 50% to about 80%, about 50% to about 70%, about 50% to about 65%, about 55% to about 65%, about 50% to about 60%, about 60% to about 65% by weight of the amorphous solid dispersion.

In some embodiments, the weight ratio of amorphous otenaproxesul, or pharmaceutically acceptable salt thereof, to the polymer in the amorphous solid dispersion is about 1:1 to about 1:4, or about 1.5 to about 1:4. In some embodiments, the weight ratio of amorphous otenaproxesul, or pharmaceutically acceptable salt thereof, to the polymer in the amorphous solid dispersion is about 1:1, about 1:2, about 1:3, about 1:4, about 1:1.5, about 1:1.75 or about 1:1.8.

In some embodiments, the polymer is selected from one or more pharmaceutically acceptable acidic, neutral or basic polymers. In some embodiments, the one or more pharmaceutically acceptable polymers are selected from acidic polymers. In some embodiments, the one or more pharmaceutically acceptable polymers are selected from neutral polymers. In some embodiments, when a neutral polymer is used, the amorphous solid dispersion further comprises one or more antioxidants.

The Applicants have investigated the preparation of amorphous solid dispersions of otenaproxesul or pharmaceutically acceptable salt thereof with various polymers, including, for example, hydroxypropylmethylcellulose acetate succinate (HPMC-AS), hydroxypropylmethylcellulose (hypromellose, HPMC), polyvinylpyrrolidone (PVP), polyvinylpyrrolidone vinyl acetate (PVP VA, copovidone), methacrylic acid-methyl methacrylate copolymers, polyvinyl caprolactam polyvinyl acetate-polyethylene glycol graft copolymer, PEG3350, PEG 8000 and polyacrylic acid and combinations thereof.

Various amorphous solid dispersions of otenaproxesul were prepared. Amorphous solid dispersions of otenaproxesul or pharmaceutically acceptable salt thereof were prepared using various polymers and combinations of polymers.

The Applicants further tested the stability of the prepared amorphous solid dispersions to crystallization. Most polymers or combinations thereof tested were stabilizing polymers capable of preventing crystallization of otenaproxesul in the amorphous solid dispersion on their own or in combination. Some polymers, such as for example, polyacrylic acid, polyvinylpyrrolidone K-30, HPMC E5 and a combination of PEG8000 and hydroxypropylmethylcellulose acetate succinate (HPMCAS) may optionally be used in combination with stabilizing agents to improve the stability of the prepared amorphous solid dispersions to crystallization.

In some embodiments, the polymer is selected from hydroxypropyl methylcellulose (HPMC), hydroxypropyl methylcellulose phthalate (HPMCP), hydroxypropylmethylcellulose acetate succinate (HPMCAS), hydroxypropyl cellulose (HPC), polyvinylpyrrolidone (PVP), poly(vinylpyrrolidone-co-vinyl acetate (PVP/VA), cellulose acetate phthalate (CAP) and polymeric polymethacrylates (e.g., EUDRAGIT® such as EUDRAGIT® L100), and mixtures thereof. In some embodiments, the polymer is selected from hydroxypropyl methylcellulose phthalate (HPMCP), hydroxypropylmethylcellulose acetate succinate (HPMCAS), hydroxypropyl cellulose (HPC), polyvinylpyrrolidone (PVP), poly(vinylpyrrolidone-co-vinyl acetate (PVP/VA) and cellulose acetate phthalate (CAP), and mixtures thereof.

In some embodiments, the polymer is selected from polymers of: cellulose optionally functionalized with combinations of alkyl ethers, alkyl esters, and phthalate esters; vinyl alcohol; vinyl acetate; propylene glycol, oxyethylene; oxypropylene; acrylic acid, methacrylic acid; methyl methacrylate; ethylene glycol; ethylene glycol glycerides; ethylene oxide; propylene oxide; 2-ethyl-2-oxazoline; maleic acid; methyl vinyl ether; vinyl caprolactam; polyvinylpyrrolidone and combinations thereof.

In some embodiments, the polymer is selected from polymers of: cellulose optionally functionalized with combinations of alkyl ethers, alkyl esters, and phthalate esters; vinyl alcohol; vinyl acetate; oxyethylene; oxypropylene; acrylic acid, methacrylic acid; methyl methacrylate; ethylene glycol; ethylene glycol glycerides; ethylene oxide; propylene oxide; 2-ethyl-2-oxazoline; maleic acid; methyl vinyl ether; vinyl caprolactam; polyvinylpyrrolidone, combinations of one or more of the preceding polymers, and propylene glycol in combination with one or more of the preceding polymers.

In some embodiments, the pharmaceutically acceptable polymer comprises polymers of: cellulose optionally functionalized with any combination of alkyl ethers, alkyl esters, and phthalate esters; vinyl alcohol; vinyl acetate; propylene glycol; pyrrolidone; vinylpyrrolidone, oxyethylene; oxypropylene; methacrylic acid; methyl methacrylate; ethylene glycol; ethylene glycol glycerides; ethylene oxide; propylene oxide; 2-ethyl-2-oxazoline; maleic acid; methyl vinyl ether; vinyl caprolactam; or combinations thereof.

In some embodiments, the polymer is selected from polyvinylpyrrolidone (PVP) based polymers, polyethylene glycol (PEG) based polymers, cellulose based polymers, acrylate based polymers, chitosan, polyvinyl alcohol/polyethylene glycol graft copolymers, polyvinyl caprolactam polyvinyl acetate-polyethylene glycol graft copolymers and polyvinyl acetate phthalate, and combinations of one or more of the listed polymers.

In some embodiments, the polymer is selected from polyvinylpyrrolidone (PVP) based polymers, cellulose based polymers, acrylate based polymers, chitosan, polyvinyl alcohol/polyethylene glycol graft copolymers, polyvinyl caprolactam polyvinyl acetate-polyethylene glycol graft copolymers and polyvinyl acetate phthalate, and combinations thereof, combinations of one or more of the listed polymers, and polyethylene glycol (PEG) based polymers in combination with one or more of the listed polymers.

In some embodiments, the polymer is selected from polyvinylpyrrolidone, polyvinylpyrrolidone vinyl acetate (PVP VA), crospovidone (PVPP), methylcellulose, hydroxypropylmethylcellulose (HPMC), hydroxypropylmethylcellulose acetate succinate (HPMCAS), hydroxypropylmethylcellulose phthalate (HPMCP), cellulose acetate phthalate (CAP), polyethylene glycol (PEG), methacrylate copolymers, polyacrylic acid, chitosan, polyvinyl alcohol/polyethylene glycol graft copolymers, polyvinyl caprolactam polyvinyl acetate-polyethylene and combinations thereof.

In some embodiments, the polymer is selected from polyvinylpyrrolidone, polyvinylpyrrolidone vinyl acetate (PVP VA), crospovidone (PVPP), methylcellulose, hydroxypropylmethylcellulose (HPMC), hydroxypropylmethylcellulose acetate succinate (HPMCAS), hydroxypropylmethylcellulose phthalate (HPMCP), cellulose acetate phthalate (CAP), methacrylate copolymers, polyacrylic acid, chitosan, polyvinyl alcohol/polyethylene glycol graft copolymers, polyvinyl caprolactam polyvinyl acetate-polyethylene, combinations of one or more of the listed polymers, and polyethylene glycol (PEG) based polymers in combination with one or more of the listed polymers, provided HPMC does not comprise E type HPMC comprising a viscosity of about 5 cP or greater.

In some embodiments, the polymer is selected from polyvinylpyrrolidone K-12, polyvinylpyrrolidone vinyl acetate (PVP VA), crospovidone (PVPP), hydroxypropylmethylcellulose E3 (HPMC E3), hydroxypropylmethylcellulose acetate succinate (HPMCAS), methacrylate copolymers, polyvinyl alcohol/polyethylene glycol graft copolymers and polyvinyl caprolactam polyvinyl acetate-polyethylene glycol graft copolymers, and combinations thereof.

In some embodiments, the polymer comprises one polymer. In some embodiments, the polymer comprises a mixture of two or more polymers. In some embodiments, the polymer is one polymer. In some embodiments, the polymer is a mixture of two or more polymers.

In some embodiments, the polyvinylpyrrolidone (PVP) based polymers are selected from polyvinylpyrrolidone, polyvinylpyrrolidone/vinylacetate copolymer (PVP VA, copovidone), and crospovidone (PVPP, cross linked polyvinylpyrrolidone) and combinations thereof.

Amorphous solid dispersions of otenaproxesul prepared with polyacrylic acid, polyvinylpyrrolidone K-30, HPMC E5 and a combination of PEG8000 and hydroxypropylmethylcellulose acetate succinate (HPMCAS) were found to provide amorphous solid dispersion showed some crystallization of the otenaproxesul in the amorphous solid dispersion and therefore the amorphous solid dispersion optionally comprise further stabilizing agents.

In some embodiments, the polyvinylpyrrolidone is polyvinylpyrrolidone comprising a K value of about 10 to less than about 30, about 10 to less than about 25, about 10 to less than about 17, about 10 to less to than about 15, about 12 to less than about 30, about 12 to less than about 25, about 12 to less than about 17, about 12 to less to than about 15. In some embodiments, the polyvinylpyrrolidone is polyvinylpyrrolidone comprising a K value of about 25, about 17, about 15, about 12 or about 10. In some embodiments, the polyvinylpyrrolidone is polyvinylpyrrolidone comprising a K value of 12 (e.g. polyvinylpyrrolidone K-12). In some embodiments, the polyvinylpyrrolidone is a polyvinylpyrrolidone with a K value of about 10 to about 30, about 10 to about 17, about 10 to about 25, about 12 to about 30, about 17 to about 30 or about 25 to about 30. In some embodiments, the polyvinylpyrrolidone (PVP) based polymers do not comprise polyvinylpyrrolidone with a K grade of 30 or greater.

In some embodiments, the K value of polyvinylpyrrolidone is a value related to the relative viscosity of PVP aqueous solution. In some embodiments, the viscosity is generally related to the molecular weight of the PVP wherein the higher the K value correlates to a higher molecular weight of the PVP.

In some embodiments, the cellulose based polymers are selected from methylcellulose, hydroxypropyl cellulose, hydroxypropylmethylcellulose (hypromellose, HPMC), hydroxypropyl methylcellulose phthalate (HPMCP), hydroxypropylmethylcellulose acetate succinate (HPMCAS) and cellulose acetate phthalate (CAP) and combinations thereof.

In some embodiments, the polymer is HPMCAS. In some embodiments, HPMCAS is a mixture of acetic acid and monosuccinic acid esters of hydroxypropylmethylcellulose. In some embodiments, HPMCAS is commercially available. For example, HPMCAS is commercially available as AquaSolve™ (Global Specialty Chemicals Inc., Delaware, U.S.).

In some embodiments, HPMCAS is available in various different grades L, H and G, (e.g., HPMCAS-H, HPMCAS-L and HPMCAS-M) differentiated by the ratio of succinyl and acetyl substituents on the HPMC backbone. In some embodiments, the HPMCAS is HPMCAS-H or HPMCAS-L. In some embodiments, the HPMCAS is HPMCAS-H. HPMCAS is soluble in a wide range of organic solvents, making it compatible with a range of different active pharmaceutical ingredients. In some embodiments, the grades of HPMCAS comprise fine (F) and granular (G). Therefore, in some embodiments, the HPMCAS is HPMCAS-H and the HPMCAS-H is selected from HPMCAS-HG and HPMCAS-HF. In some embodiments, the HPMCAS is HPMCAS-HG.

In some embodiments, HPMCAS is HPMCAS-HG.

In some embodiments, HPMC is E, F or K type HPMC. In some embodiments, HPMC is E type (HPMC E). In some embodiments, E type HPMC comprises an average content of methoxyl groups of 29% and an average content of hydroxypropyl groups of 10%. In some embodiments, HPMC is a low viscosity HPMC. In some embodiments, HPMC E is a low viscosity HPMC E. In some embodiments, the HPMC has viscosity of about 4 cP or less or about 5 cP or less. In some embodiments, the HPMC has viscosity of about 3 cP to about 6 cP or about 4 cP or lower to about 5 cP. In some embodiments, the E type HPMC is E3. In some embodiments, the E type HPMC is not E5 or E15. In some embodiments, the cellulose based polymers do not comprise E type hydroxypropylmethylcellulose comprising a viscosity of about 5 cP or greater, In some embodiments, the PEG based polymers in combination with one or more of the listed polymers is not propylene glycol 8000 in combination with hydroxypropylmethylcellulose acetate succinate in a weight ratio of about 1.2:1 of the PEG 8000 to hydroxypropylmethylcellulose acetate succinate.

In some embodiments, HPMCAS is present in the amorphous solid dispersion in an amount of about 40% to about 80%, 40% to about 70%, about 40% to about 65%, about 50% to about 80%, about 50% to about 70%, about 50% to about 65%, about 55% to about 65%, about 50% to about 60%, about 60% to about 65% by weight of the amorphous solid dispersion. In some embodiments, HPMCAS is present in the amorphous solid dispersion in an amount of about 50% to about 60%, about 55% to about 65% or about 60% to about 65% by weight of the amorphous solid dispersion. In some embodiments, the HPMCAS is present in the amorphous solid dispersion in an amount of about 40%, about 45%, about 50%, about 55%, about 60%, about 65%, about 70%, about 75%, or about 80% by weight of the amorphous solid dispersion. In some embodiments, the HPMCAS is present in the amorphous solid dispersion in an amount of about 65% by weight of the amorphous solid dispersion. In some embodiments, the weight ratio of otenaproxesul, or pharmaceutically acceptable salt thereof, to the HPMCAS in the amorphous solid dispersion is about 1:1 to about 1:4. In some embodiments, the weight ratio of otenaproxesul, or pharmaceutically acceptable salt thereof, to HPMCAS in the amorphous solid dispersion is about 1:4 or about 1:1.8.

In some embodiments, HPMCAS is present in the amorphous solid dispersion in an amount of about 50% to about 60% or about 60% to about 65% by weight of the amorphous solid dispersion and the Tg of the amorphous solid dispersion is about 62° C. to about 65° C. In some embodiments, HPMCAS is present in the amorphous solid dispersion in an amount of about 65% by weight of the amorphous solid dispersion and the Tg of the amorphous solid dispersion is about 63° C. to about 64° C.

In some embodiments, the acrylate based polymer is selected from methacrylate copolymers. In some embodiments, the methacrylate copolymers are selected from cationic, anionic and neutral methacrylate copolymers.

In some embodiments, the cationic methacrylate copolymers are aminoalkylmethacrylate copolymers. In some embodiments, the aminoalkylmethacrylate copolymers are Eudragit® E such as Eudragit® E PO.

In some embodiments, the anionic methacrylate copolymers are methacrylic acid copolymers. In some embodiments, anionic methacrylic acid copolymers are selected from Eudragit® L (such as Eudragit® L100) or Eudragit® S.

In some embodiments, the neutral methacrylate copolymer are methacrylic acid copolymers. In some embodiments, neutral methacrylic acid copolymers are selected from Eudragit® RL, Eudragit® RS and Eudragit® NE.

In some embodiments, the methacrylate copolymers are selected from cationic and anionic methacrylate copolymers.

In some embodiments, the acrylate based polymer is not polyacrylic acid.

In some embodiments, the polyethylene glycol (PEG) based polymers have a molecular weight of less than 8000 g/Mol. In some embodiments, polyethylene glycol (PEG) based polymers have a molecular weight of about 3350 to about 8000 g/Mol. In some embodiments, polyethylene glycol (PEG) based polymers are in solid form.

In some embodiments, the polyvinyl alcohol/polyethylene glycol graft copolymers is Kollicoat®.

In some embodiments, the polyvinyl caprolactam polyvinyl acetate-polyethylene glycol graft copolymer is Soluplus®.

In some embodiments, the polymer comprises a mixture of two polymers. In some embodiments, the polymer comprises a mixture of two polymers, wherein one of the two polymer is HPMCAS. In some embodiments, the other of the two polymers is selected from PVP/VA and polyvinyl caprolactam polyvinyl acetate-polyethylene glycol graft copolymer.

In some embodiments, the polymer is PVP/VA. In some embodiments, PVP/VA is a polyvinylpyrrolidone/vinylacetate copolymer. In some embodiments, PVP/VA is a copolymer of 1-vinyl-2-pyrrolidone and vinyl acetate in a ratio of 6:4 by mass (PVP/VA64). Therefore, in some embodiments, PVP/VA is PVP/VA64. In some embodiments, PVP/VA64 is commercially available. For example, PVP/VA64 is available as Kollidon® VA 64 supplied from BASF Pharma (Ludwigshafen, Germany).

In some embodiments, PVP/VA is present in the amorphous solid dispersion in an amount of about 40% to about 70%, about 40% to about 65%, about 50% to about 80%, about 50% to about 70%, about 50% to about 65%, about 50% to about 60%, about 55% to about 65%, about 60% to about 65% by weight of the amorphous solid dispersion. In some embodiments, PVP/VA is present in the amorphous solid dispersion in an amount of about 50% to about 65%, about 50% to about 55% or about 60% to about 65% by weight of the amorphous solid dispersion by weight of the amorphous solid dispersion. In some embodiments, the PVP/VA is present in the solid dispersion in an amount of about 50%, about 55%, to about 60% or about 65% by weight of the amorphous solid dispersion. In some embodiments, the PVP/VA is present in the amorphous solid dispersion in an amount of about 65% by weight of the solid dispersion. In some embodiments, the PVP/VA is present in the amorphous solid dispersion in an amount of about 50% by weight of the amorphous solid dispersion. In some embodiments, the weight ratio of otenaproxesul, or pharmaceutically acceptable salt thereof, to the PVP/VA in the amorphous solid dispersion is about 1:1 to about 1:2.

In some embodiments, the amorphous solid dispersion consists essentially of amorphous otenaproxesul, or pharmaceutically acceptable salt thereof, and a polymer. In some embodiments, the amorphous solid dispersion further comprises one or more additives known in the art. In some embodiments, the one or more additives are selected from antioxidants, solubilizers and surfactants.

In some embodiments, the one or more additives are antioxidants. Therefore, in some embodiments, the amorphous solid dispersion further comprises one or more antioxidants.

In some embodiments, the antioxidant is any antioxidant suitable for use with an amorphous solid dispersion comprising otenaproxesul or pharmaceutically acceptable salt thereof and a polymer. Therefore, in some embodiments, the amorphous solid dispersion comprises amorphous otenaproxesul or pharmaceutically acceptable salt thereof, a polymer and optionally one or more antioxidants. In some embodiment, the amorphous solid dispersion consists essentially of amorphous otenaproxesul or pharmaceutically acceptable salt thereof, a polymer and optionally one or more antioxidants.

In some embodiments, the one or more additives are antioxidants and the antioxidants are selected from ascorbic acid, tartaric acid, fumaric acid, citric acid, DL-α-tocopherol, tocopheryl polyethylene glycol succinate (TPGS, Vitamin E TPGS), vitamin A, vitamin C, vitamin D, vitamin E, carotenoids, flavanoids, isoflavanoids, beta-carotene, butylated hydroxytoluene (BHT), butylated hydroxyanisole (BHA), glutathione, lycopene, gallic acid and esters thereof (e.g. propyl gallate), salicylic acid and esters thereof, sulfites, alcohols, amines, amides, sulfoxides, surfactants, and mixtures thereof. In some embodiments, the one or more antioxidants are selected from ascorbic acid, tartaric acid, fumaric acid, citric acid, DL-α-tocopherol, vitamin E TPGS, vitamin A, vitamin C, vitamin D, vitamin E, BHT, BHA, propyl gallate, and mixtures thereof. In some embodiments, the one or more antioxidants are selected from vitamin E TPGS and BHT. In some embodiments, the antioxidant is BHT.

In some embodiments, the one or more antioxidants are present in the amorphous solid dispersion in an amount of about 0.1% to about 1%, about 0.1% to about 0.75%, about 0.1% to about 0.5%, about 0.1% to about 0.4%, about 0.2% to about 0.5%, about 0.2% to about 0.4%, about 0.3% to about 0.5%, or about 0.4% to about 0.5% by weight of the amorphous solid dispersion. In some embodiments, the one or more antioxidants are present in the amorphous solid dispersion in an amount of about 0.2% to about 0.5% about 0.3% to about 0.5%, or about 0.4% to about 0.5% by weight of the amorphous solid dispersion.

In some embodiments, the amorphous otenaproxesul or pharmaceutically acceptable salt thereof in the amorphous solid dispersion comprises a total impurity of less than about 2 wt %, less than about 1.5 wt %, less than about 1 wt %, less than about 0.9 wt %, less than about 0.8 wt %, less than about 0.7 wt %, less than about 0.6 wt %, less than about 0.5 wt %, less than about 0.4 wt %, less than about 0.3 wt %, less than about 0.2 wt % or less than about 0.1 wt %. In some embodiments, the amorphous otenaproxesul or pharmaceutically acceptable salt thereof in the amorphous solid dispersion comprises a total impurity of less than about 0.8 wt %, less than about 0.7 wt %, less than about 0.6 wt %, less than about 0.5 wt %, less than about 0.4 wt %, less than about 0.3 wt %, less than about 0.2 wt % or less than about 0.1 wt %.

In some embodiments, the amorphous otenaproxesul or pharmaceutically acceptable salt thereof in the amorphous solid dispersion comprises a total impurity of less than about 1 wt %, less than about 0.9 wt %, less than about 0.8 wt %, less than about 0.7 wt %, less than about 0.6 wt %, less than about 0.5 wt % after one month at temperature of about 30° C. to about 50° C.

In some embodiments the impurities comprise any metabolite or degradation product of otenaproxesul or pharmaceutically acceptable salt thereof.

In some embodiments, the amorphous solid dispersion comprises less 0.02 wt % (e.g., non-detectable levels) of naproxen when stored at a temperature of about 30° C. or about 50° C. for one month. In some embodiments, the amorphous solid dispersion comprises less than about 0.2 wt %, less than about 0.1 wt % or less than about 0.05 wt % impurity observed at relative retention time (RRT) 0.24 (4-hydroxybenzonitrile) when stored at a temperature of about 30° C. or about 50° C. for one month. In some embodiments, the amorphous solid dispersion comprises less than about 0.2 wt %, less than about 0.1 wt % or less than about 0.05 wt % impurity observed at RRT 0.85 when stored at a temperature of about 30° C. or about 50° C. for one month. In some embodiments, the amorphous solid dispersion comprises less than 0.02 wt % (e.g., non-detectable levels) of impurity observed at RRT 0.85 when stored at a temperature of about 30° C. or about 50° C. for one month. In some embodiments, the amorphous solid dispersion comprises less than about 0.2 wt % or less than about 0.1 wt % impurity observed at RRT 0.85 (otenaproxesul-amide) when stored at a temperature of about 30° C. or about 50° C. for one month. In some embodiments, the amorphous solid dispersion comprises less than about 0.2 wt % or less than about 0.1 wt % impurity observed at RRT 1.11 when stored at a temperature of about 30° C. or about 50° C. for one month.

It would be appreciated by a person skilled in the art that various methods of analyzing (characterization and quantification) the impurities are well established in the art. For example, various spectroscopic techniques, such as NMR, MS, IR etc. and chromatographic techniques, such as HPLC, HPLC-TLC, HPLC-CE and hyphenated methods, such as LC-MS-MS, HPLC-DAD-MS, HPLC-NMR, GC-MS & LC-MS known in the art can be used for analyzing impurities.

In some embodiments, the impurities are analyzed using HPLC. In some embodiments, the impurities are analyzed using HPLC following the protocols described in Example 13. Therefore, in some embodiments HPLC analyses are performed using a C18 150 mm×4.6 mm, 5 μm or equivalent column, with a flow rate of 1.0 mL/min, an injection volume of 10 μL, a column temperature of 25° C., a sample temperature of 5° C. and a wavelength for detection of 254 nm. In some embodiments, the eluents used are (1) A: 1.8 g K₂HPO₄·3H₂O dissolved in 1000 ml of water and pH adjusted to 5.5 using orthophosphoric acid and B: acetonitrile; or (2) A: 1.4 g K₂HPO₄.3H₂O dissolved in 1000 mL of water and pH adjusted to 7.0 with 85% phosphoric acid and B: acetonitrile.

In some embodiments, the amorphous solid dispersions are prepared by spray drying (or lyophilization), melt extrusion, freeze drying, rotary evaporation, solvent-controlled precipitation, pH-controlled precipitation, drum drying, supercritical fluid technology or other solvent removal process. In some embodiments, the amorphous solid dispersion is prepared by spray-draying. Therefore, in some embodiments, the amorphous dispersion is an amorphous spray-dried dispersion (SDD).

In some embodiments, the amorphous solid dispersion comprises particles having a D50 below about 5 μM, below about 45 μM, below about 4 μM, below about 35 UM or below about 30 μM. In some embodiment, the amorphous solid dispersion comprises particles having D50 below about 40 μM, below about 35 UM or below about 32 μM. In some embodiments, the amorphous solid dispersion comprises particles having a D90 below about 80 μM, below about 75 UM or below about 70 μM.

In some embodiments, HPMCAS is present in the amorphous solid dispersion in an amount of about 50% to about 60% or about 60% to about 65% by weight of the amorphous solid dispersion and the amorphous solid dispersion comprises particles having a D50 below about 40 μM, below about 35 UM or below about 32 μM. In some embodiments, HPMCAS is present in the amorphous solid dispersion in an amount of about 65% by weight of the amorphous solid dispersion and the amorphous solid dispersion comprises particles having a D90 below about 75 UM or below about 70 μM. In some embodiments, HPMCAS is present in the amorphous solid dispersion in an amount of about 65% by weight of the amorphous solid dispersion and the amorphous solid dispersion comprises particles having a D90 below about 70 μM. In some embodiments, HPMCAS is present in the amorphous solid dispersion in an amount of about 65% by weight of the amorphous solid dispersion and the amorphous solid dispersion comprises particles having a D90 of 60 UM to about 70 UM

In some embodiments, HPMCAS is present in the amorphous solid dispersion in an amount of about 60% to about 65% by weight of the amorphous solid dispersion and the amorphous solid dispersion comprises particles having a Sauter mean diameter (e.g., surface area mean) [D 3,2] of less than 10 μM. In some embodiments, HPMCAS is present in the amorphous solid dispersion in an amount of about 65% by weight of the amorphous solid dispersion and the amorphous solid dispersion comprises particles having a Sauter mean diameter [D 3,2] of about 8 UM to 10 μM.

In some embodiments, HPMCAS is present in the amorphous solid dispersion in an amount of about 65% by weight of the amorphous solid dispersion and the Tg of the amorphous solid dispersion is about 63° C. to about 64° C.

In some embodiments, the amorphous solid dispersion further comprises one or more sustaining agents.

In some embodiments, the one or more sustaining agents are combined with the otenaproxesul or pharmaceutically acceptable salt thereof and the polymer in the amorphous solid dispersion to form the amorphous solid dispersion comprising otenaproxesul or pharmaceutically acceptable salt thereof, the polymer and the sustaining agent. Therefore, in some embodiments, the sustaining agent is present with the polymer in the amorphous solid dispersion.

In some embodiments, the amorphous solid dispersions are the amorphous solid dispersions provided in Examples 7A and 7B. In some embodiments, the amorphous solid dispersions are the amorphous solid dispersions no. 1 to 8 in Table 5 (Example 7A).

Accordingly, in some embodiments, the amorphous solid dispersion comprises amorphous otenaproxesul or pharmaceutically acceptable salt thereof, a polymer, optionally one or more additives and optionally one or more sustaining polymers. In some embodiments, the one or more additives is one or more antioxidants. In some embodiments, the amorphous solid dispersion comprises amorphous otenaproxesul or pharmaceutically acceptable salt thereof, a polymer, optionally one or more antioxidants and optionally one or more sustaining polymers. In some embodiments, the amorphous solid dispersion consists essentially of amorphous otenaproxesul or pharmaceutically acceptable salt thereof, a polymer, optionally one or more antioxidants and optionally one or more sustaining polymers.

In some embodiments, the amorphous solid dispersions of the application are formulated with one or more conventional excipients known in the art used in the formation of pharmaceutical compositions for administration to subjects in a biologically compatible form suitable for administration in vivo.

Therefore, the present application further includes a pharmaceutical composition comprising an amorphous solid dispersion of the application and one or more pharmaceutically acceptable excipients.

In some embodiments, the amorphous solid dispersion of the application is granulated together with one or more pharmaceutically acceptable excipients to form granules. In some embodiments, the one or more pharmaceutically acceptable excipients comprise one or more intragranular pharmaceutically acceptable excipients and one or more extragranular pharmaceutically acceptable excipients.

Accordingly, the present application includes a pharmaceutical composition comprising an amorphous solid dispersion of the application, and one or more intragranular pharmaceutically acceptable excipients and/or one or more extragranular pharmaceutically acceptable excipients. In some embodiments, the amorphous solid dispersion is granulated together with the one or more intragranular pharmaceutically acceptable excipients to form granules. Accordingly, in some embodiments, the present application includes a pharmaceutical composition comprising a granule comprising an amorphous solid dispersion of the application, and one or more intragranular pharmaceutically acceptable excipients; and one or more extragranular pharmaceutically acceptable excipients.

In some embodiments, the pharmaceutical composition comprises about 25% to about 65% of the amorphous solid dispersion of the application, by weight of the composition. In some embodiments, the pharmaceutical composition comprises about 35% to about 65% of the amorphous solid dispersion of the application by weight of the composition. In some embodiments, the pharmaceutical composition comprises about 40% of the amorphous solid dispersion by weight of the composition. In some embodiments, the pharmaceutical composition comprises about 65% of the amorphous solid dispersion of the application by weight of the composition. In some embodiments, the pharmaceutical composition comprises about 30% to about 35% of the amorphous solid dispersion of the application by weight of the composition.

In some embodiments, the pharmaceutical composition optionally further comprises one or more sustaining agents external to the amorphous solid dispersion. In some embodiments, the sustaining agent is combined with the amorphous solid dispersion after the amorphous solid dispersion has been formed. In some embodiments, the amorphous solid dispersion is granulated together with the one or more intragranular pharmaceutically acceptable excipients and the one or more optional sustaining agents to form granules. Accordingly, in some embodiments, the present application includes a pharmaceutical composition comprising:
a granule comprising an amorphous solid dispersion of the application and one or more intragranular pharmaceutically acceptable excipients;
one or more extragranular pharmaceutically acceptable excipients; and
optionally one or more sustaining agents,
wherein the one or more sustaining agents are present in the granule external to the amorphous solid dispersion and/or are present in the amorphous solid dispersion.

In some embodiments, the weight ratio of the one or more sustaining agents to amorphous solid dispersion is about 1:1 to about 2:5. In some embodiments, the weight ratio of the one or more sustaining agents to amorphous solid dispersion is about 1:1 to about 2:3.

In some embodiments, the one or more sustaining agents is in an amount of about 35% to about 60%, about 35% to about 50%, about 35% to about 40% or about 40% by weight of the granule. In some embodiments, the one or more sustaining agents is in an amount about 35% to about 40% or about 40% by weight of the granule.

In some embodiments, the one or more sustaining agents are one or more sustaining agents as described above. In some embodiments, the one or more sustaining agents is a polymer. In some embodiments, the one or more sustaining agents are selected from HPMCAS and PVP.

In some embodiments, the one or more sustaining agent external to the solid dispersion is HPMCAS. In some embodiments, the amorphous solid dispersion is granulated together with the one or more intragranular pharmaceutically acceptable excipients and HPMCAS to form granules.

In some embodiments, the amorphous solid dispersion comprises PVP/VA in an amount of from 55% to about 65% by weight of the amorphous solid dispersion, and HPMCAS is present external to the amorphous solid dispersion in a weight ratio of sustaining agent to amorphous solid dispersion of about 1:1.5. In some embodiments, the amorphous solid dispersion comprises PVP/VA in an amount of from 55% to about 65% by weight of the amorphous solid dispersion and HPMCAS is present external to the amorphous solid dispersion in a weight percent ratio of sustaining agent to amorphous solid dispersion of about 1:1.5.

In some embodiments, the HPMCAS is present external to the amorphous solid dispersion in the granule in an amount of about 35% to about 60%, about 35% to about 50%, about 35% to about 40% or about 40% by weight of the granule. In some embodiments, the HPMCAS is present external to the amorphous solid dispersion in the granule in an amount about 35% to about 40% or about 40% by weight of the granule.

In some embodiments, the pharmaceutical composition comprises about 30% to about 60% of the one or more intragranular pharmaceutically acceptable excipients wherein the percentage amount is by total weight of the pharmaceutical composition. In some embodiments, the pharmaceutical composition comprises about 30% to about 35% of the one or more intragranular pharmaceutically acceptable excipients wherein the percentage amount is by weight of the pharmaceutical composition. In some embodiments, the amorphous solid dispersion of the application and the one or more intragranular pharmaceutically acceptable excipients are granulated together to form granules.

In some embodiments, the one or more intragranular pharmaceutically acceptable excipients are selected from one or more intragranular pharmaceutically acceptable additives, fillers, disintegrants, lubricants, and glidants. In some embodiments, the one or more intragranular pharmaceutically acceptable excipients are selected from one or more intragranular pharmaceutically acceptable fillers, disintegrants, lubricants and glidants.

In some embodiments, the pharmaceutical composition comprises about 0.5% to about 3% of the one or more intragranular pharmaceutically acceptable additives wherein the percentage amount is by weight of the pharmaceutical composition. In some embodiments, the pharmaceutical composition comprises about 0.5% to about 2% of the one or more intragranular pharmaceutically acceptable additives wherein the percentage amount is by weight of the pharmaceutical composition.

In some embodiments, the pharmaceutical composition comprises about 30% to about 50% of the one or more intragranular pharmaceutically acceptable fillers wherein the percentage amount is by weight of the pharmaceutical composition. In some embodiments, the pharmaceutical composition comprises about 25% to about 50% or about 25% to about 45%, about 30% to about 40% or about 30% to about 35% of the one or more intragranular pharmaceutically acceptable fillers wherein the percentage amount is by weight of the pharmaceutical composition.

In some embodiments, the pharmaceutical composition further comprises about 2% to about 8% of one or more intragranular pharmaceutically acceptable disintegrants wherein the percentage amount is by weight of the pharmaceutical composition. In some embodiments, the pharmaceutical composition further comprises about 2% to about 5% of one or more intragranular pharmaceutically acceptable disintegrants wherein the percentage amount is by weight of the pharmaceutical composition.

In some embodiments, the pharmaceutical composition further comprises about 0.1% to about 3%, about 0.1% to about 2%, about 0.1% to about 1% of one or more intragranular pharmaceutically acceptable glidants and lubricants wherein the percentage amount is by weight of the pharmaceutical composition.

In some embodiments, the pharmaceutical composition comprises about 45% to about 65% of an amorphous solid dispersion of the application;
about 25% to about 50% of one or more intragranular pharmaceutically acceptable fillers;
about 2% to about 8% of one or more intragranular pharmaceutically acceptable disintegrants,
about 0.1% to about 2% of one or more intragranular pharmaceutically acceptable glidants and lubricants;
optionally about 0.5% to about 2% of the one or more intragranular pharmaceutically acceptable additives;
wherein the percentage amount is by weight of the pharmaceutical composition.

In some embodiments, the pharmaceutical composition comprises:
about 55% to about 65% of an amorphous solid dispersion of the application;
about 25% to about 35% of one or more intragranular pharmaceutically acceptable fillers;
about 2% to about 6% of one or more intragranular pharmaceutically acceptable disintegrants,
about 0.1% to about 1% of one or more intragranular pharmaceutically acceptable glidants and lubricants, and
optionally about 1% to about 2% of the one or more intragranular pharmaceutically acceptable additives;
wherein the percentage amount is by weight of the pharmaceutical composition.

In some embodiments, the pharmaceutical composition comprises about 40% to about 60% of an amorphous solid dispersion of the application; about 25% to about 50% of one or more intragranular pharmaceutically acceptable fillers, about 2% to about 8% of one or more intragranular pharmaceutically acceptable disintegrants, about 0.1% to about 1% of one or more intragranular pharmaceutically acceptable glidants and lubricants, wherein the percentage amount is by weight of the pharmaceutical composition.

In some embodiments, the pharmaceutical composition comprises about 25% to about 40% of a solid dispersion of the application, about 15% to about 25% of one or more sustaining agents; about 1% to about 2% of the one or more intragranular pharmaceutically acceptable additives; about 10% to about 30% of one or more intragranular pharmaceutically acceptable fillers, about 2% to about 8% of one or more intragranular pharmaceutically acceptable disintegrants, about 0.1% to about 1% of one or more intragranular pharmaceutically acceptable glidants and lubricants, wherein the percentage amount is by weight of the composition.

In some embodiments, the pharmaceutical composition comprises about 25% to about 40% of a solid dispersion of the application, about 15% to about 25% of one or more sustaining agents; about 10% to about 30% of one or more intragranular pharmaceutically acceptable fillers, about 2% to about 8% of one or more intragranular pharmaceutically acceptable disintegrants, about 0.1% to about 1% of one or more intragranular pharmaceutically acceptable glidants and lubricants, wherein the percentage amount is by weight of the composition.

In some embodiments, the one or more extragranular pharmaceutically acceptable excipients are selected from one or more extragranular pharmaceutically acceptable fillers (including diluents), disintegrants, lubricants and glidants. In some embodiments, the one or more extragranular pharmaceutically acceptable excipients are selected from one or more extragranular pharmaceutically acceptable disintegrants, lubricants and glidants. In some embodiments, the pharmaceutical composition comprises about 2% to about 8% of the one or more extragranular pharmaceutically acceptable disintegrants and about 0.1% to about 1% of the one or more extragranular pharmaceutically acceptable glidants and lubricants extragranularly wherein the percentage amount is by weight of the composition.

In some embodiments, the one or more additives are selected from antioxidants, solubilizing agents and surfactants. In some embodiments, the one or more additives are selected from antioxidants and solubilizing agents.

In some embodiments, the one or more antioxidants are any antioxidants known in the art. In some embodiments, the one or more antioxidants are one or more antioxidants as described above. In some embodiments, the one or more antioxidants are selected from ascorbic acid, tartaric acid, fumaric acid, citric acid, DL-α-tocopherol, TPGS, vitamin A, vitamin C, vitamin D, vitamin E, butylated hydroxytoluene (BHT), butylated hydroxyanisole (BHA), propyl gallate, and combinations thereof. In some embodiments, the one or more antioxidants are selected from ascorbic acid and citric acid.

In some embodiments, the one or more solubilizing agents are any agents that are known to increase the solubility of an active agent. In some embodiments, the one or more solubilizing agents are selected from trehalose, methylcellulose, ethylcellulose, sodium carboxymethylcellulose, hydroxypropylmethylcellulose, sodium hyaluronate, sodium alginate, chitosan and its derivatives, polyethylene glycol (PEG) and mono-, di- and triglycerides and mono- and diesters of PEG, glycerin, propylene glycol, Triacetin, N,N-dimethylacetamide, poly(vinyl pyrrolidone), Labrasol®, pyrrolidone, dimethyl sulfoxide, N-(-beta-Hydroxyethyl)-lactamide, 1-methyl-2-pyrrolidinone, triglycerides, monothioglycerol, sorbitol, lecithin, methylparaben, propylparaben, sodium taurocholate, poloxamer, copovidone, diethylene glycol monoethyl ethe (Transcutol®), propylene glycol, polyoxyethylene sorbitan (e.g. polysorbates, Tween), sodium lauryl sulfate, polyoxyl-ethylated castor oils (e.g. Cremophor®), poloxamer (e.g. Pluronic® and Lutrol®) and meglumine. In some embodiments, the one or more solubilizing agent is selected from sodium lauryl sulfate, poloxamer and meglumine.

In some embodiments, the one or more surfactants are any agent that is known in the art to lower the surface tension between a liquid and a solid that could improve the wetting of the active agent or improve the solubility of an active agent. In some embodiments, the one or more surfactants are selected from sodium lauryl sulfate, sodium laureth sulfate, fatty acid esters of polyoxyethylene sorbitan (e.g. polysorbates, Tween), polyoxyl-ethylated castor oils (e.g. Cremophor®), poloxamer (e.g. Pluronic® and Lutrol®), polyoxyethylene esters of 12-hydroxystearic acid (e.g. Solutol®), polyethylene glycol, polyvinylpyrrolidone, hydroxypropylcellulose, hydroxypropyl cellulose ethers, hydroxypropyl methylcellulose, hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, polyvinyl alcohol (PVA), polyvinylpyrrolidone/vinyl acetate copolymer, and poloxamines. In some embodiments, the one or more surfactants is selected from sodium lauryl sulfate and poloxamer.

In some embodiments, the one or more intragranular and extragranular pharmaceutically fillers (or diluents) are independently selected from lactose, mannitol, sorbitol, isomalt, xylitol, microcrystalline cellulose, silicified microcrystalline cellulose, calcium diphosphate, and starch and mixtures thereof. In some embodiments, the one or more intragranular and extragranular pharmaceutically fillers are independently selected from lactose, mannitol, and microcrystalline cellulose and mixtures thereof. In some embodiments, the one or more intragranular and extragranular pharmaceutically fillers are independently selected from mannitol, and microcrystalline cellulose and mixtures thereof.

In some embodiments, the one or more intragranular or extragranular pharmaceutically disintegrants are any agent known in the art which facilitates the break-up of a solid preparation or disintegration after administration or use. In some embodiments, the one or more intragranular or extragranular pharmaceutically disintegrants are independently selected from sodium starch glycolate, sodium alginate, carboxymethyl cellulose sodium, methyl cellulose, croscarmellose sodium, croscarmellose calcium, guar gum, low substituted hydroxypropyl cellulose and crospovidone and mixtures thereof. In some embodiments, the one or more intragranular or extragranular pharmaceutically disintegrants are independently selected from carboxymethyl cellulose sodium and crospovidone and mixtures thereof.

In some embodiments, the one or more intragranular and extragranular pharmaceutically lubricants are any agent known in the art that is added to a powder blend to prevent the compacted powder mass from sticking to the equipment during the tableting or encapsulation process. In some embodiments, the one or more intragranular and extragranular pharmaceutically lubricants are independently selected from magnesium stearate, calcium stearate, zinc stearate, glyceryl behenate, sodium stearyl fumarate, hydrogenated vegetable oil, hydrogenated castor oil, glyceryl palmitostearate, stearic acid, sucrose fatty acid esters and sodium benzoate and mixtures thereof. In some embodiments, intragranular and extragranular pharmaceutically the lubricant is magnesium stearate.

In some embodiments, the one or more intragranular and extragranular pharmaceutically glidants are any agent known in the art that are used in tablet and capsule formulations to improve flow-properties during tablet or capsule compression. In some embodiments, the one or more intragranular and extragranular pharmaceutically glidants are independently selected from calcium phosphate tribasic, powdered cellulose, silicon dioxide, magnesium silicate, magnesium trisilicate and talc and mixtures thereof. In some embodiments, the intragranular and extragranular pharmaceutically glidant is silicon dioxide.

A person skilled in the art will appreciate that there is overlap between the above described additives and excipients used in the pharmaceutical compositions herein, since a given additive or excipients is often classified differently by different person of skilled in the art or is commonly used for any of several different functions. The above-listed additives should be taken as merely exemplary, and not limiting, of the types of additives.

In an exemplary embodiment, the pharmaceutical composition comprises:
about 55% to about 65% by weight of the composition of an amorphous solid dispersion comprising amorphous otenaproxesul or pharmaceutically acceptable salt thereof and HPMCAS wherein the HPMCAS is present in the amorphous solid dispersion in an amount of about 50% to about 60%, about 55% to about 65%, about 60% to about 65% or about 60% by weight of the amorphous solid dispersion;
about 30% to about 35% by weight of the composition of one or more intragranular pharmaceutically acceptable fillers;
about 2% to about 4% by weight of the composition of one or more intragranular pharmaceutically acceptable disintegrants;
about 0.1% to about 1% by weight of the composition of one or more intragranular pharmaceutically acceptable glidants and lubricants, and
optionally about 0.5% to about 2% by weight of the composition of one or more intragranular pharmaceutically acceptable additives.

In some embodiments, in a further exemplary embodiment, the pharmaceutical composition comprises:
about 55% to about 65% by weight of the composition of an amorphous solid dispersion comprising amorphous otenaproxesul or pharmaceutically acceptable salt thereof and HPMCAS wherein the HPMCAS is present in the amorphous solid dispersion in an amount of about 60% to about 65% by weight of the amorphous solid dispersion;
about 1% to about 2% by weight of the composition of one or more intragranular pharmaceutically acceptable additives;
about 30% to about 35% by weight of the composition of one or more intragranular pharmaceutically acceptable fillers selected from mannitol and microcrystalline cellulose and mixtures thereof;
about 2% to about 4% by weight of the composition of one or more intragranular pharmaceutically acceptable disintegrants selected from carboxymethyl cellulose sodium and crospovidone and mixtures thereof; and
about 0.1% to about 1% by weight of the composition of one or more intragranular pharmaceutically acceptable glidants and lubricants.

In some embodiments, the pharmaceutical composition comprises:
about 60% by weight of the composition of an amorphous solid dispersion comprising amorphous otenaproxesul or pharmaceutically acceptable salt thereof and HPMCAS wherein the HPMCAS is present in the amorphous solid dispersion in an amount of about 65% by weight of the amorphous solid dispersion;
about 30% to about 35% by weight of the composition of one or more intragranular pharmaceutically acceptable fillers selected from an equal mixture of mannitol and microcrystalline cellulose;
about 3% by weight of the composition of one or more intragranular pharmaceutically acceptable disintegrants wherein the disintegrant is carboxymethyl cellulose sodium;
about 0.1% to about 1% by weight of the composition of one or more intragranular pharmaceutically acceptable glidants and lubricants, and
optionally about 0.5% to about 2% by weight of the composition of one or more intragranular pharmaceutically acceptable additives selected from antioxidants and solubilizers.

In some embodiments, the pharmaceutical composition comprises: about 60% by weight of the composition of an amorphous solid dispersion comprising amorphous otenaproxesul or pharmaceutically acceptable salt thereof and HPMCAS wherein the HPMCAS is present in the amorphous solid dispersion in an amount of about 65% by weight of the amorphous solid dispersion;
about 32% by weight of the composition of one or more intragranular pharmaceutically acceptable fillers selected from an equal mixture of mannitol and microcrystalline cellulose;
about 3% by weight of the composition of one or more intragranular pharmaceutically acceptable disintegrants wherein the disintegrant is carboxymethyl cellulose sodium;
and about 0.1% to about 1% by weight of the composition of one or more intragranular pharmaceutically acceptable glidants and lubricants and
optionally about 1% to about 2% by weight of the composition of one or more intragranular pharmaceutically acceptable additives selected from antioxidants, solubilizers and surfactants.

In an exemplary embodiment, the pharmaceutical composition comprises:
about 55% to about 65% by weight of the composition of an amorphous solid dispersion comprising amorphous otenaproxesul or pharmaceutically acceptable salt thereof and HPMCAS wherein the HPMCAS is present in the amorphous solid dispersion in an amount of about 50% to about 60%, about 55% to about 65% or about 60% to about 65% by weight of the amorphous solid dispersion;
about 30% to about 35% by weight of the composition of one or more intragranular pharmaceutically acceptable fillers;
about 2% to about 4% by weight of the composition of one or more intragranular pharmaceutically acceptable disintegrants; and
about 0.1% to about 1% by weight of the composition of one or more intragranular pharmaceutically acceptable glidants and lubricants.

In some embodiments, the pharmaceutical composition comprises:
about 55% to about 65% by weight of the composition of an amorphous solid dispersion comprising amorphous otenaproxesul or pharmaceutically acceptable salt thereof and HPMCAS wherein the HPMCAS is present in the amorphous solid dispersion in an amount of about 60% to about 65% by weight of the amorphous solid dispersion;
about 30% to about 35% by weight of the composition of one or more intragranular pharmaceutically acceptable fillers selected from mannitol and microcrystalline cellulose and mixtures thereof;
about 2% to about 4% by weight of the composition of one or more intragranular pharmaceutically acceptable disintegrants selected from carboxymethyl cellulose sodium and crospovidone and mixtures thereof; and
about 0.1% to about 1% by weight of the composition of one or more intragranular pharmaceutically acceptable glidants and lubricants.

In some embodiments, the pharmaceutical composition comprises:
about 60% by weight of the composition of an amorphous solid dispersion comprising amorphous otenaproxesul or pharmaceutically acceptable salt thereof and HPMCAS wherein the HPMCAS is present in the amorphous solid dispersion in an amount of about 65% by weight of the amorphous solid dispersion;
about 32% by weight of the composition of one or more intragranular pharmaceutically acceptable fillers selected from an equal mixture of mannitol and microcrystalline cellulose;
about 3% by weight of the composition of one or more intragranular pharmaceutically acceptable disintegrants wherein the disintegrant is carboxymethyl cellulose sodium; and
about 0.1% to about 1% by weight of the composition of one or more intragranular pharmaceutically acceptable glidants and lubricants.

In some embodiments, the HPMCAS is HPMCAS-H. In some embodiments, the HPMCAS is HPMCAS-HG.

In an exemplary embodiment, the pharmaceutical composition comprises: about 35% to about 45% by weight of the composition of an amorphous solid dispersion comprising amorphous otenaproxesul or pharmaceutically acceptable salt thereof and PVP/VA wherein the PVP/VA is present in the amorphous solid dispersion in an amount of about 50% to about 60%, about 55% to about 65% or about 60% to about 65% by weight of the amorphous solid dispersion;
about 45% to about 55% by weight of the composition of one or more intragranular pharmaceutically acceptable fillers;
about 4% to about 8% by weight of the composition of one or more intragranular pharmaceutically acceptable disintegrants;
about 0.1% to about 1% by weight of the composition of one or more intragranular pharmaceutically acceptable glidants and lubricants; and
optionally about 1% to about 2% by weight of the composition of one or more intragranular pharmaceutically acceptable additives.

In an exemplary embodiment, the pharmaceutical composition comprises: about 35% to about 45% by weight of the composition of an amorphous solid dispersion comprising amorphous otenaproxesul or pharmaceutically acceptable salt thereof and PVP/VA wherein the PVP/VA is present in the amorphous solid dispersion in an amount of about 50% to about 60%, about 55% to about 65% or about 60% to about 65% by weight of the amorphous solid dispersion;
about 45% to about 55% by weight of the composition of one or more intragranular pharmaceutically acceptable fillers;
about 4% to about 8% by weight of the composition of one or more intragranular pharmaceutically acceptable disintegrants; and
about 0.1% to about 1% by weight of the composition of one or more intragranular pharmaceutically acceptable glidants and lubricants.

In some embodiments, the pharmaceutical composition comprises:
about 35% to about 45% by weight of the composition of an amorphous solid dispersion comprising amorphous otenaproxesul or pharmaceutically acceptable salt thereof and PVP/VA wherein the PVP/VA is amorphous solid dispersion in an amount of about 60% to about 65% by weight of the amorphous solid dispersion;
about 45% to about 55% by weight of the composition of one or more intragranular pharmaceutically acceptable fillers selected from mannitol and microcrystalline cellulose and mixtures thereof;
about 4% to about 8% by weight of the composition of one or more intragranular pharmaceutically acceptable disintegrants selected from carboxymethyl cellulose sodium and crospovidone and mixtures thereof;
and about 0.1% to about 1% by weight of the composition of one or more intragranular pharmaceutically acceptable glidants and lubricants. In some embodiments, the amorphous solid dispersion further comprises about 0.2% to about 0.5% of one or more antioxidants by weight of the amorphous solid dispersion.

In some embodiments, the pharmaceutical composition comprises:
about 40% by weight of the composition of an amorphous solid dispersion comprising amorphous otenaproxesul or pharmaceutically acceptable salt thereof and PVP/VA wherein the PVP/VA is amorphous solid dispersion in an amount of about 65% by weight of the amorphous solid dispersion;
about 50% by weight of the composition of one or more intragranular pharmaceutically acceptable fillers selected from an equal mixture of mannitol and microcrystalline cellulose;
about 6% by weight of the composition of one or more intragranular pharmaceutically acceptable disintegrants wherein the disintegrant is crospovidone;
and about 0.1% to about 1% by weight of the composition of one or more intragranular pharmaceutically acceptable glidants and lubricants. In some embodiments, the amorphous solid dispersion further comprises about 0.2% to about 0.5% of one or more antioxidants by weight of the amorphous solid dispersion.

In some embodiments, the pharmaceutical composition comprises:
about 25% to about 35% by weight of the composition of an amorphous solid dispersion comprising amorphous otenaproxesul or pharmaceutically acceptable salt thereof and PVP/VA wherein the PVP/VA is present in the amorphous solid dispersion in an amount of about 40% to about 65% or about 45% to about 55% by weight of the amorphous solid dispersion;
about 15% to about 25% by weight of the composition of HPMCAS;
about 30% to about 40% by weight of the composition of one or more intragranular pharmaceutically acceptable fillers; about 4% to about 8% by weight of the composition of one or more intragranular pharmaceutically acceptable disintegrants;
about 0.1% to about 1% by weight of the composition of one or more intragranular pharmaceutically acceptable glidants and lubricants; and
optionally about 1% to about 2% by weight of the composition of one or more intragranular pharmaceutically acceptable additives.

In some embodiments, the pharmaceutical composition comprises:
about 25% to about 35% by weight of the composition of an amorphous solid dispersion comprising amorphous otenaproxesul or pharmaceutically acceptable salt thereof and PVP/VA wherein the PVP/VA is present in the amorphous solid dispersion in an amount of about 40% to about 65% or about 45% to about 55% by weight of the amorphous solid dispersion;
about 15% to about 25% by weight of the composition of HPMCAS;
about 30% to about 40% by weight of the composition of one or more intragranular pharmaceutically acceptable fillers;
about 4% to about 8% by weight of the composition of one or more intragranular pharmaceutically acceptable disintegrants; and
about 0.1% to about 1% by weight of the composition of one or more intragranular pharmaceutically acceptable glidants and lubricants.

In some embodiments, the pharmaceutical composition comprises:
about 25% to about 35% by weight of the composition of an amorphous solid dispersion comprising amorphous otenaproxesul or pharmaceutically acceptable salt thereof and a PVP/VA wherein the PVP/VA is present in the amorphous solid dispersion in an amount of about 45% to about 55% by weight of the amorphous solid dispersion;
about 15% to about 25% by weight of the composition of HPMCAS;
about 30% to about 40% by weight of the composition of one or more intragranular pharmaceutically acceptable fillers selected from mannitol and microcrystalline cellulose and mixtures thereof
about 4% to about 8% by weight of the composition of one or more intragranular pharmaceutically acceptable disintegrants selected from carboxymethyl cellulose sodium and crospovidone and mixtures thereof; and
about 0.1% to about 1% by weight of the composition of one or more intragranular pharmaceutically acceptable glidants and lubricants. In some embodiments, the amorphous solid dispersion further comprises about 0.2% to about 0.5% of one or more antioxidants by weight of the amorphous solid dispersion.

In some embodiments, the pharmaceutical composition comprises:
about 31% by weight of the composition of an amorphous solid dispersion comprising amorphous otenaproxesul or pharmaceutically acceptable salt thereof and a PVP/VA wherein the PVP/VA is present in the amorphous solid dispersion in an amount of about 50% by weight of the amorphous solid dispersion:
about 21% by weight of the composition of HPMCAS;
about 36% by weight of the composition of one or more intragranular pharmaceutically acceptable fillers selected from an equal mixture of mannitol and microcrystalline cellulose;
about 6% by weight of the composition of one or more intragranular pharmaceutically acceptable disintegrants wherein the disintegrant is crospovidone;
and about 0.1% to about 1% by weight of the composition of one or more intragranular pharmaceutically acceptable glidants and lubricants. In some embodiments, the amorphous solid dispersion further comprises about 0.2% to about 0.5% of one or more antioxidants by weight of the amorphous solid dispersion.

In some embodiments, the pharmaceutical composition further comprises about 2% to about 8% of one or more extragranular pharmaceutically acceptable disintegrants selected from carboxymethyl cellulose sodium and crospovidone and mixtures thereof, and about 0.1% to about 1% of one or more extragranular pharmaceutically acceptable glidants and lubricants extragranularly by weight of the composition.

In some embodiments, the pharmaceutical composition further comprises a film coat. In some embodiments, the film coat comprises one or more film-forming substances selected from hydroxypropyl methyl cellulose, polyethylene glycol, propyl cellulose, methyl cellulose, polyvinyl alcohol, polymethacrylates and carrageen and mixtures thereof. In some embodiments, the film coat is an Opadry® film coating system.

In some embodiments, the pharmaceutical compositions are formulated for administration, or use, by oral delivery. In some embodiments, the pharmaceutical compositions are formulated as solid or semi-solid oral formulations. In some embodiments, the pharmaceutical compositions are formulated as solid oral compositions or formulations.

In some embodiments, the pharmaceutical compositions are formulated in the form of a tablet such as ingestible tablets or buccal tablets, troches, capsules, caplets, pellets, granules, lozenges, chewing gum, powders, syrups, elixirs, wafers, aqueous solutions or suspensions, and the like. In some embodiments, the pharmaceutical compositions are formulated for administration, or use, as a tablet.

In some embodiments, the present application includes a tablet comprising: amorphous otenaproxesul or a pharmaceutically acceptable salt thereof, dispersed in a polymer matrix formed from a pharmaceutically acceptable polymer; one or more pharmaceutical acceptable ingredients selected from the group consisting of one or more diluents, one or more disintegrants, one or more lubricants, one or more glidants; and optionally one or more film coating agents.

In some embodiments, the amorphous otenaproxesul or a pharmaceutically acceptable salt thereof is present in the pharmaceutical composition in an amount of about 5 mg to about 400 mg. In some embodiments, the amorphous otenaproxesul or a pharmaceutically acceptable salt thereof is present in the pharmaceutical composition in an amount of about 5 mg, about 10 mg, about 25 mg, about 50 mg, about 75 mg, about 100 mg, about 150 mg, about 200 mg, about 250 mg, about 300 mg, about 350 mg or about 400 mg. In some embodiments, the amorphous otenaproxesul or a pharmaceutically acceptable salt thereof is present in the pharmaceutical composition in an amount of about 25 mg, about 50 mg, about 75 mg, about 100 mg, about 150 mg, about 200 mg or about 250 mg. In some embodiments, the amorphous otenaproxesul or a pharmaceutically acceptable salt thereof is present in the pharmaceutical composition in an amount of about 200 mg.

In some embodiments, the pharmaceutical composition is formulated to comprise a loading dose or a maintenance dose of amorphous otenaproxesul or a pharmaceutically acceptable salt thereof.

In some embodiments, the loading dose of amorphous otenaproxesul or a pharmaceutically acceptable salt thereof is about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 400 mg, or about 100 mg to about 200 mg. In some embodiments, the loading dose of amorphous otenaproxesul or a pharmaceutically acceptable salt thereof is about 50 mg to about 600 mg, about 50 mg to about 400 mg, about 100 mg to about 400 mg, about 200 mg to about 600 mg, or about 200 mg to about 400 mg. In some embodiments, the loading dose of amorphous otenaproxesul or a pharmaceutically acceptable salt thereof is about 50 mg to about 200 mg, about 50 mg to about 100 mg, or about 100 mg to about 200 mg. In some embodiments, the loading dose of amorphous otenaproxesul or a pharmaceutically acceptable salt thereof is about 25 mg, about 50 mg, about 75 mg, about 100 mg, about 150 mg, about 200 mg, about 250 mg, about 300 mg, about 350 mg, about 400 mg, about 450 mg, about 475 mg, about 500 mg, about 525 mg, about 550 mg, about 575 mg, or about 600 mg. In some embodiments, the loading dose of amorphous otenaproxesul or a pharmaceutically acceptable salt thereof is about 150 mg, about 200 mg, about 250 mg, about 300 mg, about 350 mg, about 400 mg, about 450 mg, about 500 mg, about 550 mg or about 600 mg. In some embodiments, the loading dose of amorphous otenaproxesul or a pharmaceutically acceptable salt thereof is about 25 mg, about 50 mg, about 75 mg, about 100 mg, about 150 mg or about 200 mg. In some embodiments, the loading dose of amorphous otenaproxesul or a pharmaceutically acceptable salt thereof is about 200 mg, about 400 mg or about 600 mg.

In some embodiments, the maintenance dose of amorphous otenaproxesul or a pharmaceutically acceptable salt thereof is about 5 mg to about 100 mg, about 10 mg to about 100 mg, about 10 mg to about 50 mg, about 25 mg to about 100 mg, about 25 mg to about 50 mg, or about 50 mg to about 100 mg, about 25 mg to about 150 mg, about 25 mg to about 200 mg, or about 25 mg to about 250 mg. In some embodiments, the maintenance dose of amorphous otenaproxesul or a pharmaceutically acceptable salt thereof is about 5 mg to about 100 mg, about 10 mg to about 100 mg, about 10 mg to about 50 mg, about 25 mg to about 100 mg, about 25 mg to about 50 mg, or about 50 mg to about 100 mg. In some embodiments, the maintenance dose of amorphous otenaproxesul or a pharmaceutically acceptable salt thereof is about 25 mg to about 200 mg. In some embodiment, the maintenance dose of amorphous otenaproxesul or a pharmaceutically acceptable salt thereof is about 5 mg, about 10 mg, about 25 mg about 50 mg, about 75 mg, about 100 mg, about 125 mg, about 150 mg, about 175 mg or about 200 mg. In some embodiments, the maintenance dose of amorphous otenaproxesul or a pharmaceutically acceptable salt thereof is about 5 mg, 10 mg, about 25 mg about 50 mg, about 75 mg, or about 100 mg. In some embodiments, the maintenance dose of amorphous otenaproxesul or a pharmaceutically acceptable salt thereof is about 25 mg, about 50 mg, about 100 mg or about 200 mg.

In some embodiments, the pharmaceutical compositions of the application comprising a loading dose or a maintenance dose of amorphous otenaproxesul or a pharmaceutically acceptable salt thereof are provided as a single pharmaceutical composition of the application comprising the loading dose or a maintenance dose or as two or more pharmaceutical compositions of the application each comprising divided doses of amorphous otenaproxesul or a pharmaceutically acceptable salt thereof, wherein divided doses combined provide the loading dose or a maintenance dose. Therefore, in some embodiments, the loading dose or the maintenance dose is divided into a plurality of individual doses. In some embodiments, the plurality of individual doses is suitably formulated into a two or more pharmaceutical compositions respectively comprising a divided dose wherein the sum of the divided doses equals the loading dose or the maintenance dose. Therefore, in some embodiments, the pharmaceutical composition comprises a divided dose of amorphous otenaproxesul or a pharmaceutically acceptable salt thereof, wherein the sum of the divided doses equals the dose. In some embodiments, a dose of amorphous otenaproxesul or a pharmaceutically acceptable salt thereof is divided into 2 to 6, 2 to 5, 2 to 4, or 2 to 3 individual doses which can be suitably formulated into 2 to 6, 2 to 5, 2 to 4, or 2 to 3 pharmaceutical compositions respectively comprising one individual dose wherein the sum of the individual doses equals the dose. Therefore, in some embodiments, a pharmaceutical composition comprises a divided dose of amorphous otenaproxesul or a pharmaceutically acceptable salt thereof, and the divided dose is ⅙, ⅕, ¼, ⅓ or ½ of the dose of amorphous otenaproxesul or a pharmaceutically acceptable salt thereof.

In some embodiments, the pharmaceutical composition comprises one dose per day (e.g., a daily dose) of amorphous otenaproxesul or a pharmaceutically acceptable salt thereof. In some embodiments, a dose per day of amorphous otenaproxesul or a pharmaceutically acceptable salt thereof is divided into a plurality of individual doses. In some embodiments, the plurality of individual doses is suitably formulated into a two or more pharmaceutical compositions respectively comprising a divided dose wherein the sum of the divided doses equals the dose per day, respectively. Therefore, in some embodiments, the pharmaceutical composition comprises a divided dose of amorphous otenaproxesul or a pharmaceutically acceptable salt thereof, wherein the sum of the divided doses equals the dose per day. In some embodiments, a dose per day of amorphous otenaproxesul or a pharmaceutically acceptable salt thereof is divided into 2 to 6, 2 to 5, 2 to 4, or 2 to 3 individual doses which can be suitably formulated into 2 to 6, 2 to 5, 2 to 4, or 2 to 3 pharmaceutical compositions respectively comprising one individual dose wherein the sum of the individual doses equals the dose per day. Therefore, in some embodiments, a pharmaceutical composition comprises a divided dose of amorphous otenaproxesul or a pharmaceutically acceptable salt thereof, and the divided dose is ⅙, ⅕, ¼, ⅓ or ½ of the dose per day of amorphous otenaproxesul or a pharmaceutically acceptable salt thereof.

In an exemplary embodiment, the loading dose of amorphous otenaproxesul or a pharmaceutically acceptable salt thereof is divided into two or three individual doses which can be suitably formulated into two or three pharmaceutical compositions comprising one individual dose wherein the sum of the individual doses equals the loading dose, respectively. Therefore, in an exemplary embodiment, a pharmaceutical composition comprises a divided dose of amorphous otenaproxesul or a pharmaceutically acceptable salt thereof, and the divided dose is ⅓ or ½ of the dose of amorphous otenaproxesul or a pharmaceutically acceptable salt thereof. In some embodiments, two or three pharmaceutical compositions of the application each comprise a divided dose wherein the sum of the two or three divided doses equals the dose. Therefore, in an exemplary embodiment, a pharmaceutical composition comprises a divided dose of amorphous otenaproxesul or a pharmaceutically acceptable salt thereof, and the divided dose is ⅓ or ½ of the dose per day of amorphous otenaproxesul or a pharmaceutically acceptable salt thereof. In some embodiments, two or three pharmaceutical compositions of the application each comprise a divided dose wherein the sum of the two or three divided doses equals the dose per day.

In some embodiments, the compositions of the application comprising 15 mg/kg of amorphous otenaproxesul or a pharmaceutically acceptable salt thereof exhibit at least a 7 fold to 9 fold improvement in the area under the curve (AUC) of otenaproxesul or a pharmaceutically acceptable salt thereof in a subject after oral administration once a day compared to an identical formulation wherein crystalline otenaproxesul or a pharmaceutically acceptable salt thereof replaces the amorphous solid dispersion. In some embodiments, the compositions of the application comprising 15 mg/kg of amorphous otenaproxesul or a pharmaceutically acceptable salt thereof exhibit at least a 7 fold improvement in the area under the curve (AUC) of otenaproxesul or a pharmaceutically acceptable salt thereof in a subject after oral administration once a day compared to an identical formulation wherein crystalline otenaproxesul or a pharmaceutically acceptable salt thereof replaces the amorphous solid dispersion. In some embodiments, the subject is human. In some embodiments, the subject is canine (dog).

In some embodiments, the compositions of the application exhibit at least a 7× to 9× improvement in the area under the curve (AUC) for otenaproxesul or a pharmaceutically acceptable salt thereof in dogs compared to an otherwise identical formulation except wherein crystalline otenaproxesul or a pharmaceutically acceptable salt thereof replaces the amorphous solid dispersion.

In some embodiments, the compositions of the application comprising a loading dose provide a plasma concentration of naproxen, a metabolite of otenaproxesul, of about 10 μg/mL or greater, about 15 μg/mL or greater or about 20 μg/mL or greater in less than about 30 minutes, less than about 45 minutes, less than about 60 minutes, less than about 75 minutes or less than about 90 minutes after oral administration. In some embodiments, the compositions of the application comprising a loading dose provide a plasma concentration of naproxen of about 10 g/mL or greater, about 15 μg/mL or greater or about 20 μg/mL or greater in less than about 60 minutes after oral administration. In some embodiments, the compositions of the application comprising a loading dose provide a plasma concentration of naproxen of about 10 μg/mL or greater, about 15 μg/mL or greater or about 20 μg/mL or greater in about 30 minutes, about 45 minutes, about 60 minutes about 75 minutes or about 90 minutes after oral administration. In some embodiments, the compositions of the application comprising a loading dose provide a plasma concentration of naproxen of about 10 μg/mL or greater, about 15 μg/mL or greater or about 20 μg/mL or greater in about 60 minutes after oral administration.

In some embodiments, the compositions of the application comprising a loading dose provide a plasma concentration of naproxen that is greater than a plasma concentration of naproxen provided by crystalline otenaproxesul in about 30 minutes, about 45 minutes, about 60 minutes about 75 minutes or about 90 minutes after oral administration, under otherwise identical conditions and equivalent doses of otenaproxesul.

In some embodiments, the compositions of the application comprising a loading dose provide a plasma concentration of naproxen of about 10 μg/mL or greater, about 15 μg/mL or greater or about 20 μg/mL or greater in about 60 minutes after oral administration.

In some embodiments, the compositions of the application comprising a loading dose provide a C₆₀ of naproxen of about 10 μg/mL or greater, about 15 μg/mL or greater or about 20 μg/mL after oral administration. In some embodiments, the compositions of the application comprising a loading dose provide a C₆₀ of naproxen of about 15 μg/mL or greater or about 20 μg/mL or greater after oral administration.

In some embodiments, the compositions of the application comprising a loading dose provide a C₆₀ of naproxen that is greater compared to a C₆₀ of naproxen provided by a composition comprising crystalline otenaproxesul after oral administration under otherwise identical conditions and equivalent doses of otenaproxesul.

In some embodiments, the compositions of the application comprising a loading dose provide a C₆₀ of naproxen that is about 3 to about 12, about 4 to about 10, about 5 to about 10, about 4 to about 8, about 5 to about 9 or about 6 to about 9 times greater compared to a C₆₀ of naproxen provided by a composition comprising crystalline otenaproxesul after oral administration under otherwise identical conditions and equivalent doses of otenaproxesul. In some embodiments, the compositions of the application comprising a loading dose provide a Tmax of naproxen in a subject in less than about 60 minutes, less than about 90 minutes or less than about 120 minutes after oral administration. In some embodiments, the compositions of the application comprising a loading dose provide a Tmax of naproxen in a subject in less than about 60 minutes after oral administration of the pharmaceutical composition of the application comprising the loading dose.

In some embodiments, the compositions of the application comprising a loading dose provide a Tmax of naproxen in about 1 hour to 3 hours, about 1 hour to about 2 hours, about 30 minutes to about 90 minutes or about 30 minutes to about one hour after oral administration. In some embodiments, the compositions of the application provide a Tmax of naproxen at about 2 hours to about 8 hours, about 3 hours to about 7 hours, about 4 hours to about 6.5 hours or about 4.7 to about 6 hours, following administration to a subject.

In some embodiments, the compositions of the application comprising a loading dose provide a Cmax of naproxen of about 20 μg/mL to about 100 μg/mL, about 30 μg/mL to about 80 μg/mL, about 40 μg/mL to about 70 μg/mL, or about 50 μg/mL to about 65 μg/m, following oral administration to a subject. In some embodiments, the compositions of the application provide a Cmax of naproxen metabolite of about 20 μg/mL to about 100 μg/mL, about 30 μg/mL to about 80 μg/mL, about 40 g/mL to about 70 μg/mL, or about 50 μg/mL to about 65 μg/m, following administration to a subject.

In some embodiments, the compositions of the application comprising a loading dose provide an AUC of naproxen metabolite of about 400-600 μg*hr/mL. In some embodiments, the compositions of the application provide an AUC of naproxen of about 500 μg*hr/mL to about 3500 μg*hr/mL or about 500 μg*hr/mL to about 3000 μg*hr/m, following administration to a subject.

In some embodiment, the compositions of the application show an 80% to 125% bioequivalence to compositions comprising crystalline otenaproxesul or a pharmaceutically acceptable salt thereof.

In some embodiments, the compositions of the application provide a Day-1 AUC₀₋₂₄ of about 700 to 950 μg*hr/mL for otenaproxesul or a pharmaceutically acceptable salt thereof in Beagle dogs following administration of 15 mg/kg of an amorphous solid dispersion.

In some embodiments, the amorphous solid are the amorphous solid dispersion provided in Examples 7A and 7B. In some embodiments, the amorphous solid dispersion is ASD no. 1 or ASD no. 4 in Example 7A.

In some embodiments, the compositions of the application provide a Day-5 AUC₀₋₂₄ of about 1100 to about 1300 μg*hr/mL for otenaproxesul or a pharmaceutically acceptable salt thereof in Beagle dogs following four single daily administrations of 15 mg/kg of an amorphous solid dispersion of the application. In some embodiments, the amorphous solid dispersion is ASD no. 1 or ASD no. 4 in Example 7.

In some embodiments, the compositions of the application exhibit a Cmax of about 61 μg/mL to 68 μg/mL following administration in Beagle dogs of 15 mg/kg of an amorphous solid dispersion no. 4 and ASD no 5 relative to a Cmax of approximately 13 μg/mL for crystalline otenaproxesul.

In some embodiments of an amorphous solid dispersion no. 4 and ASD no 5, the compositions of the application exhibit a Day 1 Tmax of approximately 4.7 to 6 hours relative to a Day-1 Tmax of about 12.7 hours for crystalline otenaproxesul.

Compositions of the application may be used alone or in combination with another known agent useful for treating diseases, disorders or conditions that benefit from treatment with otenaproxesul, as described in the Methods and Uses of the Application below.

It is an embodiment that the another known agent useful for treating diseases, disorders or conditions that benefit from treatment with otenaproxesul is administered or used according to the treatment protocol for the other known agent.

### Kits of the Application

In some embodiments, the present application further includes a pharmaceutical package or kit comprising:
one or more pharmaceutical compositions of the application, and
instructions for administration of the one or more pharmaceutical compositions, to a subject in need thereof.

In some embodiments, the present application further includes a pharmaceutical package or kit comprising:
one or more pharmaceutical compositions of the application comprising a loading dose of amorphous otenaproxesul or a pharmaceutically acceptable salt thereof, and
instructions for administration of the one or more pharmaceutical compositions comprising the loading dose of the amorphous otenaproxesul or the pharmaceutically acceptable salt thereof, to a subject in need thereof.

In some embodiments, the loading dose is about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 400 mg, or about 100 mg to about 200 mg per day of the amorphous otenaproxesul or the pharmaceutically acceptable salt thereof. In some embodiments, the loading dose comprises about 50 mg to about 600 mg, about 50 mg to about 400 mg, about 100 mg to about 400 mg, about 200 mg to about 600 mg, or about 200 mg to about 400 mg of amorphous otenaproxesul or a pharmaceutically acceptable salt thereof. In some embodiments, the loading dose comprises about 200 mg to about 400 mg of amorphous otenaproxesul or a pharmaceutically acceptable salt thereof. In some embodiments, the loading dose comprises about 200 mg or about 400 mg or about 600 mg of amorphous otenaproxesul or a pharmaceutically acceptable salt thereof.

In some embodiments, the package or kits further comprise a pharmaceutical composition of the application comprising a second loading dose of amorphous otenaproxesul or a pharmaceutically acceptable salt thereof.

In some embodiments, the pharmaceutical package or kit further comprises one or more pharmaceutical compositions comprising a placebo dose.

In some embodiments, the present application further includes a pharmaceutical package or kit comprising:
a pharmaceutical composition of the application comprising a loading dose of amorphous otenaproxesul or a pharmaceutically acceptable salt thereof, and
one or more pharmaceutical compositions of the application comprising a maintenance dose of amorphous otenaproxesul or a pharmaceutically acceptable salt thereof.

In some embodiments, the present application further includes a pharmaceutical package or kit comprising:
a pharmaceutical composition of the application comprising a loading dose of amorphous otenaproxesul or a pharmaceutically acceptable salt thereof, and
one or more pharmaceutical compositions of the application comprising a tapering dose of amorphous otenaproxesul or a pharmaceutically acceptable salt thereof.

In some embodiments, the loading dose is about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 400 mg, or about 100 mg to about 200 mg per day of the amorphous otenaproxesul or the pharmaceutically acceptable salt thereof In some embodiments, the loading dose is about 50 mg to about 200 mg, about 50 mg to about 100 mg, or about 100 mg to about 200 mg per day of the amorphous otenaproxesul or the pharmaceutically acceptable salt thereof.

In some embodiments, the loading dose is about 100 mg to about 200 mg per day of the amorphous otenaproxesul or the pharmaceutically acceptable salt thereof.

In some embodiments, the loading dose is about 50 mg to about 600 mg, about 50 mg to about 400 mg, about 100 mg to about 400 mg, about 100 mg to about 600 mg, or about 200 mg to about 400 mg of amorphous otenaproxesul or a pharmaceutically acceptable salt thereof. In some embodiments, the loading dose is about 100 mg to about 600 mg or about 200 mg to about 600 mg of amorphous otenaproxesul or a pharmaceutically acceptable salt thereof. In some embodiments, the loading dose is about 200 mg, about 400 mg or about 600 mg of amorphous otenaproxesul or a pharmaceutically acceptable salt thereof.

In some embodiments, each maintenance dose is the same or different. In some embodiments, the maintenance doses is about 5 mg to about 100 mg, about 10 mg to about 100 mg, about 10 mg to about 50 mg, about 25 mg to about 100 mg, about 25 mg to about 50 mg, or about 50 mg to about 100 mg per day of the amorphous otenaproxesul or the pharmaceutically acceptable salt thereof. In some embodiments, the maintenance dose is about 5 mg, 10 mg, about 25 mg, about 50 mg, about 75 mg, or about 100 mg of the amorphous otenaproxesul or the pharmaceutically acceptable salt thereof. In some embodiments, the maintenance dose is tapered and therefore comprise a combination of one or more of about 5 mg, 10 mg, about 25 mg or about 50 mgof the amorphous otenaproxesul or the pharmaceutically acceptable salt thereof.

In some embodiments, each maintenance dose is about 5 mg to about 100 mg, about 10 mg to about 100 mg, about 10 mg to about 50 mg, about 25 mg to about 100 mg, about 25 mg to about 50 mg, about 50 mg to about 100 mg, about 25 mg to about 150 mg, about 25 mg to about 200 mg, or about 25 mg to about 250 mg of amorphous otenaproxesul or a pharmaceutically acceptable salt thereof. In some embodiments, the maintenance dose is about 25 mg to about 200 mg of amorphous otenaproxesul or a pharmaceutically acceptable salt thereof. In some embodiment, the maintenance dose is about 5 mg, about 10 mg, about 25 mg, about 50 mg, about 75 mg, about 100 mg, about 125 mg, about 150 mg, about 175 mg or about 200 mg of amorphous otenaproxesul or a pharmaceutically acceptable salt thereof. In some embodiments, the maintenance dose is about 10 mg, about 25 mg, about 50 mg, about 100 mg or about 200 mg amorphous otenaproxesul or a pharmaceutically acceptable salt thereof. In some embodiments, the maintenance dose is tapered and therefore comprises a combination of one or more of about 25 mg, about 50 mg or about 75 mg of amorphous otenaproxesul or a pharmaceutically acceptable salt thereof.

In some embodiments, the one or more pharmaceutical compositions of the application are formulated for oral delivery. In some embodiments, the loading dose is formulated in one or more pharmaceutical compositions for oral delivery. Therefore, in some embodiments, the kits comprise two or more pharmaceutical compositions of the application comprising a divided dose of amorphous otenaproxesul or a pharmaceutically acceptable salt, wherein the sum of the divided doses equals a dose per day of amorphous otenaproxesul or a pharmaceutically acceptable salt thereof. In some embodiments, the loading dose is about 100 mg to about 200 mg per day of the amorphous otenaproxesul or the pharmaceutically acceptable salt thereof. In some embodiments, the one or more pharmaceutical compositions comprising a maintenance dose are formulated as one or more pharmaceutical compositions for oral delivery and the one or more maintenance doses are about 25 mg, about 50 mg, or about 100 mg, per day of the amorphous otenaproxesul or the pharmaceutically acceptable salt thereof.

In some embodiments, the kits comprise pharmaceutical compositions of the application comprising a divided dose of amorphous otenaproxesul or a pharmaceutically acceptable salt, wherein the sum of the divided doses equals the dose of amorphous otenaproxesul or a pharmaceutically acceptable salt thereof. In some embodiments, the loading dose is about 100 mg to about 400 mg of amorphous otenaproxesul or a pharmaceutically acceptable salt thereof. In some embodiments, the one or more pharmaceutical compositions comprising a maintenance dose are formulated as one or more pharmaceutical compositions for oral delivery and the one or more maintenance doses are about 25 mg, about 50 mg, about 100 mg, or about 200 mg of amorphous otenaproxesul or a pharmaceutically acceptable salt thereof.

In some embodiments, the pharmaceutical package or kit further comprises one or more pharmaceutical compositions comprising a placebo dose.

In some embodiments, the pharmaceutical package or kit further comprises instructions for administration of the pharmaceutical composition of the application comprising the loading dose(s) and the maintenance doses of the amorphous otenaproxesul or the pharmaceutically acceptable salt thereof to a subject in need thereof and optionally the placebo doses. In some embodiments, the instructions direct administration of the pharmaceutical composition of the application to the subject according to a method of treating pain as described herein.

In some embodiments, the one or more pharmaceutical compositions of the application comprising a maintenance dose are provided on consecutive days and each maintenance dose of the amorphous otenaproxesul or the pharmaceutically acceptable salt thereof is the same or different. In some embodiments, the one or more pharmaceutical compositions of the application comprising a maintenance dose are provided on non-consecutive days and each maintenance dose of the amorphous otenaproxesul or the pharmaceutically acceptable salt thereof is the same or different and on days that the maintenance dose is not administered, the subject is administered a placebo.

In some embodiments, the one or more pharmaceutical compositions of the application comprising the maintenance dose are administered once, twice or three times a day and each maintenance dose of amorphous otenaproxesul or a pharmaceutically acceptable salt thereof is the same or different.

In some embodiments, the one or more pharmaceutical compositions of the application comprising a maintenance dose are different. Therefore, in some embodiments, the one or more pharmaceutical compositions of the application comprising a maintenance dose comprise initial maintenance doses of the amorphous otenaproxesul or the pharmaceutically acceptable salt thereof or following maintenance doses of the amorphous otenaproxesul or the pharmaceutically acceptable salt thereof.

In some embodiments, the package or kit comprises one pharmaceutical composition of the application comprising a loading dose of the amorphous otenaproxesul or the pharmaceutically acceptable salt thereof, one pharmaceutical composition of the application comprising an initial maintenance dose of the amorphous otenaproxesul or the pharmaceutically acceptable salt thereof, and two to ten pharmaceutical compositions of the application comprising following maintenance doses of the amorphous otenaproxesul or the pharmaceutically acceptable salt thereof. In some embodiments, the pharmaceutical package or kit further comprises one or more pharmaceutical compositions comprising a placebo dose.

In some embodiments, the package or kit comprises one pharmaceutical composition of the application comprising the loading dose of the amorphous otenaproxesul or the pharmaceutically acceptable salt thereof, five to seven pharmaceutical compositions of the application comprising initial maintenance doses of the amorphous otenaproxesul or the pharmaceutically acceptable salt thereof, and three to seven pharmaceutical compositions of the application comprising following maintenance doses of the amorphous otenaproxesul or a pharmaceutically acceptable salt thereof. In some embodiments, the package or kit comprises four to six pharmaceutical compositions of the application comprising following maintenance doses of the amorphous otenaproxesul or the pharmaceutically acceptable salt thereof. In some embodiments, the package or kit comprises five pharmaceutical compositions of the application comprising initial maintenance doses of the amorphous otenaproxesul or the pharmaceutically acceptable salt thereof and five pharmaceutical compositions of the application comprising following maintenance doses of the amorphous otenaproxesul or the pharmaceutically acceptable salt thereof. In some embodiments, the package or kit comprises seven pharmaceutical compositions of the application comprising initial maintenance doses of the amorphous otenaproxesul or the pharmaceutically acceptable salt thereof and three pharmaceutical compositions of the application comprising following maintenance doses of the amorphous otenaproxesul or the pharmaceutically acceptable salt thereof.

In some embodiments, the package or kit comprises one pharmaceutical compositions of the application comprising the loading dose of amorphous otenaproxesul or a pharmaceutically acceptable salt thereof, two to five pharmaceutical compositions of the application comprising an initial maintenance dose of amorphous otenaproxesul or a pharmaceutically acceptable salt thereof, and four to seven pharmaceutical compositions of the application comprising following maintenance doses of amorphous otenaproxesul or a pharmaceutically acceptable salt thereof. In some embodiments, the package or kit comprises one pharmaceutical composition of the application comprising the loading dose of amorphous otenaproxesul or a pharmaceutically acceptable salt thereof, two to three pharmaceutical compositions of the application comprising an initial maintenance dose of amorphous otenaproxesul or a pharmaceutically acceptable salt thereof, and six to seven pharmaceutical compositions of the application comprising following maintenance doses of amorphous otenaproxesul or a pharmaceutically acceptable salt thereof.

In some embodiments, the one or more pharmaceutical compositions of the application comprising following maintenance doses are tapering doses that are decreased stepwise by an amount. In some embodiments, the one or more pharmaceutical compositions of the application comprising following maintenance doses are tapering doses and comprise decreasing amounts of amorphous otenaproxesul or a pharmaceutically acceptable salt thereof for each subsequent dose. In some embodiments, the following maintenance doses are tapered once. Accordingly, in some embodiments, the pharmaceutical compositions of the application (optionally, four to seven, four to six, or six to seven pharmaceutical compositions of the application) comprising following maintenance doses of amorphous otenaproxesul or a pharmaceutically acceptable salt thereof comprise two different doses of amorphous otenaproxesul or a pharmaceutically acceptable salt thereof.

Therefore, in some embodiments, the package or kit comprises one pharmaceutical composition of the application comprising a loading dose comprising about 100 mg to about 600 mg of amorphous otenaproxesul or a pharmaceutically acceptable salt thereof, two to five pharmaceutical compositions of the application comprising an initial maintenance dose comprising about 25 mg to about 200 mg of amorphous otenaproxesul or a pharmaceutically acceptable salt thereof, and three to seven pharmaceutical compositions of the application comprising a following maintenance dose comprising about 25 mg to about 100 mg of amorphous otenaproxesul or a pharmaceutically acceptable salt thereof. IIn some embodiments, the package or kit comprises one pharmaceutical composition of the application comprising a loading dose comprising about 200 mg to about 400 mg of amorphous otenaproxesul or a pharmaceutically acceptable salt thereof, two to three pharmaceutical compositions of the application comprising an initial maintenance dose comprising about 25 mg to about 200 mg of amorphous otenaproxesul or a pharmaceutically acceptable salt thereof, and six to seven pharmaceutical compositions of the application comprising following a maintenance dose comprising about 25 mg to about 100 mg of amorphous otenaproxesul or a pharmaceutically acceptable salt thereof.

In some embodiments, the package or kit comprises one pharmaceutical composition of the application comprising a loading dose comprising about 200 mg of amorphous otenaproxesul or a pharmaceutically acceptable salt thereof, two to three pharmaceutical compositions of the application comprising an initial maintenance dose comprising about 100 mg of amorphous otenaproxesul or a pharmaceutically acceptable salt thereof, and six to seven pharmaceutical compositions of the application comprising a following maintenance dose comprising about 50 mg and about 25 mg of amorphous otenaproxesul or a pharmaceutically acceptable salt thereof, wherein a first portion of the pharmaceutical compositions of the application comprising the following maintenance dose comprises about 50 mg of amorphous otenaproxesul or a pharmaceutically acceptable salt thereof, and a second portion of the pharmaceutical compositions of the application comprising the following maintenance dose comprises about 25 mg of amorphous otenaproxesul or a pharmaceutically acceptable salt thereof, and the first portion of following doses are administered or used before the second portion of following doses.

In some embodiments, the package or kit comprises one pharmaceutical composition of the application comprising a loading dose comprising about 400 mg amorphous otenaproxesul or a pharmaceutically acceptable salt thereof, two to three pharmaceutical compositions of the application comprising an initial maintenance dose comprising about 200 mg of amorphous otenaproxesul or a pharmaceutically acceptable salt thereof, and six to seven pharmaceutical compositions of the application comprising a following maintenance dose comprising about 100 mg and about 50 mg of amorphous otenaproxesul or a pharmaceutically acceptable salt thereof. In some embodiments, a first portion of the pharmaceutical compositions of the application comprising the following maintenance dose comprises about 100 mg of amorphous otenaproxesul or a pharmaceutically acceptable salt thereof, and a second portion of the pharmaceutical compositions of the application comprising the following maintenance dose comprises about 50 mg of amorphous otenaproxesul or a pharmaceutically acceptable salt thereof, and the first portion of following maintenance doses are administered or used before the second portion of following maintenance doses.

In some embodiments, the package or kit comprises one pharmaceutical composition of the application comprising a loading dose comprising about 600 mg amorphous otenaproxesul or a pharmaceutically acceptable salt thereof, three to five pharmaceutical compositions of the application comprising an initial maintenance dose comprising about 25 mg to about 200 mg of amorphous otenaproxesul or a pharmaceutically acceptable salt thereof, and four to seven pharmaceutical compositions of the application comprising a following maintenance dose comprising about 25 mg and about 100 mg of amorphous otenaproxesul or a pharmaceutically acceptable salt thereof. In some embodiments, a first portion of the pharmaceutical compositions of the application comprising the following maintenance dose comprises about 50 mg to about 100 mg of amorphous otenaproxesul or a pharmaceutically acceptable salt thereof, and a second portion of the pharmaceutical compositions of the application comprising the following maintenance dose comprises about 25 mg to about 50 mg of amorphous otenaproxesul or a pharmaceutically acceptable salt thereof, and the first portion of following maintenance doses are administered or used before the second portion of following maintenance doses.

In some embodiments, the present application further includes a package or kit comprising:
two or more pharmaceutical compositions of the application comprising maintenance doses of amorphous otenaproxesul or a pharmaceutically acceptable salt thereof, and
instructions for administration of the two or more pharmaceutical compositions, to a subject in need thereof.

In some embodiments, the package or kit comprises two to ten or two to nine pharmaceutical compositions of the application comprising maintenance doses of the amorphous otenaproxesul or the pharmaceutically acceptable salt thereof.

In some embodiments, the loading doses and/or each maintenance doses of amorphous otenaproxesul or a pharmaceutically acceptable salt thereof are formulated into pharmaceutical compositions for administration one to six times per day, one to five times per day, one to four times per day, one to three time per day, or once or twice per day. Therefore, in some embodiments, the kit comprises two to twelve, two to eleven, two to ten, two to nine, two to eight, two to seven, two to six, two to five, two to four or two to three pharmaceutical compositions of the application comprising a divided dose of amorphous otenaproxesul or a pharmaceutically acceptable salt thereof, wherein the sum of the divided doses equals a dose of the amorphous otenaproxesul or a pharmaceutically acceptable salt thereof. In some embodiments, the kit comprises two to twelve, two to eleven, two to ten, two to nine, two to eight, two to seven, two to six, two to five, two to four or two to three pharmaceutical compositions of the application comprising a divided loading dose or maintenance dose of amorphous otenaproxesul or a pharmaceutically acceptable salt thereof, wherein the sum of the divided doses equals the loading dose or maintenance dose per day.

In some embodiments, the loading doses and/or maintenance doses of amorphous otenaproxesul or a pharmaceutically acceptable salt thereof are each independently formulated in pharmaceutical compositions for administration one to six times per day, one to five times per day, one to four times per day, one to three time per day, or once or twice per day. Therefore, in some embodiments, the kit comprises one to six, one to five, one to four, one to three or one or two pharmaceutical compositions of the application comprising a divided dose of amorphous otenaproxesul or a pharmaceutically acceptable salt thereof, wherein the sum of the divided doses equals a dose per day of the amorphous otenaproxesul or a pharmaceutically acceptable salt thereof. In some embodiments, the loading doses and/or maintenance doses of amorphous otenaproxesul or a pharmaceutically acceptable salt thereof are each independently formulated in pharmaceutical compositions for administration once or twice per day. Therefore, in some embodiments, the kit comprises one or two pharmaceutical compositions of the application comprising a divided loading dose or maintenance dose of amorphous otenaproxesul or a pharmaceutically acceptable salt thereof, wherein the sum of the divided doses equals the loading dose or maintenance dose per day.

In some embodiments, the loading doses and/or maintenance doses of amorphous otenaproxesul or a pharmaceutically acceptable salt thereof are each independently formulated in pharmaceutical compositions for administration once per day. Therefore, in some embodiments, the kit comprises one pharmaceutical composition of the application comprising a loading dose or a maintenance dose of amorphous otenaproxesul or a pharmaceutically acceptable salt thereof. In some embodiments, the loading doses and/or maintenance doses of amorphous otenaproxesul or a pharmaceutically acceptable salt thereof are each independently formulated for administration in pharmaceutical compositions twice per day. Therefore, in some embodiments, the kit comprises two pharmaceutical compositions of the application comprising a loading dose or a maintenance dose of amorphous otenaproxesul or a pharmaceutically acceptable salt thereof.

In some embodiments, the package or kit is a blister pack, a pill dispenser, a clam shell dispenser or tray. In some embodiments the package or kit will identify the loading dose(s) and/or the subsequent maintenance doses for the course of treatment period, for example, up to 5 days, up to 10 days or optionally up to 14 days. Therefore, in some embodiments, the package or kit further comprise identifiers to identify the pharmaceutical compositions. In some embodiments, the identifiers identify the pharmaceutical compositions by number or day, for example, by the day of the treatment period the pharmaceutical composition is to be administered. For example, in some embodiments, the pharmaceutical composition comprising the loading dose of amorphous otenaproxesul or a pharmaceutically acceptable salt thereof is identified as “1” or “Day 1”.

In some embodiments, each pharmaceutical composition of the application comprising a dose of amorphous otenaproxesul or a pharmaceutically acceptable salt thereof; or two or more pharmaceutical compositions of the application each comprising a divided dose of amorphous otenaproxesul or a pharmaceutically acceptable salt thereof, wherein the sum of the divided doses equals the dose, or each placebo dose, is contained within one blister in the blister pack or one compartment in the pill or clam shell dispenser or tray.

In some embodiments, each pharmaceutical composition of the application comprising the daily dose of amorphous otenaproxesul or a pharmaceutically acceptable salt thereof; or two or more pharmaceutical compositions of the application comprising a divided daily dose of amorphous otenaproxesul or a pharmaceutically acceptable salt thereof, wherein the sum of the divided doses equals a daily dose, or each placebo dose, is contained within one blister in the blister pack or one compartment in the pill or clam shell dispenser or tray. For example, in some embodiments, the pharmaceutical composition of the application comprising the loading dose of amorphous otenaproxesul or a pharmaceutically acceptable salt thereof, or the two or more pharmaceutical compositions of the application comprising a divided loading dose of amorphous otenaproxesul or a pharmaceutically acceptable salt thereof wherein the sum of the divided doses equals the loading dose is contained in a first blister in the blister pack or a first compartment in the pill or clam shell dispenser or tray; and the pharmaceutical composition of the application comprising the initial maintenance dose of amorphous otenaproxesul or a pharmaceutically acceptable salt thereof, or the two or more pharmaceutical compositions of the application comprising a divided maintenance dose of amorphous otenaproxesul or a pharmaceutically acceptable salt thereof, wherein the sum of the divided doses equals the initial maintenance dose, or a placebo dose, is contained in a second blister or second compartment in the pill or clam shell dispenser or tray, and so on. If the package or kit does not include a loading dose, then the first blister or compartment will contain the pharmaceutical composition of the application comprising a first maintenance dose.

### III. Methods of Preparation

In some embodiments, the amorphous solid dispersions of the application are prepared by any suitable microprecipitation process known in the art. In some embodiments, the amorphous solid dispersions are prepared by spray drying (or lyophilization), melt extrusion, freeze drying, rotary evaporation, solvent-controlled precipitation, pH-controlled precipitation, drum drying, supercritical fluid technology or other solvent removal process. In some embodiments, the amorphous solid dispersions are prepared by spray drying. Therefore, the amorphous solid dispersions of the application are spray-dried solid dispersions (SDDs).

Spray drying is a process well known to those skilled in the art for preparing amorphous solid dispersions. In some embodiments, the amorphous solid dispersions are prepared by spray-dried solid dispersion, fluidized bed spray-dried solid dispersion, or spray granulation solid dispersion techniques known in the art.

In some embodiments, the spray drying comprises dispersing or dissolving otenaproxesul or a pharmaceutically acceptable salt thereof and the polymer(s) and optionally sustaining agent in a suitable solvent to form a feed solution, pumping the feed solution through an atomizer into a drying chamber, and removing the solvent to form the amorphous solid dispersion in the drying chamber. In some embodiments, the drying chamber uses hot gases, such as forced air, nitrogen, nitrogen-enriched air, or argon to dry particles. In some embodiments, the feed solution is atomized by conventional means known in the art, such as a two-fluid sonicating nozzle and a two-fluid non-sonicating nozzle. In some embodiments, the spray drying comprises dissolving otenaproxesul or a pharmaceutically acceptable salt thereof and the polymer(s).

In some embodiments, spray drying is carried out using standard equipment used for spray drying. In some embodiments, spray-drying processes and spray-drying equipment are described generally in Perry's Chemical Engineers' Handbook, Sixth Edition (R. H. Perry, D. W. Green, J. O. Maloney, eds.) McGraw-Hill Book Co. 1984, page 20-54 to 20-57.

In some embodiments, the suitable solvent is any solvent or mixture of solvents in which both the drug substance and the polymer have adequate solubility, e.g. solubility that is greater than about 1 mg/ml. In some embodiments, the suitable solvent is selected from dichloromethane, chloroform, ethanol, methanol, 2-propanol, ethyl acetate, acetone, water or mixtures thereof. In some embodiments, the suitable solvent is acetone.

In some embodiments, the amorphous solid dispersion is dried following preparation to remove any residual solvent, for example to remove solvent to International Council for Harmonisation (ICH) limits. In some embodiments, the amorphous solid dispersion is dried at a temperature of about 30° C. to about 50° C., or about 40° C., for about 20 hours to about 30 hours, or about 24 hours, under reduced pressure in an inert gas atmosphere.

In some embodiments, the amorphous solid dispersion is formulated as a granule e.g., by a granulation process, and may include one or more intragranular excipients such as fillers, disintegrants, lubricants and glidants. In some embodiments, the granulation process is any suitable granulation process known in art. In some embodiments, the granulation process is by wet granulation or dry granulation (such as via roller compaction).

In some embodiments, the granules and extragranular disintegrants, lubricants and glidants are combined and compressed into a solid oral dosage form such as tablet by conventional processes known herein and as described in the Examples section below.

Otenaproxesul can be prepared by various synthetic processes. The selection of a particular process is within the purview of the person of skill in the art. For example, otenaproxesul can be prepared by methods known in the art, for example, by the methods disclosed in U.S. Pat. No. 8,541,398.

Salts of otenaproxesul may be formed by methods known to those of ordinary skill in the art, for example, by reacting otenaproxesul with an amount of acid or base, such as an equivalent amount, in a medium such as one in which the salt precipitates or in aqueous medium followed by lyophilization.

Throughout the processes described herein it is to be understood that, where appropriate, suitable protecting groups will be added to and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P.G.M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities and ways to circumvent them by carrying out appropriate transformations and synthetic steps in a suitable order, will be readily understood to one skilled in the art. Examples of transformations are given herein and it is to be understood that the described transformations are not limited only to the generic groups or substituents for which the transformations are exemplified. References and descriptions of other suitable transformations are given in “Comprehensive Organic Transformations-A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### IV. Methods and Uses of the Application

Otenaproxesul in an analgesic and anti-inflammatory H₂S-releasing analogue of naproxen which has been shown to have an enhanced ability to suppress cyclooxygenase-2 (COX-2) activity and/or cyclooxygenase 1 (COX-1) activity and also an increased anti-inflammatory activity when compared to naproxen alone. Otenaproxesul also has greatly reduced gastrointestinal (GI) damaging effects compared to naproxen.

Otenaproxesul was studied in a phase I escalating dose clinical trial wherein otenaproxesul was tested for safety by oral administration in a single dose of crystalline otenaproxesul ranging from 25 mg to 2000 mg. All dose levels were found to be very well tolerated and shown to be safe. Subsequent placebo-controlled phase 2 clinical dose-range finding efficacy studies of otenaproxesul in patients having osteoarthritis pain in the knee with treatment doses of 250 mg, 200 mg and 150 mg validated the efficacy of otenaproxesul in reducing osteoarthritis pain using standard WOMAC subscale pain score outcomes and found that, for example, 250 mg and 200 mg doses of otenaproxesul were efficacious at reducing osteoarthritis pain after 14 days of treatment.

Accordingly, the present application includes a method for inhibiting COX-1 and/or COX-2 in a cell, either in a biological sample or in a subject, comprising administering an effective amount of one or more pharmaceutical compositions of the application to the cell. The application also includes a use of one or more pharmaceutical compositions of the application for inhibiting COX-1 and/or COX-2 as well as a use of one or more pharmaceutical compositions of the application for the preparation of a medicament for inhibiting COX-1 and/or COX-2. The application further includes one or more pharmaceutical compositions of the application for inhibiting COX-1 and/or COX-2.

The present application also includes a method for treating diseases, disorders or conditions that benefit from treatment with otenaproxesul, the method comprising administering an effective amount of one or more pharmaceutical compositions of the application to a subject in need thereof. The present application further includes a use of one or more pharmaceutical compositions of the application for treating diseases, disorders or conditions that benefit from treatment with otenaproxesul, a use of one or more pharmaceutical compositions of the application for the preparation of a medicament for treating diseases, disorders or conditions that benefit from treatment with otenaproxesul, as well as one or more pharmaceutical compositions of the application for use to treat diseases, disorders or conditions that benefit from treatment with otenaproxesul.

The present application includes a method for treating diseases, disorders or conditions treatable by inhibition of COX-1 and/or COX-2 comprising administering an effective amount of one or more pharmaceutical compositions of the application to a subject in need thereof. The application also includes a use of one or more pharmaceutical compositions of the application for treating diseases, disorders or conditions treatable by inhibition of COX-1 and/or COX-2 as well as a use of one or more pharmaceutical compositions of the application for the preparation of a medicament for treating diseases, disorders or conditions treatable by inhibition of COX-1 and/or COX-2. The application further includes one or more pharmaceutical compositions of the application for treating diseases, disorders or conditions treatable by inhibition of COX-1 and/or COX-2.

In some embodiments, the disease, disorder or condition that benefits from treatment with otenaproxesul is cancer, such as melanoma and colorectal cancer. Accordingly, the present application also includes a method of treating cancer comprising administering an effective amount of one or more pharmaceutical compositions of the application to a subject in need thereof. Also included is a use of one or more pharmaceutical compositions of the application for treating cancer, a use of one or more pharmaceutical compositions of the application for preparation of a medicament for treating cancer and one or more polymorphs of otenaproxesul for use to treat cancer. In some embodiments, the compositions of the application are useful the prevention or treatment of pre-cancerous conditions such as Familial Adenomatous Polyposis.

In some embodiments, the diseases disorders or conditions treatable by inhibition of COX-1 and/or COX-2 are selected from inflammation and diseases, disorders or conditions associated with inflammation.

In some embodiments, the inflammation or disease, disorder or condition associated with inflammation is arthritis. Accordingly, the present application also includes a method of treating arthritis comprising administering an effective amount of one or more pharmaceutical compositions of the application to a subject in need thereof.

The present application also includes a use of one or more pharmaceutical compositions of the application for treatment of arthritis as well as of one or more pharmaceutical compositions of the application for the preparation of a medicament for treatment of arthritis. The application further includes one or more pharmaceutical compositions of the application for use in treating arthritis.

In some embodiments, the arthritis is, but is not limited to, rheumatoid arthritis, ankylosing spondylitis, spondyloarthropathies, osteoarthritis, gouty arthritis, systemic lupus erythematosus or juvenile idiopathic arthritis (formerly known as juvenile rheumatoid arthritis). In some embodiments, the arthritis is osteoarthritis, rheumatoid arthritis, ankylosing spondylitis or juvenile idiopathic arthritis. In some embodiments, the arthritis is osteoarthritis. In some embodiments, the arthritis is osteoarthritis of the knee.

Accordingly, the present application also includes a method of treating osteoarthritis, for example, osteoarthritis of the knee, comprising administering one or more pharmaceutical compositions of the application to a subject in need thereof.

In some embodiments, the inflammation or disease, disorder or condition associated with inflammation is selected from one or more of asthma, bronchitis, menstrual cramps, tendinitis, bursitis, skin-related conditions (such as psoriasis, eczema, burns and dermatitis), inflammatory bowel disease, Crohn's disease, gastritis, irritable bowel syndrome, ulcerative colitis and post-operative inflammation.

In some embodiments, the diseases, disorders or conditions associated with inflammation are selected from one or more of vascular diseases, migraine headaches, periarteritis nodosa, thyroiditis, aplastic anemia, Hodgkin's disease, scleroderma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like.

In some embodiments, the diseases, disorders or conditions associated with inflammation are ophthalmic diseases, including but not limited to retinitis, retinopathies, uveitis, ocular photophobia, and acute injury to the eye tissue.

In some embodiments, the inflammation or the disease, disorder or condition associated with inflammation is pulmonary inflammation, including but not limited to, that associated with viral infections and cystic fibrosis.

In some embodiments, the inflammation or the disease, disorder or condition associated with inflammation is a central nervous system disorder such as cortical dementias including Alzheimer's disease.

In some embodiments, the inflammation or the disease, disorder or condition associated with inflammation is selected from allergic rhinitis, respiratory distress syndrome, endotoxin shock syndrome, atherosclerosis and central nervous system damage resulting from stroke, ischemia and/or trauma.

In some embodiments, the methods of the application produce less gastrointestinal tract injury in a subject using or being administered otenaproxesul, compared to the use of naproxen alone.

In some embodiments, the methods of the application are useful in the treatment of inflammation. Therefore, the one or more pharmaceutical compositions of the application are useful as analgesics in the treatment of pain and fever, or as antipyretics in the treatment of fevers.

Accordingly, in some embodiments, the diseases, disorders or conditions treatable by inhibition of COX-1 and/or COX-2 are selected from one or more of pain, fever and headaches.

Accordingly, the present application also includes a method of treating pain comprising administering an effective amount of one or more pharmaceutical compositions of the application to a subject in need thereof. Also included is a use of one or more pharmaceutical compositions of the application for treating pain, a use of one or more pharmaceutical compositions of the application for preparation of a medicament for treating pain and one or more polymorphs of otenaproxesul for use to treat pain.

In some embodiments, the pain is selected from pain that is centrally mediated, pain that is peripherally mediated, pain that is caused by structural tissue injury, pain that is caused by soft tissue injury and pain that is caused by progressive disease.

In some embodiments, the pain is selected from acute pain and chronic pain. In some embodiments, the pain is selected from pain caused by acute injury, trauma, illness and surgery.

In some embodiments, the pain is acute pain that is expected to resolve in a short period of time, for example, 14 days or less, whereas chronic pain is pain that is expected to last for an extended period of time, e.g 15 days or longer.

In some embodiments, the method provides a plasma concentration of naproxen metabolite of about 10 μg/mL or greater, about 15 μg/mL or greater or about 20 μg/mL or greater in less than about 30 minutes or in less than about one hour. In some embodiments, the method provides a plasma concentration of naproxen metabolite of about 15 μg/mL or greater in less than about 30 minutes or in less than about one hour. In some embodiments, the method provides a plasma concentration of naproxen metabolite of about 10 μg/mL or greater, about 15 μg/mL or greater or about 20 μg/mL or greater for approximately for the length of the treatment period. In some embodiments, the plasma concentration of naproxen metabolite of about 10 μg/mL or greater, about 15 μg/mL or greater or about 20 μg/mL or greater is concomitant with onset of relief of acute pain.

Acute pain is distinguishable from chronic pain. In some embodiments, chronic pain includes pain associated with a chronic medical condition or pain that extends beyond the expected period of tissue injury or normal healing. In some embodiments, the chronic medical condition is osteoarthritis, Rheumatoid Arthritis (RA), and/or Ankylosing Spondylitis (AS).

In some embodiments, the acute pain is selected from, but not limited to, post-operative pain, peri-operative pain, primary dysmenorrhea, interstitial cystitis, headache including migraine, pain due to a trauma, renal or biliary colic, arthritis, dental pain, musculoskeletal pain, lower back pain, fibromyalgia, pain of infectious origin, pain resulting from cancer and pain induced by gout. In some embodiments, the post-operative pain is pain following a surgery selected from, but not limited to orthopedic, abdominal, pelvic, dental, plastic, cosmetic, neurological, urological, bariatric, gastric, cardiac, orthoscopic, vascular, endovascular, laparoscopic, oncological, colorectal, podiatric, ocular, otoplastic, rhinoplastic, and throat surgery. In some embodiments, the orthopedic surgery is selected from, but not limited to, bunionectomy total hip, total knee, bilateral total knee, spine, shoulder, ankle, soft tissue surgeries, spinal fusion, rotator cuff repair, laminectomy, fracture repair, and discectomy. In some embodiments, the abdominal and/or pelvic surgery is selected from, but not limited to, inguinal hernia repair such as open inguinal hernectomy, abdominal hysterectomy, abdominal laparotomy, cholecystectomy, vaginal hysterectomy, ventral hernia repair, myomectomy, salpingo-oophorectomy, bariatric, partial colectomy surgeries, and gynecologic or genitourinary surgery.

In some embodiments, acute pain is any corresponding version of acute pain that is identified in a pediatric population.

In some embodiments, the acute pain is headache pain including migraine, photophobia and phonophobia. In some embodiments, the acute pain is migraine pain.

In some embodiments, the post-operative pain is pain resulting from, but not limited to orthopedic, abdominal and/or pelvic, dental, plastic, cosmetic, neurological, urological, bariatric, gastrointestinal, cardiac, orthoscopic, vascular, endovascular, laparoscopic, oncological, colorectal, podiatric, ocular, otoplastic, rhinoplastic, and throat surgery. In some embodiments, the post-operative pain is pain resulting from dental surgery.

In some embodiments, the orthopedic surgery is selected from, but not limited to, bunionectomy, total hip, total knee, bilateral total knee, spine, shoulder, ankle, soft tissue surgeries, spinal fusion, rotator cuff repair, laminectomy, fracture repair, placement of hardware (of any type) and discectomy.

In some embodiments, the orthopedic surgery is bunionectomy. Therefore, in some embodiments, the acute pain is post-operative pain from a bunionectomy.

In some embodiments, the acute pain is dysmenorrhea.

In some embodiments, the acute pain is dental pain.

In some embodiments, the abdominal and/or pelvic surgery is selected from, but not limited to, inguinal hernia repair such as open inguinal hernectomy, abdominal hysterectomy, abdominal laparotomy, cholecystectomy, vaginal hysterectomy, ventral hernia repair, myomectomy, salpingo-oophorectomy, bariatric, partial colectomy surgeries, and gynecologic or genitourinary surgery. In some embodiments, the abdominal and/or pelvic surgery is inguinal hernia repair. In some embodiments, inguinal hernia repair is open inguinal herniotomy.

The methods of the application are for use to treat all severities of pain.

It would be appreciated by a person skilled in the art that pain rating scales are well known and used in daily clinical practice to measure pain intensity. For example, commonly used measurement scales include the Visual Analog Scale (VAS), the Graphic Rating Scale (GRS), the Simple Descriptor Scale (SDS), the Numerical Rating Scale (NRS), the Faces Rating Scale (FRS) and Total Pain Relief (TOTPAR) and/or Sum of Pain Intensity Difference (SPID). Particularly in acute pain, Total Pain Relief (TOTPAR) and/or Sum of Pain Intensity Difference (SPID) are the metrics commonly used to assess pain in the art. Numeric Rating Scales (NRS) which are similar to VAS in acute pain trials are also available. In some embodiments, the visual analog scale (VAS) Is a 10 cm. vertical or horizontal line with word anchors at the extremes, such as “no pain” on one end and “pain as bad as it could be” at the other. The patient is asked to make a mark along the line to represent pain intensity. In the Visual Analog Scale (VAS), moderate or severe pain is defined as a pain intensity of >about 50 mm on a 0-100 mm visual analog scale (VAS). In some embodiments, moderate pain is characterized as a pain intensity of >about 50 mm and <about 70 mm on a 1-100 mm VAS. And severe pain is characterized as a pain intensity of >about 70 mm on a 1-100 mm VAS. The graphic rating scale (GRS) is a variation of the visual scale which adds words such as include “no pain”, “mild”, “severe or numbers between the extremes. The descriptor scale (SDS) is a list of adjectives such as no pain”, “mild”, “moderate” or “severe describing different levels of pain intensity. The numerical pain rating scale (NPRS) refers to a numerical rating of 0 to 10 or 0 to 5 or to a visual scale with both words and numbers. A visual analogue scale (VAS) version of the WOMAC is also commonly used.

In some embodiments, treatment of the subject is assessed using one or more biomarkers known to represent activation of pathways that result in pain reduction, including but not limited to, plasma levels of thromboxane B₂ (TXB₂), prostaglandin E₂ (PGE2), interleukin 1 Beta (IL-1β), interleukin 6 (IL-6), tumor necrosis factor alpha (TNF-α) and/or C-reactive protein (CRP). Levels of these markers that are indicative of a pain response are known in the art as are methods to detect their levels in a subject.

Accordingly, in some embodiments, the acute pain is mild to moderate, moderate to moderately severe or moderately severe to severe.

### V. Dosing Regimens

Otenaproxesul was studied in a phase I escalating dose clinical trial wherein otenaproxesul was tested for safety (not efficacy) by oral administration in a single dose ranging from 25 mg to 2000 mg (crystalline otenaproxesul). All dose levels were found to be very well tolerated and shown to be safe. Subsequent placebo-controlled phase 2 clinical dose-range finding efficacy studies of otenaproxesul in patients having osteoarthritis pain in the knee with treatment doses of 250 mg, 200 mg and 150 mg validated the efficacy of otenaproxesul in reducing osteoarthritis pain using standard WOMAC subscale pain score outcomes and found that, for example, 250 mg and 200 mg doses of otenaproxesul were efficacious at reducing osteoarthritis pain after 14 days of treatment.

However, analysis of liver injury test results, such as alanine aminotransferase (ALT) and aspartate aminotransferase (AST) observed during one of the phase 2 studies showed that while these tests were found to be normal for the majority of patients in the study, a dose-related liver transaminase elevation (LTE), for example, of ALT and AST levels, in approximately 10% of the patients, was observed. These LTEs were detected after the patients had completed their full treatment course. For example, most of the LTEs were observed at ten days or more after treatment with otenaproxesul had been completed. It has now been found that, after further analysis of the results of all clinical studies, these LTE's are not detected after single dose administration of otenaproxesul of 25 mg to 2000 mg (crystalline) or after multiple dose administration of otenaproxesul (crystalline) at doses of 150 mg, 200 mg or 250 mg for treatment periods of 14 days or less, such as seven days or less. Additionally, analysis of the liver injury test results indicates that LTEs are not observed when the cumulative plasma drug exposure of the primary otenaproxesul metabolite, naproxen, over the prescribed treatment does not exceed about 4000 μg*hr/mL.

It has also been found that a single dose administration of otenaproxesul (crystalline) provides a more sustained plasma level of naproxen metabolite, compared to a single dose of a comparable amount naproxen administered under otherwise comparable conditions. Moreover, it has been found that the administration of a single oral dose of about 1500 mg to 2000 mg of otenaproxesul (crystalline) provides a plasma concentration of greater than about 10 μg/mL naproxen metabolite for over 72 hours. Naproxen is a major metabolite of otenaproxesul and analysis of the naproxen metabolite plasma levels during Phase 2 clinical trials reveals that a naproxen metabolite plasma concentration of about >10 to 15 μg/mL is associated with a therapeutic effect, at least in association with the associated release of H₂S from the parent compound otenaproxesul.

It has been advantageously further shown that amorphous otenaproxesul, in particular amorphous otenaproxesul in a composition of the application comprising an amorphous solid dispersion of amorphous otenaproxesul, has an increased release rate and plasma uptake of naproxen metabolite compared to crystalline otenaproxesul. For example, amorphous otenaproxesul as an amorphous solid dispersion, when provided to Beagles dogs at a dose of 15 mg/kg (approximately equivalent to 525 mg human dose) was found to provide naproxen plasma levels of greater than 15 μg/mL within 30 minutes and greater than 20 μg/mL within 60 minutes and 25 μg/mL within 120 minutes.

Further, amorphous otenaproxesul, in particular amorphous otenaproxesul in, an amorphous solid dispersion, was found to surprisingly provide a greater and sustained naproxen metabolite level compared crystalline otenaproxesul when administered to Beagle dogs. For example, a single dose of amorphous otenaproxesul as an amorphous solid dispersion was found to provide a day 1 plasma level of naproxen metabolite in dogs that was 7× to 9× greater compared to an otherwise identical formulation and conditions except wherein crystalline otenaproxesul was used in place of the solid dispersion. Further, day 4 plasma levels of naproxen metabolite in dogs that were a factor of 3 to 4.5 greater compared to an otherwise identical formulation and conditions except wherein crystalline otenaproxesul was used in place of the solid dispersion. Therefore, a lower dose of otenaproxesul that is comprised in a composition of the application is needed to achieve a beneficial effect compared to a dose of crystalline otenaproxesul needed to achieve the same beneficial effect. This results in a lower risk of negative side effects, a faster onset of action and a greater patient compliance.

The Applicants have further found that although the amorphous solid dispersions of otenaproxesul demonstrate an enhanced absorption and initial plasma exposures on Day 1 of naproxen compared to crystalline otenaproxesul, the amorphous solid dispersions of otenaproxesul surprisingly demonstrated lower steady state (e.g, Day 4) exposures compared to crystalline otenaproxesul. While not wishing to be bound by theory, these results suggest that administration or use of amorphous solid dispersions of otenaproxesul of the application would advantageously provide enhanced initial naproxen plasma exposures but overall lower naproxen plasma exposures. By extrapolation, H₂S (hydrogen sulfide) cumulative exposure in plasma over time would be expected to be lower compared to the administration or use of crystalline otenaproxesul. With a lower overall cumulative naproxen and H₂S exposure in plasma, concomitant lower liver exposure to exogenous H₂S associated with otenaproxesul and in turn fewer liver-adverse events, would be expected with the use of amorphous solid dispersions of otenaproxesul compared to administration or use of crystalline otenaproxesul.

Accordingly, the present application includes modified treatment protocols comprising otenaproxesul that are designed for the treatment of pain, including acute pain as described in the “Method and uses of the application” section above which include, for example, peri-operative pain, pain associated with trauma or injury, or pain associated with diseases, disorders or conditions, such as migraine or gout. In the modified treatment protocols the use or administration comprises dosage amounts and timings to provide a cumulative plasma naproxen metabolite (M25) exposure of less than about 4000 μg*hr/mL.

In some embodiments, the otenaproxesul is in a composition of the application, and the present application includes modified treatment protocols for the compositions of the application comprising amorphous otenaproxesul that are designed for the treatment of pain.

Therefore, the present application includes a method of treating pain in a subject in need thereof comprising administering an amount otenaproxesul or a pharmaceutically acceptable salt thereof that provides a cumulative plasma naproxen metabolite (M25) exposure of less than about 4000 μg*hr/mL

In some embodiments, otenaproxesul is in a crystalline form.

In some embodiments, otenaproxesul is an amorphous form.

In some embodiments, the otenaproxesul is suitably formulated into a solid amorphous dispersion of the application as described herein or into a composition of the application (comprising the solid amorphous dispersion) comprising an amount otenaproxesul as described in Section II above.

Therefore, the present application also includes a method of treating pain in a subject in need thereof comprising administering a composition of the application comprising an amount of amorphous otenaproxesul or a pharmaceutically acceptable salt thereof that provides a cumulative plasma naproxen metabolite (M25) exposure of less than about 4000 μg*hr/mL. In some embodiments, the methods and uses described herein provides a cumulative plasma naproxen metabolite (M25) exposure of less than about 4000 μg*hr/mL.

### Dosage Regimens for Amorphous Otenaproxesul (Compositions of the Application)

In some embodiments, the method comprises administering one or more pharmaceutical compositions of the application comprising a loading dose of amorphous otenaproxesul or a pharmaceutically acceptable salt thereof.

In some embodiments, only one loading dose is administered. In some embodiments the loading dose comprises about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 400 mg, about 100 mg to about 300 mg, or about 100 mg to about 200 mg of amorphous otenaproxesul or a pharmaceutically acceptable salt thereof. In some embodiments, the loading dose comprises about 50 mg to about 600 mg, about 50 mg to about 400 mg, about 100 mg to about 400 mg, about 100 mg to about 600 mg, about 200 mg to about 600 mg, or about 200 mg to about 400 mg of amorphous otenaproxesul or a pharmaceutically acceptable salt thereof. In some embodiments, the loading dose comprises about 50 mg to about 200 mg, about 50 mg to about 100 mg, about 100 mg to about 300 mg, or about 100 mg to about 200 mg of amorphous otenaproxesul or a pharmaceutically acceptable salt thereof. In some embodiments, the loading dose comprises about 200 mg to about 600 mg of amorphous otenaproxesul or a pharmaceutically acceptable salt thereof. In some embodiment, the loading dose comprises about 25 mg, about 50 mg, about 75 mg, about 100 mg, about 150 mg, about 200 mg, about 250 mg, about 300 mg, about 350 mg, about 400 mg, about 450 mg, about 475 mg, about 500 mg, about 525 mg, about 550 mg, about 575 mg, or about 600 mg of amorphous otenaproxesul or a pharmaceutically acceptable salt thereof. In some embodiments, the loading dose comprises about 150 mg, about 200 mg, about 250 mg, about 300 mg, about 350 mg, about 400 mg, about 450 mg, about 500 mg, about 550 mg or about 600 mg of amorphous otenaproxesul or a pharmaceutically acceptable salt thereof. In some embodiments, the loading comprises about 25 mg, about 50 mg, about 75 mg, about 100 mg, about 150 mg or about 200 mg of amorphous otenaproxesul or a pharmaceutically acceptable salt thereof. In some embodiments, the loading dose comprises about 100 mg to about 400 mg, 200 mg to about 400 mg, about 100 mg to about 600 mg, 200 mg to about 600 mg, about 200 mg, about 400 mg or about 600 mg of otenaproxesul or a pharmaceutically acceptable salt thereof. In some embodiments, the loading dose comprises about 200 mg, about 400 mg or about 600 mg of amorphous otenaproxesul or a pharmaceutically acceptable salt thereof. In some embodiments, the loading dose comprises about 100 mg to about 600 mg, about 200 mg to about 600 mg, about 200 mg to about 400 mg, about 200 mg about 400 mg or about 600 mg of otenaproxesul or a pharmaceutically acceptable salt thereof.

In some embodiments, the one or more pharmaceutical compositions of the application comprising a loading dose are formulated for oral delivery and comprise about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 400 mg, about 100 mg to about 300 mg, or about 100 mg to about 200 mg amorphous otenaproxesul or a pharmaceutically acceptable salt thereof. In some embodiments, the one or more pharmaceutical compositions of the application comprising a loading dose are formulated for oral delivery and comprise about 50 mg to about 600 mg, about 50 mg to about 400 mg, about 100 mg to about 400 mg, about 100 mg to about 600 mg, about 200 mg to about 600 mg, or about 200 mg to about 400 mg of amorphous otenaproxesul or a pharmaceutically acceptable salt thereof. In some embodiments, the one or more pharmaceutical compositions of the application comprising a loading dose are formulated for oral delivery and comprises about 100 mg to about 200 mg of amorphous otenaproxesul or a pharmaceutically acceptable salt thereof. In some embodiments, the one or more pharmaceutical compositions of the application comprising a loading dose are formulated for oral delivery and comprise about 100 mg to about 600 mg, or about 200 mg to about 600 mg of amorphous otenaproxesul or a pharmaceutically acceptable salt thereof. In some embodiments, the one or more pharmaceutical compositions of the application comprising a loading dose are formulated for oral delivery and comprise about 200 mg, about 400 mg or about 600 mg of amorphous otenaproxesul or a pharmaceutically acceptable salt thereof.

In an exemplary embodiment, the method comprises administering one or more pharmaceutical compositions of the application comprising one loading dose of amorphous otenaproxesul or a pharmaceutically acceptable salt thereof, wherein the loading dose is about 100 mg to about 200 mg of amorphous otenaproxesul or a pharmaceutically acceptable salt thereof. In an exemplary embodiment, the method comprises administering one or more pharmaceutical compositions of the application comprising one loading dose of amorphous otenaproxesul or a pharmaceutically acceptable salt thereof, wherein the loading dose is about 200 mg to about 600 mg, about 200 mg to about 400 mg or about 600 mg of amorphous otenaproxesul or a pharmaceutically acceptable salt thereof.

In an exemplary embodiment, the method comprises administering one or more pharmaceutical compositions of the application comprising one loading dose of amorphous otenaproxesul or a pharmaceutically acceptable salt thereof, wherein the loading dose is about 100 mg of amorphous otenaproxesul or a pharmaceutically acceptable salt thereof. In an exemplary embodiment, the method comprises administering one or more pharmaceutical compositions of the application comprising one loading dose of amorphous otenaproxesul or a pharmaceutically acceptable salt thereof, wherein the loading dose is about 200 mg of amorphous otenaproxesul or a pharmaceutically acceptable salt thereof. In an exemplary embodiment, the method comprises administering one or more pharmaceutical compositions of the application comprising one loading dose of amorphous otenaproxesul or a pharmaceutically acceptable salt thereof, wherein the loading dose is about 400 mg of amorphous otenaproxesul or a pharmaceutically acceptable salt thereof. In an exemplary embodiment, the method comprises administering one or more pharmaceutical compositions of the application comprising one loading dose of amorphous otenaproxesul or a pharmaceutically acceptable salt thereof, wherein the loading dose is about 600 mg of amorphous otenaproxesul or a pharmaceutically acceptable salt thereof.

In some embodiments, the pharmaceutical composition of the application comprising a loading dose provides a plasma concentration of naproxen, a metabolite of otenaproxesul, of about 10 μg/mL or greater, about 15 μg/mL or greater or about 20 μg/mL or greater in less than about 30 minutes, less than about 45 minutes, less than about 60 minutes, less than about 75 minutes or less than about 90 minutes after administration. In some embodiments, the pharmaceutical composition of the application comprising a loading dose provides a plasma concentration of naproxen of about 10 μg/mL or greater, about 15 μg/mL or greater or about 20 μg/mL or greater in less than about 60 minutes after administration.

In some embodiments, the pharmaceutical composition of the application comprising a loading dose provides a plasma concentration of naproxen that is greater than a plasma concentration of naproxen provided by a pharmaceutical composition comprising crystalline otenaproxesul under otherwise identical conditions and equivalent doses of otenaproxesul.

In some embodiments, the pharmaceutical composition of the application comprising a loading dose provides a C₆₀ of naproxen of about 10 μg/mL or greater, about 15 μg/mL or greater or about 20 μg/mL or greater after administration.

In some embodiments, the pharmaceutical composition of the application comprising a loading dose provides a C₆₀ of naproxen that is greater compared to a C₆₀ of naproxen provided by pharmaceutical composition comprising crystalline otenaproxesul under otherwise identical conditions and equivalent doses of otenaproxesul.

In some embodiments, the pharmaceutical composition of the application comprising a loading dose provide a C₆₀ of naproxen that is about 3 to about 12, about 4 to about 10, about 5 to about 10, about 4 to about 8, about 5 to about 9 or about 6 to about 9 times greater compared to a C₆₀ of naproxen provided by a pharmaceutical composition comprising crystalline otenaproxesul after oral administration under otherwise identical conditions and equivalent doses of otenaproxesul. In some embodiments, the loading dose provides a plasma concentration of naproxen metabolite of 15 μg/mL or greater within about 30 minutes to about 1 hour after administration. In some embodiments, the loading dose provides a plasma concentration of naproxen metabolite of 15 μg/mL or greater for approximately 3 days.

Any method for determination of plasma concentration of naproxen metabolite may be used to measure the effect of loading dose, maintenance doses and/or tapering doses used with the application. In some embodiments, the method used to determine plasma concentration levels is provided in Nucro-Technics Bioanalytical Report No. 345625 entitled “LC-MS/MS method development and quantification of naproxen, desmethyl naproxen (M20), and desmethyl naproxen sulfate (M26) in human plasma samples (Non-GLP)”, 2018, which discloses for Sample Preparation and Extraction: A 75 μL aliquot of each sample (calibration standards/quality controls/samples) was aliquoted into pre-labelled tubes. To each aliquoted sample, 300 μL of working internal standard solution (500 ng/ml of IS in acetonitrile/methanol, 75/25, v/v) was added (Exception: 300 μL of acetonitrile/methanol (75/25, v/v) was added to double blank). All tubes were vortexed adequately, followed by centrifugation at 13000 rpm for 5 minutes. A 150 μL aliquot of the organic supernatant was transferred to clean glass tubes. After evaporation at 40° C., the dry residues were reconstituted in 300 μL of USP Purified Water, followed by adequate vortexing. The prepared samples were injected into an LC-MS/MS system which comprised a 6400 Series MS/MS instrument coupled to an Agilent Model 1200 Series liquid chromatography pump and a CTC PAL autosampler. The sample run time was 9.5 minutes on an ACE 5 C18 column (50×2.1 mm; 5 μM) using a gradient mixture. Mobile phase A consisted of 6% methanol, 2% acetonitrile and 5 mM ammonium acetate; Mobile phase B consisted of 90% acetonitrile and 5 mM ammonium acetate.

In some embodiments, the method comprises, on the third, fourth, fifth, sixth or seventh day, optionally on the third, fourth, fifth, sixth, seventh, eighth, ninth or tenth day, after administration of one or more pharmaceutical compositions of the application comprising the loading dose, administering a second composition of the application comprising a loading dose of amorphous otenaproxesul or a pharmaceutically acceptable salt thereof to the subject. In this embodiment, the composition of the application comprising a loading dose becomes a composition of the application comprising a first loading dose. In some embodiments, the administering of the composition of the application comprising a second loading dose is on the third day or fourth day after administration of the first loading dose.

In some embodiments, on days preceding or following the administration of the composition of the application comprising the second loading dose, the subject is administered a composition comprising a placebo.

In some embodiments, the composition of the application comprising a second loading dose comprises the same amount of amorphous otenaproxesul or a pharmaceutically acceptable salt thereof as the composition of the application comprising the first loading dose. In some embodiments, the second loading dose comprises a lower amount of amorphous otenaproxesul or a pharmaceutically acceptable salt thereof as the first loading dose. In some embodiments, the second loading dose is formulated for oral delivery and comprises about 25 mg to about 600 mg of amorphous otenaproxesul or a pharmaceutically acceptable salt thereof. In some embodiments, the composition of the application comprising the second loading dose is formulated for oral delivery and comprises about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 200 mg, about 100 mg to about 400 mg, or about 100 mg to about 300 mg of amorphous otenaproxesul or a pharmaceutically acceptable salt thereof. In some embodiments, the composition comprising the second loading dose is formulated for oral delivery and comprises about 50 mg to about 400 mg, about 50 mg to about 300 mg, about 50 mg to about 200 mg, about 50 mg to about 100 mg, about 100 mg to about 200 mg, about 100 mg to about 400 mg, or about 100 mg to about 300 mg of amorphous otenaproxesul or a pharmaceutically acceptable salt thereof.

In some embodiments, the first and/or second composition of the application comprising a first and/or second loading dose of the amorphous otenaproxesul or a pharmaceutically acceptable salt thereof are each provided as a single composition of the application comprising the loading dose or as two or more a pharmaceutical compositions of the application each comprising divided doses wherein the divided doses combined provide the loading dose, and each divided loading dose may be the same or different.

In some embodiments, on the days preceding or following administration of the second composition of the application comprising the second loading dose, the subject is administered a composition comprising a placebo.

In some embodiments, instead of the second composition of the application comprising a second loading dose, the method further comprises administration of one or more pharmaceutical compositions of the application comprising a maintenance dose of the amorphous otenaproxesul or a pharmaceutically acceptable salt thereof, wherein administration of the compositions of the application comprising amorphous otenaproxesul or a pharmaceutically acceptable salt thereof is for 14 days or less.

Therefore, in some embodiments, the method comprises administering one or more pharmaceutical compositions of the application comprising a loading dose of the amorphous otenaproxesul or a pharmaceutically acceptable salt thereof, followed by administration of one or more pharmaceutical compositions of the application comprising a maintenance dose of the amorphous otenaproxesul or a pharmaceutically acceptable salt thereof, wherein administration of the compositions of the application comprising amorphous otenaproxesul or a pharmaceutically acceptable salt thereof is for a treatment period of 14 days or less. In some embodiments, the treatment period is for 12 days or less, 10 days or less, 8 days or less, 6 days or less, 4 days or less, 3 days or less or 2 days.

In some embodiments, the treatment period is for 10 days or less, 8 days or less, 6 days or less, 4 days or less. In some embodiments, the treatment period is for 6 days or less, 5 days or less or 4 days or less. In some embodiments, the treatment period is for 8 days, 7 days, 6 days, 5 days or 4 days. In some embodiments, the treatment period is for 5 days.

In some embodiments, the one or more pharmaceutical compositions of the application comprising a maintenance dose of amorphous otenaproxesul or a pharmaceutically acceptable salt thereof are provided as a single pharmaceutical composition of the application comprising the maintenance dose or as two or more a pharmaceutical compositions of the application each comprising divided doses of amorphous otenaproxesul or a pharmaceutically acceptable salt thereof, wherein divided doses combined provide the maintenance dose. In some embodiments, each divided maintenance dose is the same or different. In some embodiments, each maintenance dose is the same or different.

In some embodiments, the one or more pharmaceutical compositions of the application comprising a maintenance dose of amorphous otenaproxesul or a pharmaceutically acceptable salt thereof comprises about 5 mg to about 400 mg of amorphous otenaproxesul, or pharmaceutically acceptable salt thereof. In some embodiments, the one or more pharmaceutical compositions of the application comprising a maintenance are administered or used as a single composition comprising a single dose or as two or more compositions each comprising a divided dose wherein divided doses combined provide the maintenance dose, and each maintenance dose or each divided dose may be the same or different.

In some embodiments, the one or more pharmaceutical compositions of the application comprising a maintenance dose are administered once a day and the maintenance dose is the maintenance dose per day. In some embodiments, the maintenance dose of the amorphous otenaproxesul or a pharmaceutically acceptable salt thereof comprise about 5 mg to about 400 mg per day of amorphous otenaproxesul, or pharmaceutically acceptable salt thereof and are each provided as a single composition of the application comprising a single dose and/or as two or more compositions of the application each comprising a divided dose wherein divided doses combined provide the maintenance dose and each maintenance dose or each divided dose may be the same or different. In some embodiments, the maintenance dose of the amorphous otenaproxesul or a pharmaceutically acceptable salt thereof comprise about 5 mg to about 200 mg per day of amorphous otenaproxesul, or pharmaceutically acceptable salt thereof and are each provided as a single composition of the application comprising a single dose and/or as two or more compositions of the application each comprising a divided dose wherein divided doses combined provide the maintenance dose and each maintenance dose or each divided dose may be the same or different.

Therefore, in some embodiments, the method comprises:
administering one or more pharmaceutical compositions of the application comprising a loading dose comprising about 25 mg to about 600 mg of amorphous otenaproxesul or a pharmaceutically acceptable salt thereof, and
administering one or more pharmaceutical compositions of the application comprising a maintenance dose comprising about 5 mg to about 400 mg of amorphous otenaproxesul or a pharmaceutically acceptable salt thereof,
wherein administration of the amorphous otenaproxesul or a pharmaceutically acceptable salt thereof is for a treatment period of 14 days or less.

Therefore, in some embodiments, the method comprises:
administering one or more pharmaceutical compositions of the application comprising a loading dose comprising about 25 mg to about 600 mg of amorphous otenaproxesul or a pharmaceutically acceptable salt thereof, and
administering one or more pharmaceutical compositions of the application comprising a maintenance dose comprising about 5 mg to about 200 mg of amorphous otenaproxesul or a pharmaceutically acceptable salt thereof,
wherein administration of the amorphous otenaproxesul or a pharmaceutically acceptable salt thereof is for a treatment period of 14 days or less.

In some embodiments, the loading dose of amorphous otenaproxesul or a pharmaceutically acceptable salt thereof is greater than the maintenances doses of amorphous otenaproxesul or a pharmaceutically acceptable salt thereof. In some embodiments, each of the maintenance doses are the same, i.e., each maintenance dose comprises the same amount of amorphous otenaproxesul or a pharmaceutically acceptable salt thereof.

In some embodiments, the one or more pharmaceutical compositions of the application comprising a maintenances dose are provided as tapered doses. Therefore, in some embodiments, initial maintenance doses are higher than following maintenance doses and for doses on a first and second consecutive day, the dose on first consecutive day is always the same or greater that the dose on the second consecutive day. In some embodiments, when two or more maintenance doses are administered on the same day, the first maintenance dose of the day is always the same or greater than the following maintenance dose(s) on the same day. In some embodiments, tapering, or a tapered dose includes, for example, a dose that is decreased stepwise by an amount over a period of time, for example about every 6 hours, 12 hours, 18 hours, 24 hours, 36 hours or 72 hours or a combination thereof. In some embodiments, tapering, or a tapered dose includes, for example, a daily dose that is decreased stepwise by an amount over a period of one or more days at each step, or a daily dose that is decreased by an amount each consecutive day. Tapering does not permit an increased following maintenance dose amount compared to the initial maintenance dose amount. Tapering does not permit an increased daily dose amount compared to the previous day.

A person skilled in the art will understand that there are many possible combinations of loading doses and maintenance doses that will provide the desired cumulative plasma naproxen metabolite (M25) exposure of less than about 4000 μg*hr/mL.

In some embodiments, the one or more pharmaceutical compositions of the application comprising a maintenance dose are provided on consecutive days. In some embodiments, the one or more pharmaceutical compositions of the application comprising a maintenance dose are provided on consecutive days and/or on non-consecutive days. In some embodiments, the pharmaceutical compositions of the application comprising a maintenance dose are administered one or more times per day. In some embodiments, two pharmaceutical compositions of the application comprising a maintenance dose are administered per day. In some embodiments, the pharmaceutical compositions of the application comprising a maintenance are administered on consecutive days and/or on non-consecutive days.

In some embodiments, each maintenance dose is the same or different. In some embodiments, the one or more pharmaceutical compositions of the application comprising the maintenance dose are administered as tapered doses.

In some embodiments, on days where no compositions of the application comprising a maintenance dose is administered, the subject is administered a composition comprising a placebo.

In some embodiments, each maintenance dose independently comprises about 5 mg, about 10 mg, about 25 mg about 50 mg, about 75 mg, about 100 mg, about 125 mg, about 150 mg, about 175 mg, about 200 mg, about 225 mg, about 250 mg, about 300 mg, about 350 mg or about 400 mg of amorphous otenaproxesul, or a pharmaceutically acceptable salt thereof. In some embodiments, each maintenance dose independently comprises about 25 mg, about 50 mg, about 75 mg, about 100 mg, about 150 mg or about 200 mg of amorphous otenaproxesul, or a pharmaceutically acceptable salt thereof and are each provided a single dose and/or as divided doses. In some embodiments, each maintenance dose independently comprises about 10 mg, about 25 mg, about 50 mg, about 75 mg, or about 100 mg of amorphous otenaproxesul, or a pharmaceutically acceptable salt thereof. In some embodiments, each maintenance dose independently comprises about 5 mg to about 200 mg, about 10 mg to about 200 mg, about 25 mg to about 400 mg, about 50 mg to about 400 mg, about 10 mg to about 200 mg, about 25 mg to about 200 mg, about 25 mg to about 100 mg, about 25 mg to about 50 mg, or about 50 mg to about 100 mg of amorphous otenaproxesul, or a pharmaceutically acceptable salt thereof. In some embodiments, the one or more pharmaceutical compositions of the application comprises a maintenance dose comprising about 25 mg to about 200 mg of amorphous otenaproxesul or a pharmaceutically acceptable salt thereof. In some embodiments, each maintenance dose is provided as a single dose or as divided doses.

In some embodiments, the one or more pharmaceutical compositions of the application comprising the maintenance dose are administered once per day and are administered on consecutive days and/or on non-consecutive days. In some embodiments, when the maintenance dose is administered once per day, the maintenance dose is the maintenance dose per day. In some embodiments, each maintenance dose independently comprises about 5 mg, about 10 mg, about 25 mg, about 50 mg, about 75 mg, about 100 mg, about 125 mg, about 150 mg, about 175 mg, about 200 mg, about 225 mg, about 250 mg, about 300 mg, about 350 mg or about 400 mg per day of amorphous otenaproxesul, or a pharmaceutically acceptable salt thereof. In some embodiments, each maintenance dose independently comprises about 5 mg to about 400 mg, 5 mg to about 200 mg, about 10 mg to about 200 mg, about 10 mg to about 200 mg, about 25 mg to about 200 mg, about 25 mg to about 100 mg, about 25 mg to about 50 mg, or about 50 mg to about 100 mg of amorphous otenaproxesul, or a pharmaceutically acceptable salt thereof. In some embodiments, each maintenance dose independently comprises about 5 mg, about 10 mg, about 25 mg about 50 mg, about 75 mg, about 100 mg, about 125 mg, about 150 mg, about 175 mg, or about 200 mg per day of amorphous otenaproxesul, or a pharmaceutically acceptable salt thereof, and are each provided as a single dose and/or as divided doses. In some embodiments, each maintenance dose of the amorphous otenaproxesul or a pharmaceutically acceptable salt thereof comprises about 10 mg, about 25 mg, about 50 mg, about 75 mg, or about 100 mg per day of amorphous otenaproxesul, or a pharmaceutically acceptable salt thereof and are each provided a single dose and/or as divided doses. In some embodiments, each maintenance dose comprises about 5 mg to about 100 mg, about 10 mg to about 100 mg, about 10 mg to about 50 mg, about 25 mg to about 100 mg, about 25 mg to about 50 mg, or about 50 mg to about 100 mg per day of amorphous otenaproxesul, or a pharmaceutically acceptable salt thereof, and are each provided a single dose and/or as divided doses. In some embodiments, the one or more pharmaceutical compositions of the application comprising a maintenance dose comprising about 50 mg to about 100 mg per day of amorphous otenaproxesul or a pharmaceutically acceptable salt thereof. In some embodiments, the one or more pharmaceutical compositions of the application comprise a maintenance dose comprising about 25 mg to about 400 mg per day of amorphous otenaproxesul or a pharmaceutically acceptable salt thereof. In some embodiments, the one or more pharmaceutical compositions of the application comprise a maintenance dose comprising about 25 mg to about 200 mg per day of amorphous otenaproxesul or a pharmaceutically acceptable salt thereof.

In some embodiments, the method comprises administration or use of one composition of the application comprising a loading dose of amorphous otenaproxesul or a pharmaceutically acceptable salt thereof, and the loading dose is the loading dose per day. In some embodiments, the composition of the application comprising a loading dose is formulated for oral delivery and comprises about 25 mg to about 600 mg per day of amorphous otenaproxesul or a pharmaceutically acceptable salt thereof. In some embodiments, the composition of the application comprising a loading dose is formulated for oral delivery and comprises about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 200 mg, about 100 mg to about 400 mg, or about 100 mg to about 300 mg per day of amorphous otenaproxesul or a pharmaceutically acceptable salt thereof. In some embodiments, the composition of the application comprising a loading dose is formulated for oral delivery and comprises about 100 mg to about 200 mg per day of amorphous otenaproxesul or a pharmaceutically acceptable salt thereof. In some embodiments, the composition of the application comprising a loading dose is formulated for oral delivery and comprises about 50 mg to about 600 mg, about 50 mg to about 400 mg, about 100 mg to about 600 mg, about 200 mg to about 600 mg, or about 200 mg to about 400 mg per day of amorphous otenaproxesul or a pharmaceutically acceptable salt thereof. In some embodiments, the composition of the application comprising a loading dose is formulated for oral delivery and comprises about 200 mg, about 400 mg or about 600 mg a pharmaceutically acceptable salt thereof.

In some embodiments, the compositions of the application comprising a loading dose are formulated for intravenous delivery and comprises about 25 mg to about 100 mg per day of amorphous otenaproxesul or a pharmaceutically acceptable salt thereof. In some embodiments, the compositions of the application comprising a loading dose are formulated for intravenous delivery and comprises about 50 mg to about 200 mg per day of amorphous otenaproxesul or a pharmaceutically acceptable salt thereof.

In some embodiments, the loading dose comprises about 100 mg to about 200 mg per day of amorphous otenaproxesul, or a pharmaceutically acceptable salt thereof and each maintenance dose comprises about 25 mg to about 125 mg to per day of amorphous otenaproxesul, or a pharmaceutically acceptable salt thereof.

Therefore, in an exemplary embodiment, the method comprises:
administering one or more pharmaceutical compositions of the application comprising a loading dose comprising about 100 mg to about 200 mg of amorphous otenaproxesul or a pharmaceutically acceptable salt thereof, and
administering one or more pharmaceutical compositions of the application comprising a maintenance dose comprising about 25 mg to about 125 mg of amorphous otenaproxesul or a pharmaceutically acceptable salt thereof,
   wherein administration of the amorphous otenaproxesul or a pharmaceutically acceptable salt thereof is for a treatment period of 7 days or less.

In some embodiments, the loading dose comprises about 100 mg of amorphous otenaproxesul or a pharmaceutically acceptable salt thereof and the maintenance dose comprises about 100 mg of amorphous otenaproxesul or a pharmaceutically acceptable salt thereof and the administration of the amorphous otenaproxesul or a pharmaceutically acceptable salt thereof is for a treatment period of 5 days or less.

In some embodiments, the loading dose comprises about 200 mg of amorphous otenaproxesul or a pharmaceutically acceptable salt thereof and the maintenance dose comprises about 100 mg of amorphous otenaproxesul or a pharmaceutically acceptable salt thereof and the administration of the amorphous otenaproxesul or a pharmaceutically acceptable salt thereof is for a treatment period of 5 days or less.

In some embodiments, the loading dose comprises about 100 mg to about 600 mg, about 100 mg to about 400 mg, or about 200 mg to about 400 mg per day of amorphous otenaproxesul, or a pharmaceutically acceptable salt thereof and each maintenance dose comprises about 25 mg to about 400 mg of amorphous otenaproxesul, or a pharmaceutically acceptable salt thereof.

In another exemplary embodiment, the method comprises
administering one pharmaceutical composition of the application comprising a loading dose comprising about 100 mg to about 600 mg of amorphous otenaproxesul or a pharmaceutically acceptable salt thereof, and
administering one or more pharmaceutical compositions of the application comprising a maintenance dose comprising about 25 mg to about 400 mg of amorphous otenaproxesul or a pharmaceutically acceptable salt thereof,
   wherein administration of the amorphous otenaproxesul or a pharmaceutically acceptable salt thereof is for a treatment period of 7 days or less.

In some embodiments, the loading dose comprises about 400 mg of amorphous otenaproxesul or a pharmaceutically acceptable salt thereof and each maintenance dose independently comprises about 50 mg to about 200 mg of amorphous otenaproxesul or a pharmaceutically acceptable salt thereof. In some embodiments, the loading dose comprises about 400 mg of amorphous otenaproxesul or a pharmaceutically acceptable salt thereof and each maintenance dose independently comprises about 50 mg, about 100 mg or about 200 mg of amorphous otenaproxesul or a pharmaceutically acceptable salt.

In some embodiments, the loading dose comprises about 200 mg of amorphous otenaproxesul or a pharmaceutically acceptable salt thereof and each maintenance dose independently comprises about 25 mg to about 100 mg of amorphous otenaproxesul or a pharmaceutically acceptable salt thereof. In some embodiments, the loading dose comprises about 200 mg of amorphous otenaproxesul or a pharmaceutically acceptable salt thereof and each maintenance dose independently comprises about 25 mg, about 50 mg or about 100 mg of amorphous otenaproxesul or a pharmaceutically acceptable salt thereof.

In some embodiments, the loading dose comprises about 600 mg of amorphous otenaproxesul or a pharmaceutically acceptable salt thereof and each maintenance dose independently comprises about 25 mg to about 200 mg of amorphous otenaproxesul or a pharmaceutically acceptable salt thereof. In some embodiments, the loading dose comprises about 600 mg of amorphous otenaproxesul or a pharmaceutically acceptable salt thereof and each maintenance dose independently comprises about 25 mg, about 50 mg, about 100 mg or about 200 mg of amorphous otenaproxesul or a pharmaceutically acceptable salt thereof.

In some embodiments, each dose of amorphous otenaproxesul or a pharmaceutically acceptable salt thereof, including loading doses and maintenance doses, is divided into a plurality of individual doses wherein the sum of the individual doses equals the dose. In some embodiments, each dose of amorphous otenaproxesul or a pharmaceutically acceptable salt thereof is divided into 2 to 6 individual doses wherein the sum of the individual doses equals the dose. In some embodiments, each dose of amorphous otenaproxesul or a pharmaceutically acceptable salt thereof is divided into 2, 3 or 4 individual doses wherein the sum of the individual doses equals the dose. In some embodiments, each dose of amorphous otenaproxesul or a pharmaceutically acceptable salt thereof is divided into 2 or 3 individual doses wherein the sum of the individual doses equals the dose. In some embodiments, each dose of amorphous otenaproxesul or a pharmaceutically acceptable salt thereof is divided into 2 individual doses wherein the sum of the individual doses equals the dose. In some embodiments, each maintenance dose of amorphous otenaproxesul or a pharmaceutically acceptable salt thereof is divided into 2 individual doses wherein the sum of the individual doses equals the dose.

In some embodiments, only one dose is administered per day, and therefore, in some embodiments, each dose per day of amorphous otenaproxesul or a pharmaceutically acceptable salt thereof is divided into a plurality of individual doses wherein the sum of the individual doses equals the dose per day. In some embodiments, each dose per day of amorphous otenaproxesul or a pharmaceutically acceptable salt thereof is divided into 2 to 6 individual doses wherein the sum of the individual doses equals the dose per day. In some embodiments, each dose per day of amorphous otenaproxesul or a pharmaceutically acceptable salt thereof is divided into 2, 3 or 4 individual doses wherein the sum of the individual doses equals the dose per day. In some embodiments, each dose of amorphous otenaproxesul or a pharmaceutically acceptable salt thereof is divided into 2 or 3 individual doses wherein the sum of the individual doses equals the dose per day. In some embodiments, each daily of amorphous otenaproxesul or a pharmaceutically acceptable salt thereof is divided into 2 individual doses wherein the sum of the individual doses equals the dose per day. In some embodiments, each maintenance dose per day of amorphous otenaproxesul or a pharmaceutically acceptable salt thereof is divided into 2 individual doses wherein the sum of the individual doses equals the dose per day.

In some embodiments, the one or more compositions comprising a maintenance dose are administered on the day following the administration of a composition comprising a loading dose. In some embodiments, the one or more compositions comprising a maintenance dose are administered on the same day as the administration of the composition comprising a loading dose. In some embodiments, one composition comprising a maintenance dose is administered following the administration of the composition of the application comprising a loading dose on the same day. In some embodiments, the composition comprising a loading dose is administered in the morning and the composition comprising a maintenance dose is administered in the evening of the same day.

In some embodiments, more than one composition comprising a maintenance dose are administered per day. In some embodiments, two or three compositions comprising a maintenance dose are administered per day. In some embodiments, two compositions of the application comprising a maintenance dose are administered per day. In some embodiments, one composition comprising a maintenance dose is administered in the morning and the other is administered in the evening of the same day (e.g. every 12 hours).

In some embodiments, the composition of the application comprising a loading dose is administered in the morning and the composition of the application comprising a maintenance dose is administered in the evening on the same day, and on the following treatment days, two compositions of the application comprising a maintenance dose are administered, and wherein one composition comprising a maintenance dose is administered in the morning and the other is administered in the evening of the same day (e.g. every 12 hours).

In some embodiments, each maintenance dose is the same or different. In some embodiments, the maintenance doses are tapered after administration of the loading dose to the second, third, fourth day or fifth day after administration of the loading dose. In some embodiments, the maintenance doses are tapered from the administration of the loading dose to the last day of the treatment period. In some embodiments, the maintenance doses are tapered from the first day after administration of the loading dose to third, fourth day or fifth day after administration of the loading dose. In some embodiments, the maintenance doses are tapered from the first day after administration of the loading dose to the last day of the treatment period. When more than one maintenance doses are administered per day, the first maintenance dose of the day is always the same or greater than the following maintenance dose(s) on the same day.

In some embodiments, each maintenance dose is different and comprises initial maintenance doses and following maintenance doses. Therefore, in some embodiments, the one or more pharmaceutical compositions of the application comprising a maintenance dose are one or more pharmaceutical compositions of the application comprising an initial maintenance dose and one or more pharmaceutical compositions of the application comprising a following maintenance dose. In some embodiments, each maintenance dose is selected from an initial maintenance dose and a following maintenance doses. Therefore, in some embodiments, the method comprises administering one or more pharmaceutical compositions of the application comprising an initial maintenance dose; and administering one or more pharmaceutical compositions of the application comprising a following maintenance dose.

In some embodiments, the one or more initial maintenance doses are higher than the one or more following maintenance doses. In some embodiments, the one or more initial and/or following maintenance doses are provided on consecutive days and/or on non-consecutive days. In some embodiments, the initial and/or following maintenance doses are provided about every 6 hours to 36 hours, 12 hours to 36 hours or 12 hours to 24 hours. In some embodiments, the initial and/or following maintenance doses are provided about every 6 hours, 12 hours, 24 hours or 36 hours. In some embodiments, the initial and/or following maintenance doses are provided every 24 hours. In some embodiments, the initial and/or following maintenance doses are provided about every 12 hours.

In some embodiments, one pharmaceutical composition of the application comprising an initial maintenance dose is administered on the same day as the pharmaceutical composition of the application comprising a loading dose. In some embodiments, one or two pharmaceutical compositions of the application comprising an initial maintenance dose are administered the day following the administration of a loading dose.

In some embodiments, one pharmaceutical composition of the application comprising an initial maintenance dose is administered on the same day as the pharmaceutical composition of the application comprising a loading dose. In some embodiments, the pharmaceutical composition of the application comprising the loading dose is administered in the morning and one pharmaceutical composition of the application comprising the initial maintenance dose is administered in the evening of the same day, and on the following treatment days, one or two pharmaceutical compositions of the application comprising the initial maintenance doses are administered; or one or two pharmaceutical compositions of the application comprising following maintenance doses are administered.

In some embodiments, the pharmaceutical composition of the application comprising a loading dose is administered in the morning and one pharmaceutical composition of the application comprising an initial maintenance dose is administered in the evening of the same day, and on the following treatment day, one or two pharmaceutical composition of the application comprising an initial maintenance dose are administered, or one pharmaceutical composition of the application comprising an initial maintenance dose and one pharmaceutical composition of the application comprising a following maintenance dose is administered, and on following treatment days (e.g. day 3 onward) one or two pharmaceutical compositions of the application comprising a following maintenance dose are administered per day. In some embodiments, on the following treatment days, two pharmaceutical compositions of the application comprising a following maintenance dose are administered per day, once in the morning and once in the evening of the same day (e.g, about every 12 hours).

In some embodiments, the one or more pharmaceutical compositions of the application comprising a following maintenance dose are tapered after administration of the pharmaceutical composition of the application comprising an initial maintenance dose to the second, third, fourth day or fifth day after administration of the loading dose. In some embodiments, the one or more pharmaceutical compositions of the application comprising a following maintenance dose are tapered after administration of the pharmaceutical composition of the application comprising an initial maintenance dose to the last day of the treatment period.

In an exemplary embodiment, the pharmaceutical composition of the application comprising a loading dose is administered in the morning and one pharmaceutical composition of the application comprising an initial maintenance dose is administered in the evening of the same day, and on the following treatment day, one to three pharmaceutical compositions of the application comprising an initial maintenance dose are administered, or one pharmaceutical composition of the application comprising an initial maintenance dose and one or two pharmaceutical compositions of the application comprising a following maintenance dose are administered, and on the following treatment days one to three pharmaceutical compositions of the application comprising a following maintenance doses are administered.

In an exemplary embodiment, the pharmaceutical composition of the application comprising a loading dose is administered in the morning and one pharmaceutical composition of the application comprising an initial maintenance dose is administered in the evening of the same day, and on the following treatment day, two pharmaceutical compositions of the application comprising an initial maintenance dose are administered, or one pharmaceutical composition of the application comprising an initial maintenance dose and one pharmaceutical composition of the application comprising a following maintenance dose are administered, and on the following treatment days one or two pharmaceutical compositions of the application comprising a following maintenance dose are administered.

In some embodiments, when one or more pharmaceutical compositions of the application comprising an initial maintenance dose or one or more pharmaceutical compositions of the application comprising a following maintenance dose are administered per day, the one or more pharmaceutical compositions of the application comprising the initial or following maintenance doses are administered at equal time intervals during the day. In some embodiments, when two pharmaceutical compositions of the application comprising the initial maintenance doses or two pharmaceutical compositions of the application comprising the following maintenance doses are administered, a first pharmaceutical composition of the application comprising the initial or following maintenance dose of the day is administered in the morning and a second pharmaceutical composition of the application comprising the initial or following maintenance dose of the day is administered in the evening of the same day (e.g, about every 12 hours).

In some embodiments, each initial maintenance dose is the same or different. In some embodiments, each initial maintenance dose is the same.

In some embodiments, each following maintenance dose is the same or different. In some embodiments, the following maintenance dose are different and are tapered.

In some embodiments, the following maintenance doses are tapered after administration of the initial maintenance dose to the second, third, fourth day or fifth day after administration of the loading dose. In some embodiments, the following maintenance doses are tapered from the administration of the initial maintenance dose to the last day of the treatment period. In some embodiments, the following maintenance doses are tapered once or twice from the administration of the initial maintenance dose to the last day of the treatment period. In some embodiments, the following maintenance doses are tapered once from the administration of the initial maintenance dose to the last day of the treatment period.

In some embodiments, on days where no maintenance dose is administered, the subject is administered a composition comprising a placebo.

In exemplary embodiments, the loading dose comprises about 200 mg to about 600 mg, or 200 mg to about 400 mg of amorphous otenaproxesul or a pharmaceutically acceptable salt thereof, and each initial maintenance dose comprises about 50 mg to about 200 mg of amorphous otenaproxesul, or a pharmaceutically acceptable salt thereof, and each following maintenance dose comprises about 25 mg to about 100 mg of amorphous otenaproxesul, or a pharmaceutically acceptable salt thereof. In some embodiments, the loading dose comprises about 200 mg of amorphous otenaproxesul, or a pharmaceutically acceptable salt thereof and each initial maintenance dose comprises about 100 mg of amorphous otenaproxesul, or a pharmaceutically acceptable salt thereof and each following maintenance dose comprises about 25 mg or about 50 mg of amorphous otenaproxesul, or a pharmaceutically acceptable salt thereof. In some embodiments, the loading dose comprises about 400 mg of amorphous otenaproxesul, or a pharmaceutically acceptable salt thereof and each initial maintenance dose comprises about 200 mg of amorphous otenaproxesul, or a pharmaceutically acceptable salt thereof and each following maintenance dose comprises about 50 mg or about 100 mg of amorphous otenaproxesul, or a pharmaceutically acceptable salt thereof. In some embodiments, the loading dose comprises about 600 mg of amorphous otenaproxesul, or a pharmaceutically acceptable salt thereof and each initial maintenance dose comprises about 50 mg to about 200 mg of amorphous otenaproxesul, or a pharmaceutically acceptable salt thereof and each following maintenance dose comprises about 25 mg to about 100 mg of amorphous otenaproxesul, or a pharmaceutically acceptable salt thereof.

In exemplary embodiments, the loading dose is about 100 mg to about 200 mg per day of amorphous otenaproxesul or a pharmaceutically acceptable salt thereof, and the one or more initial maintenance doses are about 50 mg to about 150 mg per day of amorphous otenaproxesul, or a pharmaceutically acceptable salt thereof, and the one or more following maintenance doses are about 25 mg to about 75 mg per day of amorphous otenaproxesul, or a pharmaceutically acceptable salt thereof. In some embodiments, the loading dose is about 100 mg, 150 mg or about 200 mg per day of amorphous otenaproxesul, or a pharmaceutically acceptable salt thereof and the one or more initial maintenance doses are about 75 mg to about 125 mg per day of amorphous otenaproxesul, or a pharmaceutically acceptable salt thereof and the one or more following maintenance doses are about 25 mg to about 75 mg per day of amorphous otenaproxesul, or a pharmaceutically acceptable salt thereof. In some embodiments, the loading dose is about 100 mg per day of amorphous otenaproxesul, or a pharmaceutically acceptable salt thereof and the one or more initial maintenance doses are about 100 mg to per day of amorphous otenaproxesul, or a pharmaceutically acceptable salt thereof and the one or more following maintenance doses are about 50 mg per day of amorphous otenaproxesul, or a pharmaceutically acceptable salt thereof. In some embodiments, the loading dose is about 200 mg per day of amorphous otenaproxesul, or a pharmaceutically acceptable salt thereof and the one or more initial maintenance doses are about 100 mg to per day of amorphous otenaproxesul, or a pharmaceutically acceptable salt thereof and the one or more following maintenance doses are about 50 mg per day of amorphous otenaproxesul, or a pharmaceutically acceptable salt thereof.

In some embodiments, the one or more initial maintenance doses are administered for 1 to 3 days. In some embodiments, the one or more initial maintenance doses are administered for 1 to 4 days 1 to 3 days, 1 to 2 days or 2 to 3 days. In some embodiments, the initial maintenance dose is administered only once. In some embodiments, one pharmaceutical composition of the application comprising an initial maintenance dose is administered on the same day as the pharmaceutical composition of the application comprising the loading dose. In some embodiments, one pharmaceutical composition of the application comprising an initial maintenance dose is administered on the same day as the pharmaceutical composition of the application comprising the loading dose, and one or two pharmaceutical compositions of the application comprising an initial maintenance dose are administered on the following day after administration of the pharmaceutical composition of the application comprising the loading dose. In some embodiments, one or more pharmaceutical compositions of the application comprising a following maintenance doses are administered on the days after administration of the pharmaceutical composition of the application comprising the initial maintenance dose(s).

In some embodiments, the initial maintenance doses are the same, i.e., not tapered. In some embodiments, the one or more initial maintenance doses are tapered. In some embodiments, the initial maintenance doses are the same.

In some embodiments, the one or more following maintenance doses are administered for 1 to 4 days following administration of the one or more initial maintenance doses. In some embodiments, the one or more following maintenance doses are administered 1 to 4 days 1 to 3 days, 2 to 3 days or 1 to 2 days following administration of the one or more initial maintenance doses. In some embodiments, the following maintenance doses are administered for 2 to 3 days following administration of initial maintenance doses.

In some embodiments, the following maintenance doses are the same, i.e., not tapered. In some embodiments, the following maintenance doses are tapered. In some embodiments, following maintenance doses are tapered about every 6 hours, 12 hours, 24 hours, 36 hours or 48 hours. In some embodiments, following maintenance doses are tapered every 24 hours to 36 hours. In some embodiments, following maintenance doses are tapered every 24 hours or 36 hours. In some embodiments, the following maintenance doses are tapered every 24 hours. In some embodiments, the following maintenance doses are tapered every 36 hours.

In an exemplary embodiment, the method comprises:
administering one pharmaceutical composition of the application comprising a loading dose comprising about 100 mg to about 600 mg of amorphous otenaproxesul or a pharmaceutically acceptable salt thereof,
administering one or more pharmaceutical compositions of the application comprising an initial maintenance dose comprising about 25 mg to about 200 mg of amorphous otenaproxesul or a pharmaceutically acceptable salt thereof, and
administering one or more pharmaceutical compositions of the application comprising a following maintenance dose comprising about 25 mg to about 100 mg of amorphous otenaproxesul or a pharmaceutically acceptable salt thereof,
   wherein administration of the amorphous otenaproxesul or a pharmaceutically acceptable salt thereof is for a treatment period of 7 days or less, and each the initial and following maintenance doses are the same or different.

In another exemplary embodiment, the method comprises:
administering one or more pharmaceutical compositions of the application comprising a loading dose comprising about 100 mg to about 200 mg of amorphous otenaproxesul or a pharmaceutically acceptable salt thereof,
administering one or more pharmaceutical compositions of the application comprising an initial maintenance dose comprising about 25 mg to about 125 mg of amorphous otenaproxesul or a pharmaceutically acceptable salt thereof, and
administering one or more pharmaceutical compositions of the application comprising a following maintenance dose comprising about 25 mg to about 75 mg of amorphous otenaproxesul or a pharmaceutically acceptable salt thereof,
   wherein administration of the amorphous otenaproxesul or a pharmaceutically acceptable salt thereof is for a treatment period of 7 days or less.

In some embodiments, the treatment period is for 3 to 5, 4 to 5 or 5 to 6 days. In some embodiments, the treatment period is for 3, 4, 5 or 6 days. In some embodiments, the treatment period is for 4 or 5 days. In some embodiments, the treatment period is for 5 days.

In some embodiments, the loading dose comprises about 100 mg of amorphous otenaproxesul or a pharmaceutically acceptable salt thereof, the initial maintenance dose comprises about 100 mg of amorphous otenaproxesul or a pharmaceutically acceptable salt thereof, and the following maintenance dose comprises about 50 mg of amorphous otenaproxesul or a pharmaceutically acceptable salt and/or solvate wherein the initial maintenance dose is administered for 1 to 3 days following administration of the loading dose and the following maintenance dose is administered for 1 to 3 days following administration of the initial maintenance dose.

In some embodiments, the pharmaceutical composition of the application comprising the loading dose comprises about 200 mg of amorphous otenaproxesul or a pharmaceutically acceptable salt thereof, the one or more pharmaceutical compositions of the application comprising an initial maintenance dose comprises about 100 mg of amorphous otenaproxesul or a pharmaceutically acceptable salt thereof, and the one or more pharmaceutical compositions of the application comprising a following maintenance dose comprises about 25 mg to about 50 mg of amorphous otenaproxesul or a pharmaceutically acceptable salt thereof. In some embodiments, the pharmaceutical composition of the application comprising the loading dose comprises about 200 mg of amorphous otenaproxesul or a pharmaceutically acceptable salt thereof, the one or more pharmaceutical compositions of the application comprising an initial maintenance dose comprises about 100 mg of amorphous otenaproxesul or a pharmaceutically acceptable salt thereof, and the one or more pharmaceutical compositions of the application comprising the following maintenance dose comprises about 25 mg or about 50 mg of amorphous otenaproxesul or a pharmaceutically acceptable salt thereof, and
one of the pharmaceutical compositions of the application comprising the initial maintenance doses is administered on the same day as the pharmaceutical composition of the application comprising the loading dose;
one or two of the pharmaceutical compositions of the application comprising the initial maintenance dose or one pharmaceutical composition of the application comprising the initial maintenance dose and one pharmaceutical composition of the application comprising the following maintenance dose are administered on the day after administration of the pharmaceutical composition of the application comprising the loading dose,
and on subsequent treatment days (e.g., day 3 onward) one or two pharmaceutical compositions of the application comprising the following maintenance doses are administered per day.

In some embodiments, the loading dose comprises about 200 mg of amorphous otenaproxesul or a pharmaceutically acceptable salt thereof, the initial maintenance dose comprises about 100 mg of amorphous otenaproxesul or a pharmaceutically acceptable salt thereof, and the following maintenance dose comprises about 50 mg of amorphous otenaproxesul or a pharmaceutically acceptable salt and/or solvate wherein the initial maintenance dose is administered for 1 to 3 days following administration of the loading dose and the following maintenance dose is administered for 1 to 3 days following administration of the initial maintenance dose.

In some embodiments, the pharmaceutical composition of the application comprising the loading dose comprises about 400 mg of amorphous otenaproxesul or a pharmaceutically acceptable salt thereof, the one or more pharmaceutical compositions of the application comprising the initial maintenance dose comprises about 200 mg of amorphous otenaproxesul or a pharmaceutically acceptable salt thereof, and the one or more pharmaceutical compositions of the application comprising the following maintenance dose comprises about 50 mg or about 100 mg of amorphous otenaproxesul or a pharmaceutically acceptable salt thereof.

In some embodiments, the pharmaceutical composition of the application comprising the loading dose comprises about 400 mg of amorphous otenaproxesul or a pharmaceutically acceptable salt thereof, the one or more pharmaceutical compositions of the application comprising the initial maintenance dose comprises about 200 mg of amorphous otenaproxesul or a pharmaceutically acceptable salt thereof, and the one or more pharmaceutical compositions of the application comprising the following maintenance dose comprises about 50 mg or about 100 mg of amorphous otenaproxesul or a pharmaceutically acceptable salt; and
one of the pharmaceutical compositions of the application comprising the initial maintenance doses is administered on the same day as the pharmaceutical composition of the application comprising the loading dose;
one or two of the pharmaceutical compositions of the application comprising the initial maintenance dose or one pharmaceutical composition of the application comprising the initial maintenance dose and one pharmaceutical composition of the application comprising the following maintenance dose are administered on the day after administration of the pharmaceutical compositions of the application comprising the loading dose,
and on subsequent treatment days (e.g., day 3 onward) one or two pharmaceutical compositions of the application comprising the following maintenance doses are administered per day.

In some embodiments, one of the pharmaceutical compositions of the application comprising the initial maintenance doses is administered on the same day as the pharmaceutical composition of the application comprising the loading dose;
two of the pharmaceutical compositions of the application comprising the initial maintenance dose are administered on the day after administration of the pharmaceutical compositions of the application comprising the loading dose,
and on subsequent treatment days (e.g. day 3 onward) two pharmaceutical compositions of the application comprising the following maintenance doses are administered per day.

In some embodiments, one of the pharmaceutical compositions of the application comprising the initial maintenance doses is administered on the same day as the pharmaceutical composition of the application comprising the loading dose;
one pharmaceutical composition of the application comprising the initial maintenance dose and one pharmaceutical composition of the application comprising the following maintenance dose are administered on the day after administration of the pharmaceutical compositions of the application comprising the loading dose,
and on subsequent treatment days (e.g. day 3 onward) two pharmaceutical compositions of the application comprising the following maintenance doses are administered per day.

In some embodiments, the two pharmaceutical compositions of the application comprising the initial or the following maintenance dose are administered once in the morning and once in the evening of the same day.

In some embodiments, pharmaceutical compositions comprising the initial and/or following maintenance doses are provided about every 12 hours to 24 hours. In some embodiments, pharmaceutical compositions comprising the initial and/or following maintenance doses are provided about every 12 hours.

In some embodiments, each following maintenance dose is the same or different. In some embodiments, the following maintenance doses are tapered once or twice from the administration of a last initial maintenance dose to the last day of the treatment period. In some embodiments, the following maintenance doses are tapered once from the administration of a last initial maintenance dose to the last day of the treatment period.

In some embodiments, the one or more pharmaceutical compositions of the application comprising the following maintenance dose comprises about 25 mg to about 50 mg of amorphous otenaproxesul or a pharmaceutically acceptable salt, and the one or more following maintenance doses are tapered from about 50 mg to about 25 mg of amorphous otenaproxesul or a pharmaceutically acceptable salt thereof.

In some embodiments, the one or more pharmaceutical compositions of the application comprising the following maintenance dose comprises about 50 mg to about 100 mg of amorphous otenaproxesul or a pharmaceutically acceptable salt thereof and the one or more pharmaceutical compositions of the application comprising the following maintenance doses are tapered from about 100 mg to about 50 mg of amorphous otenaproxesul or a pharmaceutically acceptable salt thereof.

In some embodiments, the pharmaceutical composition of the application comprising the loading dose comprises about 600 mg of amorphous otenaproxesul or a pharmaceutically acceptable salt thereof, the one or more pharmaceutical compositions of the application comprising the initial maintenance dose comprises about 50 mg to about 200 mg of amorphous otenaproxesul or a pharmaceutically acceptable salt thereof, and the one or more pharmaceutical compositions of the application comprising the following maintenance dose comprises about 50 mg or about 100 mg of amorphous otenaproxesul or a pharmaceutically acceptable salt thereof.

In some embodiments, the pharmaceutical composition of the application comprising the loading dose comprises about 600 mg of amorphous otenaproxesul or a pharmaceutically acceptable salt thereof, the one or more pharmaceutical compositions of the application comprising the initial maintenance dose comprises about 50 mg to about 200 mg of amorphous otenaproxesul or a pharmaceutically acceptable salt thereof, and the one or more pharmaceutical compositions of the application comprising the following maintenance dose comprises about 50 mg or about 100 mg of amorphous otenaproxesul or a pharmaceutically acceptable salt; and
one or two pharmaceutical compositions of the application comprising the initial maintenance dose are administered on the day after administration of the pharmaceutical compositions of the application comprising the loading dose, and optionally on the subsequent treatment day (e.g. day 3)
on the subsequent treatment days (e.g., day 3 or 4) one or two pharmaceutical compositions of the application comprising the following maintenance doses are administered per day.

In some embodiments, the following maintenance doses are tapered after 24 hours to 36 hours after administration of the first following maintenance dose.

In some embodiments, each dose of amorphous otenaproxesul or a pharmaceutically acceptable salt thereof, including loading, initial maintenance and following maintenance doses, is divided into a plurality of individual doses wherein the sum of the individual doses equals the dose. In some embodiments, each dose of amorphous otenaproxesul or a pharmaceutically acceptable salt thereof is divided into 2 to 6 individual doses wherein the sum of the individual doses equals the dose. In some embodiments, each dose of amorphous otenaproxesul or a pharmaceutically acceptable salt thereof is divided into 2, 3 or 4 individual doses wherein the sum of the individual doses equals the dose. In some embodiments, each dose of amorphous otenaproxesul or a pharmaceutically acceptable salt thereof is divided into 2 or 3 individual doses wherein the sum of the individual doses equals the dose. In some embodiments, each dose of amorphous otenaproxesul or a pharmaceutically acceptable salt thereof is divided into 2 individual doses wherein the sum of the individual doses equals the dose.

In some embodiments, one dose of amorphous otenaproxesul or a pharmaceutically acceptable salt thereof is administered per day. Therefore, in some embodiments, each dose per day of amorphous otenaproxesul or a pharmaceutically acceptable salt thereof is divided into a plurality of individual doses wherein the sum of the individual doses equals the dose per day. In some embodiments, each dose per day of amorphous otenaproxesul or a pharmaceutically acceptable salt thereof is divided into 2 to 6 individual doses wherein the sum of the individual doses equals the dose per day. In some embodiments, each dose per day of amorphous otenaproxesul or a pharmaceutically acceptable salt thereof is divided into 2, 3 or 4 individual doses wherein the sum of the individual doses equals the dose per day. In some embodiments, each dose of amorphous otenaproxesul or a pharmaceutically acceptable salt thereof is divided into 2 or 3 individual doses wherein the sum of the individual doses equals the dose per day. In some embodiments, each daily of amorphous otenaproxesul or a pharmaceutically acceptable salt thereof is divided into 2 individual doses wherein the sum of the individual doses equals the dose per day.

In some embodiments, a single composition of the application comprises a dose of amorphous otenaproxesul or a pharmaceutically acceptable salt thereof, two or more pharmaceutical compositions of the application each comprise a divided dose wherein the sum of the divided doses equals the dose. In some embodiments, two to six pharmaceutical compositions of the application each comprise a divided dose wherein the sum of two to 6 divided doses equals the dose. In some embodiments, two, three or four pharmaceutical compositions of the application each comprise a divided dose wherein the sum of two, three or four divided doses equals the dose. In some embodiments, two or three pharmaceutical compositions of the application each comprise a divided dose wherein the sum of the two or three divided doses equals the dose. In some embodiments, two pharmaceutical compositions of the application each comprise a divided dose wherein the sum of two divided doses equals the dose.

In some embodiments, a single composition of the application comprises a dose per day of amorphous otenaproxesul or a pharmaceutically acceptable salt thereof, two or more pharmaceutical compositions of the application each comprise a divided dose wherein the sum of the divided doses equals the dose per day. In some embodiments, two to six pharmaceutical compositions of the application each comprise a divided dose wherein the sum of two to 6 divided doses equals the dose per day. In some embodiments, two, three or four pharmaceutical compositions of the application each comprise a divided dose wherein the sum of two, three or four divided doses equals the dose per day. In some embodiments, two or three pharmaceutical compositions of the application each comprise a divided dose wherein the sum of the two or three divided doses equals the dose per day. In some embodiments, two pharmaceutical compositions of the application each comprise a divided dose wherein the sum of two divided doses equals the dose per day.

In some embodiments, each composition of the application comprising a dose of amorphous otenaproxesul or a pharmaceutically acceptable salt thereof is formulated for oral delivery.

In some embodiments, each composition of the application comprising a dose of amorphous otenaproxesul or a pharmaceutically acceptable salt thereof is formulated for delivery by injection, such as by intravenous (IV) injection. Therefore, in some embodiments, each composition of the application is an intravenous formulation of the application. A person skilled in the art can convert the dosage amounts noted above for oral dosages of amorphous otenaproxesul or a pharmaceutically acceptable salt thereof using standard pharmacokinetic (PK) and pharmacodynamic (PD) data for otenaproxesul or a pharmaceutically acceptable salt thereof.

In some embodiments, the pain is acute pain. In some embodiments, the acute pain is post-operative pain, and the loading dose is administered on the day before surgery. In some embodiments, the loading dose is administered about 24 hours, about 20 hours, about 16 hours, about 14 hours or about 12 hours before surgery. In some embodiments, the acute pain is post-operative pain, and the loading dose is administered on the day of the surgery. In some embodiments, the loading dose is administered before the surgery on the day of the surgery. In some embodiments, the loading dose is administered about 1 hour to about 8 hours, about 1 hour to about 6 hours, about 1 hour to about 4 hours, about 1 hour to about 3 or about 1 hour to about 2 hours before surgery. In some embodiments, the loading dose is administered about 8 hours, about 6 hours, about 5 hours, about 4 hours, about 2 hours or about 1 hour before surgery. In some embodiments, the loading dose is administered during the surgery. In some embodiments, the loading dose is administered after the surgery on the day of the surgery. In some embodiments, the loading dose is administered immediately after surgery. In some embodiments, the loading dose is administered about 30 minutes to about 4 hours after surgery. In some embodiments, the loading dose is administered about 30 minutes, about 1 hour, about 2 hours, about 3 hours, or about 4 hours after surgery. In some embodiments, the loading dose is administered during surgery.

In some embodiments, the acute pain is trauma, and the loading dose is administered after the trauma on the day the trauma was induced. In some embodiments, the loading dose is administered 15 minutes to about 20 hours, about 15 minutes to about 25 hours, about 30 minutes to about 18 hours, about 30 minutes to about 12 hours, about 30 minutes to about 6 hours, about 30 minutes to about 4 hours, about 1 hours to about 6 hours or about 1 hour to about 4 hours after trauma.

In some embodiments, the one or more pharmaceutical compositions of the application comprising loading dose are administered in advance of expected or possible trauma, such as in the cases of entry into situations that are at high risk for trauma. In some embodiments, the one or more pharmaceutical compositions of the application comprising loading doses are administered about 24 hours, about 20 hours, about 16 hours, about 14 hours or about 12 hours before expected or possible trauma. In some embodiments, the one or more pharmaceutical compositions of the application comprising loading doses are administered before the expected or possible trauma on the day of the he expected or possible trauma. In some embodiments, the loading dose is administered about 1 hour to about 8 hours, about 1 hour to about 6 hours, about 1 hour to about 4 hours, about 1 hour to about 3 or about 1 hour to about 2 hours before he expected or possible trauma. In some embodiments, the one or more pharmaceutical compositions of the application comprising loading doses are administered about 8 hours, about 6 hours, about 5 hours, about 4 hours, about 2 hours or about 1 hour before he expected or possible trauma.

In some embodiments, the dosage regimens for the compositions of the application are for treating chronic pain conditions, for example involving episodic pain, such as osteoarthritis. In these embodiments, the dosage regimens described above further comprise a “drug holiday” or a period of no drug administration or of administration of placebos for a period of, for example 1 to 30 days, before the next dosage regimen starting with a loading dose is administered. In some embodiments for chronic conditions this cycle of drug administration followed by a drug holiday is continued for many months or years or for as long as treatment is needed.

In some embodiments, the methods of the application provide pain relief within about 30 minutes to about 4 hours, about 30 minutes to about 1 hours, about 30 minutes to about 2 hours, about 1 hour to about 4 hours, about 1 hour to about 3 hours, about 1 hour to about 3 hours, about 1 hour to about 2 hours, about 2 hours to about 3 hours after administration of the one or more pharmaceutical compositions of the application comprising loading doses. In some embodiments, the methods of the application provide pain relief within about 30 minutes to about 3 hours, about 30 minutes to about 2 hours, about 1 hour to about 2 hours, about 2 hours to about 3 hours after administration of the one or more pharmaceutical compositions of the application comprising loading doses. In some embodiments, the methods of the application provide pain relief within about 30 minutes to about 3 hours, about 30 minutes to about 2 hours, about 1 hour to about 2 hours, after administration of the one or more pharmaceutical compositions of the application comprising loading doses.

In some embodiments, the therapeutic methods and uses of the application provide a Tmax of naproxen metabolite in a subject in less than about 60 minutes, less than about 90 minutes or less than about 120 minutes after oral administration of the pharmaceutical composition of the application comprising the loading dose. In some embodiments, the therapeutic methods of the application provide a Tmax of naproxen metabolite in a subject in less than about 60 minutes after oral administration of the pharmaceutical composition of the application comprising the loading dose.

In some embodiments, the therapeutic methods and uses of the application provide a Tmax of naproxen in about 1 hour to 3 hours, about 1 hour to about 2 hours, about 30 minutes to about 90 minutes or about 30 minutes to about on after oral administration of the pharmaceutical composition of the application comprising the loading dose. In some embodiments, the therapeutic methods of the application provide a Tmax of naproxen metabolite at about 2 hours to about 8 hours, about 3 hours to about 7 hours, about 4 hours to about 6.5 hours or about 4.7 to about 6 hours.

In some embodiments, the therapeutic methods and uses of the application comprising administration of the compositions of the application (including loading doses and maintenance doses) provide a Cmax of naproxen metabolite of about 20 g/mL to about 100 μg/mL, about 30 μg/mL to about 80 μg/mL, about 40 μg/mL to about 70 μg/mL, or about 50 μg/mL to about 65 μg/mL following oral administration to a subject of pharmaceutical compositions of the application. In some embodiments, the therapeutic methods of the application provide a Cmax of naproxen metabolite of about 20 μg/mL to about 100 μg/mL, about 30 μg/mL to about 80 μg/mL, about 40 μg/mL to about 70 μg/mL, or about 50 μg/mL to about 65 μg/mL.

In some embodiments, the therapeutic methods and uses of the application comprising administration of the compositions of the application (including loading doses and maintenance doses) provide an AUC of naproxen metabolite of from about 500 μg*hr/mL to about 4000 μg*hr/mL following administration of the compositions of the application. In some embodiments, the therapeutic methods of the application provide an AUC of naproxen metabolite of about 500 μg*hr/mL to about 3500 μg*hr/mL or about 500 μg*hr/mL to about 3000 μg*hr/mL.

In some embodiments, the therapeutic methods and uses of the application comprising administration of the compositions of the application (including loading doses and maintenance doses) provide a lower steady state (e.g, Day 4) AUC of naproxen compared to otherwise identical methods and uses except comprising administering pharmaceutical composition comprising equivalent doses of crystalline otenaproxesul under identical conditions.

In some embodiments, the subject is a subject at risk of liver injury. In some embodiments, the subject is a mammal. In some embodiments, the subject is a human. In some embodiments, the subject “in need thereof” is a subject having the disease disorder or condition.

In some embodiments, the methods of the application provide sustained pain relief. Therefore, in some embodiments, one or more pharmaceutical compositions of the application is used or administered alone without other known agents useful for treating or preventing pain.

In some embodiments the methods of the application comprise intermittent or repeated cycles of administration or use of the compositions of the application. For example, the methods of the application are used to treat recurring diseases, disorders or conditions and the compositions of the application are administered or used according to the methods described herein when the diseases, disorders or conditions re-occur or flare-up. In some embodiments, such diseases, disorders or conditions are any disease, disorder or condition that is treatable by inhibition of COX-1 and/or COX-2 that can re-occur such as, but not limited to, urinary tract infections, acute exacerbations of chronic obstructive pulmonary disease, headaches (such as migraines) and dysmenorrhea.

In some embodiments, the compositions of the application are used or administered in combination with other known agents useful for treating or preventing pain.

In some embodiments, the compositions of the application are used or administered in combination with other known agents useful for treating or preventing migraine, such as a triptan, including Sumatriptan.

In some embodiments, when used with other known agents useful for treating or preventing pain, the methods of the application reduces the amount of the other known agents used or administered. In some embodiments, when used with other known agents useful for treating or preventing acute pain, the methods of the application reduces the amount of the other known agents by about 40% to 60% within about 12 hours, about 24 hours, about 36 hours, about 48 hours, about 60 hours or about 72 hours post administration the loading dose.

When used in combination with other agents or therapies, it is an embodiment that the compositions or formulations of the application are administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other and can include administering the two substances or therapies within a few hours of each other, or even administering one substance or therapy within 24 hours of administration of the other if the pharmacokinetics are suitable. Design of suitable dosing regimens is routine for one skilled in the art. In particular embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

### Dosage Regimens for Crystalline Otenaproxesul

In some embodiments, the methods of the application comprise administering one or more loading doses of crystalline otenaproxesul or a pharmaceutically acceptable salt and/or solvate thereof.

In some embodiments, only one loading dose of crystalline otenaproxesul or a pharmaceutically acceptable salt thereof is administered and the loading dose comprises about 100 mg to about 2000 mg, about 200 mg to about 1500 mg, about 300 mg to about 1250 mg, about 400 mg to about 1000 mg of crystalline otenaproxesul or a pharmaceutically acceptable salt thereof. In some embodiments, the loading dose of crystalline otenaproxesul or a pharmaceutically acceptable salt thereof is formulated for oral delivery.

In an exemplary embodiment, the method comprises administering one loading dose of crystalline otenaproxesul or a pharmaceutically acceptable salt thereof, wherein the loading dose comprises about 100 mg to about 2000 mg of crystalline otenaproxesul or a pharmaceutically acceptable salt thereof. In an exemplary embodiment, the method comprises administering one loading dose of crystalline otenaproxesul or a pharmaceutically acceptable salt thereof, wherein the loading dose is about 100 mg to about 2000 mg, or about 200 mg to about 1500 mg of crystalline otenaproxesul or a pharmaceutically acceptable salt thereof.

In some embodiments, the loading dose of crystalline otenaproxesul or a pharmaceutically acceptable salt thereof, provides a plasma concentration of naproxen, a metabolite of otenaproxesul, of about 10 μg/mL or greater, about 15 μg/mL or greater or about 20 μg/mL or greater in less than about 30 minutes, less than about 45 minutes, less than about 60 minutes, less than about 75 minutes or less than about 90 minutes after administration. In some embodiments, loading dose of crystalline otenaproxesul or a pharmaceutically acceptable salt thereof, provides a plasma concentration of naproxen of about 10 μg/mL or greater, about 15 μg/mL or greater or about 20 μg/mL or greater in less than about 60 minutes after administration.

In some embodiments, loading dose of crystalline otenaproxesul or a pharmaceutically acceptable salt thereof, provides a C₆₀ of naproxen of about 10 μg/mL or greater, about 15 μg/mL or greater or about 20 μg/mL or greater after administration.

In some embodiments, the method comprises, on the third, fourth, fifth, sixth or seventh day, optionally on the third, fourth, fifth, sixth, seventh, eighth, ninth or tenth day, after administration of one or more loading doses of crystalline otenaproxesul or a pharmaceutically acceptable salt thereof, administering a second loading dose of crystalline otenaproxesul or a pharmaceutically acceptable salt thereof to the subject. In this embodiment, the loading dose of crystalline otenaproxesul or a pharmaceutically acceptable salt thereof becomes a first loading dose of crystalline otenaproxesul or a pharmaceutically acceptable salt thereof. In some embodiments, the administering of a second loading dose is on the third day or fourth day after administration of the first loading dose.

In some embodiments, on days preceding or following the administration of the second loading dose of crystalline otenaproxesul or a pharmaceutically acceptable salt thereof, the subject is administered a composition comprising a placebo.

In some embodiments, the second loading dose of crystalline otenaproxesul or a pharmaceutically acceptable salt thereof comprises the same amount of crystalline otenaproxesul or a pharmaceutically acceptable salt thereof as the first loading dose of crystalline otenaproxesul or a pharmaceutically acceptable salt thereof. In some embodiments, the second loading dose comprises a lower amount of crystalline otenaproxesul or a pharmaceutically acceptable salt thereof as the first loading dose. In some embodiments, the second loading dose is formulated for oral delivery and comprises about 100 mg to about 2000 mg of crystalline otenaproxesul or a pharmaceutically acceptable salt thereof. In some embodiments, the second loading dose of crystalline otenaproxesul or a pharmaceutically acceptable salt thereof is formulated for oral delivery and comprises about 100 mg to about 2000 mg, about 200 mg to about 1500 mg, about 300 mg to about 1250 mg, about 400 mg to about 1000 mg of crystalline otenaproxesul or a pharmaceutically acceptable salt thereof.

In some embodiments, the first and/or second loading dose of the crystalline otenaproxesul or a pharmaceutically acceptable salt thereof are each provided as a dose or as divided doses wherein the divided doses combined provide the loading dose, and each divided loading dose may be the same or different.

In some embodiments, on the days preceding or following administration of the second loading dose of the crystalline otenaproxesul or a pharmaceutically acceptable salt thereof, the subject is administered a composition comprising a placebo.

In some embodiments, instead of the second loading dose, the method further comprises administration of one or more maintenance doses of the crystalline otenaproxesul or a pharmaceutically acceptable salt thereof, wherein administration of the compositions comprising crystalline otenaproxesul or a pharmaceutically acceptable salt thereof is for 14 days or less.

Therefore, in some embodiments, the method comprises administering one or more loading doses of the crystalline otenaproxesul or a pharmaceutically acceptable salt thereof, followed by administration of one or more maintenance doses of the crystalline otenaproxesul or a pharmaceutically acceptable salt thereof, wherein administration of the crystalline otenaproxesul or a pharmaceutically acceptable salt thereof is for a treatment period of 14 days or less. In some embodiments, the treatment period is for 12 days or less, 10 days or less, 8 days or less, 6 days or less, 4 days or less, 3 days or less or 2 days. In some embodiments, the treatment period is for 10 days or less, 8 days or less, 6 days or less, 4 days or less. In some embodiments, the treatment period is for 6 days or less, 5 days or less or 4 days or less. In some embodiments, the treatment period is for 8 days, 7 days, 6 days, 5 days or 4 days. In some embodiments, the treatment period is for 5 days.

In some embodiments, the one or more maintenance doses of crystalline otenaproxesul or a pharmaceutically acceptable salt thereof are provided as a single dose or as divided doses of crystalline otenaproxesul or a pharmaceutically acceptable salt thereof, wherein the divided doses combined provide the maintenance dose. In some embodiments, each divided maintenance dose is the same or different. In some embodiments, each maintenance dose is the same or different.

In some embodiments, the one or more maintenance doses of crystalline otenaproxesul or a pharmaceutically acceptable salt thereof comprises about 25 mg to about 600 mg of crystalline otenaproxesul, or pharmaceutically acceptable salt thereof. In some embodiments, the one or more maintenances doses are administered or used as a single dose or a divided dose wherein divided doses combined provide the maintenance dose, and each maintenance dose or each divided dose may be the same or different.

Therefore, in some embodiments, the method comprises:
administering one or more loading doses comprising about 100 mg to about 2000 mg of crystalline otenaproxesul or a pharmaceutically acceptable salt thereof, and
administering one or more maintenance doses comprising about 25 mg to about 600 mg of crystalline otenaproxesul or a pharmaceutically acceptable salt thereof,
wherein administration of the crystalline otenaproxesul or a pharmaceutically acceptable salt thereof is for a treatment period of 14 days or less.

Therefore, in some embodiments, the method comprises:
administering one or more loading doses comprising about 100 mg to about 1500 mg of crystalline otenaproxesul or a pharmaceutically acceptable salt thereof, and
administering one or more maintenance doses comprising about 25 mg to about 600 mg of crystalline otenaproxesul or a pharmaceutically acceptable salt thereof,
wherein administration of the crystalline otenaproxesul or a pharmaceutically acceptable salt thereof is for a treatment period of 14 days or less.

In some embodiments, the one or more loading doses of crystalline otenaproxesul or a pharmaceutically acceptable salt thereof are greater than the one or more maintenances doses of crystalline otenaproxesul or a pharmaceutically acceptable salt thereof. In some embodiments, each of the maintenance doses are the same, i.e., each maintenance dose comprises the same amount of crystalline otenaproxesul or a pharmaceutically acceptable salt thereof.

In some embodiments, the one or more a maintenances dose of crystalline otenaproxesul or a pharmaceutically acceptable salt thereof are provided as tapered doses. Therefore, in some embodiments, initial maintenance doses are higher than following maintenance doses and for doses on a first and second consecutive day, the dose on first consecutive day is always the same or greater that the dose on the second consecutive day. In some embodiments, when two or more maintenance doses are administered on the same day, the first maintenance dose of the day is always the same or greater than the following maintenance dose(s) on the same day. In some embodiments, tapering, or a tapered dose includes, for example, a dose that is decreased stepwise by an amount over a period of time, for example about every 6 hours, 12 hours, 18 hours, 24 hours, 36 hours or 72 hours or a combination thereof. In some embodiments, tapering, or a tapered dose includes, for example, a daily dose that is decreased stepwise by an amount over a period of one or more days at each step, or a daily dose that is decreased by an amount each consecutive day. Tapering does not permit an increased following maintenance dose amount compared to the initial maintenance dose amount. Tapering does not permit an increased daily dose amount compared to the previous day.

A person skilled in the art will understand that there are many possible combinations of loading doses and maintenance doses of crystalline otenaproxesul or a pharmaceutically acceptable salt thereof that will provide the desired cumulative plasma naproxen metabolite (M25) exposure of less than about 4000 μg*hr/mL.

In some embodiments, the one or more maintenance doses of crystalline otenaproxesul or a pharmaceutically acceptable salt thereof are provided on consecutive days. In some embodiments, the one or more maintenance doses of crystalline otenaproxesul or a pharmaceutically acceptable salt thereof are provided on consecutive days and/or on non-consecutive days. In some embodiments, the one or more maintenance doses of crystalline otenaproxesul or a pharmaceutically acceptable salt thereof are administered one or more times per day. In some embodiments, two a maintenance doses of crystalline otenaproxesul or a pharmaceutically acceptable salt thereof are administered per day. In some embodiments, the maintenance doses of crystalline otenaproxesul or a pharmaceutically acceptable salt thereof are administered on consecutive days and/or on non-consecutive days.

In some embodiments, each maintenance dose of crystalline otenaproxesul or a pharmaceutically acceptable salt thereof is the same or different. In some embodiments, the maintenance doses of crystalline otenaproxesul or a pharmaceutically acceptable salt thereof are administered as tapered doses.

In some embodiments, on days where no maintenance dose of crystalline otenaproxesul or a pharmaceutically acceptable salt thereof is administered, the subject is administered a composition comprising a placebo.

In some embodiments, each maintenance dose of crystalline otenaproxesul or a pharmaceutically acceptable salt thereof independently comprises about 25 mg about 50 mg, about 75 mg, about 100 mg, about 125 mg, about 150 mg, about 175 mg, about 200 mg, about 225 mg, about 250 mg, about 300 mg, about 350 mg, about 400 mg, about 500 mg or about 600 mg of crystalline otenaproxesul, or a pharmaceutically acceptable salt thereof. In some embodiments, each maintenance dose of crystalline otenaproxesul or a pharmaceutically acceptable salt thereof is provided as a single dose or as divided doses.

In some embodiments, the one or more loading doses of crystalline otenaproxesul or a pharmaceutically acceptable salt thereof are formulated for intravenous delivery and comprises about 25 mg to about 2000 mg per day of crystalline otenaproxesul or a pharmaceutically acceptable salt thereof. In some embodiments, the one or more loading doses are formulated for intravenous delivery and comprises about 25 mg to about 1500 mg per day of crystalline otenaproxesul or a pharmaceutically acceptable salt thereof.

In some embodiments, the one or more loading doses comprises about 100 mg to about 2000 mg per day of crystalline otenaproxesul, or a pharmaceutically acceptable salt thereof and each maintenance dose comprises about 25 mg to about 400 mg to per day of crystalline otenaproxesul, or a pharmaceutically acceptable salt thereof.

Therefore, in an exemplary embodiment, the method comprises:
administering one or more loading doses comprising about 100 mg to about 2000 mg of crystalline otenaproxesul or a pharmaceutically acceptable salt thereof, and
administering one or more maintenance doses comprising about 25 mg to about 400 mg of crystalline otenaproxesul or a pharmaceutically acceptable salt thereof,
   wherein administration of the crystalline otenaproxesul or a pharmaceutically acceptable salt thereof is for a treatment period of 10 days or less.

In some embodiments, the one or more loading dose comprises about 1500 mg or about 1000 mg of crystalline otenaproxesul or a pharmaceutically acceptable salt thereof and the one or more maintenance doses comprise about 100 mg or about 200 mg of crystalline otenaproxesul or a pharmaceutically acceptable salt thereof and the administration of the crystalline otenaproxesul or a pharmaceutically acceptable salt thereof is for a treatment period of 5 days or less.

In some embodiments, the one or more loading dose comprises about 600 mg of crystalline otenaproxesul or a pharmaceutically acceptable salt thereof and the one or more maintenance doses comprise about 200 mg of crystalline otenaproxesul or a pharmaceutically acceptable salt thereof and the administration of the crystalline otenaproxesul or a pharmaceutically acceptable salt thereof is for a treatment period of 5 days or less.

In some embodiments, the one or more loading doses comprise about 100 mg to about 1500 mg, about 100 mg to about 1000 mg, about 100 mg to about 600 mg, or about 200 mg to about 400 mg per day of crystalline otenaproxesul, or a pharmaceutically acceptable salt thereof and each maintenance dose comprises about 50 mg to about 400 mg of crystalline otenaproxesul, or a pharmaceutically acceptable salt thereof.

In another exemplary embodiment, the method comprises
administering one loading dose comprising about 100 mg to about 2000 mg of crystalline otenaproxesul or a pharmaceutically acceptable salt thereof, and
administering one or more maintenance doses comprising about 50 mg to about 400 mg of crystalline otenaproxesul or a pharmaceutically acceptable salt thereof,
   wherein administration of the crystalline otenaproxesul or a pharmaceutically acceptable salt thereof is for a treatment period of 7 days or less.

In some embodiments, the loading dose comprises about 1000 mg to about 2000 mg of crystalline otenaproxesul or a pharmaceutically acceptable salt thereof and each maintenance dose independently comprises about 50 mg to about 400 mg of crystalline otenaproxesul or a pharmaceutically acceptable salt thereof. In some embodiments, the loading dose comprises about 1000 mg to about 2000 mg of crystalline otenaproxesul or a pharmaceutically acceptable salt thereof and each maintenance dose independently comprises about 100 mg to about 400 mg of crystalline otenaproxesul or a pharmaceutically acceptable salt.

In some embodiments, the loading dose comprises about 600 mg of crystalline otenaproxesul or a pharmaceutically acceptable salt thereof and each maintenance dose independently comprises about 50 mg to about 200 mg of crystalline otenaproxesul or a pharmaceutically acceptable salt thereof. In some embodiments, the loading dose comprises about 400 mg of crystalline otenaproxesul or a pharmaceutically acceptable salt thereof and each maintenance dose independently comprises about 50 mg, about 100 mg or about 200 mg of crystalline otenaproxesul or a pharmaceutically acceptable salt.

In some embodiments, the loading dose comprises about 200 mg of crystalline otenaproxesul or a pharmaceutically acceptable salt thereof and each maintenance dose independently comprises about 25 mg to about 100 mg of crystalline otenaproxesul or a pharmaceutically acceptable salt thereof. In some embodiments, the loading dose comprises about 200 mg of crystalline otenaproxesul or a pharmaceutically acceptable salt thereof and each maintenance dose independently comprises about 25 mg, about 50 mg or about 100 mg of crystalline otenaproxesul or a pharmaceutically acceptable salt thereof.

In some embodiments, the loading dose comprises about 600 mg of crystalline otenaproxesul or a pharmaceutically acceptable salt thereof and each maintenance dose independently comprises about 25 mg to about 200 mg of crystalline otenaproxesul or a pharmaceutically acceptable salt thereof. In some embodiments, the loading dose comprises about 600 mg of crystalline otenaproxesul or a pharmaceutically acceptable salt thereof and each maintenance dose independently comprises about 25 mg, about 50 mg, about 100 mg or about 200 mg of crystalline otenaproxesul or a pharmaceutically acceptable salt thereof.

In some embodiments, each dose of crystalline otenaproxesul or a pharmaceutically acceptable salt thereof, including loading doses and maintenance doses, is divided into a plurality of individual doses wherein the sum of the individual doses equals the dose. In some embodiments, each dose of crystalline otenaproxesul or a pharmaceutically acceptable salt thereof is divided into 2 to 6 individual doses wherein the sum of the individual doses equals the dose. In some embodiments, each dose of crystalline otenaproxesul or a pharmaceutically acceptable salt thereof is divided into 2, 3 or 4 individual doses wherein the sum of the individual doses equals the dose. In some embodiments, each dose of crystalline otenaproxesul or a pharmaceutically acceptable salt thereof is divided into 2 or 3 individual doses wherein the sum of the individual doses equals the dose. In some embodiments, each dose of crystalline otenaproxesul or a pharmaceutically acceptable salt thereof is divided into 2 individual doses wherein the sum of the individual doses equals the dose. In some embodiments, each maintenance dose of crystalline otenaproxesul or a pharmaceutically acceptable salt thereof is divided into 2 individual doses wherein the sum of the individual doses equals the dose.

In some embodiments, the one or more maintenance doses of crystalline otenaproxesul or a pharmaceutically acceptable salt thereof are administered on the day following the administration the loading dose of crystalline otenaproxesul or a pharmaceutically acceptable salt thereof. In some embodiments, the one or more maintenance doses of crystalline otenaproxesul or a pharmaceutically acceptable salt thereof are administered on the same day as the administration of the loading dose of crystalline otenaproxesul or a pharmaceutically acceptable salt thereof. In some embodiments, one maintenance dose of crystalline otenaproxesul or a pharmaceutically acceptable salt thereof is administered following the administration of the loading dose of crystalline otenaproxesul or a pharmaceutically acceptable salt thereof on the same day. In some embodiments, the loading dose of crystalline otenaproxesul or a pharmaceutically acceptable salt thereof is administered in the morning and the maintenance dose of crystalline otenaproxesul or a pharmaceutically acceptable salt thereof is administered in the evening of the same day.

In some embodiments, more than one maintenance dose of crystalline otenaproxesul or a pharmaceutically acceptable salt thereof are administered per day. In some embodiments, two or maintenance doses of crystalline otenaproxesul or a pharmaceutically acceptable salt thereof are administered per day. In some embodiments, two maintenance doses are administered per day. In some embodiments, one maintenance dose of crystalline otenaproxesul or a pharmaceutically acceptable salt thereof is administered in the morning and the other is administered in the evening of the same day (e.g. every 12 hours).

In some embodiments, the loading dose of crystalline otenaproxesul or a pharmaceutically acceptable salt thereof is administered in the morning and the maintenance dose of crystalline otenaproxesul or a pharmaceutically acceptable salt thereof is administered in the evening on the same day, and on the following treatment days, two maintenance doses of crystalline otenaproxesul or a pharmaceutically acceptable salt thereof are administered, and wherein one maintenance dose of crystalline otenaproxesul or a pharmaceutically acceptable salt thereof is administered in the morning and the other is administered in the evening of the same day (e.g. every 12 hours).

In some embodiments, each maintenance dose of crystalline otenaproxesul or a pharmaceutically acceptable salt thereof is the same or different. In some embodiments, the maintenance doses of crystalline otenaproxesul or a pharmaceutically acceptable salt thereof are tapered after administration of the loading dose of crystalline otenaproxesul or a pharmaceutically acceptable salt thereof to the second, third, fourth day fifth, six or seventh (last) day after administration of the loading dose. In some embodiments, the maintenance doses of crystalline otenaproxesul or a pharmaceutically acceptable salt thereof are tapered from the administration of the loading dose of crystalline otenaproxesul or a pharmaceutically acceptable salt thereof to the last day of the treatment period. In some embodiments, the maintenance doses of crystalline otenaproxesul or a pharmaceutically acceptable salt thereof are tapered from the first day after administration of the loading dose of crystalline otenaproxesul or a pharmaceutically acceptable salt thereof to third, fourth day or fifth day after administration of the loading dose of crystalline otenaproxesul or a pharmaceutically acceptable salt thereof. In some embodiments, the maintenance doses of crystalline otenaproxesul or a pharmaceutically acceptable salt thereof are tapered from the first day after administration of the loading dose of crystalline otenaproxesul or a pharmaceutically acceptable salt thereof to the last day of the treatment period. When more than one maintenance doses are administered per day, the first maintenance dose of the day is always the same or greater than the following maintenance dose(s) on the same day.

In some embodiments, each maintenance dose of crystalline otenaproxesul or a pharmaceutically acceptable salt thereof is different and comprises initial maintenance doses and following maintenance doses. Therefore, in some embodiments, the one or more maintenance doses of crystalline otenaproxesul or a pharmaceutically acceptable salt thereof are one or more initial maintenance doses of crystalline otenaproxesul or a pharmaceutically acceptable salt thereof and one or more following maintenance doses of crystalline otenaproxesul or a pharmaceutically acceptable salt thereof. In some embodiments, each maintenance dose of crystalline otenaproxesul or a pharmaceutically acceptable salt thereof is selected from an initial maintenance dose and a following maintenance dose. Therefore, in some embodiments, the method comprises administering one or more initial maintenance doses of crystalline otenaproxesul or a pharmaceutically acceptable salt thereof; and administering one or more following maintenance doses of crystalline otenaproxesul or a pharmaceutically acceptable salt thereof.

In some embodiments, the one or more initial maintenance doses of crystalline otenaproxesul or a pharmaceutically acceptable salt thereof are higher than the one or more following maintenance doses of crystalline otenaproxesul or a pharmaceutically acceptable salt thereof. In some embodiments, the one or more initial and/or following maintenance doses of crystalline otenaproxesul or a pharmaceutically acceptable salt thereof are provided on consecutive days and/or on non-consecutive days. In some embodiments, the one or more initial and/or following maintenance doses of crystalline otenaproxesul or a pharmaceutically acceptable salt thereof are provided about every 6 hours to 36 hours, 12 hours to 36 hours or 12 hours to 24 hours.

In some embodiments, one initial maintenance dose of crystalline otenaproxesul or a pharmaceutically acceptable salt thereof is administered on the same day as the loading dose of crystalline otenaproxesul or a pharmaceutically acceptable salt thereof. In some embodiments, one or two initial maintenance doses of crystalline otenaproxesul or a pharmaceutically acceptable salt thereof are administered the day following the administration of a loading dose of crystalline otenaproxesul or a pharmaceutically acceptable salt thereof.

In some embodiments, one initial maintenance dose of crystalline otenaproxesul or a pharmaceutically acceptable salt thereof is administered on the same day as the loading dose of crystalline otenaproxesul or a pharmaceutically acceptable salt thereof. In some embodiments, the loading dose of crystalline otenaproxesul or a pharmaceutically acceptable salt thereof is administered in the morning and one initial maintenance dose of crystalline otenaproxesul or a pharmaceutically acceptable salt thereof is administered in the evening of the same day, and on the following treatment days, one or two initial maintenance doses of crystalline otenaproxesul or a pharmaceutically acceptable salt thereof are administered; or one or two a following maintenance dose of crystalline otenaproxesul or a pharmaceutically acceptable salt thereof are administered.

In some embodiments, the loading dose of crystalline otenaproxesul or a pharmaceutically acceptable salt thereof is administered in the morning and one initial maintenance dose of crystalline otenaproxesul or a pharmaceutically acceptable salt thereof is administered in the evening of the same day, and on the following treatment day, one or two initial maintenance doses of crystalline otenaproxesul or a pharmaceutically acceptable salt thereof are administered, or one initial maintenance dose of crystalline otenaproxesul or a pharmaceutically acceptable salt thereof and one following maintenance dose of crystalline otenaproxesul or a pharmaceutically acceptable salt thereof is administered, and on following treatment days (e.g. day 3 onward) one or two following maintenance doses of crystalline otenaproxesul or a pharmaceutically acceptable salt thereof are administered per day. In some embodiments, on the following treatment days, two following maintenance doses of crystalline otenaproxesul or a pharmaceutically acceptable salt thereof are administered per day, once in the morning and once in the evening of the same day (e.g, about every 12 hours).

In some embodiments, the one or more following maintenance doses of crystalline otenaproxesul or a pharmaceutically acceptable salt thereof are tapered after administration of the initial maintenance dose of crystalline otenaproxesul or a pharmaceutically acceptable salt thereof to the second, third, fourth day fifth, six or seventh (last) day after administration of the loading dose of crystalline otenaproxesul or a pharmaceutically acceptable salt thereof. In some embodiments, the one or more following maintenance doses of crystalline otenaproxesul or a pharmaceutically acceptable salt thereof are tapered after administration of the initial maintenance dose of crystalline otenaproxesul or a pharmaceutically acceptable salt thereof to the last day of the treatment period.

In an exemplary embodiment, the loading dose of crystalline otenaproxesul or a pharmaceutically acceptable salt thereof is administered in the morning and one initial maintenance dose of crystalline otenaproxesul or a pharmaceutically acceptable salt thereof is administered in the evening of the same day, and on the following treatment day, one to three initial maintenance doses of crystalline otenaproxesul or a pharmaceutically acceptable salt thereof are administered, or one initial maintenance dose of crystalline otenaproxesul or a pharmaceutically acceptable salt thereof and one or two following maintenance doses of crystalline otenaproxesul or a pharmaceutically acceptable salt thereof are administered, and on the following treatment days one to three following maintenance doses of crystalline otenaproxesul or a pharmaceutically acceptable salt thereof are administered.

In an exemplary embodiment, the loading dose of crystalline otenaproxesul or a pharmaceutically acceptable salt thereof is administered in the morning and initial maintenance dose of crystalline otenaproxesul or a pharmaceutically acceptable salt thereof is administered in the evening of the same day, and on the following treatment day, two initial maintenance doses of crystalline otenaproxesul or a pharmaceutically acceptable salt thereof are administered, or one initial maintenance dose of crystalline otenaproxesul or a pharmaceutically acceptable salt thereof and one following maintenance dose of crystalline otenaproxesul or a pharmaceutically acceptable salt thereof are administered, and on the following treatment days one or two following maintenance doses of crystalline otenaproxesul or a pharmaceutically acceptable salt thereof are administered.

In some embodiments, each initial maintenance dose of crystalline otenaproxesul or a pharmaceutically acceptable salt thereof is the same or different. In some embodiments, each initial maintenance dose of crystalline otenaproxesul or a pharmaceutically acceptable salt thereof is the same.

In some embodiments, each following maintenance dose of crystalline otenaproxesul or a pharmaceutically acceptable salt thereof is the same or different. In some embodiments, each following maintenance dose of crystalline otenaproxesul or a pharmaceutically acceptable salt thereof dose are different and are tapered.

In some embodiments, the following maintenance doses of crystalline otenaproxesul or a pharmaceutically acceptable salt thereof are tapered after administration of the initial maintenance dose of crystalline otenaproxesul or a pharmaceutically acceptable salt thereof to the second, third, fourth day, fifth day, sixth or seventh (or last) after administration of the loading dose of crystalline otenaproxesul or a pharmaceutically acceptable salt thereof. In some embodiments, the following maintenance doses of crystalline otenaproxesul or a pharmaceutically acceptable salt thereof are tapered from the administration of the initial maintenance dose of crystalline otenaproxesul or a pharmaceutically acceptable salt thereof to the last day of the treatment period. In some embodiments, the following maintenance doses of crystalline otenaproxesul or a pharmaceutically acceptable salt thereof are tapered once or twice from the administration of the initial maintenance dose of crystalline otenaproxesul or a pharmaceutically acceptable salt thereof to the last day of the treatment period. In some embodiments, the following maintenance doses of crystalline otenaproxesul or a pharmaceutically acceptable salt thereof are tapered once from the administration of the initial maintenance dose of crystalline otenaproxesul or a pharmaceutically acceptable salt thereof to the last day of the treatment period.

In some embodiments, on days where no maintenance dose of crystalline otenaproxesul or a pharmaceutically acceptable salt thereof is administered, the subject is administered a composition comprising a placebo.

In exemplary embodiments, the loading dose comprises about 200 mg to about 2000 mg, or 200 mg to about 1000 mg of crystalline otenaproxesul or a pharmaceutically acceptable salt thereof, and each initial maintenance dose comprises about 50 mg to about 600 mg of crystalline otenaproxesul, or a pharmaceutically acceptable salt thereof, and each following maintenance dose comprises about 25 mg to about 400 mg of crystalline otenaproxesul, or a pharmaceutically acceptable salt thereof.

In some embodiments, the one or more initial maintenance doses of crystalline otenaproxesul or a pharmaceutically acceptable salt thereof are administered for 1 to 3 days. In some embodiments, the one or more initial maintenance doses of crystalline otenaproxesul or a pharmaceutically acceptable salt thereof are administered for 1 to 4 days 1 to 3 days, 1 to 2 days or 2 to 3 days. In some embodiments, the initial maintenance dose of crystalline otenaproxesul or a pharmaceutically acceptable salt thereof is administered only once. In some embodiments, one n initial maintenance dose of crystalline otenaproxesul or a pharmaceutically acceptable salt thereof is administered on the same day as the loading dose of crystalline otenaproxesul or a pharmaceutically acceptable salt thereof. In some embodiments, one initial maintenance dose of crystalline otenaproxesul or a pharmaceutically acceptable salt thereof is administered on the same day as the loading dose of crystalline otenaproxesul or a pharmaceutically acceptable salt thereof, and one or two initial maintenance doses of crystalline otenaproxesul or a pharmaceutically acceptable salt thereof are administered on the following day after administration of the loading dose of crystalline otenaproxesul or a pharmaceutically acceptable salt thereof. In some embodiments, one or more following maintenance doses of crystalline otenaproxesul or a pharmaceutically acceptable salt thereof are administered on the days after administration of the initial maintenance dose(s) of crystalline otenaproxesul or a pharmaceutically acceptable salt thereof.

In some embodiments, the initial maintenance doses of crystalline otenaproxesul or a pharmaceutically acceptable salt thereof are the same, i.e., not tapered. In some embodiments, the one or more initial maintenance doses of crystalline otenaproxesul or a pharmaceutically acceptable salt thereof are tapered. In some embodiments, the initial maintenance doses of crystalline otenaproxesul or a pharmaceutically acceptable salt thereof are the same.

In some embodiments, the one or more following maintenance doses of crystalline otenaproxesul or a pharmaceutically acceptable salt thereof are administered for 1 to 4 days following administration of the one or more initial maintenance doses of crystalline otenaproxesul or a pharmaceutically acceptable salt thereof. In some embodiments, the one or more following maintenance doses of crystalline otenaproxesul or a pharmaceutically acceptable salt thereof are administered 1 to 4 days 1 to 3 days, 2 to 3 days or 1 to 2 days following administration of the one or more initial maintenance doses of crystalline otenaproxesul or a pharmaceutically acceptable salt thereof.

In some embodiments, the following maintenance doses of crystalline otenaproxesul or a pharmaceutically acceptable salt thereof are the same, i.e., not tapered. In some embodiments, the following maintenance doses are tapered. In some embodiments, following maintenance doses of crystalline otenaproxesul or a pharmaceutically acceptable salt thereof are tapered about every 6 hours, 12 hours, 24 hours, 36 hours or 48 hours. In some embodiments, following maintenance doses of crystalline otenaproxesul or a pharmaceutically acceptable salt thereof are tapered every 24 hours to 36 hours. In some embodiments, following maintenance doses of crystalline otenaproxesul or a pharmaceutically acceptable salt thereof are tapered every 24 hours or 36 hours.

In an exemplary embodiment, the method comprises:
administering one loading dose comprising about 100 mg to about 2000 mg of crystalline otenaproxesul or a pharmaceutically acceptable salt thereof,
administering one or more initial maintenance doses comprising about 50 mg to about 600 mg of crystalline otenaproxesul or a pharmaceutically acceptable salt thereof, and
administering one or more following maintenance doses comprising about 25 mg to about 200 mg of crystalline otenaproxesul or a pharmaceutically acceptable salt thereof,
   wherein administration of the crystalline otenaproxesul or a pharmaceutically acceptable salt thereof is for a treatment period of 7 to 10 days or less, and each the initial and following maintenance doses are the same or different.

In some embodiments, the one loading dose comprising about 1000 mg to about 2000 mg of crystalline otenaproxesul or a pharmaceutically acceptable salt thereof.

In an exemplary embodiment, the method comprises:
administering one loading dose comprising about 100 mg to about 1000 mg of crystalline otenaproxesul or a pharmaceutically acceptable salt thereof,
administering one or more initial maintenance doses comprising about 50 mg to about 400 mg of crystalline otenaproxesul or a pharmaceutically acceptable salt thereof, and
administering one or more following maintenance doses comprising about 25 mg to about 200 mg of crystalline otenaproxesul or a pharmaceutically acceptable salt thereof,
   wherein administration of the crystalline otenaproxesul or a pharmaceutically acceptable salt thereof is for a treatment period of 7 to 10 days or less, and each the initial and following maintenance doses are the same or different.

In another exemplary embodiment, the method comprises:
administering one or more loading doses comprising about 100 mg to about 600 mg of crystalline otenaproxesul or a pharmaceutically acceptable salt thereof,
administering one or more initial maintenance doses comprising about 25 mg to about 200 mg of crystalline otenaproxesul or a pharmaceutically acceptable salt thereof, and
administering one or more following maintenance doses comprising about 25 mg to about 100 mg of crystalline otenaproxesul or a pharmaceutically acceptable salt thereof,
   wherein administration of the crystalline otenaproxesul or a pharmaceutically acceptable salt thereof is for a treatment period of 7 to 10 days or less.

In some embodiments, the treatment period is for 3 to 5, 4 to 5 or 5 to 6 days. In some embodiments, the treatment period is for 3, 4, 5 or 6 days. In some embodiments, the treatment period is for 4 or 5 days. In some embodiments, the treatment period is for 5 days.

In some embodiments, the loading dose comprises about 1000 mg of crystalline otenaproxesul or a pharmaceutically acceptable salt thereof, the initial maintenance dose comprises about 200 mg of crystalline otenaproxesul or a pharmaceutically acceptable salt thereof, and the following maintenance dose comprises about 100 mg of crystalline otenaproxesul or a pharmaceutically acceptable salt and/or solvate wherein the initial maintenance dose is administered for 1 to 3 days following administration of the loading dose and the following maintenance dose is administered for 1 to 3 days following administration of the initial maintenance dose. In some embodiments, the loading dose comprises about 1000 mg to about 2000 mg of crystalline otenaproxesul or a pharmaceutically acceptable salt thereof, the one or more initial maintenance doses comprise about 100 mg to about 400 mg of crystalline otenaproxesul or a pharmaceutically acceptable salt thereof, and the one or more following maintenance doses comprise about 25 mg to about 200 mg of crystalline otenaproxesul or a pharmaceutically acceptable salt thereof,
one of the initial maintenance doses is administered on the same day as the loading dose;
one or two of the initial maintenance doses or one initial maintenance dose and one following maintenance dose are administered on the day after administration of the loading dose,
and on subsequent treatment days (e.g., day 3 onward) one or two following maintenance doses are administered per day.

In some embodiments, the loading dose comprises about 600 mg of crystalline otenaproxesul or a pharmaceutically acceptable salt thereof, the one or more initial maintenance doses comprise about 200 mg of crystalline otenaproxesul or a pharmaceutically acceptable salt thereof, and the one or more following maintenance doses comprise about 50 mg to about 100 mg of crystalline otenaproxesul or a pharmaceutically acceptable salt thereof. In some embodiments, the loading dose comprises about 400 mg of crystalline otenaproxesul or a pharmaceutically acceptable salt thereof, the one or more initial maintenance doses comprise about 100 mg of crystalline otenaproxesul or a pharmaceutically acceptable salt thereof, and the one or more following maintenance doses comprises about 25 mg or about 50 mg of crystalline otenaproxesul or a pharmaceutically acceptable salt thereof, and
one of the initial maintenance doses is administered on the same day as the loading dose;
one or two of the initial maintenance doses or one initial maintenance dose and one following maintenance dose are administered on the day after administration of the loading dose,
and on subsequent treatment days (e.g., day 3 onward) one or two following maintenance doses are administered per day.

In some embodiments, the two initial or following maintenance dose of crystalline otenaproxesul or a pharmaceutically acceptable salt thereof are administered once in the morning and once in the evening of the same day.

In some embodiments, the initial and/or following maintenance doses of crystalline otenaproxesul or a pharmaceutically acceptable salt thereof are provided about every 12 hours to 24 hours. In some embodiments, the initial and/or following maintenance doses of crystalline otenaproxesul or a pharmaceutically acceptable salt thereof are provided about every 12 hours.

In some embodiments, each following maintenance dose of crystalline otenaproxesul or a pharmaceutically acceptable salt thereof is the same or different. In some embodiments, the following maintenance doses of crystalline otenaproxesul or a pharmaceutically acceptable salt thereof are tapered once or twice from the administration of a last initial maintenance dose of crystalline otenaproxesul or a pharmaceutically acceptable salt thereof to the last day of the treatment period. In some embodiments, the following maintenance doses of crystalline otenaproxesul or a pharmaceutically acceptable salt thereof are tapered once from the administration of a last initial maintenance dose of crystalline otenaproxesul or a pharmaceutically acceptable salt thereof to the last day of the treatment period.

In some embodiments, the following maintenance doses of crystalline otenaproxesul or a pharmaceutically acceptable salt thereof are tapered after 24 hours to 36 hours after administration of the first following maintenance dose of crystalline otenaproxesul or a pharmaceutically acceptable salt thereof.

In some embodiments, each dose of crystalline otenaproxesul or a pharmaceutically acceptable salt thereof, including loading, initial maintenance and following maintenance doses, is divided into a plurality of individual doses wherein the sum of the individual doses equals the dose. In some embodiments, each dose of crystalline otenaproxesul or a pharmaceutically acceptable salt thereof is divided into 2 to 6 individual doses wherein the sum of the individual doses equals the dose. In some embodiments, each dose of crystalline otenaproxesul or a pharmaceutically acceptable salt thereof is divided into 2, 3 or 4 individual doses wherein the sum of the individual doses equals the dose. In some embodiments, each dose of crystalline otenaproxesul or a pharmaceutically acceptable salt thereof is divided into 2 or 3 individual doses wherein the sum of the individual doses equals the dose. In some embodiments, each dose of crystalline otenaproxesul or a pharmaceutically acceptable salt thereof is divided into 2 individual doses wherein the sum of the individual doses equals the dose.

In some embodiments, each dose of crystalline otenaproxesul or a pharmaceutically acceptable salt and/or solvate thereof is suitably formulated into one or more pharmaceutical compositions for administration to subjects in a biologically compatible form suitable for administration in vivo. Accordingly, in some embodiments, each dose of crystalline otenaproxesul or a pharmaceutically acceptable salt and/or solvate thereof is formulated into a pharmaceutical composition comprising the dose of crystalline otenaproxesul or a pharmaceutically acceptable salt and/or solvate thereof and a pharmaceutically acceptable carrier. In some embodiments, the pharmaceutical composition comprising the dose of crystalline otenaproxesul or a pharmaceutically acceptable salt and/or solvate thereof is formulated for oral or intravenous administration.

In some embodiments, each dose of crystalline otenaproxesul or a pharmaceutically acceptable salt thereof is formulated for oral delivery.

In some embodiments, each dose of crystalline otenaproxesul or a pharmaceutically acceptable salt thereof is formulated for delivery by injection, such as by intravenous (IV) injection. A person skilled in the art can convert the dosage amounts noted above for oral dosages of crystalline otenaproxesul or a pharmaceutically acceptable salt thereof using standard pharmacokinetic (PK) and pharmacodynamic (PD) data for otenaproxesul or a pharmaceutically acceptable salt thereof.

In some embodiment, the pain is as defined in the “Method and uses of the application” section above including embodiments thereof. In some embodiments, the pain is acute pain. In some embodiments, the acute pain is post-operative pain.

In some embodiments, the acute pain is post-operative pain, and the loading dose of crystalline otenaproxesul or a pharmaceutically acceptable salt thereof is administered on the day before surgery. In some embodiments, the loading dose of crystalline otenaproxesul or a pharmaceutically acceptable salt thereof is administered about 24 hours, about 20 hours, about 16 hours, about 14 hours or about 12 hours before surgery. In some embodiments, the acute pain is post-operative pain, and the loading dose of crystalline otenaproxesul or a pharmaceutically acceptable salt thereof is administered on the day of the surgery. In some embodiments, the loading dose of crystalline otenaproxesul or a pharmaceutically acceptable salt thereof is administered before the surgery on the day of the surgery. In some embodiments, the loading dose of crystalline otenaproxesul or a pharmaceutically acceptable salt thereof is administered about 1 hour to about 8 hours, about 1 hour to about 6 hours, about 1 hour to about 4 hours, about 1 hour to about 3 or about 1 hour to about 2 hours before surgery. In some embodiments, the loading dose of crystalline otenaproxesul or a pharmaceutically acceptable salt thereof is administered about 8 hours, about 6 hours, about 5 hours, about 4 hours, about 2 hours or about 1 hour before surgery. In some embodiments, the loading dose of crystalline otenaproxesul or a pharmaceutically acceptable salt thereof is administered during the surgery. In some embodiments, the loading dose of crystalline otenaproxesul or a pharmaceutically acceptable salt thereof is administered after the surgery on the day of the surgery. In some embodiments, the loading dose of crystalline otenaproxesul or a pharmaceutically acceptable salt thereof is administered immediately after surgery. In some embodiments, the loading dose of crystalline otenaproxesul or a pharmaceutically acceptable salt thereof is administered about 30 minutes to about 4 hours after surgery. In some embodiments, the loading dose of crystalline otenaproxesul or a pharmaceutically acceptable salt thereof is administered about 30 minutes, about 1 hour, about 2 hours, about 3 hours, or about 4 hours after surgery. In some embodiments, the loading dose of crystalline otenaproxesul or a pharmaceutically acceptable salt thereof is administered during surgery.

In some embodiments, the acute pain is trauma, and the loading dose of crystalline otenaproxesul or a pharmaceutically acceptable salt thereof is administered after the trauma on the day the trauma was induced. In some embodiments, the loading dose of crystalline otenaproxesul or a pharmaceutically acceptable salt thereof is administered 15 minutes to about 20 hours, about 15 minutes to about 25 hours, about 30 minutes to about 18 hours, about 30 minutes to about 12 hours, about 30 minutes to about 6 hours, about 30 minutes to about 4 hours, about 1 hours to about 6 hours or about 1 hour to about 4 hours after trauma.

In some embodiments, the one or more loading doses of crystalline otenaproxesul or a pharmaceutically acceptable salt thereof are administered in advance of expected or possible trauma, such as in the cases of entry into situations that are at high risk for trauma. In some embodiments, the one or more loading doses of crystalline otenaproxesul or a pharmaceutically acceptable salt thereof are administered about 24 hours, about 20 hours, about 16 hours, about 14 hours or about 12 hours before expected or possible trauma. In some embodiments, the one or more loading doses of crystalline otenaproxesul or a pharmaceutically acceptable salt thereof are administered before the expected or possible trauma on the day of the he expected or possible trauma. In some embodiments, the loading dose of crystalline otenaproxesul or a pharmaceutically acceptable salt thereof is administered about 1 hour to about 8 hours, about 1 hour to about 6 hours, about 1 hour to about 4 hours, about 1 hour to about 3 or about 1 hour to about 2 hours before he expected or possible trauma. In some embodiments, the one or more loading doses of crystalline otenaproxesul or a pharmaceutically acceptable salt thereof are administered about 8 hours, about 6 hours, about 5 hours, about 4 hours, about 2 hours or about 1 hour before he expected or possible trauma.

In some embodiments, the dosage regimens for crystalline otenaproxesul or a pharmaceutically acceptable salt thereof are for treating chronic pain conditions, for example involving episodic pain, such as osteoarthritis. In these embodiments, the dosage regimens described above further comprise a “drug holiday” or a period of no drug administration or of administration of placebos for a period of, for example 1 to 30 days, before the next dosage regimen starting with a loading dose is administered. In some embodiments for chronic conditions this cycle of drug administration followed by a drug holiday is continued for many months or years or for as long as treatment is needed.

In some embodiments, the methods of the application provide pain relief within about 30 minutes to about 4 hours, about 30 minutes to about 1 hours, about 30 minutes to about 2 hours, about 1 hour to about 4 hours, about 1 hour to about 3 hours, about 1 hour to about 3 hours, about 1 hour to about 2 hours, about 2 hours to about 3 hours after administration of the one or more loading doses of crystalline otenaproxesul or a pharmaceutically acceptable salt thereof. In some embodiments, the methods of the application provide pain relief within about 30 minutes to about 3 hours, about 30 minutes to about 2 hours, about 1 hour to about 2 hours, about 2 hours to about 3 hours after administration of the one or more loading doses of crystalline otenaproxesul or a pharmaceutically acceptable salt thereof.

In some embodiments, the therapeutic methods and uses of the application comprising administration the loading doses and maintenance doses of crystalline otenaproxesul or a pharmaceutically acceptable salt thereof provide an AUC of naproxen metabolite of from about 500 μg*hr/mL to about 4000 μg*hr/mL following administration.

In some embodiments, the subject is a subject at risk of liver injury. In some embodiments, the subject is a mammal. In some embodiments, the subject is a human. In some embodiments, the subject “in need thereof” is a subject having the disease disorder or condition.

In some embodiments, the methods of the application provide sustained pain relief. Therefore, in some embodiments, crystalline otenaproxesul or a pharmaceutically acceptable salt thereof is used or administered alone without other known agents useful for treating or preventing pain.

In some embodiments the methods of the application comprise intermittent or repeated cycles of administration or use of the crystalline otenaproxesul or a pharmaceutically acceptable salt thereof. For example, the methods of the application are used to treat recurring diseases, disorders or conditions and the crystalline otenaproxesul or a pharmaceutically acceptable salt thereof are administered or used according to the methods described herein when the diseases, disorders or conditions re-occur or flare-up. In some embodiments, such diseases, disorders or conditions are any disease, disorder or condition that is treatable by inhibition of COX-1 and/or COX-2 that can re-occur such as, but not limited to, urinary tract infections, acute exacerbations of chronic obstructive pulmonary disease, headaches (such as migraines), gout and dysmenorrhea.

In some embodiments, the crystalline otenaproxesul or a pharmaceutically acceptable salt thereof is used or administered in combination with other known agents useful for treating or preventing pain.

In some embodiments, the crystalline otenaproxesul or a pharmaceutically acceptable salt thereof are used or administered in combination with other known agents useful for treating or preventing migraine, such as a triptan, including sumatriptan.

In some embodiments, when used with other known agents useful for treating or preventing pain, the methods of the application reduces the amount of the other known agents used or administered. In some embodiments, when used with other known agents useful for treating or preventing acute pain, the methods of the application reduces the amount of the other known agents by about 40% to 60% within about 12 hours, about 24 hours, about 36 hours, about 48 hours, about 60 hours or about 72 hours post administration the loading dose.

When used in combination with other agents or therapies, it is an embodiment that the crystalline otenaproxesul or a pharmaceutically acceptable salt thereof is administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other and can include administering the two substances or therapies within a few hours of each other, or even administering one substance or therapy within 24 hours of administration of the other if the pharmacokinetics are suitable. Design of suitable dosing regimens is routine for one skilled in the art. In particular embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

### EXAMPLES

The following non-limiting examples are illustrative of the present application:

### Example 1: Phase 1 Single Ascending Dose (SAD) Clinical Trial (State of the Art)

Otenaproxesul (crystalline form) was orally administered at a range of doses to healthy volunteers, both as a single dose, and as a daily (or twice-daily) dose for 14 days. Each cohort consisted of 8 subjects, with 6 receiving otenaproxesul and 2 receiving placebo. A total of 8 cohorts were studied in the single-dose study (25 mg, 75 mg, 150 mg, 300 mg, 500 mg, 1000 mg, 1500 mg and 2000 mg), and 3 cohorts in the multiple-dose study (250 and 750 once daily, and 750 mg bid (e.g., 1500 mg/day) for 14 days). All adverse events were recorded, and blood samples were drawn prior to and for at least 72 h after drug administration for measurement of otenaproxesul-derived naproxen levels. Routine cardiovascular, renal, hepatic and hematological biomarkers were monitored, along with clinical signs. Multiple ascending dose studies examined otenaproxesul at doses of 250 and 750 mg once daily, and 750 mg twice daily.

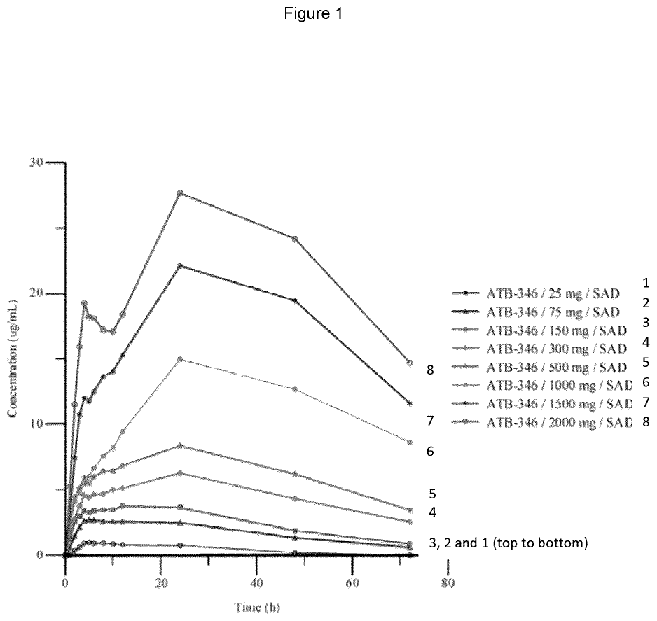

Otenaproxesul single dose treatment did not produce any serious adverse events across the dose-ranges studied, nor were there any significant gastrointestinal, cardiovascular renal, or hematological findings associated with administration of this drug. As initially observed in animal pharmacokinetics, otenaproxesul administration to humans resulted in a marked prolongation of therapeutically relevant levels of naproxen in the blood, supporting the possibility of once daily dosing for both acute and chronic conditions (FIG. 1).

Otenaproxesul was very well tolerated and shown to be safe at all single, once daily administered dose levels. These studies suggest a high level of safety of this drug.

### Example 2: a Double-Blind, Placebo-Controlled, 4-Arm, Phase 2b Study to Assess the Efficacy and Safety of a 14-Day Dosing Regimen of 3 Doses of Otenaproxesul Versus Placebo, Orally Administered Once Daily to Male and Female Patients Diagnosed with Osteoarthritis of the Knee (State of the Art)

This study evaluated the efficacy of a 14-day dosing regimen of crystalline otenaproxesul at doses of 150 mg, 200 mg and 250 mg compared to placebo in reducing osteoarthritis knee pain (a chronic pain condition) as measured by changes in the post-treatment Western Ontario and McMaster Universities Osteoarthritis Index (WOMAC) subscale pain score relative to each patient's pretreatment baseline WOMAC assessment. In patients with bilateral knee osteoarthritis, the most painful knee (index knee) or if in both knees of equal pain, then 1 index knee was noted and has been used for the WOMAC assessments throughout the study. The three doses were selected based on state-of-the-art knowledge of efficacy and safety for otenaproxesul at the time of the trial.

### Methodology:

This study was a double-blind, placebo-controlled Phase 2B study that explored 3 different doses (150 mg, 200 mg, and 250 mg) of orally administered crystalline otenaproxesul in male and female patients diagnosed with osteoarthritis of the knee. Patients were randomly assigned in a 2:2:1:1 allocation ratio to otenaproxesul 250 mg, 200 mg, 150 mg, or placebo.

Screening evaluations to determine patient eligibility for the study were performed within 28 days prior to the first treatment administration. To be eligible for enrolment, patients had to complete a 5-day washout period for NSAIDS or simple non-NSAID containing analgesics during the screening phase and have a baseline visual analog scale (VAS) score of >50 mm to 100 mm after a brief walking activity. Screening data were reviewed to determine patient eligibility and patients who met all inclusion criteria and none of the exclusion criteria were randomized into the study.

All patients were to take their assigned, blinded treatment once daily with water in the morning after an overnight fast. All study patients received training regarding placebo response to mitigate placebo response and enhance the accuracy of the clinical trial results.

Study visits occurred on Day 1, Day 4, and Day 14 of the 14-day treatment phase and included WOMAC evaluations, blood samples for thromboxane B2 TXB₂ and pharmacokinetic (PK) testing. The safety of otenaproxesul was evaluated through monitoring for adverse events (AEs), changes in physical examinations, vital signs measurements, and clinical laboratory evaluations.

Patients were followed for safety for up to 24 days following initiation of treatment. A final visit for safety assessments occurred upon study completion on Day 24 or early termination.

384 patients were enrolled and treated during the study including 61, 125, and 132 patients in the otenaproxesul 150 mg, 200 mg, and 250 mg treatment groups, respectively and 66 patients in the placebo group.

The study enrolled adult patients between 40 and 75 years of age, who had a clinical diagnosis of osteoarthritis of the index knee and with supporting radiological evidence and had a diagnosis greater than 2 years in duration requiring the use of regular therapies, e.g., oral or topical anti-inflammatoireacetaminophen, topical capsaicin. Patients must not have used aspirin or naproxen-containing medications for 7 days prior to randomization and not have used any NSAIDs or simple non-NSAID containing analgesics for 5 days prior to randomization. Patients must have showed a ≥10-point increase in WOMAC VAS score between their screening visit and baseline study entry visit.

Otenaproxesul was supplied as 150 mg, 200 mg, and 250 mg white, film-coated tablets in which the otenaproxesul was in crystalline form. Patients were instructed to take 1 tablet of otenaproxesul or placebo in the morning after an overnight fast. Food intake was restricted until at least 30 minutes after the morning dose.

The placebo product was a white, film-coated tablet with the same excipients as otenaproxesul tablets but no active ingredient. Both test product and matching placebo were over-encapsulated within Swedish Orange opaque size “A” DB capsule to maintain the blind between doses and placebo.

The efficacy of treatment with otenaproxesul was assessed by measuring the change in the WOMAC VAS version 3.1 subscale pain, stiffness, and difficulties performing daily activities (DPDA) scores after 14 days relative to each patient's pretreatment baseline WOMAC score.

Single time point plasma samples were taken on Days 1, 4 and 14 of treatment for the measurement of the principal otenaproxesul metabolite, naproxen.

Whole blood samples were collected pre-treatment for the measurement of genetic variants (polymorphic forms) of the naproxen metabolizing enzyme CYP2C9.

Whole blood samples were taken pre-treatment and on Days 1, 4 and 14 for the measurement of thromboxane B2 as an index of cyclo-oxygenase enzyme inhibition. Thromboxane B₂ levels were measured using the Thromboxane B₂ Elisa Kit from Cayman Chemical, Ann Arbor Michigan. Instructions provided with the Kit were followed.

Blood samples for measurement of thromboxane concentrations were collected at room temperature in 10 mL red top, no additive Vacutainers. Blood samples were taken approximately 4 hours after dosing and divided into 2 aliquots. The first aliquot (1 mL) was prepared for the measurement of otenaproxesul-derived plasma naproxen concentrations. The second aliquot (2 mL) was further divided into 2 polypropylene tubes for the measurement of plasma thromboxane concentrations.

### Safety Analyses

All patients receiving any amount of study treatment (otenaproxesul or placebo) were included in the safety analysis. Each treatment arm was evaluated separately. In addition to the evaluation and categorization of treatment-emergent adverse events (TEAEs), listings of laboratory test results collected at Baseline and during the study were generated. Descriptive statistics summarizing the changes in those laboratory tests over time were presented.

### Efficacy Analysis

All patients who received 1 dose of study drug (otenaproxesul or placebo) and had at least 1 post-baseline WOMAC pain subscale score observation were included in the primary population for the analysis of efficacy (modified-intent-to-treat population [mITT]). Patients who met the criteria for the primary efficacy analyses and had no major protocol violations that could have impacted the assessment of efficacy (per protocol population [PP]) were evaluated as a supportive analysis. The primary and secondary endpoints were analyzed using a maximum likelihood mixed model repeated measure (MMRM). The model included treatment, visit, baseline WOMAC pain score, and treatment-by-visit interaction as fixed effects, and patient as a random effect. Dose-response analysis was conducted by using a contrast statement to the least squares (LS) means on Day 14 between the dose levels of otenaproxesul and placebo. For patients who received rescue medication within 12 hours before an efficacy assessment, the assessments at that visit were disregarded and considered missing. Analysis of the WOMAC pain subscale score, including the MMRM analysis and descriptive summary, were also presented by sex (male, female) in a subgroup analysis.

### Pharmacokinetics and Pharmacodynamics Analyses

The average plasma concentrations for all patients in each treatment group as well as a comparison of the plasma naproxen concentrations for male and female patients within each of the three otenaproxesul treatment arms was assessed.

The genetic distribution of all genetic variants (polymorphic forms) of the naproxen metabolizing enzyme CYP2C9 are listed for patients with samples available in each treatment group as well as variant distribution for male and female patients within each of the 4 treatment arms.

Thromboxane B2 levels for all patients in each treatment group were presented in a listing.

### Patient Characteristics at Study Entry

There were no significant differences in the distributions of age, sex, or body mass index (BMI) between the otenaproxesul and placebo groups. The majority of patients enrolled in the otenaproxesul and placebo groups were female, (201 [63.2%] otenaproxesul-treated patients, and 41 [62.1%] placebo patients) and white (266 [83.6%] otenaproxesul-treated patients, and 55 [83.3%] placebo patients). The mean age in both groups was 64 years and ranged from 40 years to 75 years.

### Efficacy Results

Treatment with crystalline otenaproxesul demonstrated clinically and statistically significant improvement in WOMAC pain, stiffness, and Difficulties Performing Daily Activities (DPDA) subscale scores from Baseline to Day 14 in patients with osteoarthritis of the knee. The study met its primary endpoint for the otenaproxesul 250 mg and 200 mg treatment group comparisons that were powered for formal hypothesis testing. Note, the p-values for the analysis of the secondary efficacy endpoints are to be considered nominal, as no additional alpha adjustments were made.

In the otenaproxesul 250 mg and 200 mg treatment groups:
Decreases in WOMAC pain scores from Baseline to Day 14 reached statistical significance (p=0.0144 and p=0.0071, in the otenaproxesul 250 mg and 200 mg treatment groups respectively) when compared to placebo with LS mean changes of 135.3 mm (95% Cl: −154.0, −116.6) and −139.9 mm (95% Cl: −159.3, −120.6), respectively in each treatment group compared to −94.7 mm (95% Cl: −121.2, −68) in the placebo group.
Decreases in the WOMAC stiffness score compared to placebo from Baseline to Day 14 were statistically significant (p=<0.001, p=<0.001, respectively) with LS mean changes of −55.6 mm (95% Cl: −63.5, −47.8) and −54.4 mm (95% Cl: −62.6, −46.3), respectively in the otenaproxesul 250 mg and 200 mg treatment groups compared to −30.7 mm (−42.0, −19.5) in the placebo group.
Decreases in the WOMAC DPDA scores also reached statistical significance (p=0.0042 and p=0.0010, in the otenaproxesul 250 mg and 200 mg treatment groups respectively) compared to placebo from Baseline to Day 14 with LS mean changes of −412.9 mm (95% Cl: −475.1, −350.7) and −438.5 mm (95% Cl: −502.9, 374.1), in each treatment group respectively compared to −254.5 mm (95% Cl: −343.0, −166.1) in the placebo group.

In the otenaproxesul 150 mg treatment group:
While not statistically significant, a decrease in WOMAC pain and DPDA scores from Baseline to Day 14 between the otenaproxesul 150 mg and placebo groups was also observed. Decreases in the WOMAC stiffness score in the 150 mg treatment group compared to placebo from Baseline to Day 14 were statistically significant with an actual mean change −48.4 mm (95% Cl: −60.3, −36.6) compared to −30.7 mm (95% Cl: −42.0, −19.5) in the placebo group.

Additional post hoc efficacy endpoint analyses supported the assessment that clinically meaningful decreases in normalized pain subscale scores from Baseline to Day 14 were achieved for patients who received otenaproxesul 200 mg or 250 mg compared to patients who received placebo with 62.3% and 63.4% of otenaproxesul 200 mg and 250 mg-treated patients having at least a 30% reduction in WOMAC pain scores, respectively compared to 45.3% of placebo patients and 41.8% and 43.5% of otenaproxesul 200 mg and 250 mg-treated patients having at least 50% a reduction in WOMAC pain scores compared to 34.4% of placebo patients.

### Plasma Naproxen Measurement Results

Plasma samples were taken at a single time point approximately four hours after dosing on Days 1, 4, and 14 and analyzed for otenaproxesul metabolite naproxen concentrations. Comparison of plasma naproxen was made for all patients across the three otenaproxesul treatment groups and by gender within each otenaproxesul treatment group.

It was noted that female patients had consistently higher levels of plasma naproxen than male patients on Days 1, 4 and 14 of drug administration regardless of treatment dose.

### CYP2C9 Polymorphism Results

Genetic polymorphism of the cytochrome P450 enzyme, CYP2C9, a principal metabolizing enzyme of naproxen was assessed. Analyses of blood samples collected at Baseline, prior to treatment, revealed the presence of 11 CYP2C9 polymorphic variants among the 384 patients in the safety population. Across the 4 treatment groups 3 major variants represented between 91.2% and 93.4% of all measured CYP2C9 polymorphs, specifically CYP2C9 *1/*1, *1/*2 and *1/*3. Minor CYP2C9 variants noted across the 4 treatment groups included *1/*8 (0%-1.5%); * 1/*11 (0%-0.8%); * 1/*12 (0%-1.5%); * 2/*2 (0%-1.5%); * 2/*3 (1.5%-6.6%); * 3/*3 (0%-0.8%); * 3/*8 (0%-0.8%) and *12/*3 (0%-0.8%). There were no significant differences in CYP2C9 polymorph variant distributions among patients across the four study treatment groups.

### Pharmacodynamic Results

Blood samples taken pre-drug administration showed no significant differences among the 4 groups with respect to plasma TXB2 concentrations. On Day 1, significant suppression of TXB2 levels was seen across the 3 otenaproxesul treatment groups. Similar, profound inhibition of TXB₂ synthesis was also evident on Days 4 (p<0.001) and 14 (p<0.001) in the groups treated with otenaproxesul.

### Safety Results

A similar percentage of patients, including 40.9% of patients in the otenaproxesul treatment groups and 36.4% of patients in the placebo dose group experienced at least 1 Treatment Emergent Adverse Event (TEAE) during the study. In the otenaproxesul group, the incidence of TEAEs between the otenaproxesul treatment groups was comparable, ranging from 37.7% patients in the 150 mg treatment group to 43.9% patients in the 250 mg treatment group. The most common TEAEs included hepatic enzyme increased (8.2%) and headache (5.0%). In the placebo group, the most commonly reported TEAE was diarrhea (7.6%).

The majority of otenaproxesul liver associated TEAEs were alanine aminotransferase (ALT) and/or aspartate aminotransferase (AST) liver transaminase elevations. None of these TEAEs were observed during the 14-day treatment period but were captured at the Day 24/Early Termination (ET) follow up assessment. Across the otenaproxesul treatment groups, treatment-related increases in ALT and/or AST transaminase levels, the majority of which were rated as mild, were noted in 9.8% (6/61) of patients in the 150 mg arm, 7.2% (9/125) of patients in the 200 mg arm and 12.9% (17/132) patients in the 250 mg arm.

The higher treatment-related transaminase increases were found to occur more frequently among female patients across the three otenaproxesul treatment groups.

There were no abnormal data trends seen in the hematology, chemistry (excluding liver enzymes), or urinalysis laboratory test results during the study. No patients died during the study. No patients were discontinued or withdrawn from the study due to liver associated TEAEs and no patient in any treatment arm met the criteria for a Hy's law case.

### Gastrointestinal (GI) Safety Results

Otenaproxesul was well tolerated at all three administered doses over the 14-day treatment period. Gastrointestinal disorder TEAEs were comparable among patients receiving placebo (18.2%) and patients receiving otenaproxesul (14.5%) and of the 15 patients across the three otenaproxesul treatment arms who were prematurely discontinued from the study, none were discontinued due to TEAEs related to gastrointestinal adverse effects. This GI safety result is a significant advantage of otenaproxesul over naproxen. It is known in the art that an efficacious dose of naproxen causes noticeable GI injury in approximately 30% of patients.

### Renal Safety Results

No clinically meaningful renal adverse events were reported for any patient. Six patients experienced a urinary tract infection. Pollakiuria and hematuria were noted in one patient each. With one exception, all on-study measures of renal function including serum creatinine, uric acid and electrolytes remained within the normal range or exhibited transient, non-clinically significant changes during the study drug administration period. One 70-year-old female patient treated with otenaproxesul 200 mg for 14 days exhibited a 1.5×ULN value for serum creatinine at the Day 24 assessment.

### Cardiovascular Safety Results

Mean changes from Baseline to Day 24/End of Treatment in systolic and diastolic blood pressure and heart rate were small, similar across treatment groups, and not clinically relevant. Single nonserious events of hypertension or blood pressure increased were reported in 2 (1.6%) patients and 2 (1.5%) patients in the otenaproxesul 200 mg and 250 mg treatment groups, respectively. All events of hypertension or blood pressure increased were reported as resolved during the study. A single event of hypotension was noted in 1 (0.8%) patient in the otenaproxesul 200 mg treatment group and was also reported as resolved during the study. No patients in the otenaproxesul 150 mg treatment group or in placebo group experienced an event of hypertension or hypotension.

### Summary and Conclusions

The primary objective of this study was to evaluate the efficacy of a 14-day dosing regimen of otenaproxesul (ATB-346), where the otenaproxesul in the composition was in crystalline form, at doses of 150 mg, 200 mg and 250 mg compared to placebo, selected based on state-of-the-art knowledge of the efficacy and safety profiles for otenaproxesul known at the time of the trial. This Phase 2B study enrolled a total of 387 patients diagnosed with osteoarthritis of the knee, randomized in a 2:2:1:1 allocation ratio to receive otenaproxesul 250 mg, 200 mg, 150 mg, or placebo. In the otenaproxesul group, 61 patients, 125 patients, and 132 patients received otenaproxesul 150 mg, 200 mg, or 250 mg, respectively. Sixty-six patients received placebo. Most patients (96.1%) completed the study.

Treatment with otenaproxesul, where the otenaproxesul in the composition was in crystalline form, demonstrated clinically and statistically significant improvement in WOMAC pain, stiffness, and DPDA subscale scores from Baseline to Day 14. Women who received otenaproxesul compared to those who received placebo showed greater decreases in WOMAC pain, stiffness, and DPDA subscale scores than men. In women, decreases in WOMAC pain, stiffness, and DPDA scores from Baseline to Day 14 reached statistical significance in the otenaproxesul 250 mg and 200 mg treatment groups compared to placebo. For men, decreases in WOMAC subscale scores from Baseline to Day 14 that reached statistical significance included the stiffness and DPDA scores in the 200 mg treatment group. Exploratory efficacy endpoint analyses show that clinically meaningful decreases in normalized pain subscale scores from Baseline to Day 14 were achieved for patients who received otenaproxesul compared to patients who received placebo. Notable decreases in WOMAC pain scores from Baseline to Day 14 were seen among patients in the otenaproxesul 200 mg treatment group with approximately 62% of otenaproxesul 200 mg-treated patients having at least a 30% reduction in WOMAC pain scores compared to 45% of placebo patients and approximately 42% of otenaproxesul 200 mg-treated patients having at least 50% a reduction in WOMAC pain scores compared to 34% of placebo patients.

Safety was evaluated throughout the study by monitoring Aes, clinical laboratory assessments, physical examination data, vital signs, and concomitant medications. In some instances, patients resumed NSAID treatment after they completed two weeks of study drug administration and their ten-day post study safety visit. An assessment of these patients showed the incidence of TEAEs among patients who did not receive NSAIDs at any time during the study was similar to the incidence of TEAEs in the overall Safety Population. In addition, acetaminophen (up to 2000 mg daily intake) was permitted to be used as a rescue medication during the study. Investigators prescribed this medication, in most cases, to be taken as needed and therefore, it was not possible to determine the exact amount of acetaminophen taken by each patient at any time during the study, including the time between Study Visits 5 and 6 when patients were no longer taking otenaproxesul.

A single time point measurement of naproxen, the principal otenaproxesul metabolite in plasma was conducted on patients in all otenaproxesul treatment groups on Day 1, Day 4 and Day 14 of treatment. To assess the effect of otenaproxesul on the inhibition of cyclo-oxygenase enzymes, circulating plasma levels of the inflammatory biomarker, thromboxane B2 were measured pre-treatment and at Days 1, 4 and 14 of treatment. Finally, to assess the potential effect of cytochrome P450 polymorphism on the metabolism of otenaproxesul-derived naproxen, blood samples were taken from all patients to characterize the polymorphic variation of CYP2C9.

As noted above, regardless of treatment dose, female patients were found to have consistently higher levels of plasma naproxen than male patients on Days 1, 4 and 14 of drug administration.

Blood samples taken pre-drug administration showed no significant differences among the 4 groups with respect to plasma thromboxane B2 concentrations. On Day 1, significant suppression of thromboxane B2 levels was seen across the 3 otenaproxesul treatment groups (p<0.001). Similar, profound inhibition of thromboxane B2 synthesis was also evident on Days 4 and 14 (p<0.001) in the groups treated with otenaproxesul.

Genetic polymorphism of the cytochrome P450 enzyme, CYP2C9, a principal metabolizing enzyme of naproxen was assessed. Analyses of blood samples collected at Baseline, prior to treatment, revealed the presence of 11 CYP2C9 polymorphic variants among patients in the safety population. Across the 4 treatment groups 3 major variants represented between 91.2% and 93.4% of all measured CYP2C9 polymorphs, specifically CYP2C9 *1/*1, *1/*2 and *1/*3. There were no significant differences in CYP2C9 polymorph variant distributions among patients across the four study treatment groups suggesting that naproxen metabolism via CYP2C9 is unlikely to explain the observed differences in plasma naproxen concentrations or AST/ALT between male and female patients.

In summary, treatment with otenaproxesul, where the otenaproxesul in the composition was in crystalline form, at 200 mg and 250 mg once daily demonstrated statistically significant efficacy in reducing WOMAC pain, stiffness, and DPDA subscale scores in patients with osteoarthritis of the knee. Daily treatment with the otenaproxesul at 150 mg demonstrated a positive reduction in pain, stiffness and DPDA (reaching statistical significance on Day 4 for stiffness), as well as marked reduction in plasma thromboxane B2 levels on par with reductions observed in patients treated with 200 mg and 250 mg.

### Example 3: Plasma Naproxen Metabolite Measurements after Otenaproxesul Administration (State of the Art)

Plasma samples were taken at a single time point approximately four hours after dosing on Days 1, 4, and 14 with 150 mg, 200 mg and 250 mg of otenaproxesul (where the otenaproxesul in the composition was in crystalline form) and analyzed for otenaproxesul metabolite naproxen concentrations. Sample Preparation and Extraction: A 75 μL aliquot of each sample (calibration standards/quality controls/samples) was aliquoted into pre-labelled tubes. To each aliquoted sample, 300 μL of working internal standard solution (500 ng/ml of IS in acetonitrile/methanol, 75/25, v/v) was added (Exception: 300 μL of acetonitrile/methanol (75/25, v/v) was added to double blank). All tubes were vortexed adequately, followed by centrifugation at 13000 rpm for 5 minutes. A 150 μL aliquot of the organic supernatant was transferred to clean glass tubes. After evaporation at 40° C., the dry residues were reconstituted in 300 μL of USP Purified Water, followed by adequate vortexing. The prepared samples were injected into an LC-MS/MS system which comprised a 6400 Series MS/MS instrument coupled to an Agilent Model 1200 Series liquid chromatography pump and a CTC PAL autosampler.

Comparison of plasma naproxen metabolite was made for all patients across the three otenaproxesul treatment groups and by gender within each otenaproxesul treatment group.

Female patients were found to have consistently higher levels of plasma naproxen than male patients on Days 1, 4 and 14 of drug administration. Blood samples for metabolite analyses were collected in coded pre-cooled 4 mL sodium heparin Vacutainers by direct venipuncture. Samples were measured by LC-MS using a validated assay for the measurement of naproxen in human plasma samples.

This study also looked at levels of TXB₂ and found no significant differences in TXB₂ levels between males and females at each dose and time point.

### Example 4: Investigation of Sex-Based Dosage for Otenaproxesul Via Pharmacokinetics (PK)/Pharmacodynamics and Absorption, Metabolism, Excretion (AME) Clinical Study (State of the Art)

The trial described in this example was designed to test the efficacy (via thromboxane inhibition as a proxy biomarker) and safety of ≤150 mg/day (for example 75 mg/day, 100 mg/day or 125 mg/day) of otenaproxesul, where the otenaproxesul in the composition was in crystalline form, in otherwise healthy older adults, ages 40-75, and also to identify ideal sex-based dosages for otenaproxesul. The study was designed in part on the observation that across the otenaproxesul treatment groups, treatment-related >3×ULN (“upper limit of normal”) ALT and/or AST transaminase levels were noted (almost exclusively after the 14-day dosing interval) in 9.8% (6/61) of patients in the 150 mg arm, 7.2% (9/125) of patients in the 200 mg arm and 12.9% (17/132) patients in the 250 mg arm, with this issue being substantially biased towards females),

In this clinical trial, 28-day human safety/PK data was generated to complement the 14-day DRF data from Example 2. Biomarker data/dose (e.g., thromboxane B2) were also generated using standard ELISA assay kits.

### Research Population

Healthy older volunteers (for example, age 40-70) were enrolled. Patients with well-managed, depression, hypertension, type-2 diabetes are allowed. None demonstrated acute pain conditions. All serum chemistry must be WNL at study start. At least 50% of all females across both Study A and Study B were post-menopausal.

Healthy older volunteers (age 40-70) were enrolled and treated during the study, including 60 female volunteers (Study Group A) and 60 male volunteers (Study Group A).

Otenaproxesul was supplied as 75 mg, 100 mg and 125 mg, brown, film-coated tablets and was in crystalline form. Patients were instructed to take 1 tablet of otenaproxesul in the morning after an overnight fast. Food intake was restricted until at least 30 minutes after the morning dose.

### Study Groups and Doses

The study enrolled a total of 42 subjects on either a 75 mg or 100 mg dose per day of otenaproxesul, of whom 35 had completed the 28-day drug administration period, with seven subjects having been administered the drug for 21 days before the study was paused. Whole blood was taken pre-treatment and on set days after treatment for the measurement of biomarkers, for example, thromboxane B2, alanine aminotransferase, aspartate aminotransferase, bilirubin, alkaline phosphatase, and indicators such as otenaproxesul metabolites.

Three subjects in the 100 mg cohort, who had completed the full drug administration period, exhibited liver transaminase elevations exceeding five times the upper limit of normal, triggering the required pause. Other indicators of liver function for these subjects were normal. The study incorporated an in-clinic post-administration observation period of 14 days.

### Example 5: Liver Transaminase Elevations (LTE) for Crystalline Otenaproxesul at Various Dosing Schedules

As noted above, the plasma naproxen metabolite levels from the clinical trials were examined and it was determined that a plasma naproxen metabolite (M25) of >10 to 15 μg/mL is associated with a therapeutic effect. Ideally, these plasma levels of M25 should be reached within one hour after administration of otenaproxesul. Detailed analysis of the plasma naproxen concentrations from the clinical trial indicated that single doses of, for example, about 75 mg to about 250 mg of otenaproxesul, where the otenaproxesul in the composition was in crystalline form, in a treatment naïve subject would likely not have the desired pharmacokinetic properties to enable rapid attainment of a therapeutic M25 concentration of >10 to 15 μg/mL.

Analysis of the clinical data showed that there were no observed adverse liver effects, specifically transient elevations of liver transaminases at any single dose of the otenaproxesul between 25 mg to 2000 mg or at multiple dose administrations of the otenaproxesul at doses of 150 mg, 200 mg or 250 mg for seven days. When liver safety findings are observed, cumulative naproxen metabolite plasma drug exposure over the prescribed treatment period exceeded 4000 μg*hr/mL.

Table 1 summarizes the plasma naproxen metabolite exposure data for both liver-safe and liver-adverse otenaproxesul treatment protocols.

TABLE 1

| | Duration | AUC₀₋ₜ | AUC0_{-∞} | Total Exposure |
|---|---|---|---|---|
| Dose (mg) | (days) | (μg*hr/mL) | (μg*hr/mL) | (μg*hr/mL) |
| Liver safe | | | | |
| 150 | 7 | 283 | 396 | 2094¹ |
| 200 | 7 | 316 | 442 | 2338 |
| 250 | 7 | 475 | 660 | 3510 |
| 2000 | 1 | 1550 | 465² | 2015 |
| Liver adverse | | | | |
| 75 | 21 | 207 | 270 | 4617 |
| 100 | 28 | 215 | 280 | 6300 |
| 150 | 14 | 283 | 396 | 4075 |
| 200 | 14 | 316 | 442 | 4550 |
| 250 | 10 | 475 | 660 | 4935 |
| 250 | 14 | 475 | 660 | 6835 |

¹Total = [(283 μg*hr/mL × 6 days) + 396 μg*hr/mL terminal phase] = 1698 + 396 = 2094 μg*hr/mL
²Italicized numbers are imputed or extrapolated from existing pharmacokinetic data.

Based on the surprising results in Table 1, it was proposed that dosing schemes for otenaproxesul would need to generate M25 plasma AUC_{0-∞} level below 4000 μg*hr/mL to enhance liver safety. Additionally, desired dosing schemes should achieve a plasma naproxen metabolite (M25) of >15 to 20 μg/mL within 60 minutes of dosing, or about >10 to 20 μg/mL within about 30 min to about 60 min of dosing.

Liver safety related to treatment regimens not exceeding the proposed 4000 μg*hr/mL total AUC plasma M25 exposure were confirmed in a clinical study in which three cohorts consisting of 16 healthy subjects (8 males/8 females) per group were administered one of three crystalline otenaproxesul treatment regimens. Data were collected over a 312-hour period at protocol-specified intervals. Sampling was most frequent during the first 2 hours post drug administration. Treatment regimens were (i) otenaproxesul single dose 1500 mg (Total 1500 mg); (ii) crystalline otenaproxesul Day 1 single dose 1500 mg plus 100 mg on Days 2-5 (Total 1900 mg) or (iii) crystalline otenaproxesul single dose 2000 mg (Total 2000 mg). No subjects in any of the three treatment arms exhibited any liver adverse effects. Plasma exposure calculations are shown in Table 2 below.

TABLE 2

| | Duration | AUC0-t | AUC0-^{∞} | Total Exposure |
|---|---|---|---|---|
| Dose (mg) | (days) | (μg*hr/mL) | (μg*hr/mL) | (μg*hr/mL) |
| Liver safe | | | | |
| 1500 | 1 | 750 | 2292 | 3042 |
| 1500 + 400 | 5 | 639 | 2761 | 3400 |
| 2000 | 1 | 893 | 2886 | 3779 |

### Example 6: Analysis of 2000 mg Doses—Regimens for Safety and Efficacy

It is noted from the pharmacokinetic curves in FIG. 1 showing naproxen metabolite (M25) plasma levels following single dose crystalline otenaproxesul administrations in healthy subjects from the Phase 1 single ascending dose (SAD) study in Example 1, the time to attain maximal plasma concentration of M25 varies widely between 6 and 48 hours across the dosage spectrum. However, while the Cmax of the 2000 mg dose is 24 hours, it is noted that it also exhibited a “pseudo-Cmax” at 2 hours of greater than >10 μg/mL to 15 μg/mL that would provide sufficient M25 concentrations within the first two hours and potentially alleviate pain (these values for the optimal plasma naproxen metabolite levels were determined from an analysis of the Phase I and Phase II clinical trials). Further, it is noted that the naproxen metabolite plasma levels greater than >10 to 15 μg/mL were maintained for the entire 72 hours test period. Therefore, while not being bound by theory, a single 2000 mg treatment is predicted to provide pain relief for 3 days and possibly to 5 days based upon measured and projected M25 plasma concentrations. If a supplemental dose were required after day 3, any single dosage strength 1500 mg could be administered without exceeding the cumulative naproxen (M25) metabolite plasma drug exposure of 4000 μg*hr/mL.

Table 3 summarizes the plasma concentration levels of naproxen metabolite over time following administration of 2000 mg of crystalline otenaproxesul.

TABLE 3

| Time (hours) | M25 (naproxen) (μg/mL) |
|---|---|
| 0.5 | 2 |
| 1 | 5 |
| 2 | 12 |
| 3 | 16 |
| 4 | 19 |
| 5 to 12 | 18 |
| 24 | 28 |
| 48 | 24 |
| 72 | 14 |
| 120 | <2 (estimated) |

The above 2000 mg dose was provided to subjects as 8×250 mg tablets. A further study was conducted where the 2000 mg dose of crystalline otenaproxesul was provided to 16 subjects as smaller 10×200 mg tablets. Table 4 summarizes the plasma concentration levels of naproxen metabolite over time following administration of the smaller tablets 200 mg tablets. It can be seen that administration of 2000 mg of crystalline otenaproxesul in smaller tablets resulted in a faster dissolution, with a Cmax at approximately 4 hours.

TABLE 4

| Time (hours) | M25 (naproxen) (μg/mL) |
|---|---|
| 0.5 | 13.97 |
| 1 | 23.40 |
| 2 | 32.17 |
| 4 | 38.15 |
| 8 | 33.92 |
| 12 | 36.35 |
| 24 | 46.36 |
| 72 | 14.74 |
| 96 | 6.40 |
| 120 | 2.87 |
| 144 | 1.41 |

These results confirm the proposal that a faster dissolution rate can be achieved by lowering the concentration of otenaproxesul in each individual tablet. However, the administration of 10 tablets to a subject as to obtain the desired dosage amount is not clinically viable.

### Example 7A: Exemplary Amorphous Solid Dispersions and Compositions of the Application

Exemplary amorphous solid dispersions 1-8 were prepared by spray-drying. The weight ratio of otenaproxesul (or ATB-346) to polymer (and optionally to antioxidant) in the exemplary amorphous solid dispersions (ASD) as well as parameters used to manufacture the exemplary amorphous solid dispersions (ASD) are provided in Table 5.

TABLE 5

| ASD No. | 1 | 2 | 3 | 4 | 5 | 6 | 7 | 8 |
|---|---|---|---|---|---|---|---|---|
| Ratio of | 35/65 | 35/64.5/0.5 | 50/49.5/0.5 | 20/80 | 35/65 | 30/65/5 | 60/40 | 35/65 |
| ATB-346/ | ATB-346/ | ATB-346/ | ATB-346/ | ATB-346/ | ATB-346/ | ATB-346/ | ATB-346/ | ATB-346/ |
| polymer/ | HPMCAS- | PVPVA6 | PVPVA6 | HPMCAS- | HPMCAS- | PVPVA6 | Eudragit | HPMC- |
| antioxidant | HG | 4/BHT | 4/ | H | L | 4/ | L100 | 2910 |
| | | | BHT | | | TPGS | | (E3) |
| Solvent | 100% | 100% | 100% | 100% | 100% | 100% | 97/3 | 80/20 |
| | Acetone | Acetone | Acetone | Acetone | Acetone | Acetone | acetone/ | Acetone |
| | | | | | | | water | Water |
| Solids | 5.0 | 5.0 | 5.0 | 5.0 | 5.0 | 5.0 | 5.0 | 5.0 |
| Loading | | | | | | | | |
| (wt %) | | | | | | | | |
| Atomizer | Pressure Swirl: Schlick 2.0 | | | | | | | |
| Batch Size | 45 | 20 | 20 | 75 | 45 | 50 | 25 | 20 |
| (g) | | | | | | | | |
| Atomizer | 120 | 120 | 120 | 120 | 120 | 120 | 120 | 120 |
| Pressure | | | | | | | | |
| (psi) | | | | | | | | |
| Drying Gas | 500 | 500 | 500 | 500 | 500 | 500 | 500 | 500 |
| Flow Rate | | | | | | | | |
| (g/min) | | | | | | | | |
| Inlet Temp | 94 | 88 | 84 | 94 | 94 | 94 | 94 | 112 |
| (°C) | | | | | | | | |
| Outlet Temp | 40 | 40 | 40 | 40 | 40 | 40 | 40 | 40 |
| (°C) | | | | | | | | |
| Solution | 28 | 25 | 25 | 28 | 28 | 28 | 28 | 25 |
| Flow Rate | | | | | | | | |
| (g/min) | | | | | | | | |
| Secondary | 35 °C, 16 | 40 °C, 18 | 40 °C, 18 | 35 °C, 16 | 35 °C, 16 | 35 °C, 16 | 35 °C, 16 | 40 °C, 18 |
| Drying | hr, | hr, | hr, | hr, | hr, | hr, | hr, | hr, |
| Conditions | Vacuum | Vacuum | Vacuum | Vacuum | Vacuum | Vacuum | Vacuum | Vacuum |

### Example 7B: Additional Exemplary Amorphous Solid Dispersions with Otenaproxesul Amorphous Solid Dispersions

Additional polymers were evaluated for their use to form amorphous solid dispersions of otenaproxesul as shown in tables 5A to 50

Amorphous solid dispersions were obtained by spray-drying using a Yamato Pulvis Basic Unit, model GB-210 Lab Spray Dryer with two-fluid nozzle model GF-300, internal nozzle diameter 711 μm at batch size of 10, 50 and 100 g/lot.

Spray drying solution: The polymer was dispersed into the solvent and mixed by magnetic stirring. Otenaproxesul (ATB-364) was then added and mixed until completely dissolved. For the amorphous solid dispersion in which the polymer was insoluble in acetone, two solutions were separately prepared: 1) otenaproxesul in acetone and 2) polymer in DCM, MeOH or mixtures of DCM/MeOH then both solutions were mixed, and no recrystallization occurred.
Spray drying process: The liquid flow rate was 15±2 g/min; inlet temperature 70±5° C. except for PEG containing dispersions which was done at lower temperature, outlet temperature was NMT 50° C.; atomization air pressure 0.20±0.05 MPa and drying gas flow rate 0.3-0.4 m³/min. After all liquid was sprayed the heating was stopped and the spray dried material was allowed to cool down to 35° C. before stopping the blower. The spray-dried material was transferred from the spray-dryer to a vacuum oven at 45±5° C. and −25±2 in Hg for secondary drying. For PEG8000 only amorphous solid dispersion secondary drying was done at RT/vacuum −25 in Hg.

TABLE 5 A
ASD No 1

| | | | Sub lot | Sub lot |
|---|---|---|---|---|
| | | Sub lot | 01/02 | 01/02 |
| | Ingredient | 01/02 | % | % w/w dry |
| Functionality | name | g/batch | Solution | basis |
| API | ATB-364 | 35/35 | 3.8/3.4 | 35/35 |
| Polymer | HPMCAS-HG | 65/65 | 7.0/6.3 | 65/35 |
| Solvent | Acetone | 830/930 | 89.2/90.3 | — |

| | | | |
|---|---|---|---|
| Total: | 930/1030 | 100 | 100 |

TABLE 5 B
ASD No 9

| | Ingredient | g/ | % | % w/w |
|---|---|---|---|---|
| Functionality | name | batch | Solution | dry basis |
| API | ATB-364 | 17.5 | 3.2 | 35 |
| Polymer | HPMCAS-HG | 32.25 | 5.9 | 64.5 |
| Antioxidant | BHT | 0.25 | 0.05 | 0.5 |
| Solvent | Acetone | 500 | 90.9 | — |

| | | | |
|---|---|---|---|
| Total: | 550 | 100 | 100 |

TABLE 5 C
ASD No. 10

| | Ingredient | g/ | % | % w/w |
|---|---|---|---|---|
| Functionality | name | batch | Solution | dry basis |
| API | ATB-364 | 17.5 | 3.2 | 35 |
| Polymer | HPMCAS-HG | 32 | 5.8 | 64 |
| Antioxidant/Solubilizer | Vit E. TPGS | 0.5 | 0.1 | 1 |
| Solvent | Acetone | 500 | 90.9 | — |

| | | | |
|---|---|---|---|
| Total: | 550 | 100 | 100 |

TABLE 5 D
ASD No. 11

| | Ingredient | | % | % w/w |
|---|---|---|---|---|
| Functionality | name | g/batch | solution | dry basis |
| API | ATB-364 | 17.5 | 3.2 | 35 |
| Polymer | Kollidon VA 64 | 32.5 | 5.9 | 65 |
| Solvent | Acetone | 500 | 90.9 | — |

| | | | |
|---|---|---|---|
| Total: | 550 | 100 | 100 |

TABLE 5 E
ASD No 12

| | Ingredient | g/ | % | % w/w |
|---|---|---|---|---|
| Functionality | name | batch | Solution | dry basis |
| API | ATB-364 | 17.5 | 3.2 | 35 |
| Polymer | PVP K-12 | 32.5 | 5.9 | 65 |
| Solvent | Acetone | 500 | 90.9 | — |

| | | | |
|---|---|---|---|
| Total: | 550 | 100 | 100 |

TABLE 5 F
ASD No 13

| | Ingredient | g/ | % | % w/w |
|---|---|---|---|---|
| Functionality | name | batch | Solution | dry basis |
| API | ATB-364 | 17.5 | 3.2 | 35 |
| Polymer | HPMCAS-HG | 15 | 2.7 | 30 |
| Solubilizer | Soluplus | 17.5 | 3.2 | 35 |
| Solvent | Acetone | 500 | 90.9 | — |

| | | | |
|---|---|---|---|
| Total: | 550 | 100 | 100 |

TABLE 5 G
ASD No. 8

| | Ingredient | g/ | % | % w/w |
|---|---|---|---|---|
| Functionality | name | batch | Solution | dry basis |
| API | ATB-364 | 17.5 | 1.7 | 35 |
| Polymer | HPMC E3 | 32.5 | 3.1 | 65 |
| Solvent | Acetone* | 700 | 66.7 | — |
| Solvent | DCM* | 150 | 14.3 | — |
| Solvent | MeOH* | 150 | 14.3 | — |

| | | | |
|---|---|---|---|
| Total: | 1050 | 100 | 100 |

*HPMC E3 insoluble in Acetone, ATB-364 partially soluble in MeOH and in DCM, both are soluble in 70:15:15 w/w solvent mixture

TABLE 5 H
ASD No 14

| | Ingredient | g/ | % | % w/w |
|---|---|---|---|---|
| Functionality | name | batch | Solution | dry basis |
| API | ATB-364 | 17.5 | 1.7 | 35 |
| Polymer | Eudragit E PO | 32.5 | 3.1 | 65 |
| Solvent | Acetone | 1000 | 95 | — |

| | | | |
|---|---|---|---|
| Total: | 1050 | 100 | 100 |

TABLE 5 I
ASD No 15

| | Ingredient | g/ | % | % w/w |
|---|---|---|---|---|
| Functionality | name | batch | Solution | dry basis |
| API | ATB-364 | 17.5 | 1.7 | 35 |
| Polymer | PVP K-30 | 32.5 | 3.1 | 65 |
| Solvent | Acetone* | 600/700 | 57.1/66.7 | — |
| Solvent | DCM* | 400/300 | 38.1/28.6 | — |

| | | | |
|---|---|---|---|
| Total: | 1050 | 100 | 100 |

*PVP K-30 insoluble in Acetone, ATB-364 partially soluble in DCM, both are soluble in 70:30 w/w solvent mixture

TABLE 5 J
ASD No 16

| | Ingredient | g/ | % | % w/w |
|---|---|---|---|---|
| Functionality | name | batch | Solution | dry basis |
| API | ATB-364 | 17.5 | 1.7 | 35 |
| Polymer | Poly(acrylic acid) | 32.5 | 3.1 | 65 |
| Solvent | Acetone* | 700 | 66.7 | — |
| Solvent | MeOH* | 300 | 28.6 | — |

| | | | |
|---|---|---|---|
| Total: | 1050 | 100 | 100 |

*PAA insoluble in Acetone, ATB-364 partially soluble in MeOH, both are soluble in 70:30 w/w solvent mixture

TABLE 5 K
ASD No 17

| | Ingredient | g/ | % | % w/w |
|---|---|---|---|---|
| Functionality | name | batch | Solution | dry basis |
| API | ATB-364 | 17.5 | 1.7 | 35 |
| Polymer | HPMC E5 | 32.5 | 3.1 | 65 |
| Solvent | Acetone* | 700 | 66.7 | — |
| Solvent | DCM* | 150 | 14.3 | — |
| Solvent | MeOH* | 150 | 14.3 | — |

| | | | |
|---|---|---|---|
| Total: | 1050 | 100 | 100 |

*Hypromellose insoluble in Acetone, ATB-364 partially soluble in MeOH and in DCM, both are soluble in 70:15:15 w/w solvent mixture.

TABLE 5 L
ASD No 18

| | Ingredient | g/ | % | % w/w |
|---|---|---|---|---|
| Functionality | name | batch | Solution | dry basis |
| API | ATB-364 | 17.5 | 1.7 | 35 |
| Polymer | HPMCAS-HG | 15 | 1.4 | 30 |
| Polymer | PEG 8000 | 17.5 | 1.7 | 35 |
| Solvent | DCM | 300 | 28.6 | — |
| Solvent | Acetone | 700 | 66.7 | — |

| | | | |
|---|---|---|---|
| Total: | 1050 | 100 | 100 |

TABLE 5 M
Formulation No. 1

| | Ingredient | g/ | % | % w/w |
|---|---|---|---|---|
| Functionality | name | batch | Solution | dry basis |
| API | ATB-364 | 17.5 | 1.7 | 35 |
| Polymer | PEG8000 | 32.5 | 3.1 | 65 |
| Solvent | Acetone* | 700 | 66.7 | — |
| Solvent | DCM* | 300 | 28.6 | — |

| | | | |
|---|---|---|---|
| Total: | 1050 | 100 | 100 |

*PEG800 insoluble in Acetone, ATB-364 partially soluble in DCM, both are soluble in 70:30 w/w solvent mixture

TABLE 5 N
Formulation No. 2

| | Ingredient | | | % w/w dry |
|---|---|---|---|---|
| Functionality | name | g/batch | % Solution | basis |
| API | ATB-364 | 17.5 | 1.7 | 35 |
| Polymer | HPMC E15 | 32.5 | 3.1 | 65 |
| Solvent | Acetone* | 700 | 66.7 | — |
| Solvent | DCM* | 150 | 14.3 | — |
| Solvent | MeOH* | 150 | 14.3 | |

| | | | |
|---|---|---|---|
| Total: | 1050 | 100 | 100 |

*HPMC E15 insoluble in Acetone, agglomeration of polymer by contact with API-acetone solution

TABLE 5 O
Formulation No. 3

| | Ingredient | | | % w/w dry |
|---|---|---|---|---|
| Functionality | name | g/batch | % Solution | basis |
| API | ATB-364 | 3.5 | 1.7 | 35 |
| Polymer | PEG 3350 | 6.5 | 3.1 | 65 |
| Solvent | Acetone* | 140 | 66.7 | — |
| Solvent | DCM* | 60 | 28.6 | — |
| Total: | | 210 | 100 | 100 |

*PEG 3350 insoluble in Acetone, ATB-364 partially soluble in DCM, both are soluble in 70:30 w/w solvent mixture

Completely dissolved otenaproxesul and polymer spray-drying solutions were obtained using PEG3350 (Table 50) and PEG8000 65% (Table 5M) however no spray dried material was obtained. A sticking film on the cyclone was obtained from both polymers at all inlet temperatures tested (PEG8000, 70° C.; PEG3350, 65° C.; and PEG8000, 52° C.). Formulations with HPMCE15 (Table 5 N), precipitation of the polymer HPMCE15 was observed when the polymeric DCM/MeOH solution was mixed with the API-acetone solution. The spray-drying was not done. Formulation with 35% PEG8000, Table 5 L) spray-dried material was sticky after spray-drying but became non-sticky powder after secondary drying (22 hours at RT/−25 in Hg).

### Physical Characterization of Amorphous Solid Dispersions

### i) Residual Solvent:

Thermal Gravimetric Analysis (TGA) was used to determine the level of residual solvent and moisture (volatile content) after spray drying and secondary drying. The analysis was performed using a TA Instrument Q50 thermogravimetric analyzer at scanning speed of 10° C./min over a temperature range of 25 to 120° C. The samples were heated in a platinum open crucible in nitrogen atmosphere 60 mL/min.

Volatile content: Residual solvent and moisture on spray-dried material was determined by TGA. It was found that after 60 minutes at 45±5° C. and vacuum-25 in Hg the volatile content of the ASD with HPMCAS as polymer is 0.3-0.4% w/w. For solid with hygroscopic polymers the final water content depends on the initial moisture content of the polymer. For ASDs using Poly acrylic acid (PAA) (Table 5 J), the solvent system was Acetone 70%/Methanol 30%. ASD was dried at 45° C. and −25 in Hg after 90 and 180 min. TGA results were 3.850% and 3.508%, respectively, higher than observed for the PAA (0.877%) compared to all other samples. The secondary drying temperature was increased to 60° C. and after 60 minutes the volatile content by TGA was still higher (3.307%) than expected (≤0.877%). Formulation with Eudragit E PO (Table 5 H) showed TGA 0.3% independently of the batch size 10/50 gram and with or without secondary drying applied.

TABLE 6
Volatile Content of A by TGA and Yield

| Sample ID | TGA (%) | Polymer/TGA | Dry Yield |
|---|---|---|---|
| (Batch size) | of ASD | of Polymer (%) | (%) |
| ASD No. 1 | 0.3231 | HPMCAS- | 71 |
| ASD No. 1 | 0.4575 | HG/0.86 | 80 |
| ASD No. 9 | 0.4808 | | 72 |
| ASD No. 10 | 0.3561 | | 78 |
| ASD No.11 | 1.307 | Kollidon VA64/ | ND |
| | | 2.163 | |
| ASD No. 12 | 2.527 | PVP K-12/5.511 | 70 |
| ASD No. 13 | 0.5523 | HPMCAS/0.861 | 79 |
| | | Soluplus/2.112 | |
| ASD No. 15 | 3.800 | PVP K-30/5.538 | 52 |
| Formulation No. 1 | No ASD obtained | — | — |
| T = 70 °C | | | |
| Formulation No. 7 | No ASD obtained | — | — |
| T = 65 °C | | | |
| Formulation No. 1 | No ASD obtained | — | — |
| T = 52 °C | | | |
| ASD No. 16 | 3.508 | PAA/0.877 | 64 |
| | (270 min 45 °C/ | | |
| | −25 inHg) | | |
| | 3.307 | | |
| | (+60 min 60 °C/ | | |
| | −25 inHg) | | |
| ASD No. 17 | 0.794 | HPMC E5/1.839 | 64 |
| ASD No.8 (10 g) | 0.956 | HPMC E3/2.459 | 64 |
| ASD No. 8 (50 g) | 0.999 | | 76 |
| Formulation No. 2 | No ASD obtained | — | — |
| ASD No. 14 (10 g) | 0.2625 | Eudragit E PO/ | 44 |
| ASD No. 14 (50 g) | 0.3126 (after | 0.296 | 67 |
| | spray drying) | | |
| | 0.2934 (75 min | | |
| | 45 °C/−25 in Hg) | | |
| ASD No. 18 | 0.4436 | HPMCAS-HG/0.86 | 75 |
| | | PEG 8000/0.183 | |

### ii) X-Ray Powder Diffraction

The crystal state was verified by X-Ray Powder Diffraction (XRPD) using a Bruker X-ray diffractometer model D2 Phaser (Karlsruhe, Germany), with Cu Kα radiation (λ=1.5406 Å) at an increment of 0.01° 2θ with a 0.2 s step time (scan rate of 3.0° 2θ/min) over a range of 3-56° 2θ, an 0.6 mm opening slit, a 1.0 mm scatter plate and a 2.5 mm detector window. The samples were analyzed using a low volume sample holder and were kept under a constant rotation of 15 rpm during the analysis.

### Results

After manufacturing the ASDs were examined by XRPD. The X-ray pattern of ASD No. 1 and ASD No. 9 to 18 are typical of amorphous materials.

In the formulation with PVP K-30, the acetone in the solvent mixture was increased to 70% and the solution was passed through a 60-mesh screen. The resulting ASD showed amorphous X-ray diffraction patterns. All other spray drying solutions in multi-solvent systems were similarly prepared using 70% acetone and passing the solution through a 60-mesh screen before spraying and no crystalline peaks were observed.

PEG8000 is a semicrystalline polymer with two intense characteristic peaks at 19.3 and 23.4° 20. The powder from ASD No. 18 showed the two peaks characteristic of PEG 8000.

iii) Thermal Analysis:

Thermal analysis was done by TGA and Differential Scanning calorimetry (DSC). TGA was performed using a TA Instrument Q50 at scanning speed of 20° C./min over a temperature range of RT to 800° C. The samples were heated in a platinum open crucible in nitrogen atmosphere 60 mL/min. DSC analysis was completed with a TA Instrument Q20. The samples were heated in a Tzero Aluminum pan with lid from TA Instruments at 5° C./min under a nitrogen purge of 50 mL/min. C.

### Example 8: Exemplary Compositions Comprising Amorphous Solid Dispersions of the Application Granulated with Sustaining Agents External to the Amorphous Solid Dispersions

Exemplary compositions comprising amorphous solid dispersions granulated with sustaining agents external to the amorphous solid dispersion to form granules comprising the solid dispersion and an external sustaining agent were further prepared. For example, the following compositions comprising a granule comprising an exemplary amorphous solid dispersion and one or more external sustaining agents were prepared.

Exemplary composition no. 1: Exemplary ASD No 3 (31.25 wt %) and external HPMCAS-HF (20.83 wt %) by weight of total pharmaceutical formulation (i.e., tablet). Therefore, exemplary ASD No. 3 and external HPMCAS-HF were present in a weight ratio of 1.5:1 of ASD to external HPMCAS-HF

Exemplary composition no 2: Exemplary ASD No. 8 and external HPMCAS-HF (1.5:1 ratio of ASD:external HPMCAS-HF)

### Example 9A: Polymorphic Stability Studies of Exemplary Amorphous Solid Dispersion of the Application

Exemplary amorphous solid dispersions of the application were maintained under various temperature and relative humidity (RH) to assess the stability of the amorphous solid dispersion (e.g., resistance to crystallization).

In one study, exemplary amorphous solid dispersions nos. 1, 4, 5, and 6 (see Example 7A) were maintained at 30° C. and 65% RH and 50° C. and 11% RH under open conditions and all found be fully amorphous after one month by XRPD.

In a further study, the formulations were maintained at 40° C. and 75% relative humidity (RH) under closed conditions for one month. After one month, all formulations were found to be fully amorphous except exemplary ASD no. 7 comprising Eudragit and exemplary ASD no. 8 comprising HPMC-2910 (HPMC E3). While not being bound by theory, water was used as a co-solvent in the process of preparing ASD no. 7 and ASD no. 8. The presence of water may have contributed to the crystallization of these exemplary ASD's. The high percentage of otenaproxesul in ASD no. 7 comprising Eudragit could have further contributed to crystallization.

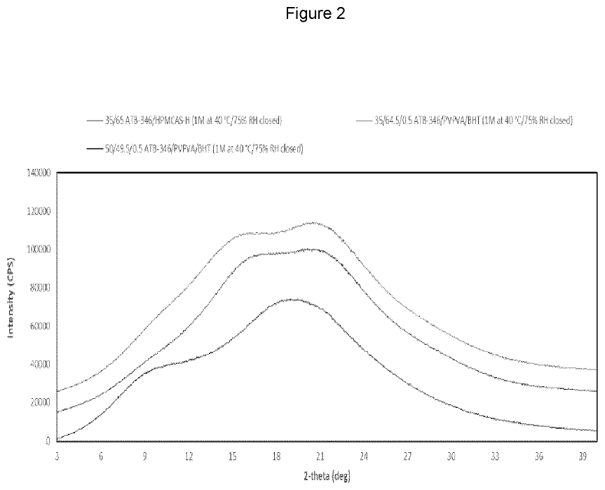

Exemplary XRPDs of ASD nos. 1, 2 and 3 after being maintained at 40° C. and 75% relative humidity (RH) under closed condition for one month is shown in FIG. 2.

In a subsequent study, exemplary ASD no. 1 was analyzed by scanning electron microscopy (SEM) to assess particle morphology and visual assessment of crystallinity. Exemplary ASD no. 1 showed typical particle morphology with spherical and collapsed sphere particles. Some surface roughness and possible onset of particle fusing were observed. No sign of crystals present. No change in particle morphology was observed in the 1-month samples.

### Example 9B: Polymorphic Stability Studies of Exemplary Amorphous Solid Dispersion of the Application

Additional polymorphic stability studies were conducted with the exemplary amorphous solid dispersion of the application described in Example 7B.

Amorphous ASD samples were stored in open and closed cap high density polyethylene (HDPE) bottles at 40° C./75% RH conditions to evaluate the amorphous state stability in 35% drug load ASDs.

ASD powders were placed into opened and closed plastic (HDPE) bottles at 40° C./75% RH. The samples were analyzed for Appearance and by XRPD. Samples from closed bottle were analyzed only if open bottle sample failed (recrystallization of ATB-346 or deliquescence).

The results for 1, 2 and 4 weeks of storage are summarized in Table 7. Table 1 indicates the observed appearance of the ASD and morphology by XRPD. XRPD patterns of the ASDs after 4 weeks of storage in open bottles were taken. 7 of 11 ASD that were placed at 40° C./75% RH remained amorphous after 4 weeks in open container, while recrystallisation of otenaproxesul was observed from formulations comprising poly acrylic acid (PAA, ASD No 16), Polyvinylpyrrolidone K30 (PVP K30, ASD No 15) and HPMC E5 (ASD No 175) after 1 and 4 weeks, respectively.

For PVP K12 containing ASD (ASD No. 12) deliquescence was observed when directly exposed to high humidity (open cap) but amorphous state was verified for samples in closed cap bottle.

After 1 week at 40° C./75% RH otenaproxesul was found to recrystallize from ASD comprising HPMCAS-HG+PEG 8000 (35%) (ASD No. 18

TABLE 7
Further results of stability testing of ASDs at 40 °C/75% RH

| Formulation | OPEN Bottle | | | CLOSED Bottle | | |
|---|---|---|---|---|---|---|
| (Polymer) | 1 week | 2 weeks | 4 weeks | 1 week | 2 weeks | 4 weeks |
| ASD No. 1 | Soft | Soft | Powder, | N/A | N/A | N/A |
| (HPMCAS) | agglomerates/ | agglomerates/ | granules, | | | |
| | Amorphous | Amorphous | hard | | | |
| | (XRPD) | (XRPD) | agglomerates/ | | | |
| | | | Amorphous | | | |
| | | | (XRPD) | | | |
| ASD No. 9 | Soft | Soft | Amorphous | N/A | N/A | N/A |
| (HPMCAS/B | agglomerates/ | agglomerates/ | (XRPD) | | | |
| HT) | Amorphous | Amorphous | | | | |
| | (XRPD) | (XRPD) | | | | |
| ASD No. 10 | Soft | Soft | Amorphous | N/A | N/A | N/A |
| (HPMCAS/Vit | agglomerates/ | agglomerates/ | (XRPD) | | | |
| E) | Amorphous | Amorphous | | | | |
| | (XRPD) | (XRPD) | | | | |
| ASD No. 11 | Soft slightly | Soft sticky | Crystals like | N/A | N/A | N/A |
| (Kollidon | sticky | agglomerates | sugar | | | |
| VA64) | agglomerates/ | Amorphous | crystals/ | | | |
| | Amorphous | (XRPD) | Amorphous | | | |
| | (XRPD) | | | | | |
| ASD No. 12 | Delique- | Delique- | Delique- | Amorphous | Amorphous | Amorphous |
| (PVP-K12) | scence | scence | scence | | | |
| | N/A | N/A | N/A | | | |
| ASD No. 13 | Soft | Soft | Amorphous | N/A | N/A | N/A |
| (HPMCAS/ | agglomerates/ | agglomerates/ | (XRPD) | | | |
| Soluplus) | Amorphous | Amorphous | | | | |
| | (XRPD) | (XRPD) | | | | |
| ASD No. 15 | Sticky | — | — | Recrystal- | — | — |
| (PVP-K30) | agglomerates | | | lization | | |
| | Recrystal- | | | | | |
| | lization | | | | | |
| ASD No. 16 | Some | — | — | Amorphous | Recrystal- | — |
| (PAA) | deliquescence | | | (XRPD) | lization | |
| | Recrystal- | | | | | |
| | lization | | | | | |
| ASD No. 17 | Soft | Soft | Recrystal- | N/A | N/A | Recrystal- |
| (HPMCE5) | agglomerates/ | agglomerates | lization | | | lization |
| | Amorphous | / | | | | |
| | (XRPD) | Amorphous | | | | |
| | | (XRPD) | | | | |
| ASD No. 8 | Soft | Soft | Amorphous | N/A | N/A | N/A |
| (HPMCE3) | agglomerates/ | agglomerates/ | (XRPD) | | | |
| | Amorphous | Amorphous | | | | |
| | (XRPD) | (XRPD) | | | | |
| ASD No. 14 | Hard layer | Hard layer | Hard layer | N/A | N/A | N/A |
| (Eudragit E | stuck on the | stuck on the | stuck on the | | | |
| PO) | bottom/ | bottom/ | bottom/ | | | |
| | Amorphous | Amorphous | Amorphous | | | |
| | (XRPD) | (XRPD) | (XRPD) | | | |
| ASD No. 18 | Recrystallization | — | — | Recrystallization | — | — |

### Example 10: Dissolution Studies of Exemplary Amorphous Solid Dispersions of the Application of Example 7A

An in vitro gastric to intestinal transfer test was performed for biorelevant dissolution of exemplary ASD no 6 except with varying amounts of external HPMCAS-HF and using 0.1 N HCl gastric media with an addition of intestinal media after 30 minutes for a final composition of 0.5% simulated intestinal fluid (SIF) in phosphate buffer saline (PBS) at pH 6.5. Otenaproxesul concentration was monitored by absorbance using Pion UV-vis probes and standard curve. The crystalline otenaproxesul and exemplary ASD no. 6 were run as controls.

Exemplary ASD no. 6 showed comparable dissolution performance to samples with external HPMCAS-HF until 80 minutes (50 minutes in IB). After 80 minutes, the exemplary ASD no. 6 started to precipitate and drop in concentration, while the exemplary composition no. 1 with external HPMCAS-HF sustained Cmax for the rest of the test. A higher ratio of HPMCAS-HF to polymer appeared to slightly increase Cmax but showed comparable sustainment to the lower ratios of external polymer.

Exemplary ASD no. 3 (50/49.5/0.5 ATB-346/PVPVA64/BHT) and 8 (35/65 ATB-346/HPMC-2910 (E3)) were also evaluated alone and with a 1.5:1 ratio of ASD:external HPMCAS-HF polymer.

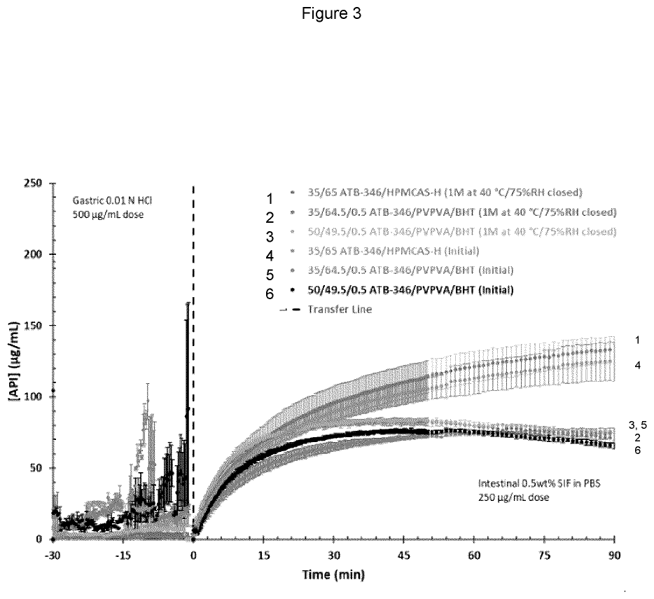

Exemplary ASD no. 1, ASD no 2 and ASD No 3 were further evaluated. Dissolution profiles of various exemplary ASD of the application (e.g, exemplary ASD no. 1, ASD no 2 and ASD No 3) on day 1 (initial) and after 1-month 40° C. at 75% relative humidity under closed conditions using the in vitro gastric to intestinal transfer test using 0.1 N HCl are provided in FIG. 3. As can be seen from FIG. 3, exemplary ASD No. 1 showed no change in dissolution performance after 1 month of closed storage conditions (curves 1 and 4). For PVP exemplary ASD No. 3 the 1-month stability formulation showed slightly faster dissolution kinetics than the originally prepared PVP formulation (curves 3 and 6) but comparable sustainment of drug concentration with other PVP formulations, (ASD No. 3, curves 2 and 5) over the 90-minute test period. No substantive differences in dissolution characteristics were noted among the ASD formulations containing 35% or 50% PVP.

### Example 11: Glass Transitions Temperatures of Amorphous Solid Dispersions of the Application

The glass transition temperatures (Tg) of the exemplary amorphous solid dispersions of the application were evaluated over a temperature range of −20° C. to 160° C. using modulated differential scanning calorimetry (DSC) at a heating rate of 2.5° C./min and are provided in Table 8. The Tg for exemplary amorphous solid dispersions of the application obtained using modulated DSC at 2.5° C./min are provided in Table 9. The Tg for exemplary amorphous solid dispersions of the application after one month at 40° C./75% RH under closed conditions obtained using modulated DSC at 2.5° C./min are provided in Table 18. Samples were placed in Tzero Aluminum hermetic/non-hermitic pans and typically contained between 1 to 6 mg of amorphous solid dispersion. The Tg value was determined by measuring the mid-point by half-height of the DSC response.

TABLE 8

| | | T_{g} @ <5% | T_{g} @ 75% |
|---|---|---|---|
| ASD No. | Description | RH (°C) | RH (°C) |
| 4 | 20/80% | 65.6 ± 0.6 | 42.7 ± 1.2 |
| | ATB-346/HPMCAS-H | | |
| 1 | 35/65% | 57.5 ± 3.1 | 39.6 ± 0.0 |
| | ATB-346/HPMCAS-HG | | |
| 5 | 35/65% | 55.9 ± 1.2 | 37 ± 00 |
| | ATB-346/HPMCAS-L | | |
| 6 | 30/65/5% | 60.3 ± 1.6 | 31 ± 0.1 |
| | ATB-346/PVP-VA64/TPGS | | |
| 7 | 60/40% | 47.8 ± 6.1 | 22.2 ± 0.8 |
| | ATB-346/Eudragit-L100 | | |

TABLE 9

| ASD No. | Description | T_{g} 1 (°C) |
|---|---|---|
| 8 | 35/65 | 72.0 |
| | ATB-346/HPMC E3 | |
| 1 | 35/65 | 63.0 |
| | ATB-346/HPMCAS-H | |
| 2 | 35/64.5/0.5 | 83.0 |
| | ATB-346/PVPVA64/BHT | |
| 3 | 50/49.5/0.5 | 71.0 |
| | ATB-346/PVPVA64/BHT | |

TABLE 10

| | | T_{g} | | T_{g} | |
|---|---|---|---|---|---|
| ASD | | onset | T_{g} | end | Delta Cp |
| No. | Description | (°C) | (°C) | (°C) | (J/(g* °C)) |
| 1 | 35/65 ATB-346/HPMCAS-HG | 60.0 | 65.0 | 71.0 | 0.4 |
| | (1M 40 °C/75% RH closed | | | | |
| 2 | 35/64.5/0.5 ATB- | 81.0 | 85.0 | 90.0 | 0.5 |
| | 346/PVPVA64/BHT (1M | | | | |
| | 40 °C/75% RH closed) | | | | |
| 3 | 50/49.5/0.5 ATB- | 72.0 | 75.0 | 78.0 | 0.5 |
| | 346/PVPVA64/BHT (1M | | | | |
| | 40 °C/75% RH closed) | | | | |

The Tg of exemplary amorphous solid dispersion no. 1 (35/65 ATB-346/HPMCAS-H) was found to be 62.8±0.2° C. (batch 1, Example 12) and 64.0±0.9° C. (batch 2, Example 12) using modulated DSC.

### Example 12: Solubility Data of Crystalline Otenaproxesul Compared to Exemplary an Amorphous Solid Dispersions of the Application

Table 11 shows the solubility enhancement of exemplary amorphous solid dispersion no. 1 compared to crystalline otenaproxesul in simulated intestinal fluid (SIF).

TABLE 11

| | | Solubility at 2 hours |
|---|---|---|
| Form | Media | (μg/ml) |
| crystalline | 0.5 wt % SIF | 51 |
| ASD no. 1 | 0.5 wt % SIF | 234 |

### Example 13: Particle-Size Distribution Characterization of Exemplary Amorphous Solid Dispersions

The particle-size distribution from an approximate 600 g batch of exemplary ASD no. 1 was evaluated and results provided in Table 12.

TABLE 12

| ASD | D(v, | D(v, | D(v, | D[3, | D[4, | |
|---|---|---|---|---|---|---|
| No. 1 | 0.1) | 0.5) | 0.9) | 2] | 3] | Span |
| Batch 1 | 6.6 | 27.9 | 61.5 | 8.3 | 31.5 | 2.0 |
| Batch 2 | 7.8 | 32.2 | 69.8 | 9.7 | 36.2 | 1.9 |

Particle size data from a 3 kilogram demonstration batch of exemplary ASD No. 1 measured at different spray drying pressures ranging from 0.5 bar to 4 bar are shown in the Table 13.

TABLE 13

| Sample | D(v, | D(v, | D(v, | D[3, | D[4, | |
|---|---|---|---|---|---|---|
| Des- | 0.1) | 0.5) | 0.9) | 2] | 3] | |
| cription | μm | μm | μm | μm | μm | Span |
| ASD | 10.0 | 36.2 | 73.2 | 11.2 | 39.5 | 1.7 |
| No. 1 | | | | | | |
| 0.5 bar | | | | | | |
| ASD | 9.7 | 35.6 | 71.1 | 11.0 | 38.6 | 1.7 |
| No. 1 | | | | | | |
| 1 bar | | | | | | |
| ASD | 9.4 | 34.1 | 67.6 | 11.0 | 36.9 | 1.7 |
| No. 1 | | | | | | |
| 2 bar | | | | | | |
| ASD | 9.1 | 33.3 | 66.0 | 10.4 | 36.0 | 1.7 |
| No. 1 | | | | | | |
| 3 bar | | | | | | |
| ASD | 8.8 | 32.4 | 64.2 | 10.1 | 35.0 | 1.7 |
| No. 1 | | | | | | |
| 4 bar | | | | | | |

### Example 14: Purity Studies of Exemplary Solid Dispersions of the Application

The stability of the exemplary amorphous solid dispersions of the application were evaluated using HPLC and are shown in Tables 14 A and B.

TABLE 14 A

| | | RRT- | | | | | |
|---|---|---|---|---|---|---|---|
| | | 0.24 | | | RRT-0.85 | | |
| | | 4-Hydroxy- | RRT-0.41 | | ATB-346- | ATB-346 | |
| Formulation | Conditions | benzonitrile | Naproxen | RRT-0.84 | Amide | API | RRT-1.10 |
| ATB-346 | | ND | ND | 0.18 | 0.07 | 99.45 | 0.25 |
| Crystalline | | | | | | | |
| ASD no. 4 : | 5 °C/0% RH | ND | ND | <LOQ | 0.16 | 99.73 | 0.11 |
| 20/80 ATB- | 30 °C/65% RH | 0.16 | 0.28 | 0.06 | 0.18 | 99.20 | 0.13 |
| 346/ | 50 °C/11% RH | 0.09 | 0.19 | <LOQ | 0.18 | 99.38 | 0.16 |
| HPMCAS-H | | | | | | | |
| ASD no. 1 : | 5 °C/0% RH | ND | ND | <LOQ | 0.13 | 99.78 | 0.09 |
| 35/65 ATB- | 30 °C/65% RH | 0.08 | 0.14 | 0.06 | 0.16 | 99.44 | 0.11 |
| 346/ | 50 °C/11% RH | 0.05 | 0.11 | 0.06 | .021 | 99.40 | 0.19 |
| HPMCAS-HG | | | | | | | |
| ASD no. 6: | 5 °C/0% RH | ND | ND | 0.20 | 0.14 | 99.56 | 0.10 |
| 30/65/5 ATB- | 30 °C/65% RH | ND | <LOQ | 0.19 | 0.17 | 99.48 | 0.15 |
| 346/PVP- | 50 °C/11% RH | ND | <LOQ | 0.18 | 0.20 | 99.41 | 0.21 |
| VA64/TPGS | | | | | | | |

*ND = not detected, <LOQ = below limit of quantitation, .05% of WS, <LOD = below limit of detection set at 0.02% calculated from LOQ, RRT = Relative Retention Time

TABLE 14B

| | | RRT-0.87 | ATB-346 | |
|---|---|---|---|---|
| | | ATB-346- | API | |
| Formulation | RRT-0.85 | Amide | 1.00 | RRT-1.11 |
| ASD no. 8: | 0.38 | 0.12 | 99.16 | 0.29 |
| 35/65 ATB-346/HPMC | | | | |
| E3 | | | | |
| ASD no. 1: | 0.43 | 0.17 | 99.07 | 0.30 |
| 35/65 ATB-346/HP | | | | |
| MCAS-HG | | | | |
| ASD no. 2: | 0.41 | 0.43 | 98.64 | 0.29 |
| 35/64.5/0.5 ATB- | | | | |
| 346/PVPVA64/BHT | | | | |
| ASD no. 3: | 0.41 | 0.28 | 98.81 | 0.29 |
| 50/49.5/0.5/ATB- | | | | |
| 346/PVPV.BHT | | | | |

### HPLC Methods for Purity/Impurity Determinations

HPLC analyses were performed on a Waters 2695 or Agilent 1200, or equivalent, instrument. The column was an ACE-5 C18 150 mm×4.6 mm, 5 μm or equivalent column. The flow rate was 1.0 mL/min, the injection volume was 10 μL, the column temperature was 25° C., the sample temperature was 5° C. and the wavelength for detection was 254 nm. Two eluents were used which were either (1) A: 1.8 g K₂HPO₄·3H₂O dissolved in 1000 ml of water and pH adjusted to 5.5 using orthophosphoric acid and B: acetonitrile; or (2) A: 1.4 g K₂HPO₄·3H₂O dissolved in 1000 ml of water and pH adjusted to 7.0 with 85% phosphoric acid and B: acetonitrile.

The gradient used for (1) is presented in Table 15.

TABLE 15

| t (min) | % A | % B |
|---|---|---|
| 0.0 | 95 | 5 |
| 5.0 | 65 | 35 |
| 10.0 | 65 | 35 |
| 25.0 | 20 | 80 |
| 35.0 | 20 | 80 |
| 35.1 | 95 | 5 |
| 45.0 | 95 | 5 |

The gradient used for (2) is presented in Table 16.

TABLE 16

| t (min) | % A | % B |
|---|---|---|
| 0.0 | 95 | 5 |
| 0.01 | 95 | 5 |
| 20.0 | 10 | 90 |
| 30.0 | 10 | 90 |
| 30.1 | 95 | 5 |
| 40 | 95 | 5 |

Under conditions (1) the retention times and RRT's that were obtained are provided in Table 17.

TABLE 17

| Compound | Retention Time (min) | RRT |
|---|---|---|
| 4-hydroxybenzonitrile | 7.85 | 0.35 |
| Naproxen | 9.78 | 0.44 |
| ATB-346 | 22.26 | 1 |
| ATB-346 Amide | 19.06 | 0.86 |
| Naproxen Ethyl Ester | 23.42 | 1.05 |

Under conditions (2) the retention times and RRT's that were obtained are shown in Table 18.

TABLE 18

| Compound | Retention Time (min) | RRT |
|---|---|---|
| Naproxen | 8.74 | 0.49 |
| ATB-346 | 17.69 | 1 |

### Example 15: Pharmacokinetic Studies in Dogs

The pharmacokinetics of five different formulations of otenaproxesul (formerly ATB-346) were evaluated in Beagle dogs. Five groups of dogs (n=3/group) were orally administered their assigned formulations once daily for four days. Blood samples were taken for a full 24-hour PK profile on Day 1 and on Day 4.

Group 1 was administered crystalline otenaproxesul, Group 2 was administered milled crystalline otenaproxesul, Group 3 was administered micro-milled crystalline otenaproxesul, Group 4 was administered exemplary ASD no. 1 (see Example 7A above) and Group 5 was administered exemplary ASD no. 4 (see Example 7A above). Dose administered was 15 mg/kg (human equivalent dose of 525 mg).

Crystalline otenaproxesul was administered to dog Groups 1-3 in gelatin capsules by oral gavage. Exemplary ASD no. 1 and exemplary ASD no. 4 were administered as oral suspensions to dog Groups 4-5.

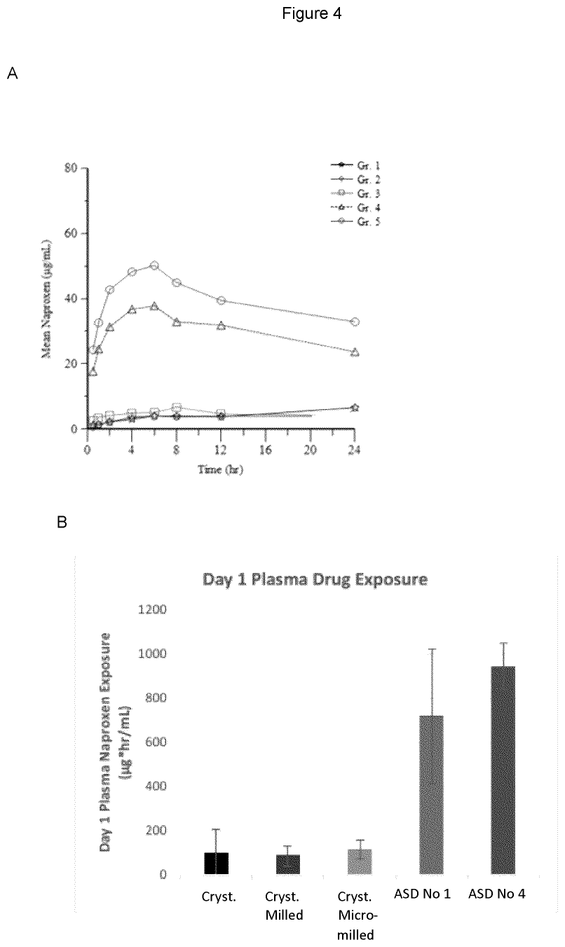

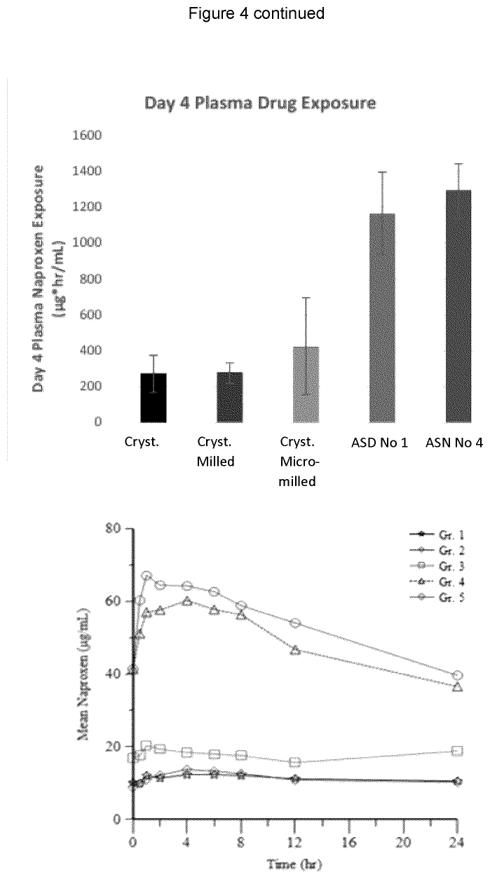

The pharmacokinetic profiles (mean naproxen/M25 concentration) after a single dose administration are shown in FIG. 4A.

The Day 1 24-hour naproxen (M25) exposure after a single dose administration of each formulation is shown in FIG. 4 B.

The mean Day 1 AUC₀₋₂₄ plasma naproxen (M25) exposures for the crystalline otenaproxesul formulations is from about 87 to 114 μg*hr/mL. In contrast, the mean naproxen (M25) exposures generated by the amorphous dispersions (exemplary ASD no. 1 and exemplary ASD no. 4) are 7 to 9-fold higher than the crystalline otenaproxesul formulations with values of 720 μg*hr/mL for exemplary ASD no. 1 and 945 μg*hr/mL for exemplary ASD no. 4.

The Day 4 24-hour plasma naproxen (M25) exposure after the fourth single dose administration of each formulation is shown in FIG. 4 C.

Day 4 AUC₀₋₂₄ plasma exposures for the crystalline otenaproxesul formulations are about 272 to 400 μg*hr/mL. In contrast, the mean exposures generated by the amorphous dispersions (exemplary ASD no. 1 and exemplary ASD no. 4) are approximately 3 to 4.5-fold higher than the crystalline otenaproxesul formulations with values of 1163 μg*hr/mL for exemplary ASD no. 1 and 1238 μg*hr/mL for exemplary ASD no. 4.

Day 4 pharmacokinetic curves showing naproxen metabolite (M25) plasma levels of crystalline otenaproxesul (Group 1), milled crystalline otenaproxesul (Group 2), micro-milled crystalline otenaproxesul (Group 3), exemplary ASD no. 1 (Group 4) and exemplary ASD no. 4 (Group 5) are shown in FIG. 4 D.

Table 19 provides the plasma naproxen metabolite (M25) concentrations for crystalline otenaproxesul (Group 1), milled crystalline otenaproxesul (Group 2), micromilled crystalline otenaproxesul (Group 3), exemplary ASD no. 1 (Group 4) and exemplary ASD no. 4 (Group 5) measured on Days 1 and 4

TABLE 19

| | Plasma M25 (naproxen) Concentration (μg/mL) | | | | |
|---|---|---|---|---|---|
| | | milled | micro-milled | | |
| | crystalline | crystalline | crystalline | ASD | ASD |
| Sampling | otena- | otena- | otena- | No. 1 | No. 4 |
| Time | proxesul | proxesul | proxesul | AD 35/65 | AD 20/80 |
| Day 1: 1 | 1.4 | 1.4 | 3.6 | 24.5 | 32.6 |
| Hour | | | | | |
| Day 1: 12 | 3.8 | 3.9 | 4.8 | 31.9 | 39.4 |
| hours | | | | | |
| Day 4: Pre- | 10.2 | 8.9 | 16.8 | 41.3 | 41.4 |
| treatment | | | | | |
| Day 4: 1 | 12.1 | 11 | 20.3 | 57 | 67.1 |
| Hour | | | | | |
| Day 4: 12 | 11.2 | 11 | 15.7 | 46.7 | 54.4 |
| hours | | | | | |

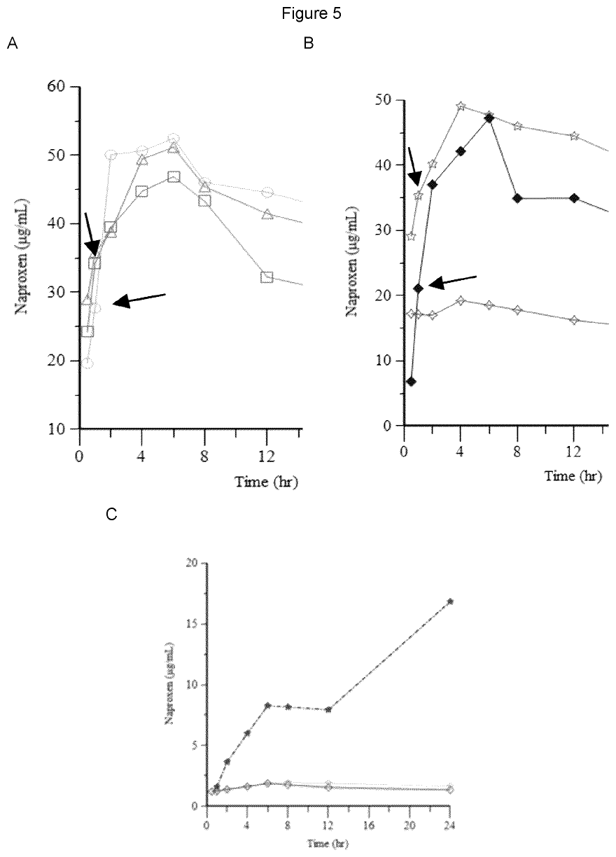

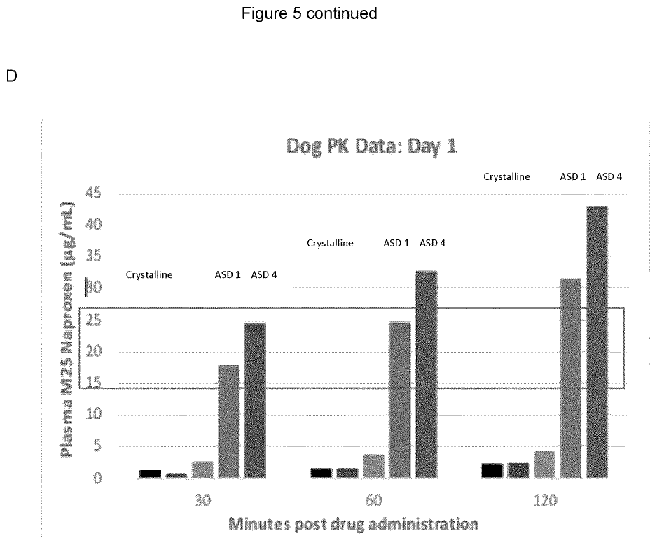

FIG. 5 (A, B) displays the rapid onset of higher plasma naproxen metabolite (M25) concentrations for exemplary ASD no. 1 (Group 4) and exemplary ASD no. 4 (Group 5) measured on Day 1 compared to crystalline otenaproxesul administrations on Day 1 (FIG. 5 C). The 1-hour average naproxen concentration after administration of exemplary ASD no. 4 is 32 μg/mL (FIG. 5A) and the 1-hour average naproxen concentration after administration of exemplary ASD no. 1 is 29 μg/mL (FIG. 5 B). Whereas 1-hour average naproxen concentration after administration of crystalline otenaproxesul is 1.4 μg/mL (FIG. 5 C).

The pharmacokinetic curves shows that treatment of Beagle dogs with exemplary amorphous solid dispersion no. 1 and 4 provide a 21.8 fold increase in naproxen metabolite (M25) plasma concentration one hour after administration compared to crystalline otenaproxesul.

For an acute pain indication in particular, a more rapid plasma uptake of drug especially within the initial 30 to 90 minutes post drug administration is desired. The accumulation of plasma naproxen (again as a surrogate for total otenaproxesul) at certain time intervals (30 minutes, 60 minutes and 120 minutes) after administration of crystalline otenaproxesul, milled crystalline otenaproxesul, micro-milled crystalline otenaproxesul, exemplary ASD no. 1, exemplary ASD no. 4 in Beagle dogs is shown in FIG. 5 D. The highlighted box in FIG. 5 D indicates the target plasma drug concentration for naproxen (M25) (e.g, 15 to 25 μg/mL) shown to be effective using plasma drug concentrations measured at steady state in the clinical trials described above.

The data from the Beagle dog pharmacokinetic study clearly show that at the drug administration of 15 mg/kg (approximately 525 mg human dose), the amorphous dispersion formulations have a better capacity to deliver plasma naproxen drug concentrations required for effective COX-inhibition and rapid onset of pain relief compared to crystalline otenaproxesul, even crystalline otenaproxesul that has been milled or micro-milled to increase its surface area (a known technique to increase bioavailability).

Pharmacodynamic assessment of TXB₂ and PGE₂ plasma concentrations were assessed on:
Day 1, one hour post treatment
Day 1, 12 hours post treatment
Day 4, pre-treatment
Day 4, one hour post treatment
Day 4, 12 hours post treatment.

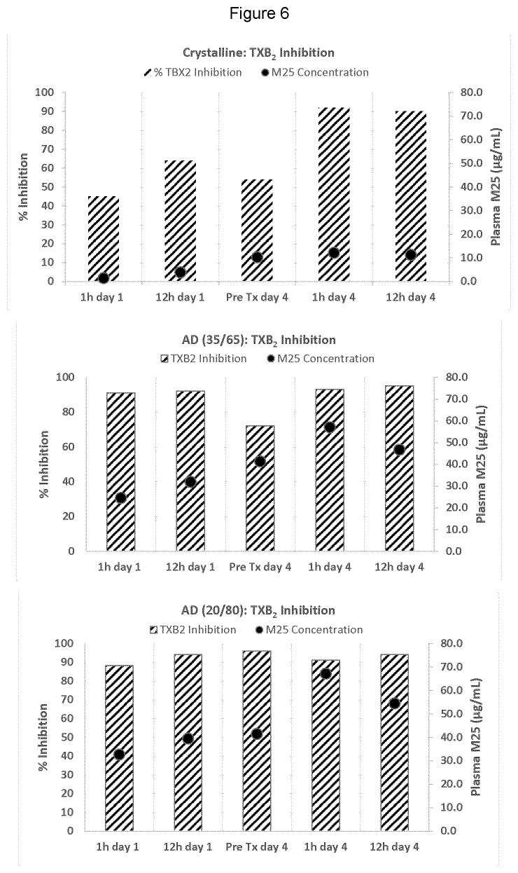

The pharmacodynamic effect of crystalline otenaproxesul (Group 1, top graph), exemplary ASD no. 1 (Group 4, middle graph) and exemplary ASD no. 4 (Group 5, bottom graph) on TXB2 levels is shown in FIG. 6.

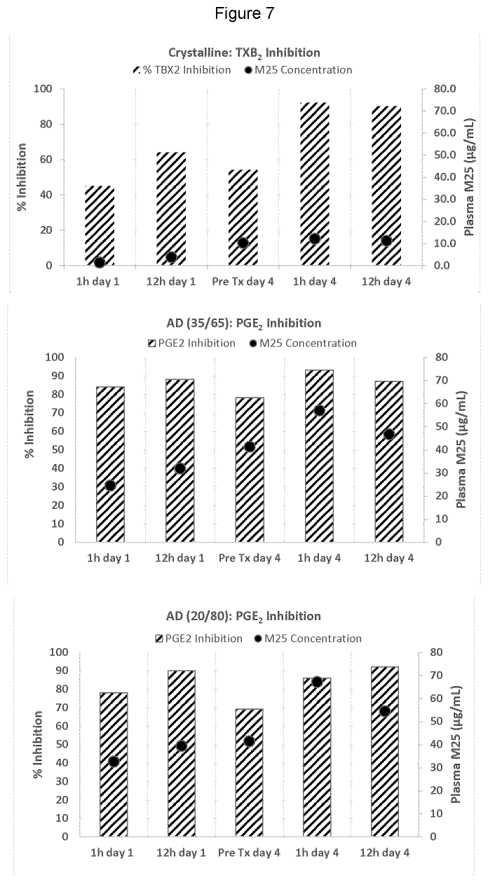

The pharmacodynamic effect of crystalline otenaproxesul (Group 1, top graph), exemplary ASD no. 1 (Group 4, middle graph) and exemplary ASD no. 4 (Group 5, bottom graph) on PGE₂ levels is shown in FIG. 7.

The objective of designing the various amorphous solid dispersions otenaproxesul was to examine the rate of release of the naproxen metabolite (M25) as a surrogate for onset of plasma concentrations of otenaproxesul metabolites and to correlate the resulting plasma M25 concentrations to COX inhibition as seen by decreased plasma levels of TXB₂ and PGE₂.

Table 19 lists the plasma M25 concentrations at the times of plasma sampling for TXB₂ and PGE₂ measurements.

### Thromboxane B2

All formulations tested inhibited plasma levels of TXB₂ by >80% on Day 4.

Comparative composition comprising crystalline otenaproxesul yielded lower Day 1 (1- and 12-hour time points) M25 plasma concentrations than the exemplary ASDs comprising amorphous otenaproxesul by a 10×. As a result, inhibition of TXB₂ was lower on Day 1 and at the Day 4 pre-treatment time point than the corresponding degree of inhibition by the exemplary ASD's comprising amorphous otenaproxesul which remained at approximately 90% for all sampling time points.

### Prostaglandin E₂

All formulations tested decreased plasma levels of PGE₂ by 70% to 90% on Day 4.

Notably, all formulations decreased PGE₂ levels by approximately 80% within an hour of administration. This level of inhibition, however, was not maintained by comparative composition comprising crystalline otenaproxesul at the Day 1 12-hour time point but had recovered to a large extent by the Day 4 pre-treatment sampling time point. In contrast to the crystalline formulation, the exemplary ASDs comprising amorphous otenaproxesul generally maintained >80% reductions in circulating plasma concentration of PGE₂ throughout the study.

The data suggest that early attainment of plasma M25 concentrations≥25 μg/mL results in rapid (<60 minutes) and sustained decreases in plasma TXB₂ and PGE₂ concentrations.

### Example 16: Exemplary Oral Pharmaceutical Compositions of the Application

### A. Exemplary Pharmaceutical Compositions Comprising Exemplary ASDs of the Application.

Exemplary oral dosage form pharmaceutical compositions of the application (for example, exemplary composition of the application no. 3 (comprising exemplary ASD no. 1), exemplary composition of the application no. 4 (comprising exemplary ASD no. 2) and exemplary composition of the application no. 5 (comprising exemplary ASD no. 3) were prepared. Table 20 shows the ingredient lists of exemplary oral dosage form pharmaceutical compositions 3, 4 and 5.

TABLE 20

| | | Com- | Com- | Com- |
|---|---|---|---|---|
| | | position | position | position |
| Function | Ingredient | 3 | 4 | 5 |

Intragranular

| | | | | |
|---|---|---|---|---|
| ASD | 35% ATB-346: | 60.00% | | |
| Composition | HPMCAS-HG | | | |
| No. 1 | | | | |
| ASD | 35/64.5/0.5 ATB- | | 40.00% | |
| Composition | 346/PVPVA64/BHT | | | |
| No. 2 | | | | |
| ASD | 50/49.5/0.5 ATB- | | | 31.25% |
| Composition | 346/PVPVA64/BHT | | | |
| No. 3 | | | | |
| Sustaining | HPMCAS-HF | | | 20.83% |
| agent | | | | |
| Filler | Microcrystalline | 16.25% | 24.25% | 18.21% |
| | Cellulose | | | |
| | (Avicel PH102) | | | |
| Filler | Mannitol | 16.25% | 24.25% | 18.21% |
| | (Mannogem EZ) | | | |
| Disintegrant | Croscarmellose Sodium | 3.00% | | |
| | (Ac-Di-Sol) | | | |
| Disintegrant | Crospovidone (PVP XL) | | 6.00% | 6.00% |
| Glidant | Silicone Dioxide | 0.50% | 0.50% | 0.50% |
| | (Syloid 244 FP) | | | |
| Lubricant | Magnesium Stearate | 0.25% | 0.25% | 0.25% |

Extragranular

| | | | | |
|---|---|---|---|---|
| Disintegrant | Croscarmellose Sodium | 3.00% | | |
| | (Ac-Di-Sol) | | | |
| Disintegrant | Crospovidone (PVP XL) | | 4.00% | 4.00% |
| Glidant | Silicon Dioxide (Syloid | 0.50% | 0.50% | 0.50% |
| | 244FP) | | | |
| Lubricant | Magnesium Stearate | 0.25% | 0.25% | 0.25% |

| | | | |
|---|---|---|---|
| Tablet Dose (mg) | 200 | 125 | 125 |
| Tablet Weight (mg) | 952.4 | 892.9 | 800.0 |

### Manufacturing Process of Exemplary Oral Dosage Form Pharmaceutical Compositions

Blending: The exemplary ASD, filler(s), intragranular disintegrant, intragranular glidant and optionally sustaining agent were blended in a Turbula bench top Blender for 2 min @ 32 rpm

The blend was then passed through No. 20 Sieve for delumping followed by further blending for 10 min @ 32 rpm. Lubricant was then added and the mixture blended for 4 min @ 32 rpm.

Granulation: Slugging was performed via single station, manual press. Slugs were milled via mortar and pestle and passed through a No. 20 sieve.

Extragranular disintegrant and extragranular glidant were the added and blended for 10 min @ 32 rpm, after which extragranular lubricant (Sieved) was added and further blended for 4 min @ 32 rpm.

Compression: The blend was compressed using a single station, manual tablet press with a target tensile strength of 2.5 Mpa. The tablet was then tested for rate of disintegration in 0.01N HCl (pH 2.0) media and dissolution.

### Formulation Dissolution and Stability

The rate of dissolution of exemplary oral dosage form pharmaceutical compositions 3, 4 and 5 were tested. Results are provided in Table 21.

TABLE 21

| Compositions | 3 | 4* | 5 |
|---|---|---|---|
| Replicate 1 | 1:19 | 30+ min | 1:58 |
| (min:sec) | | | |
| Replicate 2 | 1:23 | 30+ min | 1:44 |
| (min:sec) | | | |
| Replicate 3 | 1:07 | 30+ min | 1:36 |
| (min:sec) | | | |
| Average (min:sec) | 1:16 | 30+ min | 1:46 |

*Tablets gelled and then stop disintegrating. Disintegration testing was stopped 30 minutes after observable disintegration progress has stopped.

Exemplary final oral pharmaceutical compositions 3 and 5 disintegrated quickly at 1:16 [min: sec] and 1:46 [min: sec] respectively. Exemplary oral pharmaceutical composition 4 gelled, and the disintegration test was stopped at 30 minutes when appreciable disintegration progress has stopped. Tablets comprising HPMCAS had good disintegration time.

A single tablet of exemplary final oral pharmaceutical compositions 3 and 5 were dosed into a USP II dissolution vessel (800 mL and 500 mL final volume for 200 mg and 125 mg tablets respectively) for a 0.25 mg/ml dose, consistent with SDD dosing. Exemplary composition 3 was found to perform better than exemplary composition 5. Exemplary composition 5 showed comparable Cmax and sustainment to exemplary composition 3 but slightly slower dissolution rate. Exemplary composition 3 showed comparable dissolution rate and sustainment to its ASD (exemplary ASD no. 1).

### B: Additional Exemplary Oral Pharmaceutical Compositions-Comprising Additives

The addition of additives such as antioxidants, buffering agents, alkalinizing agents, solubilizers, surfactants was investigated.

Exemplary composition of the application no. 3 (Exemplary composition of the application no. 3-a comprising exemplary ASD no. 1) was modified to include one or more further additives to provide the following pharmaceutical composition of the application.

The final blends were prepared by dry granulation. To obtain granules to mimic the dry granulation was done by slugging. The intragranular ingredients except the magnesium stearate were mixed for 2 minutes at 28 rpm using a Globe Pharma MiniBlend™ V-Blender with 0.5-quart stainless steel shell. The mixture was screened using a No 20 (850 μm openings) sieve, poured back into the blender, and mixed for 2 minutes at 28 rpm. The magnesium stearate was passed through a No 40 (425 μm) sieve premixed with a volume equivalent of blend from the blender and mixed for 2 minutes at 28 rpm minutes. The blend was compressed to slug using Korsch XP1 single station tablet press equipped with 11 mm round flat tooling. The slugs were compiled at speed 1750 rpm using a Quadro Comil model U5 fitted with a 9525 μm square screen then the pieces of slugs were comilled using a 1016 μm grated round screen. Extragranular excipients except the magnesium stearate were passed through a No 40 sieve then mixed with the granules using the V-blender for 2 min at 28 rpm. The magnesium stearate was screened using a No 40 sieve and premixed with an equivalent volume quantity of blend. The magnesium stearate premix was loaded into the V-blender and mixed for 2 min at 28 rpm. 200 mg tablets were compressed using the single station tablet press Korsch XP1 equipped with 9.91×18.97 mm oval shaped chrome tipped tooling.

TABLE 21B
Exemplary composition 3-a (control)

| ID | Ingredient name | % w/w |
|---|---|---|
| ASD No. 1 | ATB-346/HPMCAS-HG | 60 |
| | (35/65) | |
| Intragranular | Microcrystalline | 16 |
| Excipients | Cellulose | |
| | Mannitol | 16 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |

| | |
|---|---|
| Total: | 100.0 |

TABLE 21 C
Exemplary Composition 6

| ID | Ingredient name | % w/w |
|---|---|---|
| ASD No. 1 | ATB-346/HPMCAS-HG | 60 |
| | (35/65) | |
| Intragranular | Microcrystalline | 16 |
| Excipients | Cellulose | |
| | Mannitol | 16 |
| | Ascorbic Acid | 1 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |

| | |
|---|---|
| Total: | 100.0 |

TABLE 21 D
Exemplary Composition 7

| ID | Ingredient name | % w/w |
|---|---|---|
| ASD No. 1 | ATB-346/HPMCAS-HG | 60 |
| | (35/65) | |
| Intragranular | Microcrystalline | 16 |
| Excipients | Cellulose | |
| | Mannitol | 16 |
| | Citric Acid | 1 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 E
Exemplary Composition 8

| \ID | Ingredient name | % w/w |
|---|---|---|
| ASD No. 1 | ATB-346/HPMCAS-HG | 60 |
| | (35/65) | |
| Intragranular | Microcrystalline | 16 |
| Excipients | Cellulose | |
| | Mannitol | 16 |
| | Meglumine | 1 |
| | Croscarmellose Sodium | 3 |
| | Colloidal Silicon Dioxide | 0.5 |
| | Magnesium Stearate | 0. |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 F
Exemplary Composition 9

| ID | Ingredient name | % w/w |
|---|---|---|
| ASD No. 1 | ATB-346/HPMCAS-HG | 60 |
| | (35/65) | |
| Intragranular | Microcrystalline | 16 |
| Excipients | Cellulose | |
| | Mannitol | 16 |
| | SLS | 1 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 G
Exemplary Composition 10

| ID | Ingredient name | % w/w |
|---|---|---|
| ASD No. 1I | ATB-346/HPMCAS- | 60 |
| | HG (35/65 | |
| Intragranular | Microcrystalline | 16 |
| Excipients | Cellulose | |
| | Mannitol | 16 |
| | Poloxamer | 1 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

### C. Additional Exemplary Oral Pharmaceutical Compositions Comprising Exemplary ASDs of the Application

The formulations were prepared as described in Part B above.

TABLE 21 H
Exemplary composition 11

| ID | Ingredient name | % w/w |
|---|---|---|
| ASD No. 9 | ATB-346/HPMCAS- | 60 |
| | HG/BHT (35/64.5/0.5) | |
| Intragranular | Microcrystalline | 16 |
| Excipients | Cellulose | |
| | Mannitol | 16 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 I
Exemplary composition 12

| ID | Ingredient name | % w/w |
|---|---|---|
| ASD No. 10 | ATB-346/HPMCAS- | 60 |
| | HG/Vit E (35/64/1) | |
| Intragranular | Microcrystalline | 16 |
| Excipients | Cellulose | |
| | Mannitol | 16 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 J
Exemplary composition 13

| ID | Ingredient name | % w/w |
|---|---|---|
| ASD No. 11 | ATB-346/Kollidon VA64 | 60 |
| | (35/65) | |
| Intragranular | Microcrystalline | 16 |
| Excipients | Cellulose | |
| | Mannitol | 16 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 K
Exemplary composition 14

| ID | Ingredient name | % w/w |
|---|---|---|
| ASD No. 12 | ATB-346/PVPK12 | 60 |
| | (35/65) | |
| Intragranular | Microcrystalline | 16 |
| Excipients | Cellulose | |
| | Mannitol | 16 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 L
Exemplary composition 15

| ID | Ingredient name | % w/w |
|---|---|---|
| ASD No. 13 | ATB-346/HPMCAS- | 60 |
| | HG/Soluplus (35/30/35) | |
| Intragranular | Microcrystalline | 16 |
| Excipients | Cellulose | |
| | Mannitol | 16 |
| | Croscarmellose Sodium | 3 |
| | Colloidal Silicon Dioxide | 0.5 |
| | Magnesium Stearate | 0.3 |
| Extragranular | Croscarmellose Sodium | 3 |
| Excipients | Colloidal Silicon Dioxide | 0.5 |
| | Magnesium Stearate | 0.3 |
| Total: | | 100.0 |

TABLE 21 M
Exemplary composition 16

| ID | Ingredient name | % w/w |
|---|---|---|
| ASD No. 8 | ATB-346/HPMC E3 | 60 |
| | (35/65) | |
| Intragranular | Microcrystalline | 16 |
| Excipients | Cellulose | |
| | Mannitol | 16 |
| | Croscarmellose Sodium | 3. |
| | Colloidal Silicon Dioxide | 0.5 |
| | Magnesium Stearate | 0.3 |
| Extragranular | Croscarmellose Sodium | 3. |
| Excipients | Colloidal Silicon Dioxide | 0.5 |
| | Magnesium Stearate | 0.3 |
| Total: | | 100.0 |

TABLE 21 N
Exemplary composition 17

| ID | Ingredient name | % w/w |
|---|---|---|
| ASD No. 14 | ATB-346/Eudragit E PO | 60 |
| | (35/65) | |
| Intragranular | Microcrystalline | 16 |
| Excipients | Cellulose | |
| | Mannitol | 16 |
| | Croscarmellose Sodium | 3 |
| | Colloidal Silicon Dioxide | 0.50 |
| | Magnesium Stearate | 0.3 |
| Extragranular | Croscarmellose Sodium | 3. |
| Excipients | Colloidal Silicon Dioxide | 0.50 |
| | Magnesium Stearate | 0.3 |
| Total: | | 100.0 |

### In Vitro Dissolution Rate

Dissolution profiles of the exemplary compositions showed that some exemplary formulations dissolved faster than 200 mg tablets exemplary composition 3. Complete and faster release (70% dissolved within 10 minutes) was observed for exemplary composition 15 from ASD No. 13 containing Soluplus 35%. Approx 50% dissolution were reached with formulations exemplary composition 10 using Poloxamer as surfactant and Eudragit as polymer. Very poor dissolution profiles were observed for formulations exemplary composition 13, exemplary composition 14 and exemplary composition 16 using Kollidon VA64, PVPK12 and HPMC E3 as lower molecular weights polymers in ASDs.

### Example 17: Clinical Trial Designs

### A. Abdominoplasty Trial Design Using Exemplary Composition of the Application

A 3-arm, 5-day, multi-center, randomized, double-blind, placebo-controlled, trial is proposed.

A total of approximately 375 patients undergoing abdominoplasty will be randomized 2:1:2 to 1 of 3 treatment arms (high dose: low dose: placebo). Subjects will receive study drug as oral treatment every 12 hours starting within 4 hours after abdominoplasty. Treatment arms and dosing sequence are defined Table 22A.

TABLE 22
A: Acute Pain Trial Treatment Arms

| | | Dose | Dose | Dose | Dose | Dose | Dose | Dose | Dose | Dose | Dose |
|---|---|---|---|---|---|---|---|---|---|---|---|
| | # of | 1 | 2 | 3 | 4 | 5 | 6 | 7 | 8 | 9 | 10 |
| Arm | subjects | (Hour 0) | (Hour 12) | (Hour 24) | (Hour 36) | (Hour 48) | (Hour 60) | (Hour 72) | (Hour 84) | (Hour 96) | (Hour 108) |
| High | 150 | 400 mg | 200 mg | 200 mg | 200 mg | 100 mg | 100 mg | 50 mg | 50 mg | 50 mg | 50 mg |
| Dose | | | | | | | | | | | |
| Low | 75 | 200 mg | 100 mg | 100 mg | 100 mg | 50 mg | 50 mg | 25 mg | 25 mg | 25 mg | 25 mg |
| Dose | | | | | | | | | | | |
| Placebo | 150 | 400 mg | 300 mg | 300 mg | 300 mg | 150 mg | 150 mg | 75 mg | 75 mg | 75 mg | g |

Exemplary compositions of the application comprising ASD No. 1 will be prepared comprising dose strengths of 15 mg to 200 mg of otenaproxesul, e.g., 25 mg, 50 mg, 100 mg and 200 mg.

The purpose of the trial will be to evaluate the analgesic efficacy of treatment with of exemplary dosing regimens with compositions of amorphous solid dispersions of otenaproxesul on acute post-operative pain following abdominoplasty compared with placebo. The trial will also determine the safety and tolerability dosing regimens with compositions of amorphous solid dispersions of otenaproxesul in patients following abdominoplasty

Patients will be assessed for pain intensity using the Numerical Pain Rating Scale (NPRS) at various time intervals following administration of the first dose.

Time weighted sum of pain intensity difference (SPID) at, for example, 24 hours, 48-hours after administration of the first composition will be evaluated.

Adverse events (e.g., gastrointestinal, cardiovascular, central nervous system, and renal and hepatic events) will also be assessed and evaluated.

### B. Bunionectomy Trial Design Using Exemplary Composition of the Application

Exemplary compositions of the application comprising ASD No. 1 (e.g. exemplary composition 3)—will be prepared comprising dose strengths of 15 mg to 200 mg of otenaproxesul, e.g., 25 mg, 50 mg, 100 mg and 200 mg.

This trial will enroll about 80% females in all treatment arms based on the epidemiological female prevalence for the surgical procedure.

The PK/PD endpoints (Arms 1-2) will be plasma M25 naproxen concentration versus time profile and overall plasma exposure calculation, hydrogen sulfide concentration versus time profile, plasma concentration measures of Thromboxane B2 and Prostaglandin E2 as indicators of COX-1 and COX-2 inhibition.

All “effectiveness”, i.e., exploratory-graded dose-response endpoints would be the Sum of Pain Intensity Difference (SPID) at 48-hours, i.e., SPID-48 as the “primary” of these four treatments (Arms 3-4-5-6). Table 22 B shows the proposed acute pain trial treatment arms.

TABLE 22 B
Acute Pain Trial Treatment Arms

| | | Treatment | | |
|---|---|---|---|---|
| | | Exemplary tablet | | Otenaproxesul |
| | | composition of the | Sam- | Day 1 |
| | | application comprising | ple | Loading Dose |
| Cohort | Arm | ASD No.1) | Size | (mg) |
| 1 | 1 | Otenaproxesul single dose | 20 | 100 mg |
| (Primary | 2 | Otenaproxesul single dose | 20 | 200 mg |
| PK/PD | | | | |
| endpoints) | | | | |
| 2 | 3 | Otenaproxesul single dose | 50 | 100 mg |
| (Exploratory/ | | (Day 1) | | |
| effectiveness | | Followed by | | |
| endpoints) | | 50 mg otenaproxesul *q12 | | |
| | | Days 2-5 | | |
| | 4 | Otenaproxesul single dose | 50 | 100 mg |
| | | (Day 1) | | |
| | | Followed by | | |
| | | 50 mg otenaproxesul q12 | | |
| | | Days 2-3 | | |
| | | plus | | |
| | | 25 mg otenaproxesul | | |
| | | q12 x Days 4-5 | | |
| | 5 | Otenaproxesul single dose | 50 | 200 mg |
| | | Followed by | | |
| | | 50 mg otenaproxesul q12 | | |
| | | Days 2-5 | | |
| | 6 | Otenaproxesul single dose | 50 | 200 mg |
| | | Followed by | | |
| | | 50 mg otenaproxesul q12 | | |
| | | Days 2-3 | | |
| | | plus | | |
| | | 25 mg q12 Days 4-5 | | |

*q12 means twice (two times) a day

Therefore, Patients in Cohort 1 (Arms 1 and 2) will be included in the primary and safety analyses only. Patients in Cohort 2 (Arms 3, 4, 5, 6) will be included in the exploratory and safety analyses only

### Example 18: Stability and H₂S Studies

### Formulation Stability in Simulated Intestinal Fluid (SIF)

Assay design: 1 mg of exemplary ASD no. 1 and crystalline otenaproxesul were weighed in separate glass vials. SIF was added to prepare a 1 mg/ml solution which was incubated at 37° C. with 5% CO₂. 120 μl was removed at 0.5, 1, 2, 3, 4 and 8 hrs and quenched in DPD (N,N-dimethyl-p-phenylenediamine sulfate) and FeCl₃ solutions. A calibration curve was prepared to quantify amount of H₂S released. A blank and 10 mM stock was also analyzed. The methylene blue was analyzed using HPLC.

### Observations:

Both the formulations displayed low solubility.

Exemplary ASD no. 1 showed a rapid and significant amount of H₂S release. 3.3 μM in 30 minutes with increasing amounts over time with 12.5 μM at the 8 h timepoint.

Crystalline otenaproxesul did not release significant H₂S initially but did so over time reaching 8.5 μM at the 8 h timepoint (Table 23).

TABLE 23

| | Time (hrs) | | | | | |
|---|---|---|---|---|---|---|
| | 0.5 | 1 | 2 | 3 | 4 | 8 |
| 35/65 ASD | 3.3 | 3.8 | 5.5 | 8.2 | 10.8 | 12.5 |
| No. 1 | | | | | | |
| (μM H₂S | | | | | | |
| release) | | | | | | |
| Crystalline | 0.7 | 0.7 | 1.2 | 2.2 | 3.7 | 8.5 |
| (μM | | | | | | |
| H2S release) | | | | | | |

### Formulation Stability in Simulated Gastric Fluid (SGF)

Assay design: 1 mg of exemplary ASD no. 1 and crystalline otenaproxesul were weighed in separate glass vials. SGF was added to prepare a 1 mg/ml solution which was incubated at 37° C. with 5% CO₂. 120 μl was removed at 0, 0.5, 1, 2, 3, 4 and 8 hrs and quenched in DPD and FeCl₃ solutions. A blank and 10 mM stock was also analyzed. The methylene blue was analyzed using HPLC.

### Observations:

As H₂S is volatile in the acidic form the calibration curve was not plotted, however H₂S release was compared to T₀.

Both the formulations displayed low solubility.

Exemplary ASD no. 1 showed faster and larger amount of H₂S release compared to crystalline otenaproxesul (Table 24).

TABLE 24

| | Time (hrs) | | | | | | |
|---|---|---|---|---|---|---|---|
| | 0 | 0.5 | 1 | 2 | 3 | 4 | 8 |
| 35/65 ASD No. 1 | 3.1 | 5.8 | 6.2 | 6.4 | 5.8 | 5.6 | 3.5 |
| (Peak area of | | | | | | | |
| methylene blue) | | | | | | | |
| Crystalline | 1.6 | 2.4 | 2 | 2.3 | 1.8 | 1.9 | 2.4 |
| (Peak area of | | | | | | | |
| methylene blue) | | | | | | | |

### Example 19: Rat Pharmacokinetics of Otenaproxesul Amorphous Dispersion Treatments Assessment of Steady State and C₆₀ M25 Plasma Concentrations and AUC Exposures

### Summary

Rats were administered five treatment doses of the otenaproxesul amorphous solid dispersion (i.e., exemplary ASD no. 1 (35% drug: 65% polymer by weight)) and a crystalline otenaproxesul control dose. Treatments corresponding to human equivalent doses of 18.2 mg, 36.4 mg, 55.1 mg, 109.2 mg and 208 mg were administered once daily to male (n=3) and female (n=3) rats. One group of rats was administered a human equivalent dose of 208 mg twice daily (416 mg total daily dose). Crystalline otenaproxesul was administered at the no observed adverse effect level (NOAEL) dose equivalent to a human dose of 260 mg once daily.

Pharmacokinetic data was evaluated on M25 plasma naproxen concentrations at 60 minutes post administration, Day 1 M25 plasma exposure (AUC₀₋₂₄) and Day 4 M25 plasma exposure (AUC_{0-Last}). Assessment of dose proportional characteristics for both M25 plasma concentration and AUC exposure were measured.

The results are summarized as follows:
M25 plasma concentrations at 60 minutes post administration (C₆₀) and at maximal concentration (Cₘₐₓ) displayed linear, dose-proportional kinetics over the dose range tested.
Female rats generally displayed higher M25 concentrations than male rats.
Except for 1 female rat treated with a human equivalent 208 mg otenaproxesul single dose in an ASD, none of the remaining administered doses of otenaproxesul formulations in rats attained a Day 1 C₆₀ value>15.5 μg/mL.
Relative to a crystalline otenaproxesul, a human equivalent dose of 260 mg otenaproxesul in an ASD, human equivalent treatments of 109 mg and 208 mg otenaproxesul in an ASD had marginally higher M25 plasma concentrations at 1-hour post-treatment but with group average concentrations<16 μg/mL at 1-hour post-treatment.
A Day 4 (steady state) C₆₀ concentration measured after the second dose of 208 mg in an ASD was 28.5 μg/mL in male rats (average of n=3) and 25.9 μg/mL in female rats (average of n=3).
Extrapolation of the rat data suggest that a single 400 mg of otenaproxesul ASD treatment dose on Day 1 would attain a Coo plasma M25 concentration>20 μg/mL.
M25 plasma AUC exposures on Day 1 and Day 4 exhibited linear, dose-proportional kinetics over the dose range tested.
No meaningful gender differences in AUC exposure were evident between male and female rats over the dose range tested either on Day 1 or on Day 4.
The highest treatment exposures of otenaproxesul ASD from 208 mg once daily or 208 mg otenaproxesul in an ASD twice daily were less than or equal to the exposure resulting from 260 mg daily of crystalline otenaproxesul, the NOAEL rat treatment dose.
Gender combined (male+female rats) drug exposure from 208 mg otenaproxesul in an ASD twice daily was expectedly higher (approximately 44%) than exposure from a single 208 mg otenaproxesul in an ASD treatment on Day 1 but was only marginally higher (approximately 13%) on Day 4
AUC at steady state suggests no drug accumulation is occurring in rats when using amorphous solid dispersion (exemplary ASD no. 1) treatment doses up to and including at a human equivalent treatment of 208 mg otenaproxesul once-daily. In contrast, use of crystalline otenaproxesul drug showed AUC increases.

### Results

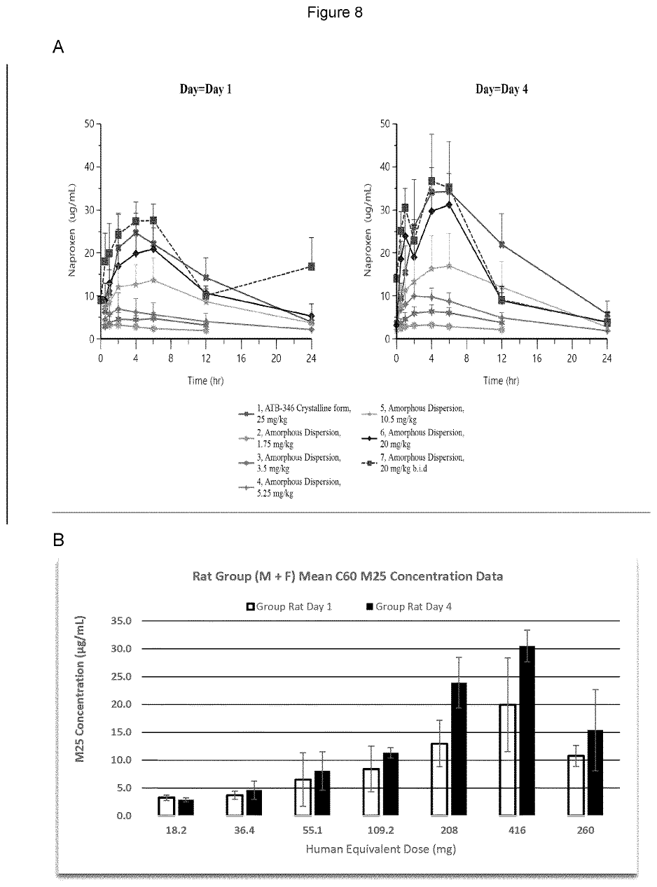

The pharmacokinetic concentration versus time profiles for a range of amorphous solid dispersion otenaproxesul (i.e., exemplary ASD no. 1) treatments in comparison to a control crystalline otenaproxesul treatment are shown in FIG. 8A while the group mean M25 plasma concentrations are shown in FIG. 8B.

FIG. 8A shows the mean plasma concentrations versus time following oral administration of crystalline otenaproxesul and otenaproxesul amorphous solid dispersion (exemplary ASD no. 1) at Day 1 and Day 4 in Rats-Gender Combined. FIG. 8B shows the group mean plasma concentrations of M25 at 60 minutes post drug administration of otenaproxesul amorphous solid dispersion (exemplary ASD no. 1) (solid colour bars) or crystalline otenaproxesul (diagonal striped bars) on Day 1 and Day 4. The human equivalent dose (HED) is related to the administered rat dose by an allometric scaling formula, for example: HED (mg)=([rat dose in mg/kg×0.16]×65 kg).

Male plus female group mean Coo values for the human equivalent 208 mg once or twice daily otenaproxesul amorphous solid dispersion (exemplary ASD no. 1) drug administration exceeded comparable mean C₆₀ values for the 260 human equivalent dose of crystalline otenaproxesul on both Day 1 and on Day 4 (steady state). Note that the Day 1 C₆₀ measure for the 208 mg twice daily treatment was measured 1-hour after the second dose administration or 13-hours after the original 208 mg dose.

It was also observed that at 60 minutes post drug administration on Day 1 of treatment only 1 female rat in the 208 mg once-daily cohort exhibited an M25 plasma concentration>18 μg/mL, specifically 25.9 μg/mL (group range of 6.8 to 25.9 μg/mL). However, no other male or female rat exceeded a value of 15.5 μg/mL M25 concentration at the C₆₀ time point on Day 1. As noted above, rats in the 208 mg twice daily cohort reported a group mean C₆₀ value of 20 μg/mL driven by an average female drug concentration of 25.9 μg/mL versus a 14 μg/mL average for male rats.

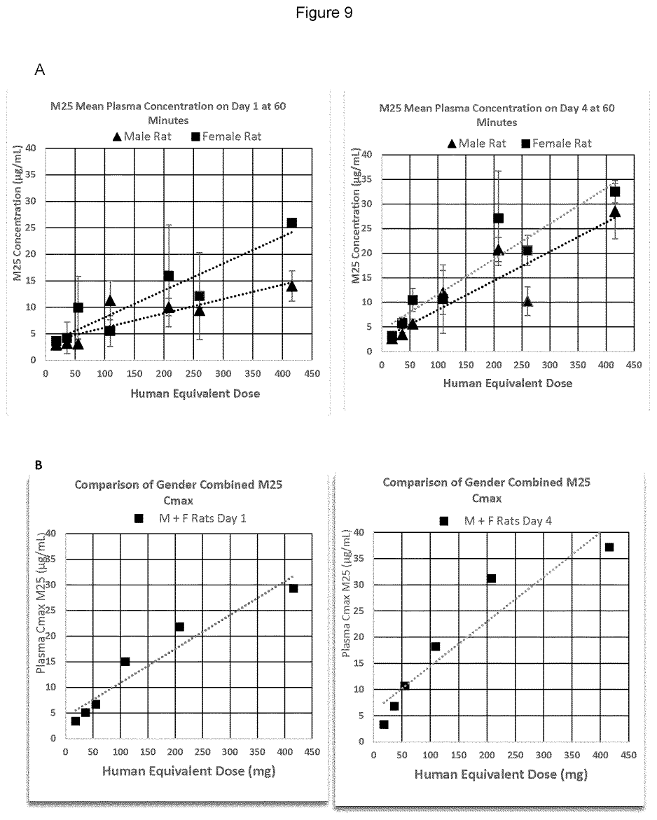

The dose-proportionality of the M25 maximal plasma concentrations (Cmax) for the otenaproxesul amorphous solid dispersion (exemplary ASD no. 1) treatments over a human equivalent dose range of approximately 20 to 425 mg are shown by gender in FIG. 9A and by gender combined cohorts in FIG. 9 B for both Day 1 and Day 4 (steady state) of treatment. One hour after dosing on Day 1, and one hour after dosing on Day 4, the Cmax values for the 109 mg once daily and 208 mg once or twice daily administrations equal or exceed the corresponding Cmax values for crystalline form (hashed or striped data points). The results demonstrate improved absorption of the amorphous drug form of the invention over the previous crystalline form (FIG. 9 B).

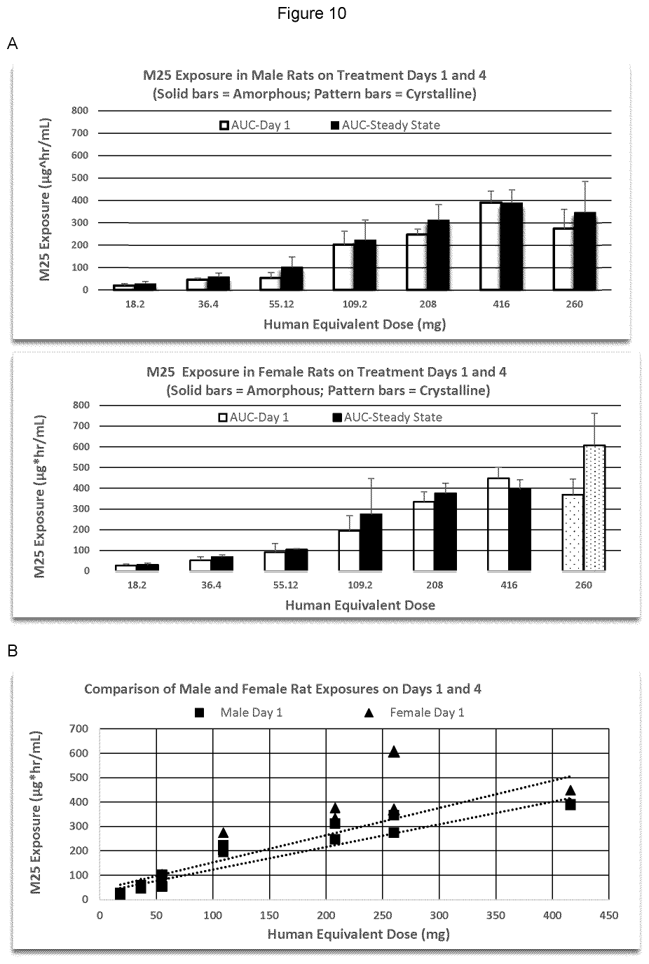

Plasma AUC exposure calculations for male and female rats are shown in FIG. 10A. Dose proportional behaviour is noted and shown in FIG. 10A for male and female rats on Day 1 and at Day 4 steady state.

Doubling the administration of 208 mg from once-daily to twice-daily increased the Day 1 AUC₀₋₂₄ exposure in male rats by approximately 60% and in female rats by approximately 34%. Substantially lower increases in Day 4 AUC₀₋₂₄ were noted in male rats with a 25% increased exposure from 208 mg twice-daily versus once-daily with a minor 5% increase for female rats receiving twice-daily 208 mg versus once-daily treatment.

While all otenaproxesul amorphous solid dispersion (exemplary ASD no. 1) treatment doses up to and including 208 mg otenaproxesul once-daily exhibited increasing AUC exposure between Day 1 and Day 4, the highest treatment regimen of 208 mg twice-daily showed no such AUC increase between Day 1 and Day 4 suggesting that no drug accumulation in plasma is occurring. In contrast, the reference crystalline otenaproxesul drug showed AUC increases of 30% and 60% for males and females, respectively between Day 1 and Day 4.

### Conclusions

The administration of otenaproxesul amorphous solid dispersion (i.e., exemplary ASD no. 1) at higher treatment doses of 208 mg otenaproxesul once or twice daily resulted in comparable Cmax M25 plasma concentrations to that observed with control crystalline otenaproxesul administrations of 260 mg otenaproxesul once daily.

Group mean C₆₀ plasma M25 concentrations for the two highest exemplary ASD no. 1 treatment regimens were consistently higher than the mean C₆₀ values for the crystalline otenaproxesul control both on Day 1 and Day 4. However, for single dose administrations, no Day 1 C₆₀ group mean measure exceeded an M25 concentration of 13 μg/mL. Administration of 208 mg otenaproxesul twice daily attained a C₆₀ value of 20.0 μg/mL suggesting that, based on these rat data, about 400 mg otenaproxesul human dose would attain appropriate plasma concentrations of M25.

Plasma AUC exposures are increased in rat cohorts receiving twice-daily otenaproxesul amorphous solid dispersion formulations (exemplary ASD no. 1) treatment relative to the same dose level administered once daily. It was advantageously noted that although the AUC increase on Day 1 between the twice-daily and once-daily regimens is approximately 44%, the exposure difference is reduced to approximately 13% at steady state when using otenaproxesul amorphous solid dispersion formulations (exemplary ASD no. 1) suggesting no drug accumulation is occurring in rats at a human equivalent treatment of 208 mg otenaproxesul twice-daily.

### Example 20: Dog Pharmacokinetics of Otenaproxesul Solid Amorphous Dispersion Treatments

Summary: Over a 4-day treatment period dogs were administered four treatment doses of the otenaproxesul amorphous solid dispersion formulation (exemplary ASD no. 1) and a crystalline otenaproxesul control dose. Treatments corresponding to human equivalent doses of 70.2 mg, 140.4 mg, 280.8 mg and 561.6 mg otenaproxesul were administered once daily to male (n=4) dogs. Crystalline otenaproxesul was administered at the NOAEL dose equivalent to a human dose of 526.5 mg otenaproxesul once daily.

Pharmacokinetic data were evaluated on M25 plasma naproxen concentrations at 60 minutes post administration (C₆₀), Day 1 M25 plasma exposure (AUC₀₋₂₄) and Day 4 M25 plasma exposure (AUC_{0-Last}). Assessment of dose proportional characteristics for M25 plasma concentration at 60 minutes post administration (C₆₀) and at maximal concentration (Cₘₐₓ) and AUC exposure were measured on Day 1 and Day 4 of treatment.

The results are summarized as follows:
M25 plasma concentrations at 60 minutes post administration (C₆₀) and at maximal concentration (Cₘₐₓ) displayed linear, dose-proportional kinetics over the dose range tested.
Assessment of group average plasma naproxen concentrations revealed no administration of otenaproxesul formulation in dogs attained a Day 1 C₆₀ value>18.5 μg/mL.
It is noted that one dog in the 561.6 mg otenaproxesul human equivalent treatment group had an inexplicably low C₆₀ plasma naproxen value of 0.41 μg/mL. Removal of this dog from the analysis elevates the group average Coo measure to 24.5 μg/mL.
Relative to a crystalline otenaproxesul human equivalent dose of 526.5 mg otenaproxesul, all solid amorphous dispersion otenaproxesul human equivalent treatments had 1.8 to 8.8-fold (or 11.7-fold excluding the outlier dog) higher M25 plasma concentrations at 1-hour post-treatment on Day 1.
Day 4 (steady state) C₆₀ concentrations for control crystalline otenaproxesul and all amorphous solid dispersion otenaproxesul doses except for the 70.2 mg otenaproxesul human equivalent dose exceeded 20 μg/mL
Extrapolation of the dog data suggest that a single 600 mg otenaproxesul treatment dose on Day 1 would attain a Coo plasma M25 concentration>20 μg/mL
M25 plasma AUC exposures on Day 1 and Day 4 exhibited linear, dose-proportional kinetics over the dose range tested.
The highest treatment exposure of amorphous solid dispersion formulation i.e., 561.6 mg otenaproxesul, yielded Day 4 (steady state) AUC exposure (1007 μg*hr/mL) comparable to the AUC exposure of 526.5 human equivalent dose of crystalline otenaproxesul, the NOAEL dog treatment dose (1058 μg*hr/mL)
All remaining amorphous solid dispersion otenaproxesul treatments had Day 4 (steady state) AUC exposures approximately 30% to 60% lower than crystalline otenaproxesul,

At comparable human equivalent doses of crystalline otenaproxesul (526.5 mg) or amorphous otenaproxesul (561.6 mg) the steady state pharmacokinetics of the respectively derived M25 naproxen were virtually identical.

### Results

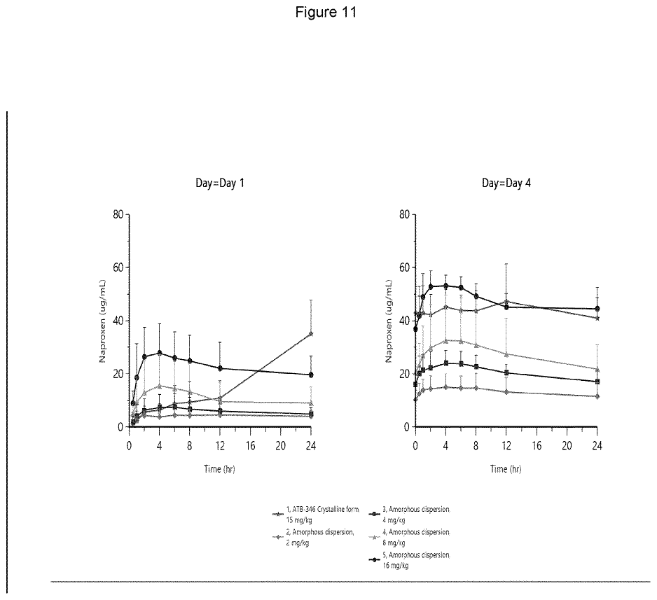

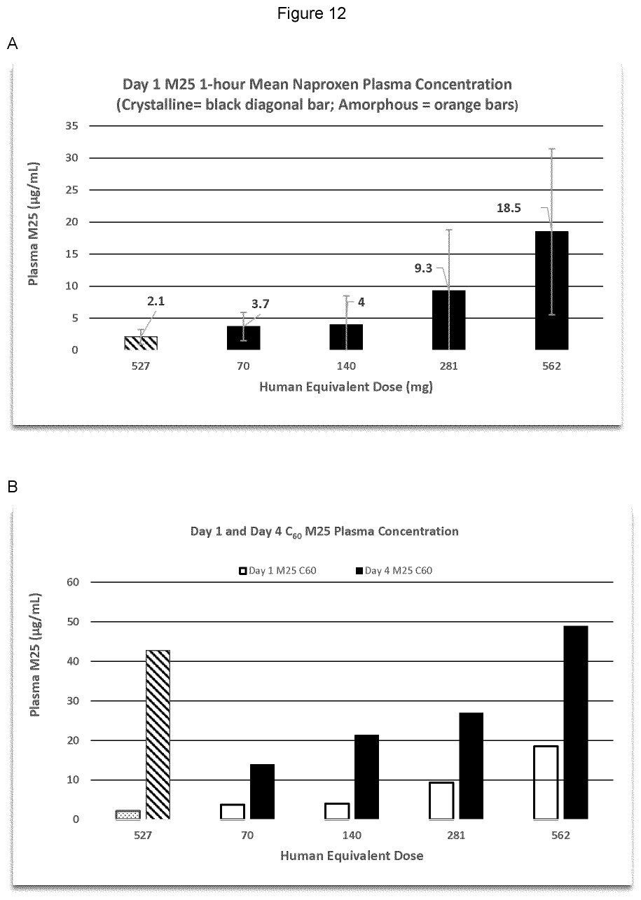

The pharmacokinetic concentration versus time profiles for a range of amorphous solid dispersion otenaproxesul treatments (i.e., exemplary ASD no. 1) in comparison to a control crystalline otenaproxesul treatment at Day 1 and Day 4 are shown in FIG. 11 while the group mean M25 plasma concentrations are shown in FIGS. 12A and B.

Amorphous solid dispersion otenaproxesul treatments all attained greater C₆₀ M25 plasma concentrations than crystalline otenaproxesul. The individual dog data showed that one dog in the 561.6 mg human equivalent treatment group had an unexplained low Coo plasma naproxen value of 0.41 μg/mL. Removal of this dog from the analysis elevates the group average C₆₀ measure to 24.5 μg/mL. Large standard deviations in M25 naproxen were seen in dogs receiving the two highest otenaproxesul doses such that one dog receiving the 281 mg human equivalent dose had a C₆₀ value of 22.8 μg/mL while three dogs in the 562 mg human equivalent dose had C₆₀ values of 18.3 μg/mL, 25.5 μg/mL and 29.8 μg/mL.

At steady state both the human equivalent 527 mg dose of crystalline otenaproxesul and the amorphous solid dispersion of otenaproxesul human equivalent dose of 562 mg exhibited comparable M25 plasma concentrations at 1-hour post-treatment on Day 4.

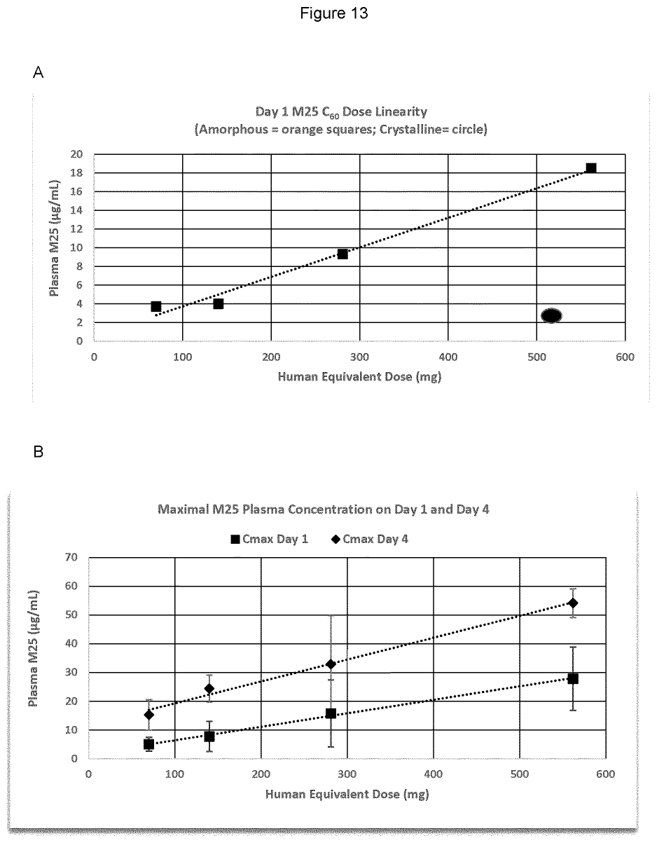

Plasma C₆₀ M25 concentrations exhibited a high degree of dose linearity on Day 1 with M25 concentrations from all amorphous solid dispersion formulation treatments exceeding the M25 plasma levels displayed by crystalline otenaproxesul (FIG. 13A).

The dose-proportionality of the M25 maximal plasma concentrations (Cₘₐₓ) for the amorphous solid dispersion otenaproxesul treatments over a human equivalent dose range of approximately 70 to 560 mg are shown in FIG. 13 B. Dose linearity remained unchanged during the treatment period. The Cₘₐₓ Day 4/Day 1 ratio for crystalline otenaproxesul was 1.4 whereas the Day 4/Day 1 Cₘₐₓ ratio for amorphous solid dispersion otenaproxesul treatments ranged from 1.9 to 3.2 suggesting a more rapid but continuous absorption of M25 naproxen.

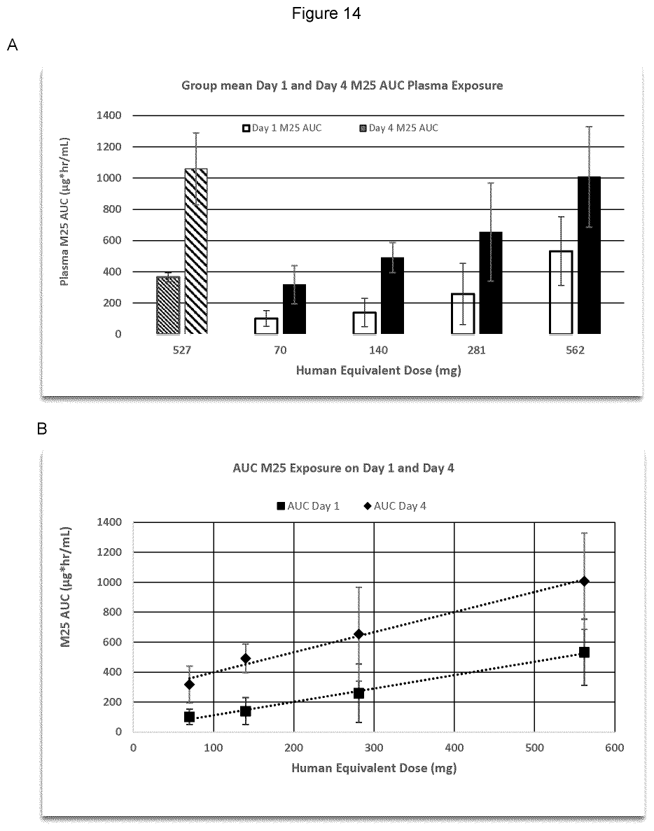

The Day 1 and Day 4 M25 AUC exposures for the amorphous solid dispersion otenaproxesul treatments over a human equivalent dose range of approximately 70 to 560 mg are shown in FIG. 14A. Ratios of Day 4/Day 1 AUCs for crystalline otenaproxesul and the two lower otenaproxesul amorphous solid dispersion formulation treatments (70 mg and 140 mg human equivalent doses) range from 2.9 to 3.5 whereas the Day 4/Day 1 ratios for the two higher otenaproxesul amorphous solid dispersion formulation treatments (281 mg and 562 mg human equivalent doses) are lower at 2.5 and 1.9, respectively. These data are surprisingly possibly indicative of lower amorphous drug accumulation in plasma over the measured treatment time driven by enhanced absorption and higher plasma exposures of the amorphous solid dispersion otenaproxesul formulation on Day 1 relative to crystalline otenaproxesul.

The dose-proportionality of the M25 AUC plasma exposure for the amorphous solid dispersion otenaproxesul treatments over a human equivalent dose range of approximately 70 to 560 mg are shown in FIG. 14 B. Exposure dose linearity remained unchanged during the treatment period.

The above data demonstrates multiple surprising advantages of the solid dispersion comprising amorphous otenaproxesul, including, but not limited to: 1) the rapid onset of the otenaproxesul amorphous solid dispersion formulation allows for the use of otenaproxesul to treat acute pain conditions which would not be viable using a crystalline form; 2) the rapid onset of the otenaproxesul amorphous solid dispersion formulation changes overall AUC exposure to hydrogen sulfide, which would in turn reduce or eliminate the previously unknown risk of liver injury associated with H₂S from otenaproxesul; 3) the rapid release the otenaproxesul amorphous solid dispersion formulation results in less drug accumulation in plasma between doses, resulting in consistent PK profiles for each dose and the viability of loading/tapering doses; and 4) the rapid onset the otenaproxesul amorphous solid dispersion formulation would permit the use of lower overall doses of otenaproxesul.

### Example 21A—Proposed Human Clinical Treatment Regimens Based on Dog Data

Using dog plasma AUC exposures derived from the amorphous otenaproxesul dosing ranges in the examples above, four human treatment regimens have been proposed (Table 21). The following considerations should be noted:

The AUC values reported here are based upon the calculated or estimated daily average (Days 1-4) AUC values (AUC₀₋₂₄) or the terminal (Day 5) AUC values (AUC_{0-inf}). The Total AUC is an estimate of the cumulative drug exposure across the 5-day treatment regimen factoring in higher exposure for a given drug dose administered on consecutive occasions and accounting for terminal drug exposure.

TABLE 25
Proposed human clinical treatment regimens

| | | | | | | Total |
|---|---|---|---|---|---|---|
| | Day 1 | Day 2 | Day 3 | Day 4 | Day 5 | AUC |
| Treatment | 600 mg | 200 mg | 100 mg | 100 mg | 50 mg | |
| 1 | + | + | + | + | + | |
| | 200 mg | 200 mg | 100 mg | 100 mg | 50 mg | |
| Exposure | 800 | 1060 | 510 | 800 | 291 | 3461 |
| Treatment | 600 mg | 200 mg | 100 mg | 50 mg | 50 mg | |
| 2 | + | + | + | + | + | |
| | 200 mg | 200 mg | 100 mg | 50 mg | 50 mg | |
| Exposure | 800 | 1060 | 510 | 291 | 452 | 3113 |
| Treatment | 600 mg | 200 mg | 50 mg | 50 mg | 50 mg | |
| 3 | + | + | + | + | + | |
| | 200 mg | 200 mg | 50 mg | 50 mg | 50 mg | |
| Exposure | 800 | 1060 | 291 | 452 | 452 | 3055 |
| Treatment | 600 mg | 200 mg | 25 mg | 25 mg | 25 mg | |
| 4 | + | + | + | + | + | |
| | 200 mg | 200 mg | 25 mg | 25 mg | 25 mg | |
| Exposure | 800 | 1060 | 158 | 226 | 226 | 2470 |

### Conclusions

The administration of all amorphous solid dispersion otenaproxesul formulations produced group mean Coo plasma M25 concentrations that were higher than the mean Coo values for the crystalline otenaproxesul control on Day 1. On Day 4 only the highest treatment dose of amorphous solid dispersion otenaproxesul had a comparable C₆₀ plasma naproxen concentration to that measured from crystalline otenaproxesul treatment. Additionally, the highest amorphous solid dispersion otenaproxesul treatment, corresponding to a human equivalent dose of 561.6 mg otenaproxesul had a Day 1 C₆₀ group mean measure that exceeded an M25 concentration of 18 μg/mL.

Pharmacokinetic extrapolations based on the dog pharmacokinetic (PK) study suggest that a single 600 mg otenaproxesul human dose of the amorphous solid dispersion formulation would attain appropriate plasma concentrations of M25 at the 1-hour post-treatment timepoint.

Plasma M25 naproxen concentrations and AUC exposures on both Day 1 and Day 4 (steady state) exhibit dose-linear profiles over the range of treatment doses tested. All amorphous solid dispersion otenaproxesul treatment regimens had treatment AUC exposures equal to or less than the human equivalent 526.5 mg crystalline otenaproxesul dose.

### Example 21B—Proposed Human Clinical Treatment Regimens Based on Simulation Data

Informed by the reproducible, and dose-proportional pharmacokinetics of otenaproxesul and noting that the otenaproxesul amorphous solid dispersion treatment exposures in animals align well with their specific human equivalent dose exposures seen with crystalline form drug administrations, a set of six different, short course treatment regimens were selected for liver safety analysis

TABLE 26
Potential Human Acute Pain Treatment Regimens Selected from Rat and Dog
Pharmacokinetic Assessments of Otenaproxesul Amorphous Dispersion Drug Administrations.

| | Day 1 | Day 2 | Day 3 | Day 4 | Day 5 | Total AUC |
|---|---|---|---|---|---|---|
| Treatment 1D | 600 mg | 200 mg | 100 mg | 100 mg | 50 mg | 3461 |
| | + | + | + | + | + | |
| | 200 mg | 200 mg | 100 mg | 100 mg | 50 mg | |
| Treatment 2D | 600 mg | 200 mg | 25 mg | 25 mg | 25 mg | 2470 |
| | + | + | + | + | + | |
| | 200 mg | 200 mg | 25 mg | 25 mg | 25 mg | |
| Treatment 3R | 400 mg | 200 mg | 200 mg | 100 mg | 100 mg | 3028 |
| | + | + | + | + | + | |
| | 200 mg | 200 mg | 200 mg | 100 mg | 100 mg | |
| Treatment 4R | 400 mg | 200 mg | 200 mg | 50 mg | 50 mg | 2464 |
| | + | + | + | + | + | |
| | 200 mg | 200 mg | 200 mg | 50 mg | 50 mg | |
| | 400 mg | 200 mg | 100 mg | 50 mg | 50 mg | 2224 |
| Treatment 5R | + | + | + | + | + | |
| | 200 mg | 200 mg | 100 mg | 50 mg | 50 mg | |
| Treatment 6R | 200 mg | 100 mg | 50 mg | 25 mg | 25 mg | 1403 |
| | + | + | + | + | + | |
| | 100 mg | 100 mg | 50 mg | 25 mg | 25 mg | |

Each of the treatment regimens, two based on dog PK (1D and 2D) and four based on rat PK (3R, 4R, 5R and 6R) has a calculated total 5-day AUC exposure<4000 μg*hr/mL. The selected regimens, and their predicted AUC exposures are summarized in Table 26. The in-silico population PK simulations of between 250 to 265 healthy individuals per arm were designed to assess the potential for liver damage via induction of liver transaminase elevations during a 5-day treatment course plus a 10-day follow up period immediately after cessation of drug administration.

The results of the simulation analyses, shown in Table 27 confirm that all the proposed treatment regimens using the amounts of otenaproxesul as described in Table 26 are predicted to be liver safe, i.e., essentially no observed LTEs, both during treatment and at the follow up assessment period, 10 days post-treatment.

TABLE 27
Simulated ALT Excursions During and After the New Acute
Treatments and the Severity of ALT Increases.

| | ALT ≥ 2X ULN | | ALT ≥ 3X ULN | |
|---|---|---|---|---|
| Otenaproxesul | During | Post | During | Post |
| Dose | Treatment | Treatment | Treatment | Treatment |
| Untreated | 0 | 0 | 0 | 0 |
| 1D (N = 265) | 0 | 1 | 0 | 1 |
| 2D (N = 253) | 0 | 1 | 0 | 1 |
| 3D (N = 259) | 0 | 1 | 0 | 1 |
| 4D (N = 259) | 0 | 1 | 0 | 1 |
| 5D (N = 250) | 0 | 0 | 0 | 0 |
| 6D (N = 263) | 0 | 1 | 0 | 1 |

### Example 22: Comparative Assessment of the Pharmacokinetics (PK) and Pharmacodynamics (PD) of Selected Treatment Regimens of an Amorphous Solid Dispersion Formulation of Otenaproxesul in Healthy Human Subjects

Design: This is an open-label, randomized, three-arm PK study. Arm(s) of this study may be conducted in groups. The same protocol requirements and procedures will be followed for each group.

The recruitment start will correspond with the study's Informed Consent Form (ICF) approval date, at which time study specific recruitment may begin. The anticipated end of study is the last scheduled procedure. Unscheduled visits and adverse event (AE) follow-up may occur after the end of study procedures. The expected duration of subject participation in this study (from voluntary signing of the ICF to the last scheduled study procedure) will be approximately 41 days for Arm 1. The expected duration of subject participation in this study (from voluntary signing of the ICF to the last scheduled study procedure) will be approximately 43 days for Arms 2 and 3.

Treatments: Subjects will receive one of the following treatments as outlined in the following table:

Test Product 1: otenaproxesul 25 mg Amorphous Dispersion (AD) tablets (e.g., compositions comprising ASD No. 1, for example, exemplary composition 3, with 25 mg otenaproxesul)

Test Product 2: otenaproxesul 100 mg Amorphous Dispersion (AD) tablets (e.g., compositions comprising ASD No. 1, for example, exemplary composition 3, 1 with 100 mg otenaproxesul)

TABLE 28
Dosing in all Arms

| Arm | Treatments | Time (e.g.) | Day 1 | Day 2 | Day 3 | Day 4 | Day 5 |
|---|---|---|---|---|---|---|---|
| 1 | A | 08:00 | 600 mg | NA | NA | NA | NA |
| | | | otenaproxes | | | | |
| | | | ul 6 × 100 mg | | | | |
| | | | AD tablets | | | | |
| | | 20:00 | NA | NA | NA | NA | NA |
| 2 | B | 08:00 | 200 mg | 100 mg | 50 mg | 25 mg | 25 mg |
| | | | otenaproxes | otenaproxes | otenaproxes | otenaproxesul | otenaproxes |
| | | | ul 2 × 100 mg | ul 1 × 100 mg | ul | 1 × 25 mg | ul |
| | | | AD tablets | AD tablet | 2 × 25 mg | AD tablet | 1 × 25 mg |
| | | | | | AD tablet | | AD tablet |
| | | 20:00 | 100 mg | 100 mg | 50 mg | 25 mg | 25 mg |
| | | | otenaproxes | otenaproxes | otenaproxes | otenaproxesul | otenaproxes |
| | | | ul 1 × 100 mg | ul | ul | 1 × 25 mg | ul |
| | | | AD tablet | 1 × 100 mg | 2 × 25 mg | AD tablet | 1 × 25 mg |
| | | | | AD tablets | AD tablets | | AD tablet |
| 3 | C | 08:00 | 400 mg | 200 mg | 100 mg | 50 mg | 50 mg |
| | | | otenaproxes | otenaproxes | otenaproxes | otenaproxesul | otenaproxes |
| | | | ul 4x100 mg | ul 2 × 100 mg | ul 1 × 100 mg | 2 × 25 mg | ul |
| | | | AD tablets | AD tablets | AD tablet | AD tablets | 2 × 25 mg |
| | | | | | | | AD tablets |
| | | 20:00 | 200 mg | 200 mg | 100 mg | 50 mg | 50 mg |
| | | | otenaproxes | otenaproxes | otenaproxes | otenaproxesul | otenaproxes |
| | | | ul 2 × 100 mg | ul | ul 1 × 100 mg | 2 × 25 mg | ul |
| | | | AD tablets | 2 × 100 mg | AD tablet | AD tablets | 2 × 25 mg |
| | | | | AD tablets | | | AD tablets |

Randomization: Subjects will be randomly assigned to one treatment arm according to a predetermined computer-generated randomization scheme (procedure PLAN in SAS® [version 9.4 or later]). Subjects will be assigned consecutive subject numbers in an ascending order. Each number will identify a subject and determine the sequence of drug product administration according to the randomization scheme.

Sample Size: Thirty-six (36) subjects will be enrolled into this PK study. Each arm will include 4 male subjects and 8 female subjects, with a minimum of 50% Caucasian subjects. Only volunteers who are treated will be considered enrolled.

Study Population: The study population will consist of healthy, non-smoking, male and female volunteers from 18 to 59 years of age. Subjects who have participated in one arm of the study will not be permitted to participate in a subsequent arm. Subjects enrolled in the study will satisfy the subject selection criteria no more than 30 days prior to the first drug administration.

While the present application has been described with reference to examples, it is to be understood that the scope of the claims should not be limited by the embodiments set forth in the examples but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A pharmaceutical composition comprising otenaproxesul or pharmaceutically acceptable salt thereof and a pharmaceutically acceptable excipient, wherein at least about 95 wt % of the otenaproxesul, or the pharmaceutically acceptable salt thereof, is in amorphous form.

2. An amorphous solid dispersion comprising amorphous otenaproxesul or pharmaceutically acceptable salt thereof and a polymer.

3. The amorphous solid dispersion of claim 2, wherein the polymer is a stabilizing polymer.

4. The amorphous solid dispersion of claim 2 or 3, wherein the amorphous otenaproxesul is in a neutral form.

5. The amorphous solid dispersion of any one of claims 2 to 4, wherein the amorphous otenaproxesul or pharmaceutically acceptable salt thereof is present in the amorphous solid dispersion in a form that is about 90 wt % or more, about 92 wt % or more, about 94 wt % or more, about 95 wt % or more, about 96 wt %, about 97 wt %, about 98 wt %, or about 99 wt % or more amorphous form.

6. The amorphous solid dispersion of any one of claims 2 to 5, wherein the amorphous otenaproxesul or pharmaceutically acceptable salt thereof is present in the amorphous solid dispersion in an amount of about 15% to about 60%, about 15% to about 50%, about 15% to about 50%, about 20% to about 50%, about 30% to about 50% or about 35% to about 50% by weight of the amorphous solid dispersion.

7. The amorphous solid dispersion of any one of claims 2 to 6, wherein the polymer is present in the amorphous solid dispersion in an amount of about 40% to about 80%, 40% to about 70%, about 40% to about 65%, about 50% to about 80%, about 50% to about 70%, about 50% to about 65%, about 55% to about 65%, about 50% to about 60%, or about 60% to about 65% by weight of the amorphous solid dispersion.

8. The amorphous solid dispersion of any one of claims 2 to 7, wherein the weight ratio of amorphous otenaproxesul, or pharmaceutically acceptable salt thereof, to the polymer in the amorphous solid dispersion is about 1:1 to about 1:4, about 1:1.5, about 1:1.75 or about 1:1.8.

9. The amorphous solid dispersion of any one of claims 3 to 8, wherein the polymer is selected from polyvinylpyrrolidone (PVP) based polymers, polyethylene glycol (PEG) based polymers, cellulose based polymers, acrylate based polymers, chitosan, polyvinyl alcohol/polyethylene glycol graft copolymers, polyvinyl caprolactam polyvinyl acetate-polyethylene glycol graft copolymers and polyvinyl acetate phthalate, and combinations of one or more of the listed polymers.

10. The amorphous solid dispersion of any one of claims 3 to 8, wherein the polymer is selected from polyvinylpyrrolidone, polyvinylpyrrolidone vinyl acetate (PVP VA), crospovidone (PVPP), methylcellulose, hydroxypropylmethylcellulose (HPMC), hydroxypropylmethylcellulose acetate succinate (HPMCAS), hydroxypropylmethylcellulose phthalate (HPMCP), cellulose acetate phthalate (CAP), methacrylate copolymers, polyacrylic acid, chitosan, polyvinyl alcohol/polyethylene glycol graft copolymers, polyvinyl caprolactam polyvinyl acetate-polyethylene, combinations of one or more of the listed polymers, and polyethylene glycol (PEG) based polymers in combination with one or more of the listed polymers,
provided HPMC does not comprise E type HPMC comprising a viscosity of about 5 cP or greater.

11. The amorphous solid dispersion of any one of claims 3 to 8, wherein the polymer is selected from polyvinylpyrrolidone K-12, polyvinylpyrrolidone vinyl acetate (PVP VA), crospovidone (PVPP), hydroxypropylmethylcellulose E3 (HPMC E3), hydroxypropylmethylcellulose acetate succinate (HPMCAS), methacrylate copolymers, polyvinyl alcohol/polyethylene glycol graft copolymers and polyvinyl caprolactam polyvinyl acetate-polyethylene glycol graft copolymers, and combinations thereof.

12. The amorphous solid dispersion of claim 11, wherein the polymer is HPMCAS.

13. The amorphous solid dispersion of any one of claims 2 to 12, wherein the amorphous solid dispersion further comprises one or more additives and the one or more additives are one or more antioxidants and the one or more antioxidants are present in the amorphous solid dispersion in an amount of about 0.1% to about 1%, about 0.1% to about 0.75%, about 0.1% to about 0.5%, about 0.1% to about 0.4%, about 0.2% to about 0.5%, about 0.2% to about 0.4%, about 0.3% to about 0.5%, or about 0.4% to about 0.5% by weight of the amorphous solid dispersion.

14. The amorphous solid dispersion of any one of claims 2 to 13, wherein the amorphous solid dispersion comprises less 0.02 wt % of naproxen when stored at a temperature of about 30° C. or about 50° C. for one month, and/or
the amorphous solid dispersion comprises less than about 0.2 wt %, less than about 0.1 wt % or less than about 0.05 wt % impurity observed at relative retention time (RRT) 0.24 (4-hydroxybenzonitrile) when stored at a temperature of about 30° C. or about 50° C. for one month, and/or
the amorphous solid dispersion comprises less than about 0.2 wt %, less than about 0.1 wt % or less than about 0.05 wt % impurity observed at RRT 0.85 when stored at a temperature of about 30° C. or about 50° C. for one month.

15. The amorphous solid dispersion of any one of claims 2 to 14, further comprising one or more sustaining agents.

16. The amorphous solid dispersion of claim 15, wherein the one or more sustaining agents are combined with the otenaproxesul or pharmaceutically acceptable salt thereof and the polymer in the solid dispersion to form the amorphous solid dispersion comprising otenaproxesul or pharmaceutically acceptable salt thereof, the polymer and the sustaining agent.

17. A pharmaceutical composition comprising an amorphous solid dispersion of any one of claims 2 to 16, and one or more pharmaceutically acceptable excipients.

18. The pharmaceutical composition of claim 17, wherein the one or more pharmaceutically acceptable excipients comprise one or more intragranular pharmaceutically acceptable excipients and one or more extragranular pharmaceutically acceptable excipients.

19. The pharmaceutical composition of claim 18, wherein the amorphous solid dispersion is granulated together with the one or more intragranular pharmaceutically acceptable excipients to form granules.

20. The pharmaceutical composition of claim 18 or claim 19, wherein the one or more intragranular pharmaceutically acceptable excipients are selected from one or more intragranular pharmaceutically acceptable additives, fillers, disintegrants, lubricants, and glidants, and the one or more extragranular pharmaceutically acceptable excipients are selected from one or more extragranular pharmaceutically acceptable fillers, disintegrants, lubricants and glidants.

21. The pharmaceutical composition of any one of claims 17 to 20, wherein the pharmaceutical composition comprises about 25% to about 65% of the amorphous solid dispersion, by weight of the composition.

22. The pharmaceutical composition of any one of claims 17 to 21, wherein the pharmaceutical composition comprises:
about 45% to about 65% of the amorphous solid dispersion of any one of claims 2 to 16;
about 25% to about 50% of the one or more intragranular pharmaceutically acceptable fillers;
about 2% to about 8% of the one or more intragranular pharmaceutically acceptable disintegrants,
about 0.1% to about 2% of the one or more intragranular pharmaceutically acceptable glidants and lubricants; and
optionally about 0.5% to about 2% of one or more intragranular pharmaceutically acceptable additives;
wherein the percentage amount is by weight of the pharmaceutical composition.

23. The pharmaceutical composition of any one of claims 17 to 22, further comprising one or more sustaining agents external to the amorphous solid dispersion.

24. The pharmaceutical composition of claim 23, wherein the weight ratio of the one or more sustaining agents to amorphous solid dispersion is about 1:1 to about 2:5.

25. The pharmaceutical composition of claim 23 or 24, wherein the one or more sustaining agents is a polymer.

26. The pharmaceutical composition of any one of claims 17 to 25, wherein the pharmaceutical is formulated as a solid oral composition.

27. The pharmaceutical composition of claim 26, wherein the amorphous otenaproxesul or a pharmaceutically acceptable salt thereof is present in the pharmaceutical composition in an amount of about 5 mg to about 400 mg.

28. A pharmaceutical package or kit comprising:
one or more pharmaceutical compositions of any one of claims 17 to 26 comprising a loading dose of amorphous otenaproxesul or a pharmaceutically acceptable salt thereof, and
instructions for administration of the one or more pharmaceutical compositions comprising the loading dose of the amorphous otenaproxesul or the pharmaceutically acceptable salt thereof, to a subject in need thereof.

29. The pharmaceutical package or kit oof claim 28, wherein the loading dose is about 25 mg to about 600 mg, about 25 mg to about 500 mg, about 25 mg to about 400 mg, about 25 mg to about 300 mg, about 25 mg to about 200 mg, about 25 mg to about 100 mg, about 50 mg to about 500 mg, about 50 mg to about 400 mg, about 50 mg to about 300 mg, about 50 mg to about 200 mg, about 50 mg to about 100 mg, about 100 mg to about 400 mg, or about 100 mg to about 200 mg of the amorphous otenaproxesul or the pharmaceutically acceptable salt thereof.

30. The pharmaceutical package or kit of claim 28 or 29, wherein the package or kit further comprises a pharmaceutical composition of any one of claims 17 to 26 comprising a second loading dose of amorphous otenaproxesul or a pharmaceutically acceptable salt thereof.

31. The pharmaceutical package or kit of any one of claims 28 to 30, wherein the pharmaceutical package or kit further comprises one or more pharmaceutical compositions comprising a placebo dose.

32. A pharmaceutical package or kit comprising:
a pharmaceutical composition of any one of claims 17 to 26 comprising a loading dose of amorphous otenaproxesul or a pharmaceutically acceptable salt thereof, and
one or more pharmaceutical compositions of any one of claims 17 to 26 comprising a maintenance dose of amorphous otenaproxesul or a pharmaceutically acceptable salt thereof.

33. The pharmaceutical package or kit of claim 32, wherein the maintenance dose is about 5 mg, about 10 mg, about 25 mg about 50 mg, about 75 mg, about 100 mg, about 125 mg, about 150 mg, about 175 mg, or about 200 mg of the amorphous otenaproxesul, or a pharmaceutically acceptable salt thereof.

34. The pharmaceutical package or kit of claim 32 or 33, wherein the one or more pharmaceutical compositions comprising the one or more maintenance doses comprises one or more initial maintenance doses of the amorphous otenaproxesul or the pharmaceutically acceptable salt thereof, and one or more following maintenance doses of the amorphous otenaproxesul or the pharmaceutically acceptable salt thereof.

35. The pharmaceutical package or kit of claim 34, wherein the kit comprises one pharmaceutical composition comprising the loading dose of the amorphous otenaproxesul or the pharmaceutically acceptable salt thereof, two to five pharmaceutical compositions comprising the initial maintenance dose of amorphous otenaproxesul or a pharmaceutically acceptable salt thereof, and four to seven pharmaceutical compositions comprising the following maintenance dose of amorphous otenaproxesul or a pharmaceutically acceptable salt thereof.

36. The pharmaceutical package or kit of claim 34, wherein the kit comprises one pharmaceutical composition comprising the loading dose of the amorphous otenaproxesul or the pharmaceutically acceptable salt thereof, five to seven pharmaceutical compositions comprising the initial maintenance doses of the amorphous otenaproxesul or the pharmaceutically acceptable salt thereof, and three to seven pharmaceutical compositions comprising following maintenance doses of the amorphous otenaproxesul or a pharmaceutically acceptable salt thereof.

37. The pharmaceutical package or kit of any one of claims 29 to 33, wherein the pharmaceutical package or kit further comprises one or more pharmaceutical compositions comprising a placebo dose.

38. The pharmaceutical package or kit of any one of claims 32 to 37, wherein the package or kit is a blister pack, a pill dispenser, a clam shell dispenser or tray.

39. A method for treating diseases, disorders or conditions treatable by inhibition of COX-1 and/or COX-2 comprising administering an effective amount of one or more pharmaceutical compositions of any one of claims 17 to 27 to a subject in need thereof.

40. The method of claim 39, wherein the diseases disorders or conditions treatable by inhibition of COX-1 and/or COX-2 are selected from inflammation and diseases, disorders or conditions associated with inflammation.

41. The method of claim 40, wherein the inflammation or disease, disorder or condition associated with inflammation is arthritis.

42. The method of claim 41, wherein the arthritis is rheumatoid arthritis, ankylosing spondylitis, spondyloarthropathies, osteoarthritis, gouty arthritis, systemic lupus erythematosus or juvenile idiopathic arthritis.

43. The method of claim 39, wherein the diseases, disorders or conditions treatable by inhibition of COX-1 and/or COX-2 are selected from one or more of pain, fever and headaches.

44. The method of claim 43, wherein the disease, disorder or condition treatable by inhibition of COX-1 and/or COX-2 is pain.

45. The method of claim 44, wherein the method provides a cumulative plasma naproxen metabolite (M25) exposure of less than about 4000 μg*hr/mL.

46. The method of claim 45, wherein the pain is acute pain.

47. The method of claim 46, wherein the acute pain is selected from pain caused by acute injury, trauma, illness and surgery.

48. The method of claim 46, wherein the acute pain selected from post-operative pain, peri-operative pain, primary dysmenorrhea, interstitial cystitis, headache, migraine, pain due to a trauma, renal colic, biliary colic, arthritis, dental pain, musculoskeletal pain, lower back pain, fibromyalgia, pain of infectious origin, pain resulting from cancer and pain induced by gout.

49. The method of claim 48, wherein the post-operative pain is selected from pain following a surgery selected from orthopedic, abdominal, pelvic, dental, plastic, cosmetic, neurological, urological, bariatric, gastric, cardiac, orthoscopic, vascular, endovascular, laparoscopic, oncological, colorectal, podiatric, ocular, otoplastic, rhinoplastic, and throat surgery.

50. The method of claim 49, wherein the orthopedic surgery is selected from bunionectomy, total hip, total knee, bilateral total knee, spine, shoulder, ankle, soft tissue surgeries, spinal fusion, rotator cuff repair, laminectomy, fracture repair, and discectomy; and
the abdominal and/or pelvic surgery is selected from inguinal hernia repair, abdominal hysterectomy, abdominal laparotomy, cholecystectomy, vaginal hysterectomy, ventral hernia repair, myomectomy, salpingo-oophorectomy, bariatric, partial colectomy surgeries, gynecologic surgery and genitourinary surgery.

51. The method of claim 46, wherein the acute pain is headache pain.

52. The method of claim 51, wherein the headache pain is selected from migraine, photophobia and phonophobia.

53. The method of claim 46, wherein the acute pain is dysmenorrhea.

54. The method of claim 46, wherein the acute pain is dental pain.

55. A method of treating pain in a subject in need thereof comprising administering an amount otenaproxesul or a pharmaceutically acceptable salt thereof that provides a cumulative plasma naproxen metabolite (M25) exposure of less than about 4000 μg*hr/mL.

56. The method of claim 55, wherein the method comprises administering one or more pharmaceutical compositions of any one of claims 17 to 26 comprising a loading dose of amorphous otenaproxesul or a pharmaceutically acceptable salt thereof.

57. The method of claim 56, wherein one pharmaceutical composition comprising the loading dose is administered.

58. The method of claim 56 or claim 57, wherein the loading dose comprises about 150 mg, about 200 mg, about 250 mg, about 300 mg, about 350 mg, about 400 mg, about 450 mg, about 500 mg, about 550 mg or about 600 mg of amorphous otenaproxesul or a pharmaceutically acceptable salt thereof.

59. The method of any one of claims 56 to 58, wherein the pharmaceutical compositions comprising the loading dose provides a plasma concentration of naproxen of about 10 μg/mL or greater, about 15 μg/mL or greater or about 20 μg/mL or greater in less than about 60 minutes after administration.

60. The method of claim 59, wherein the method comprises, on the third, fourth, fifth, sixth or seventh day after administration of the one or more pharmaceutical compositions comprising a first loading dose, administering a second composition of any one of claims 17 to 26 comprising a second loading dose of amorphous otenaproxesul or a pharmaceutically acceptable salt thereof to the subject.

61. The method of claim 60, wherein on days preceding or following administration of the composition comprising the second loading dose, the subject is administered a composition comprising a placebo.

62. The method of claim 61 or claim 61, wherein the composition comprising the second loading dose comprises a lower amount of amorphous otenaproxesul or a pharmaceutically acceptable salt thereof as the composition comprising the first loading dose.

63. The method of any one of claims 56 to 59, wherein the method further comprises, following administering of the one or more pharmaceutical compositions comprising a loading dose of amorphous otenaproxesul or a pharmaceutically acceptable salt thereof, administration of one or more pharmaceutical compositions comprising a maintenance dose of amorphous otenaproxesul or a pharmaceutically acceptable salt thereof, wherein administration of the compositions comprising amorphous otenaproxesul or a pharmaceutically acceptable salt thereof is for a treatment period of 14 days or less.

64. The method of claim 63, wherein each maintenance dose of the amorphous otenaproxesul or a pharmaceutically acceptable salt thereof comprises about 5 mg to about 400 mg per day of amorphous otenaproxesul, or pharmaceutically acceptable salt thereof.

65. The method of claim 63 or claim 64, wherein the loading doses of amorphous otenaproxesul or a pharmaceutically acceptable salt thereof are greater than each maintenance dose of amorphous otenaproxesul or a pharmaceutically acceptable salt thereof.

66. The method of any one of claims 63 to 65, wherein the maintenances doses are provided as tapered doses.

67. The method of claim 66, wherein the tapered doses comprise a dose that is decreased stepwise by an amount over a period of time.

68. The method of any one of claims 63 to 66, wherein the loading doses comprise about 100 mg to about 600 mg, about 100 mg to about 400 mg, or about 200 mg to about 400 mg per day of amorphous otenaproxesul, or a pharmaceutically acceptable salt thereof and each maintenance dose comprises about 25 mg to about 400 mg of amorphous otenaproxesul, or a pharmaceutically acceptable salt thereof.

69. The method of claim 68, comprising:
administering one pharmaceutical composition of any one of claims 17 to 26 comprising a loading dose comprising about 100 mg to about 600 mg of amorphous otenaproxesul or a pharmaceutically acceptable salt thereof, and
administering one or more pharmaceutical compositions of any one of claims 17 to 26 comprising a maintenance dose comprising about 25 mg to about 400 mg of amorphous otenaproxesul or a pharmaceutically acceptable salt thereof,
wherein administration of the amorphous otenaproxesul or a pharmaceutically acceptable salt thereof is for a treatment period of 7 days or less.

70. The method of any one of claims 63 to 69, wherein each maintenance dose is different and is selected from an initial maintenance dose and a following maintenance doses, and the method further comprises administering one or more pharmaceutical compositions of any one of claims 17 to 26 comprising an initial maintenance dose and administering one or more pharmaceutical compositions any one of claims 17 to 26 comprising a following maintenance dose.

71. The method of claim 70 comprising:
administering one pharmaceutical composition of any one of claims 17 to 26 comprising a loading dose comprising about 100 mg to about 600 mg of amorphous otenaproxesul or a pharmaceutically acceptable salt thereof,
administering one or more pharmaceutical compositions of any one of claims 17 to 26 comprising an initial maintenance dose comprising about 25 mg to about 200 mg of amorphous otenaproxesul or a pharmaceutically acceptable salt thereof, and
administering one or more pharmaceutical compositions of any one of claims 17 to 26 comprising a following maintenance dose comprising about 25 mg to about 100 mg of amorphous otenaproxesul or a pharmaceutically acceptable salt thereof,
wherein administration of the amorphous otenaproxesul or a pharmaceutically acceptable salt thereof is for a treatment period of 7 days or less, and each the initial and following maintenance doses are the same or different.

72. The method of claim 71, wherein the composition comprising the loading dose comprises about 600 mg of amorphous otenaproxesul or a pharmaceutically acceptable salt thereof, the one or more compositions comprising the initial maintenance dose comprise about 50 mg to about 200 mg of amorphous otenaproxesul or a pharmaceutically acceptable salt thereof, and the one or more compositions comprising the following maintenance dose comprise about 50 mg or about 100 mg of amorphous otenaproxesul or a pharmaceutically acceptable salt thereof.

73. The method of claim 71, wherein the composition comprising the loading dose comprises about 400 mg of amorphous otenaproxesul or a pharmaceutically acceptable salt thereof, the one or more compositions comprising the initial maintenance dose comprise about 200 mg of amorphous otenaproxesul or a pharmaceutically acceptable salt thereof, and the one or more compositions comprising the following maintenance dose comprise about 50 mg or about 100 mg of amorphous otenaproxesul or a pharmaceutically acceptable salt thereof.

74. The method of claim 71, wherein the composition comprising the loading dose comprises about 200 mg of amorphous otenaproxesul or a pharmaceutically acceptable salt thereof, the one or more compositions comprising an initial maintenance dose comprises about 100 mg of amorphous otenaproxesul or a pharmaceutically acceptable salt thereof, and the one or more compositions comprising a following maintenance dose comprise about 25 mg to about 50 mg of amorphous otenaproxesul or a pharmaceutically acceptable salt thereof.

75. The method of any one of claims 71 to 74, wherein the treatment period is for 5 days.

76. The method of claim 74, wherein the composition comprising the loading dose comprises about 200 mg of amorphous otenaproxesul or a pharmaceutically acceptable salt thereof, the one or more compositions comprising initial maintenance doses comprise about 100 mg of amorphous otenaproxesul or a pharmaceutically acceptable salt thereof, and the one or more compositions comprising following maintenance doses comprise about 25 mg or about 50 mg of amorphous otenaproxesul or a pharmaceutically acceptable salt thereof, and
one of the pharmaceutical compositions comprising the initial maintenance dose is administered on the same day as the pharmaceutical composition comprising the loading dose;
one or two of the pharmaceutical compositions comprising the initial maintenance dose or one pharmaceutical composition comprising the initial maintenance dose and one pharmaceutical composition comprising the maintenance dose are administered on the day after administration of the pharmaceutical composition comprising the loading dose,
and on subsequent treatment days one or two pharmaceutical compositions comprising the following maintenance dose are administered per day.

77. The method of claim 73, wherein the composition comprising the loading dose comprises about 400 mg of amorphous otenaproxesul or a pharmaceutically acceptable salt thereof, the one or more compositions comprising the initial maintenance dose comprise about 200 mg of amorphous otenaproxesul or a pharmaceutically acceptable salt thereof, and the one or more compositions comprising the following maintenance dose comprise about 50 mg or about 100 mg of amorphous otenaproxesul or a pharmaceutically acceptable salt; and
one of the pharmaceutical compositions comprising the initial maintenance dose is administered on the same day as the pharmaceutical composition comprising the loading dose;
one or two of the pharmaceutical compositions comprising the initial maintenance dose or one pharmaceutical composition comprising the initial maintenance dose and one pharmaceutical composition comprising the following maintenance dose are administered on the day after administration of the pharmaceutical composition comprising the loading dose,
and on subsequent treatment days one or two pharmaceutical compositions comprising the following maintenance doses are administered per day.

78. The method of claim 76 or 77, wherein one of the pharmaceutical compositions comprising the initial maintenance dose is administered on the same day as the pharmaceutical composition comprising the loading dose;
two of the pharmaceutical compositions comprising the initial maintenance dose are administered on the day after administration of the pharmaceutical composition comprising the loading dose,
and on subsequent treatment days two pharmaceutical compositions comprising the following maintenance dose are administered per day.

79. The method of claim 76 or 77, wherein one of the pharmaceutical compositions comprising the initial maintenance doses is administered on the same day as the pharmaceutical composition comprising the loading dose;
one pharmaceutical composition comprising the initial maintenance dose and one pharmaceutical composition comprising the following maintenance dose are administered on the day after administration of the pharmaceutical composition comprising the loading dose,
and on subsequent treatment days two pharmaceutical compositions comprising the following maintenance dose are administered per day.

80. The method of any one of claims 76 to 78, wherein the two pharmaceutical compositions comprising the initial or the following maintenance dose are administered once in the morning and once in the evening of the same day.

81. The method of any one of claims 76 to 78, wherein the pharmaceutical compositions comprising the initial and/or following maintenance doses are provided about every 12 hours.

82. The method of any one of claims 76 to 81, wherein the following maintenance doses are tapered once from the administration of a last initial maintenance dose to the last day of the treatment period.

83. The method of any one of claims 56 to 82, wherein the method provides a Tmax of naproxen metabolite in about 1 hour to 3 hours, about 1 hour to about 2 hours, about 30 minutes to about 90 minutes or about 30 minutes to about on after oral administration of the pharmaceutical composition comprising the loading dose.

84. The method of any one of claims 56 to 82, wherein the method provide a Cₘₐₓ of naproxen metabolite of about 20 μg/mL to about 100 μg/mL, about 30 μg/mL to about 80 μg/mL, about 40 μg/mL to about 70 μg/mL, or about 50 μg/mL to about 65 μg/mL following oral administration of the pharmaceutical compositions.

85. The method of any one of claims 56 to 82, wherein the method provides an AUC of naproxen of from about 500 μg*hr/mL to about 4000 μg*hr/mL following administration of the pharmaceutical compositions.

86. The method of claim 55, wherein the method comprises administering one or more loading doses of crystalline otenaproxesul or a pharmaceutically acceptable salt thereof.

87. The method of claim 86, wherein the loading dose comprises about 100 mg to about 2000 mg of crystalline otenaproxesul or a pharmaceutically acceptable salt thereof.

88. The method of claim 86 or 87, wherein only one loading dose of crystalline otenaproxesul or a pharmaceutically acceptable salt thereof is administered.

89. The method of claim 86 or 87, wherein the loading dose of crystalline otenaproxesul or a pharmaceutically acceptable salt thereof, provides a plasma concentration of naproxen, a metabolite of otenaproxesul, of about 10 μg/mL or greater, about 15 μg/mL or greater or about 20 μg/mL or greater in less than about 30 minutes, less than about 45 minutes, less than about 60 minutes, less than about 75 minutes or less than about 90 minutes after administration.

90. The method of claim 89, wherein the method comprises, on the third, fourth, fifth, sixth or seventh day after administration of the one or more loading doses of crystalline otenaproxesul or a pharmaceutically acceptable salt thereof, administering a second loading dose of crystalline otenaproxesul or a pharmaceutically acceptable salt thereof to the subject.

91. The method of claim 90, wherein on days preceding or following the administration of the second loading dose of crystalline otenaproxesul or a pharmaceutically acceptable salt thereof, the subject is administered a composition comprising a placebo.

92. The method of claim 91, wherein the second loading dose of crystalline otenaproxesul or a pharmaceutically acceptable salt thereof comprises a lower amount of crystalline otenaproxesul or a pharmaceutically acceptable salt thereof as the first loading dose of crystalline otenaproxesul or a pharmaceutically acceptable salt thereof.

93. The method of any one of claims 86 to 89, wherein the method further comprises, following administering the one or more loading doses of the crystalline otenaproxesul or a pharmaceutically acceptable salt thereof, administering one or more maintenance doses of the crystalline otenaproxesul or a pharmaceutically acceptable salt thereof, wherein administration of the compositions comprising crystalline otenaproxesul or a pharmaceutically acceptable salt thereof is for a treatment period of 14 days or less.

94. The method of claim 93, wherein the method comprises
administering one or more loading dose comprising about 100 mg to about 2000 mg of crystalline otenaproxesul or a pharmaceutically acceptable salt thereof, and
administering one or more a maintenance doses comprising about 25 mg to about 600 mg of crystalline otenaproxesul or a pharmaceutically acceptable salt thereof,
wherein administration of the crystalline otenaproxesul or a pharmaceutically acceptable salt thereof is for a treatment period of 14 days or less.

95. The method of claim 94, wherein each maintenance dose of crystalline otenaproxesul or a pharmaceutically acceptable salt thereof independently comprises about 25 mg, about 50 mg, about 75 mg, about 100 mg, about 125 mg, about 150 mg, about 175 mg, about 200 mg, about 225 mg, about 250 mg, about 300 mg, about 350 mg, about 400 mg, about 500 mg or about 600 mg of crystalline otenaproxesul, or a pharmaceutically acceptable salt thereof.

96. The method of any one of claims 93 to 95, wherein the one or more loading doses of crystalline otenaproxesul or a pharmaceutically acceptable salt thereof are greater than the one or more maintenances doses of crystalline otenaproxesul or a pharmaceutically acceptable salt thereof.

97. The method of any one of claims 93 to 96, wherein the one or more a maintenances dose of crystalline otenaproxesul or a pharmaceutically acceptable salt thereof are provided as tapered doses.

98. The method of one of claims 93 to 97, wherein the method comprises:
administering one or more pharmaceutical compositions comprising a loading dose comprising about 100 mg to about 2000 mg of crystalline otenaproxesul or a pharmaceutically acceptable salt thereof, and
administering one or more pharmaceutical compositions comprising a maintenance dose comprising about 25 mg to about 400 mg of crystalline otenaproxesul or a pharmaceutically acceptable salt thereof,
wherein administration of the crystalline otenaproxesul or a pharmaceutically acceptable salt thereof is for a treatment period of 10 days or less.

99. The method of any one of claims 93 to 97, wherein the one or more loading doses comprise about 100 mg to about 1500 mg, 100 mg to about 1000 mg, about 100 mg to about 600 mg, or about 200 mg to about 400 mg per day of crystalline otenaproxesul, or a pharmaceutically acceptable salt thereof and each maintenance dose comprises about 50 mg to about 400 mg of crystalline otenaproxesul, or a pharmaceutically acceptable salt thereof.

100. The method of one of claims 93 to 97, comprising:
administering one loading dose comprising about 100 mg to about 2000 mg of crystalline otenaproxesul or a pharmaceutically acceptable salt thereof, and
administering one or more maintenance doses comprising about 50 mg to about 400 mg of crystalline otenaproxesul or a pharmaceutically acceptable salt thereof,
wherein administration of the crystalline otenaproxesul or a pharmaceutically acceptable salt thereof is for a treatment period of 7 days or less.

101. The method of claim 100, wherein the one or more maintenance doses of crystalline otenaproxesul or a pharmaceutically acceptable salt thereof are one or more initial maintenance doses of crystalline otenaproxesul or a pharmaceutically acceptable salt thereof and one or more following maintenance doses of crystalline otenaproxesul or a pharmaceutically acceptable salt thereof, and the method comprises administering one or more initial maintenance doses of crystalline otenaproxesul or a pharmaceutically acceptable salt thereof; and administering one or more following maintenance doses of crystalline otenaproxesul or a pharmaceutically acceptable salt thereof.

102. The method of claim 101, wherein the one or more initial maintenance doses of crystalline otenaproxesul or a pharmaceutically acceptable salt thereof are higher than the one or more following maintenance doses of crystalline otenaproxesul or a pharmaceutically acceptable salt thereof.

103. The method of claim 101 or 102, wherein the one or more initial and/or following maintenance doses of crystalline otenaproxesul or a pharmaceutically acceptable salt thereof are provided about every 6 hours to 36 hours, 12 hours to 36 hours or 12 hours to 24 hours.

104. The method of claim 101 or 102, wherein one initial maintenance dose of crystalline otenaproxesul or a pharmaceutically acceptable salt thereof is administered on the same day as the loading dose of crystalline otenaproxesul or a pharmaceutically acceptable salt thereof.

105. The method of claim 101 or 102, wherein one or two initial maintenance doses of crystalline otenaproxesul or a pharmaceutically acceptable salt thereof are administered the day following the administration of a loading dose of crystalline otenaproxesul or a pharmaceutically acceptable salt thereof.

106. The method of claim 101 or 102, wherein the loading dose of crystalline otenaproxesul or a pharmaceutically acceptable salt thereof is administered in the morning and one initial maintenance dose of crystalline otenaproxesul or a pharmaceutically acceptable salt thereof is administered in the evening of the same day, and on the following treatment day, one or two initial maintenance doses of crystalline otenaproxesul or a pharmaceutically acceptable salt thereof are administered, or one initial maintenance dose of crystalline otenaproxesul or a pharmaceutically acceptable salt thereof and one following maintenance dose of crystalline otenaproxesul or a pharmaceutically acceptable salt thereof is administered, and on following treatment days one or two following maintenance doses of crystalline otenaproxesul or a pharmaceutically acceptable salt thereof are administered per day.

107. The method of claim 101 or 102, wherein the one or more following maintenance doses of crystalline otenaproxesul or a pharmaceutically acceptable salt thereof are tapered after administration the initial maintenance doses of crystalline otenaproxesul or a pharmaceutically acceptable salt thereof to the second, third, fourth day fifth, six or seventh day after administration of the loading dose of crystalline otenaproxesul or a pharmaceutically acceptable salt thereof.

108. The method of claim 101 or 102, wherein the method comprises:
administering one loading dose comprising about 100 mg to about 2000 mg of crystalline otenaproxesul or a pharmaceutically acceptable salt thereof,
administering one or more initial maintenance doses comprising about 50 mg to about 600 mg of crystalline otenaproxesul or a pharmaceutically acceptable salt thereof, and
administering one or more following maintenance doses comprising about 25 mg to about 200 mg of crystalline otenaproxesul or a pharmaceutically acceptable salt thereof,
wherein administration of the crystalline otenaproxesul or a pharmaceutically acceptable salt thereof is for a treatment period of 7 to 10 days or less, and each the initial and following maintenance doses are the same or different.

109. The method of any one of claims 101 to 108, wherein the following maintenance doses of crystalline otenaproxesul or a pharmaceutically acceptable salt thereof are tapered once or twice from the administration of a last initial maintenance dose of crystalline otenaproxesul or a pharmaceutically acceptable salt thereof to the last day of the treatment period.

110. The method of any one of claims 86 to 109, wherein each dose of crystalline otenaproxesul or a pharmaceutically acceptable salt and/or solvate thereof is formulated into a pharmaceutical composition comprising the dose of crystalline otenaproxesul or a pharmaceutically acceptable salt and/or solvate thereof and a pharmaceutically acceptable carrier.

111. The method of claim 110, wherein the pharmaceutical composition is formulated for oral administration or intravenous administration.
